SEQUENCE LISTING (350 sequences)

SEQ ID NO: 1 - protein, 153 aa, Artificial Sequence
  Description of Artificial Sequence: Synthetic polypeptide
LKPREVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSAKTTAPSVYPLAPVCGDTTGSSVTLGCLVKASQ

SEQ ID NO: 2 - protein, 111 aa, Artificial Sequence
  Description of Artificial Sequence: Synthetic polypeptide
ASDIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 3 - protein, 447 aa, Artificial Sequence
  Description of Artificial Sequence: Synthetic polypeptide
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 218 aa, Artificial Sequence
  Description of Artificial Sequence: Synthetic polypeptide
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, Artificial Sequence
  Description of Artificial Sequence: Synthetic polypeptide
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 217 aa, Artificial Sequence
  Description of Artificial Sequence: Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 7 - protein, 453 aa, Artificial Sequence
  Description of Artificial Sequence: Synthetic polypeptide
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  Description of Artificial Sequence: Synthetic polypeptide
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 657 bp, Artificial Sequence
  Description of Artificial Sequence: Synthetic polynucleotide
gatgtgctgatgacccaggcccccctgacactgcctgtgagcctgggcgaccaggcctctatcagctgcaggagctcccaggccatcgtgcacgccaacggcaatacctacctggagtggtatctgcagaagccaggacagtcccccgccctgctgatctacaaggtggccaaccggttctctggcgtgcccgacagattttccggctctggcagcggcaccgatttcacactgaagatctcccgggtggaggcagaggatctgggcgtgtactattgttttcagggagcacacgcaccatacaccttcgggggaggaactaaactggaaatcaagaggaccgtcgcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 12 - protein, 112 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIK

SEQ ID NO: 13 - protein, 11 aa, Artificial Sequence
  Description of Artificial Sequence: Synthetic peptide
QAIVHANGNTY

SEQ ID NO: 14 - protein, 9 aa, Artificial Sequence
  Description of Artificial Sequence: Synthetic peptide
FQGAHAPYT

SEQ ID NO: 15 - protein, 3 aa, Artificial Sequence
  Description of Artificial Sequence: Synthetic peptide
KVA

SEQ ID NO: 16 - protein, 107 aa, Artificial Sequence [sequence omitted]

SEQ ID NO: 17 - protein, 216 aa, Artificial Sequence
  Description of Artificial Sequence: Synthetic polypeptide
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 648 bp, Artificial Sequence
  Description of Artificial Sequence: Synthetic polynucleotide
gatattgtgctgacccagtctcctgccagcctggccgtgtccctgggccagagggccacaatctcttgcagagccagcgagtccgtggacgattacggcatctctttcatgaactggtttcagcagaagccaggccagccccctaagctgctgatctatgccgccccaaatcagggcagcggagtgccagcacggttctctggcagcggctccggcaccgacttttccctgaacatccaccccatggaggaggacgatacagccatgtacttctgtcagcagagcaaggatgtgagatggagacaccaggcaggggaccagacaggaagaaccgtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 19 - protein, 109 aa, Artificial Sequence
  Description of Artificial Sequence: Synthetic polypeptide
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 20 - protein, 10 aa, Artificial Sequence
  Description of Artificial Sequence: Synthetic peptide
ESVDDYGISF

SEQ ID NO: 21 - protein, 12 aa, Artificial Sequence
  Description of Artificial Sequence: Synthetic peptide
QQSKDVRWRHQA

SEQ ID NO: 22 - protein, 3 aa, Artificial Sequence
  Description of Artificial Sequence: Synthetic peptide
AAP

SEQ ID NO: 23 - protein, 107 aa, Artificial Sequence [sequence omitted]

SEQ ID NO: 24 - protein, 494 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIKGGGGSGGGGSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVEGGSGGSGGSGGSGGVDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 25 - DNA, 1482 bp, Artificial Sequence
  Description of Artificial Sequence: Synthetic polynucleotide
gatgtgctgatgacccaggccccactgacactgcccgtgtccctgggcgaccaggcctccatctcttgccggagctcccaggcaatcgtgcacgcaaacggcaatacctatctggagtggtacctgcagaagcctggccagtccccagccctgctgatctataaggtggccaaccggttcagcggagtgcctgaccggttcagcggctccggctctggaaccgatttcacactgaagatctccagagtggaggccgaggatctgggcgtgtactattgcttccagggagcccacgcaccatacacctttggcggaggaacaaagctggagatcaagggaggaggaggcagcggcggaggaggctccggcggcggcggctctgaggtgcagctgcagcagagcggaggagagctggccaagccaggggccagcgtgaagatgtcctgtaagtctagcggctataccttcacagcctacgccatccactgggcaaagcaggccgccggggcagggctggagtggatcggatatatcgcccccgccgccggagccgccgcctacaatgccgcctttaagggcaaggccaccctggccgccgacaagtcctctagcacagcatatatggccgccgccgccctgaccagcgaggactctgccgtgtactattgcgcaagggccgccgccgccggagccgattactggggccagggcaccacactgaccgtgtcctctggaggaggaggcagcgaggtgaagctgcaggagtccggaccaggcctggtggcccctagccagtccctgtctgtgacctgtacagtgagcggcgtgtccctgcccgattacggcgtgtcctggatcagacagccccctagaaagggcctggagtggctgggcgtgatctggggcagcgagacaacatactataactctgccctgaagagcagactgaccatcatcaaggacaacagcaagtcccaggtgtttctgaagatgaatagcctgcagaccgacgatacagccatctactattgtgccaagcactactattacggcggctcttatgccatggactattggggccagggcaccagcgtgacagtgagctccgtggagggaggctctggaggcagcggaggctccggaggctctggaggagtggacgatatccagatgacacagaccacatctagcctgtctgccagcctgggcgacagggtgaccatctcctgcagggcctctcaggatatcagcaagtatctgaattggtaccagcagaagccagacggcaccgtgaagctgctgatctaccacacatccaggctgcactctggagtgccaagccgcttctccggctctggcagcggcaccgactattccctgacaatctctaacctggagcaggaggatatcgccacctacttttgtcagcagggcaatacactgccatacaccttcgggggaggaacaaaactggaaatcacc

SEQ ID NO: 26 - protein, 112 aa, Artificial Sequence; identical to SEQ ID NO: 12
  Description of Artificial Sequence: Synthetic polypeptide

SEQ ID NO: 27 - protein, 11 aa, Artificial Sequence
  Description of Artificial Sequence: Synthetic peptide
QAIVHANGNTY

SEQ ID NO: 28 - protein, 9 aa, Artificial Sequence
  Description of Artificial Sequence: Synthetic peptide
FQGAHAPYT

SEQ ID NO: 29 - protein, 3 aa, Artificial Sequence
  Description of Artificial Sequence: Synthetic peptide
KVA

SEQ ID NO: 30 - protein, 117 aa, Artificial Sequence
  Description of Artificial Sequence: Synthetic polypeptide
EVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSS

SEQ ID NO: 31 - protein, 8 aa, Artificial Sequence
  Description of Artificial Sequence: Synthetic peptide
GYTFTAYA

SEQ ID NO: 32 - protein, 10 aa, Artificial Sequence
  Description of Artificial Sequence: Synthetic peptide
ARAAAAGADY

SEQ ID NO: 33 - protein, 8 aa, Artificial Sequence
  Description of Artificial Sequence: Synthetic peptide
IAPAAGAA

SEQ ID NO: 34 - protein, 120 aa, Artificial Sequence
  Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 35 - protein, 8 aa, Artificial Sequence
  Description of Artificial Sequence: Synthetic peptide
GVSLPDYG

SEQ ID NO: 36 - protein, 14 aa, Artificial Sequence
  Description of Artificial Sequence: Synthetic peptide
AKHYYYGGSYAMDY

SEQ ID NO: 37 - protein, 7 aa, Artificial Sequence
  Description of Artificial Sequence: Synthetic peptide
IWGSETT

SEQ ID NO: 38 - protein, 107 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 39 - protein, 6 aa, Artificial Sequence
  Description of Artificial Sequence: Synthetic peptide
QDISKY

SEQ ID NO: 40 - protein, 9 aa, Artificial Sequence
  Description of Artificial Sequence: Synthetic peptide
QQGNTLPYT

SEQ ID NO: 41 - protein, 3 aa, Artificial Sequence
  Description of Artificial Sequence: Synthetic peptide
HTS

SEQ ID NO: 42 - protein, 495 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIKGGGGSGGGGSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSVEGGSGGSGGSGGSGGVDDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIK

SEQ ID NO: 43 - DNA, 1485 bp, Artificial Sequence
  Description of Artificial Sequence: Synthetic polynucleotide
gatgtgctgatgacccaggcccccctgacactgcctgtgagcctgggcgatcaggcctctatcagctgcaggagctcccaggccatcgtgcacgccaacggcaatacctacctggagtggtatctgcagaagccaggccagtctcccgccctgctgatctacaaggtggccaacaggttctccggcgtgcctgaccgcttttccggctctggcagcggcaccgatttcacactgaagatcagccgcgtggaggcagaggacctgggcgtgtactattgcttccagggagcccacgccccatatacctttggcggcggcacaaagctggagatcaagggaggaggaggcagcggcggaggaggctccggaggcggcggctctgaggtgcagctgcagcagtccggaggagagctggccaagccaggggccagcgtgaagatgagctgtaagtctagcggctacaccttcacagcctatgccatccactgggcaaagcaggccgccggggcagggctggagtggatcggatacatcgcccccgccgccggagccgccgcctataatgccgcctttaagggcaaggccaccctggccgccgataagtcctctagcacagcatacatggccgccgccgccctgaccagcgaggatagcgccgtgtactattgcgcaagggccgccgccgccggagccgactattggggccagggcaccacactgacagtgtcctctggcggcggcggcagcgaggtgcagctggtggagtccggaggaggcctggtgcagcctggaggctccctgaggctgtcttgtgcagccagcggctacacctttagctcctattggatcgagtgggtgcgccaggcccccggcaagggcctggagtggatcggagagatcctgcctggaggaggcgatacaaactacaatgagatcttcaagggcagagccaccttttccgccgacacctctaagaacacagcctatctgcagatgaatagcctgcgggccgaggataccgccgtgtactattgcacacggagagtgccaatcagactggactactggggccagggcaccctggtgacagtgtctagcgtggagggaggctccggaggctctggaggcagcggaggctccggaggcgtggacgatatccagctgacccagagcccatcctctctgtccgcctctgtgggcgaccgggtgaccatcacctgtaaggccagccagtccgtggactacgagggcgattccttcctgaactggtatcagcagaagcctggcaaggccccaaagctgctgatctacgcagccagcaatctggagtccggagtgccatctagattctctggcagcggctccggcacagactttaccctgacaatcagctccctgcagcccgaggattttgccacctactattgtcagcagagcaacgaggaccctctgacattcggacaggggactaaggtggaaatcaag

SEQ ID NO: 44 - protein, 112 aa, Artificial Sequence; identical to SEQ ID NO: 12
  Description of Artificial Sequence: Synthetic polypeptide

SEQ ID NO: 45 - protein, 11 aa, Artificial Sequence
  Description of Artificial Sequence: Synthetic peptide
QAIVHANGNTY

SEQ ID NO: 46 - protein, 9 aa, Artificial Sequence
  Description of Artificial Sequence: Synthetic peptide
FQGAHAPYT

SEQ ID NO: 47 - protein, 3 aa, Artificial Sequence
  Description of Artificial Sequence: Synthetic peptide
KVA

SEQ ID NO: 48 - protein, 117 aa, Artificial Sequence; identical to SEQ ID NO: 30
  Description of Artificial Sequence: Synthetic polypeptide

SEQ ID NO: 49 - protein, 8 aa, Artificial Sequence
  Description of Artificial Sequence: Synthetic peptide
GYTFTAYA

SEQ ID NO: 50 - protein, 10 aa, Artificial Sequence
  Description of Artificial Sequence: Synthetic peptide
ARAAAAGADY

SEQ ID NO: 51 - protein, 8 aa, Artificial Sequence
  Description of Artificial Sequence: Synthetic peptide
IAPAAGAA

SEQ ID NO: 52 - protein, 117 aa, Artificial Sequence [sequence omitted]

SEQ ID NO: 53 - protein, 8 aa, Artificial Sequence
  Description of Artificial Sequence: Synthetic peptide
GYTFSSYW

SEQ ID NO: 54 - protein, 10 aa, Artificial Sequence
  Description of Artificial Sequence: Synthetic peptide
TRRVPIRLDY

SEQ ID NO: 55 - protein, 8 aa, Artificial Sequence
  Description of Artificial Sequence: Synthetic peptide
ILPGGGDT

SEQ ID NO: 56 - protein, 111 aa, Artificial Sequence [sequence omitted]

SEQ ID NO: 57 - protein, 10 aa, Artificial Sequence
  Description of Artificial Sequence: Synthetic peptide
QSVDYEGDSF

SEQ ID NO: 58 - protein, 9 aa, Artificial Sequence
  Description of Artificial Sequence: Synthetic peptide
QQSNEDPLT

SEQ ID NO: 59 - protein, 3 aa, Artificial Sequence
  Description of Artificial Sequence: Synthetic peptide
AAS

SEQ ID NO: 60 - protein, 500 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIKGGGGSGGGGSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSGGGGSEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSVEGGSGGSGGSGGSGGVDQSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVL

SEQ ID NO: 61 - DNA, 1500 bp, Artificial Sequence
  Description of Artificial Sequence: Synthetic polynucleotide
gatgtgctgatgacccaggccccactgacactgcccgtgtccctgggcgaccaggcctctatcagctgcaggagctcccaggccatcgtgcacgccaacggcaatacctacctggagtggtatctgcagaagcctggccagagcccagccctgctgatctacaaggtggccaacaggttctccggagtgccagaccgcttttccggctctggcagcggcaccgatttcacactgaagatctcccgcgtggaggcagaggatctgggcgtgtactattgcttccagggagcccacgccccttatacctttggcggcggcacaaagctggagatcaagggcggcggcggctctggaggaggaggcagcggcggaggaggctccgaggtgcagctgcagcagagcggcggcgagctggccaagccaggggccagcgtgaagatgtcctgtaagtctagcggctacaccttcacagcctatgccatccactgggcaaagcaggccgccggggcagggctggagtggatcggatacatcgcccccgccgccggagccgccgcctataatgccgcctttaagggcaaggccaccctggccgccgacaagtcctctagcacagcatacatggccgccgccgccctgaccagcgaggactccgccgtgtactattgcgcaagggccgccgccgccggagccgattattggggccagggcaccacactgacagtgtcctctggaggaggaggctctgaggtgcagctggtggagagcggaggaggcctggtgaagcctggaggctctctgagactgagctgtgccgcctccggcttcacctttggcgactacgccctgtcctggttcaggcaggccccaggcaagggcctggagtgggtgggcgtgtcccgctctaaggcatacggaggcaccacagattatgccgcctccgtgaagggccggtttacaatctctagagacgatagcaagtccaccgcctacctgcagatgaacagcctgaagaccgaggacacagccgtgtactattgcgccagctccggctactctagcggctggacaccttttgattactggggacagggcaccctggtgacagtgtcctctgtggagggaggctctggaggcagcggaggctccggcggctctggaggagtggaccagtccgtgctgacccagccaccttctgccagcggaaccccaggccagcgggtgacaatctcctgttctggcagctcctctaacatcggctctaacacagtgaattggtaccagcagctgccaggaaccgcccctaagctgctgatcttcaattatcaccagcggccaagcggagtgccagatcggttcagcggctccaagtctggcagctccgcctctctggccatcagcggcctgcagtccgaggacgaggcagattactattgtgccgcctgggacgatagcctgaatgggtgggtcttcgggggagggacaaaactgactgtgctg

SEQ ID NO: 62 - protein, 112 aa, Artificial Sequence; identical to SEQ ID NO: 12
  Description of Artificial Sequence: Synthetic polypeptide

SEQ ID NO: 63 - protein, 11 aa, Artificial Sequence
  Description of Artificial Sequence: Synthetic peptide
QAIVHANGNTY

SEQ ID NO: 64 - protein, 9 aa, Artificial Sequence
  Description of Artificial Sequence: Synthetic peptide
FQGAHAPYT

SEQ ID NO: 65 - protein, 3 aa, Artificial Sequence
  Description of Artificial Sequence: Synthetic peptide
KVA

SEQ ID NO: 66 - protein, 117 aa, Artificial Sequence; identical to SEQ ID NO: 30
  Description of Artificial Sequence: Synthetic polypeptide

SEQ ID NO: 67 - protein, 8 aa, Artificial Sequence
  Description of Artificial Sequence: Synthetic peptide
GYTFTAYA

SEQ ID NO: 68 - protein, 10 aa, Artificial Sequence
  Description of Artificial Sequence: Synthetic peptide
ARAAAAGADY

SEQ ID NO: 69 - protein, 8 aa, Artificial Sequence
  Description of Artificial Sequence: Synthetic peptide
IAPAAGAA

SEQ ID NO: 70 - protein, 123 aa, Artificial Sequence
  Description of Artificial Sequence: Synthetic polypeptide
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 71 - protein, 8 aa, Artificial Sequence
  Description of Artificial Sequence: Synthetic peptide
GFTFGDYA

SEQ ID NO: 72 - protein, 14 aa, Artificial Sequence
  Description of Artificial Sequence: Synthetic peptide
ASSGYSSGWTPFDY

SEQ ID NO: 73 - protein, 10 aa, Artificial Sequence
  Description of Artificial Sequence: Synthetic peptide
SRSKAYGGTT

SEQ ID NO: 74 - protein, 110 aa, Artificial Sequence
  Description of Artificial Sequence: Synthetic polypeptide
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVL

SEQ ID NO: 75 - protein, 8 aa, Artificial Sequence
  Description of Artificial Sequence: Synthetic peptide
SSNIGSNT

SEQ ID NO: 76 - protein, 11 aa, Artificial Sequence
  Description of Artificial Sequence: Synthetic peptide
AAWDDSLNGWV

SEQ ID NO: 77 - protein, 3 aa, Artificial Sequence
  Description of Artificial Sequence: Synthetic peptide
NYH

SEQ ID NO: 78 - protein, 498 aa, Artificial Sequence
  Description of Artificial Sequence: Synthetic polypeptide
DVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIKGGGGSGGGGSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSVEGGSGGSGGSGGSGGVDDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 79 - DNA, 1494 bp, Artificial Sequence
  Description of Artificial Sequence: Synthetic polynucleotide
gatgtcctgatgacccaggcccccctgacactgcctgtgagcctgggcgaccaggcctctatcagctgcaggagctcccaggccatcgtgcacgccaacggcaatacctacctggagtggtatctgcagaagccaggacagtcccccgccctgctgatctacaaggtggccaacaggttctctggagtgccagaccgcttttccggctctggcagcggcaccgatttcacactgaagatcagccgcgtggaggcagaggatctgggcgtgtactattgcttccagggagcccacgcaccttacacctttggcggaggaacaaagctggagatcaagggcggcggcggctctggaggaggaggcagcggcggaggaggctccgaggtgcagctgcagcagtccggcggcgagctggccaagccaggggccagcgtgaagatgtcctgtaagtctagcggctacaccttcacagcctatgccatccactgggcaaagcaggccgccggggcagggctggagtggatcggatacatcgcccccgccgccggagccgccgcctataatgccgcctttaagggcaaggccaccctggccgccgacaagtcctctagcacagcatacatggccgccgccgccctgaccagcgaggactctgccgtgtactattgcgcaagagccgccgccgccggagccgattattggggacagggcaccacactgaccgtgtcctctggaggaggaggctctcaggtggagctggtgcagagcggagccgaggtgaagaagcctggcgagtctctgaagatcagctgtaagggcagcggctactccttcacatcttattggatcggatgggtgcggcaggccccaggcaagggcctggagtggatgggcatcatcgacccaggcgatagccggaccagatactccccctcttttcagggccaggtgacaatctccgccgacaagagcatctccaccgcctatctgcagtggagctccctgaaggccagcgatacagccatgtactattgcgccagaggccagctgtacggaggaacctatatggacggatggggacagggcaccctggtgacagtgtctagcgtggagggaggcagcggaggctccggaggctctggaggcagcggaggagtggacgatatcgccctgacacagcccgcctctgtgagcggctcccctggacagtccatcaccatctcttgtaccggcacatcctctgatatcggcggctacaactctgtgagctggtatcagcagcaccctggcaaggccccaaagctgatgatctacggcgtgaacaatcggccttccggcgtgtctaacagattttccggctctaagagcggcaataccgccagcctgacaatctccggcctgcaggcagaggacgaggcagattactattgtagctcctatgatatcgagtccgccactcctgtctttggcgggggcactaaactgactgtcctg

SEQ ID NO: 80 - protein, 112 aa, Artificial Sequence; identical to SEQ ID NO: 12
  Description of Artificial Sequence: Synthetic polypeptide

SEQ ID NO: 81 - protein, 11 aa, Artificial Sequence
  Description of Artificial Sequence: Synthetic peptide
QAIVHANGNTY

SEQ ID NO: 82 - protein, 9 aa, Artificial Sequence
  Description of Artificial Sequence: Synthetic peptide
FQGAHAPYT

SEQ ID NO: 83 - protein, 3 aa, Artificial Sequence
  Description of Artificial Sequence: Synthetic peptide
KVA

SEQ ID NO: 84 - protein, 117 aa, Artificial Sequence; identical to SEQ ID NO: 30
  Description of Artificial Sequence: Synthetic polypeptide

SEQ ID NO: 85 - protein, 8 aa, Artificial Sequence
  Description of Artificial Sequence: Synthetic peptide
GYTFTAYA

SEQ ID NO: 86 - protein, 10 aa, Artificial Sequence
  Description of Artificial Sequence: Synthetic peptide
ARAAAAGADY

SEQ ID NO: 87 - protein, 8 aa, Artificial Sequence
  Description of Artificial Sequence: Synthetic peptide
IAPAAGAA

SEQ ID NO: 88 - protein, 120 aa, Artificial Sequence
  Description of Artificial Sequence: Synthetic polypeptide
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 89 - protein, 8 aa, Artificial Sequence
  Description of Artificial Sequence: Synthetic peptide
GYSFTSYW

SEQ ID NO: 90 - protein, 13 aa, Artificial Sequence
  Description of Artificial Sequence: Synthetic peptide
ARGQLYGGTYMDG

SEQ ID NO: 91 - protein, 8 aa, Artificial Sequence
  Description of Artificial Sequence: Synthetic peptide
IDPGDSRT

SEQ ID NO: 92 - protein, 111 aa, Artificial Sequence
  Description of Artificial Sequence: Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 93 - protein, 9 aa, Artificial Sequence
  Description of Artificial Sequence: Synthetic peptide
SSDIGGYNS

SEQ ID NO: 94 - protein, 11 aa, Artificial Sequence
  Description of Artificial Sequence: Synthetic peptide
SSYDIESATPV

SEQ ID NO: 95 - protein, 3 aa, Artificial Sequence
  Description of Artificial Sequence: Synthetic peptide
GVN

SEQ ID NO: 96 - protein, 491 aa, Artificial Sequence
  Description of Artificial Sequence: Synthetic polypeptide
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTGGGGGSGGGGSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSVEGGSGGSGGSGGSGGVDDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIK

SEQ ID NO: 97 - DNA, 1473 bp, Artificial Sequence
  Description of Artificial Sequence: Synthetic polynucleotide
gatattgtgctgacccagagccccgcctccctggccgtgtctctgggccagagggcaacaatcagctgcagggccagcgagtccgtggacgattacggcatcagcttcatgaactggtttcagcagaagcctggccagccccctaagctgctgatctatgccgcccctaatcagggcagcggagtgccagccaggttctctggcagcggctccggaaccgatttttccctgaacatccaccctatggaggaggacgatacagccatgtacttctgccagcagagcaaggacgtgcggtggagacaccaggccggggaccagaccggaggaggaggaggctccggaggaggaggctctggcggcggcggcagcgaggtgaagctggtggagtccggaggaggcctggtgcagccaggaggcagcctgaagctgtcctgtgcagcctctggcttcgatttttcccggtattggatgtcttgggtgagacaggccccaggcaagggcctggagtggatcggcgagatcaacctggacagctccaccatcaattacacaccctccctgaaggacaagttcatcatctctagggataacgccaagaataccctgtatctgcagatgagcaaggtgcgctccgaggacacagccctgtactattgcgcccggagatacgacgccatggattattggggccagggcaccagcgtgacagtgtcttccggaggaggcggcagcgaggtgcagctggtcgaaagcggcggcggcctggtccagccaggaggctctctgaggctgagctgtgccgcctccggctacaccttttcctcttattggatcgagtgggtgcgccaggcccccggcaagggcctggaatggatcggagagatcctgcctggaggaggcgataccaactacaatgagatcttcaagggcagagccacattttctgccgacaccagcaagaacacagcctatctgcagatgaacagcctgcgggccgaggataccgccgtgtactattgcacaaggcgcgtgccaatcagactggactactggggccagggcaccctggtgacagtgagctccgtggagggaggctctggaggcagcggaggctccggaggctctggaggagtggacgatatccagctgacccagtctccctctagcctgtctgccagcgtgggcgatcgggtgaccatcacctgtaaggcctcccagtctgtggactacgagggcgattccttcctgaactggtatcagcagaagccaggcaaggcccccaagctgctgatctacgccgcctccaatctggagtctggcgtgcctagcagattcagcggctccggctctggcaccgactttaccctgacaatctcctctctgcagccagaggattttgccacatactattgtcagcagagcaatgaggaccctctgacattcggacagggaactaaggtggaaatcaaa

SEQ ID NO: 98 - protein, 109 aa, Artificial Sequence; identical to SEQ ID NO: 19
  Description of Artificial Sequence: Synthetic polypeptide

SEQ ID NO: 99 - protein, 10 aa, Artificial Sequence
  Description of Artificial Sequence: Synthetic peptide
ESVDDYGISF

SEQ ID NO: 100 - protein, 12 aa, Artificial Sequence
  Description of Artificial Sequence: Synthetic peptide
QQSKDVRWRHQA

SEQ ID NO: 101 - protein, 3 aa, Artificial Sequence
  Description of Artificial Sequence: Synthetic peptide
AAP

SEQ ID NO: 102 - protein, 116 aa, Artificial Sequence
  Description of Artificial Sequence: Synthetic polypeptide
EVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSS

SEQ ID NO: 103 - protein, 8 aa, Artificial Sequence
  Description of Artificial Sequence: Synthetic peptide
GFDFSRYW

SEQ ID NO: 104 - protein, 9 aa, Artificial Sequence
  Description of Artificial Sequence: Synthetic peptide
ARRYDAMDY

SEQ ID NO: 105 - protein, 8 aa, Artificial Sequence
  Description of Artificial Sequence: Synthetic peptide
INLDSSTI

SEQ ID NO: 106 - protein, 117 aa, Artificial Sequence [sequence omitted]

SEQ ID NO: 107 - protein, 8 aa, Artificial Sequence
  Description of Artificial Sequence: Synthetic peptide
GYTFSSYW

SEQ ID NO: 108 - protein, 10 aa, Artificial Sequence
  Description of Artificial Sequence: Synthetic peptide
TRRVPIRLDY

SEQ ID NO: 109 - protein, 8 aa, Artificial Sequence
  Description of Artificial Sequence: Synthetic peptide
ILPGGGDT

SEQ ID NO: 110 - protein, 111 aa, Artificial Sequence [sequence omitted]

SEQ ID NO: 111 - protein, 10 aa, Artificial Sequence
  Description of Artificial Sequence: Synthetic peptide
QSVDYEGDSF

SEQ ID NO: 112 - protein, 9 aa, Artificial Sequence
  Description of Artificial Sequence: Synthetic peptide
QQSNEDPLT

SEQ ID NO: 113 - protein, 3 aa, Artificial Sequence
  Description of Artificial Sequence: Synthetic peptide
AAS

SEQ ID NO: 114 - protein, 496 aa, Artificial Sequence
  Description of Artificial Sequence: Synthetic polypeptide
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTGGGGGSGGGGSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSGGGGSEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSVEGGSGGSGGSGGSGGVDQSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVL

SEQ ID NO: 115 - DNA, 1488 bp, Artificial Sequence
  Description of Artificial Sequence: Synthetic polynucleotide
gatattgtgctgacccagtccccagcctctctggccgtgtccctgggccagagggccacaatctcttgccgcgccagcgagtccgtggacgattacggcatcagcttcatgaactggtttcagcagaagcccggccagccccctaagctgctgatctatgccgccccaaatcagggctccggagtgcccgcccggttctctggcagcggctccggcaccgacttttctctgaacatccaccccatggaggaggacgatacagccatgtacttctgccagcagtccaaggacgtgaggtggcggcaccaggccggggaccagaccggaggaggaggaggcagcggaggaggaggctccggcggcggcggctctgaggtgaagctggtggagagcggaggaggcctggtgcagcctggaggctccctgaagctgtcttgtgccgccagcggcttcgactttagccggtactggatgtcctgggtgagacaggcccctggcaagggcctggagtggatcggcgagatcaacctggatagctccaccatcaattacacaccaagcctgaaggacaagtttatcatctccagggataacgccaagaataccctgtatctgcagatgtccaaggtgcgctctgaggatacagccctgtactattgcgcccggagatacgacgccatggattattggggccagggcacctccgtgacagtgtctagcggaggaggaggctctgaggtgcagctggtcgaatccggcggaggcctggtgaagccaggaggcagcctgcggctgtcctgtgccgcctctggcttcacctttggcgactacgccctgagctggttcaggcaggcccctggcaagggcctggaatgggtgggcgtgtctagaagcaaggcctacggcggcaccacagattatgccgcctctgtgaagggccggtttaccatcagcagagacgattccaagtctacagcctatctgcagatgaactccctgaagaccgaggacacagccgtgtactattgcgcctcctctggctacagctccggctggacccctttcgattactggggacagggcaccctggtgacagtgtctagcgtggagggaggcagcggaggctccggaggctctggcggcagcggaggagtggaccagagcgtgctgacacagccaccaagcgcctccggaaccccaggacagagggtgacaatctcttgtagcggctcctctagcaacatcggctccaacaccgtgaattggtaccagcagctgcctggcacagccccaaagctgctgatcttcaattatcaccagaggcccagcggagtgcctgatcgcttttccggctctaagagcggctcctctgccagcctggccatctccggcctgcagtctgaggacgaggccgattactattgtgccgcctgggacgatagcctgaatggctgggtctttgggggggggactaaactgactgtgctg

SEQ ID NO: 116 - protein, 109 aa, Artificial Sequence; identical to SEQ ID NO: 19
  Description of Artificial Sequence: Synthetic polypeptide

SEQ ID NO: 117 - protein, 10 aa, Artificial Sequence
  Description of Artificial Sequence: Synthetic peptide
ESVDDYGISF

SEQ ID NO: 118 - protein, 12 aa, Artificial Sequence
  Description of Artificial Sequence: Synthetic peptide
QQSKDVRWRHQA

SEQ ID NO: 119 - protein, 3 aa, Artificial Sequence
  Description of Artificial Sequence: Synthetic peptide
AAP

SEQ ID NO: 120 - protein, 116 aa, Artificial Sequence; identical to SEQ ID NO: 102
  Description of Artificial Sequence: Synthetic polypeptide

SEQ ID NO: 121 - protein, 8 aa, Artificial Sequence
  Description of Artificial Sequence: Synthetic peptide
GFDFSRYW

SEQ ID NO: 122 - protein, 9 aa, Artificial Sequence
  Description of Artificial Sequence: Synthetic peptide
ARRYDAMDY

SEQ ID NO: 123 - protein, 8 aa, Artificial Sequence
  Description of Artificial Sequence: Synthetic peptide
INLDSSTI

SEQ ID NO: 124 - protein, 123 aa, Artificial Sequence; identical to SEQ ID NO: 70
  Description of Artificial Sequence: Synthetic polypeptide

SEQ ID NO: 125 - protein, 8 aa, Artificial Sequence
  Description of Artificial Sequence: Synthetic peptide
GFTFGDYA

SEQ ID NO: 126 - protein, 14 aa, Artificial Sequence
  Description of Artificial Sequence: Synthetic peptide
ASSGYSSGWTPFDY

SEQ ID NO: 127 - protein, 10 aa, Artificial Sequence
  Description of Artificial Sequence: Synthetic peptide
SRSKAYGGTT

SEQ ID NO: 128 - protein, 110 aa, Artificial Sequence; identical to SEQ ID NO: 74
  Description of Artificial Sequence: Synthetic polypeptide

SEQ ID NO: 129 - protein, 8 aa, Artificial Sequence
  Description of Artificial Sequence: Synthetic peptide
SSNIGSNT

SEQ ID NO: 130 - protein, 11 aa, Artificial Sequence
  Description of Artificial Sequence: Synthetic peptide
AAWDDSLNGWV

SEQ ID NO: 131-133 - skipped (3 entries)

SEQ ID NO: 134 - protein, 3 aa, Artificial Sequence
  Description of Artificial Sequence: Synthetic peptide
NYH

SEQ ID NO: 135 - protein, 494 aa, Artificial Sequence
  Description of Artificial Sequence: Synthetic polypeptide
DIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTGGGGGSGGGGSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSVEGGSGGSGGSGGSGGVDDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 136 - DNA, 1482 bp, Artificial Sequence
  Description of Artificial Sequence: Synthetic polynucleotide
gacattgtgctgacccagtctccagccagcctggccgtgtccctgggccagagggccacaatctcttgccgcgccagcgagtccgtggacgattacggcatcagcttcatgaactggtttcagcagaagcccggccagccccctaagctgctgatctatgccgcccctaatcagggcagcggagtgccagcccggttctctggcagcggctccggcaccgacttttccctgaacatccaccctatggaggaggacgatacagccatgtacttctgccagcagagcaaggacgtgaggtggcggcaccaggccggggaccagaccggaggaggaggaggcagcggaggaggaggctccggcggcggcggctctgaggtgaagctggtggagtccggaggaggcctggtgcagccaggaggctccctgaagctgtcttgtgccgccagcggcttcgactttagccggtactggatgtcctgggtgagacaggcccctggcaagggcctggagtggatcggcgagatcaacctggatagctccaccatcaattacacaccaagcctgaaggacaagtttatcatctcccgggataacgccaagaataccctgtatctgcagatgtccaaggtgagatctgaggatacagccctgtactattgcgcccggagatacgacgccatggattattggggccagggcaccagcgtgacagtgtctagcggaggaggaggctctcaggtggagctggtgcagagcggagccgaggtgaagaagcccggcgagagcctgaagatctcctgtaagggctccggctactctttcaccagctattggatcggatgggtgaggcaggcccctggcaagggcctggaatggatgggcatcatcgacccaggcgattctcggaccagatactctcccagctttcagggccaggtgaccatctccgccgacaagtccatctctacagcctatctgcagtggtcctctctgaaggcctccgataccgccatgtactattgcgccagaggccagctgtacggcggcacatatatggacggatggggacagggcaccctggtgacagtgagctccgtggagggaggctccggaggctctggaggcagcggcggctccggaggagtggacgatatcgccctgacccagcccgccagcgtgtccggctctcctggccagtctatcacaatcagctgtaccggcacatctagcgatatcggcggctacaatagcgtgtcctggtatcagcagcacccaggcaaggcccccaagctgatgatctacggcgtgaacaataggccctctggcgtgagcaaccgcttctctggcagcaagtccggcaataccgcctccctgacaatctctggcctgcaggcagaggacgaggcagattactattgttcctcttatgacatcgagagcgccacacccgtcttcggaggaggaaccaaactgaccgtgctg

SEQ ID NO: 137 - protein, 109 aa, Artificial Sequence; identical to SEQ ID NO: 19
  Description of Artificial Sequence: Synthetic polypeptide

SEQ ID NO: 138 - protein, 10 aa, Artificial Sequence
  Description of Artificial Sequence: Synthetic peptide
ESVDDYGISF

SEQ ID NO: 139 - protein, 12 aa, Artificial Sequence
  Description of Artificial Sequence: Synthetic peptide
QQSKDVRWRHQA

SEQ ID NO: 140 - protein, 3 aa, Artificial Sequence
  Description of Artificial Sequence: Synthetic peptide
AAP

SEQ ID NO: 141 - protein, 116 aa, Artificial Sequence; identical to SEQ ID NO: 102
  Description of Artificial Sequence: Synthetic polypeptide

SEQ ID NO: 142 - protein, 8 aa, Artificial Sequence
  Description of Artificial Sequence: Synthetic peptide
GFDFSRYW

SEQ ID NO: 143 - protein, 9 aa, Artificial Sequence
  Description of Artificial Sequence: Synthetic peptide
ARRYDAMDY

SEQ ID NO: 144 - protein, 8 aa, Artificial Sequence
  Description of Artificial Sequence: Synthetic peptide
INLDSSTI

SEQ ID NO: 145 - protein, 120 aa, Artificial Sequence; identical to SEQ ID NO: 88
  Description of Artificial Sequence: Synthetic polypeptide

SEQ ID NO: 146 - protein, 8 aa, Artificial Sequence
  Description of Artificial Sequence: Synthetic peptide
GYSFTSYW

SEQ ID NO: 147 - protein, 13 aa, Artificial Sequence
  Description of Artificial Sequence: Synthetic peptide
ARGQLYGGTYMDG

SEQ ID NO: 148 - protein, 8 aa, Artificial Sequence
  Description of Artificial Sequence: Synthetic peptide
IDPGDSRT

SEQ ID NO: 149 - protein, 111 aa, Artificial Sequence; identical to SEQ ID NO: 92
  Description of Artificial Sequence: Synthetic polypeptide

SEQ ID NO: 150 - protein, 9 aa, Artificial Sequence
  Description of Artificial Sequence: Synthetic peptide
SSDIGGYNS

SEQ ID NO: 151 - protein, 11 aa, Artificial Sequence
  Description of Artificial Sequence: Synthetic peptide
SSYDIESATPV

SEQ ID NO: 152 - protein, 3 aa, Artificial Sequence
  Description of Artificial Sequence: Synthetic peptide
GVN

SEQ ID NO: 153 - protein, 494 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSVEGGSGGSGGSGGSGGVDDVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIK

SEQ ID NO: 154 - DNA, 1482 bp, Artificial Sequence
  Description of Artificial Sequence: Synthetic polynucleotide
gatattcagatgacacagaccacaagctccctgtccgcctctctgggcgacagggtgaccatcagctgcagggcctcccaggatatctctaagtatctgaactggtaccagcagaagccagacggcaccgtgaagctgctgatctatcacacaagcaggctgcactccggagtgccatctcgcttcagcggctccggctctggaaccgactacagcctgacaatctccaacctggagcaggaggatatcgccacctatttctgccagcagggcaataccctgccctacacatttggcggcggcaccaagctggagatcacaggaggaggaggcagcggcggaggaggctccggcggcggcggctctgaggtgaagctgcaggagtccggaccaggcctggtggcccctagccagtccctgtctgtgacctgtacagtgtccggcgtgtctctgcctgattacggcgtgtcctggatcagacagccccctagaaagggcctggagtggctgggcgtgatctggggcagcgagacaacatactataactctgccctgaagagcaggctgaccatcatcaaggacaacagcaagtcccaggtgtttctgaagatgaatagcctgcagaccgacgatacagccatctactattgcgccaagcactactattacggcggctcttatgccatggattactggggccagggcaccagcgtgacagtgtctagcggaggaggaggcagcgaggtgcagctgcagcagtccggcggcgagctggccaagcctggggccagcgtgaagatgtcttgtaagtcctctggctataccttcacagcctacgccatccactgggcaaagcaggccgccggggcagggctggagtggatcggatatatcgcccccgccgccggagccgccgcctacaatgccgcctttaagggcaaggccaccctggccgccgacaagagctcctctacagcatatatggccgccgccgccctgaccagcgaggactccgccgtgtattactgcgcaagggccgccgccgccggagccgactattggggccagggcaccacactgacagtgagctccgtggagggaggctctggaggcagcggaggctccggcggctctggcggcgtggacgatgtgctgatgacccaggccccactgacactgcccgtgtccctgggcgaccaggcctctatcagctgtcggtctagccaggccatcgtgcacgccaacggcaatacctatctggagtggtacctgcagaagcctggccagtccccagccctgctgatctacaaggtggccaatcggttcagcggcgtgcccgacagattttccggctctggcagcggcaccgatttcacactgaagatcagcagagtggaggccgaggatctgggcgtgtattactgttttcagggagcccacgccccctacaccttcgggggaggaactaaactggaaatcaag

SEQ ID NO: 155 - protein, 107 aa, Artificial Sequence; identical to SEQ ID NO: 38
  Description of Artificial Sequence: Synthetic polypeptide

SEQ ID NO: 156 - protein, 6 aa, Artificial Sequence
  Description of Artificial Sequence: Synthetic peptide
QDISKY

SEQ ID NO: 157 - protein, 9 aa, Artificial Sequence
  Description of Artificial Sequence: Synthetic peptide
QQGNTLPYT

SEQ ID NO: 158 - protein, 3 aa, Artificial Sequence
  Description of Artificial Sequence: Synthetic peptide
HTS

SEQ ID NO: 159 - protein, 120 aa, Artificial Sequence; identical to SEQ ID NO: 34
  Description of Artificial Sequence: Synthetic polypeptide

SEQ ID NO: 160 - protein, 8 aa, Artificial Sequence
  Description of Artificial Sequence: Synthetic peptide
GVSLPDYG

SEQ ID NO: 161 - protein, 14 aa, Artificial Sequence
  Description of Artificial Sequence: Synthetic peptide
AKHYYYGGSYAMDY

SEQ ID NO: 162 - protein, 7 aa, Artificial Sequence
  Description of Artificial Sequence: Synthetic peptide
IWGSETT

SEQ ID NO: 163 - protein, 117 aa, Artificial Sequence; identical to SEQ ID NO: 30
  Description of Artificial Sequence: Synthetic polypeptide

SEQ ID NO: 164 - protein, 8 aa, Artificial Sequence
  Description of Artificial Sequence: Synthetic peptide
GYTFTAYA

SEQ ID NO: 165 - protein, 10 aa, Artificial Sequence
  Description of Artificial Sequence: Synthetic peptide
ARAAAAGADY

SEQ ID NO: 166 - protein, 8 aa, Artificial Sequence
  Description of Artificial Sequence: Synthetic peptide
IAPAAGAA

SEQ ID NO: 167 - protein, 112 aa, Artificial Sequence; identical to SEQ ID NO: 12
  Description of Artificial Sequence: Synthetic polypeptide

SEQ ID NO: 168 - protein, 11 aa, Artificial Sequence
  Description of Artificial Sequence: Synthetic peptide
QAIVHANGNTY

SEQ ID NO: 169 - protein, 9 aa, Artificial Sequence
  Description of Artificial Sequence: Synthetic peptide
FQGAHAPYT

SEQ ID NO: 170 - protein, 3 aa, Artificial Sequence
  Description of Artificial Sequence: Synthetic peptide
KVA

SEQ ID NO: 171 - protein, 495 aa, Artificial Sequence
  Description of Artificial Sequence: Synthetic polypeptide
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSVEGGSGGSGGSGGSGGVDDVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIK

SEQ ID NO: 172 - DNA, 1485 bp, Artificial Sequence
  Description of Artificial Sequence: Synthetic polynucleotide
gatattcagctgacccagagcccaagctccctgtctgccagcgtgggcgatcgggtgaccatcacatgcaaggcctcccagtctgtggactacgagggcgattccttcctgaactggtatcagcagaagcccggcaaggcccctaagctgctgatctacgccgcctctaatctggagagcggcgtgccttccagattcagcggctccggctctggcacagactttaccctgacaatctctagcctgcagccagaggatttcgccacctactattgccagcagagcaacgaggaccccctgacctttggccagggcacaaaggtggagatcaagggaggaggaggcagcggcggaggaggctccggcggcggcggctctgaggtgcagctggtggagtccggaggaggcctggtgcagcctggaggctctctgaggctgagctgtgcagcctccggctacaccttttcctcttattggatcgagtgggtgcgccaggcccccggcaagggcctggagtggatcggagagatcctgcctggaggaggcgatacaaactacaatgagatcttcaagggccgggccaccttttctgccgacaccagcaagaacacagcctatctgcagatgaatagcctgcgggccgaggataccgccgtgtactattgcacacggagagtgcctatcagactggactactggggccagggcaccctggtgacagtgagctccggaggaggaggcagcgaggtgcagctgcagcagtccggcggcgagctggccaagccaggggccagcgtgaagatgtcttgtaagtctagcggctacaccttcacagcctatgccatccactgggcaaagcaggccgccggggcagggctggagtggatcggatacatcgcccccgccgccggagccgccgcctataacgccgcctttaagggcaaggccaccctggccgccgacaagtcctctagcacagcatacatggccgccgccgccctgaccagcgaggatagcgccgtgtactattgcgcaagggccgccgccgccggagccgactattggggccagggcaccacactgacagtgtcctctgtggagggaggctccggaggctctggaggcagcggaggctccggaggcgtggacgatgtgctgatgacccaggccccactgacactgcccgtgagcctgggcgatcaggccagcatctcctgtaggagctcccaggccatcgtgcacgccaacggcaatacctacctggagtggtatctgcagaagcctggccagtctccagccctgctgatctacaaggtggccaataggttctccggagtgccagaccgcttttctggcagcggctccggcaccgatttcacactgaagatcagccgcgtggaggcagaggacctgggcgtgtactattgttttcagggagcccacgccccctacacctttgggggaggaactaaactggaaatcaag

SEQ ID NO: 173 - protein, 111 aa, Artificial Sequence [sequence omitted]

SEQ ID NO: 174 - protein, 10 aa, Artificial Sequence
  Description of Artificial Sequence: Synthetic peptide
QSVDYEGDSF

SEQ ID NO: 175 - protein, 9 aa, Artificial Sequence
  Description of Artificial Sequence: Synthetic peptide
QQSNEDPLT

SEQ ID NO: 176 - protein, 3 aa, Artificial Sequence
  Description of Artificial Sequence: Synthetic peptide
AAS

SEQ ID NO: 177 - protein, 117 aa, Artificial Sequence [sequence omitted]

SEQ ID NO: 178 - protein, 8 aa, Artificial Sequence
  Description of Artificial Sequence: Synthetic peptide
GYTFSSYW

SEQ ID NO: 179 - protein, 10 aa, Artificial Sequence
  Description of Artificial Sequence: Synthetic peptide
TRRVPIRLDY

SEQ ID NO: 180 - protein, 8 aa, Artificial Sequence
  Description of Artificial Sequence: Synthetic peptide
ILPGGGDT

SEQ ID NO: 181 - protein, 117 aa, Artificial Sequence; identical to SEQ ID NO: 30
  Description of Artificial Sequence: Synthetic polypeptide

SEQ ID NO: 182 - protein, 8 aa, Artificial Sequence
  Description of Artificial Sequence: Synthetic peptide
GYTFTAYA

SEQ ID NO: 183 - protein, 10 aa, Artificial Sequence
  Description of Artificial Sequence: Synthetic peptide
ARAAAAGADY

SEQ ID NO: 184 - protein, 8 aa, Artificial Sequence
  Description of Artificial Sequence: Synthetic peptide
IAPAAGAA

SEQ ID NO: 185 - protein, 112 aa, Artificial Sequence; identical to SEQ ID NO: 12
  Description of Artificial Sequence: Synthetic polypeptide

SEQ ID NO: 186 - protein, 11 aa, Artificial Sequence
  Description of Artificial Sequence: Synthetic peptide
QAIVHANGNTY

SEQ ID NO: 187 - protein, 9 aa, Artificial Sequence
  Description of Artificial Sequence: Synthetic peptide
FQGAHAPYT

SEQ ID NO: 188 - protein, 3 aa, Artificial Sequence
  Description of Artificial Sequence: Synthetic peptide
KVA

SEQ ID NO: 189 - protein, 500 aa, Artificial Sequence
  Description of Artificial Sequence: Synthetic polypeptide
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGGGGSGGGGSGGGGSEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSVEGGSGGSGGSGGSGGVDDVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIK

SEQ ID NO: 190 - DNA, 1500 bp, Artificial Sequence
  Description of Artificial Sequence: Synthetic polynucleotide
cagagtgtgctgacccagccaccttctgccagcggaacccctggacagagggtgacaatctcctgctctggcagctcctctaacatcggctctaacacagtgaattggtaccagcagctgccaggaaccgcccccaagctgctgatcttcaattatcaccagaggcctagcggagtgccagaccgctttagcggctccaagtctggcagctccgccagcctggccatctccggcctgcagtctgaggacgaggccgattactattgcgccgcctgggacgattccctgaacggatgggtgttcggaggaggaaccaagctgacagtgctgggcggcggcggctctggaggaggaggcagcggcggaggaggctccgaggtgcagctggtggagtccggcggcggcctggtgaagcctggaggcagcctgcgcctgtcctgtgcagcctctggcttcacatttggcgactacgccctgagctggttcaggcaggccccaggcaagggcctggagtgggtgggcgtgagccgctccaaggcatacggaggaaccacagattatgccgcctccgtgaagggccggtttaccatctctagagacgattctaagagcacagcctacctgcagatgaacagcctgaagaccgaggacacagccgtgtactattgcgcctctagcggctactcctctggctggaccccctttgattattggggccagggcaccctggtgacagtgagctccggaggaggaggctctgaggtgcagctgcagcagagcggaggagagctggccaagcctggggccagcgtgaagatgtcctgtaagtctagcggctacaccttcacagcctatgccatccactgggcaaagcaggccgccggggcagggctggagtggatcggatacatcgcccccgccgccggagccgccgcctataatgccgcctttaagggcaaggccaccctggccgccgataagtcctctagcacagcatacatggccgccgccgccctgaccagcgaggactccgccgtgtactattgcgcaagggccgccgccgccggagccgactactggggccagggcaccacactgacagtgtcctctgtggagggaggctctggaggcagcggaggctccggcggctctggcggcgtggacgatgtgctgatgacccaggcccccctgacactgcccgtgagcctgggcgaccaggcctccatctcttgtcggagctcccaggccatcgtgcacgccaacggcaatacctacctggagtggtatctgcagaagccaggacagagccccgccctgctgatctacaaggtggccaatcggttctccggagtgccagaccggttcagcggctccggctctggcaccgatttcacactgaagatcagcagagtggaggccgaggatctgggcgtgtactattgttttcagggagcccacgccccatacaccttcgggggcgggaccaaactggaaatcaag

SEQ ID NO: 191 - protein, 110 aa, Artificial Sequence; identical to SEQ ID NO: 74
  Description of Artificial Sequence: Synthetic polypeptide

SEQ ID NO: 192 - protein, 8 aa, Artificial Sequence
  Description of Artificial Sequence: Synthetic peptide
SSNIGSNT

SEQ ID NO: 193 - protein, 11 aa, Artificial Sequence
  Description of Artificial Sequence: Synthetic peptide
AAWDDSLNGWV

SEQ ID NO: 194 - protein, 3 aa, Artificial Sequence
  Description of Artificial Sequence: Synthetic peptide
NYH

SEQ ID NO: 195 - protein, 123 aa, Artificial Sequence; identical to SEQ ID NO: 70
  Description of Artificial Sequence: Synthetic polypeptide

SEQ ID NO: 196 - protein, 8 aa, Artificial Sequence
  Description of Artificial Sequence: Synthetic peptide
GFTFGDYA

SEQ ID NO: 197 - protein, 14 aa, Artificial Sequence
  Description of Artificial Sequence: Synthetic peptide
ASSGYSSGWTPFDY

SEQ ID NO: 198 - protein, 10 aa, Artificial Sequence
  Description of Artificial Sequence: Synthetic peptide
SRSKAYGGTT

SEQ ID NO: 199 - protein, 117 aa, Artificial Sequence; identical to SEQ ID NO: 30
  Description of Artificial Sequence: Synthetic polypeptide

SEQ ID NO: 200 - protein, 8 aa, Artificial Sequence
  Description of Artificial Sequence: Synthetic peptide
GYTFTAYA

SEQ ID NO: 201 - protein, 10 aa, Artificial Sequence
  Description of Artificial Sequence: Synthetic peptide
ARAAAAGADY

SEQ ID NO: 202 - protein, 8 aa, Artificial Sequence
  Description of Artificial Sequence: Synthetic peptide
IAPAAGAA

SEQ ID NO: 203 - protein, 112 aa, Artificial Sequence; identical to SEQ ID NO: 12
  Description of Artificial Sequence: Synthetic polypeptide

SEQ ID NO: 204 - protein, 11 aa, Artificial Sequence
  Description of Artificial Sequence: Synthetic peptide
QAIVHANGNTY

SEQ ID NO: 205 - protein, 9 aa, Artificial Sequence
  Description of Artificial Sequence: Synthetic peptide
FQGAHAPYT

SEQ ID NO: 206 - protein, 3 aa, Artificial Sequence
  Description of Artificial Sequence: Synthetic peptide
KVA

SEQ ID NO: 207 - protein, 498 aa, Artificial Sequence
  Description of Artificial Sequence: Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGGGGSGGGGSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGGGGSEVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSVEGGSGGSGGSGGSGGVDDVLMTQAPLTLPVSLGDQASISCRSSQAIVHANGNTYLEWYLQKPGQSPALLIYKVANRFSGVPDRFSGSGSGTDFTLKISRVEAEDLGVYYCFQGAHAPYTFGGGTKLEIK

SEQ ID NO: 208 - DNA, 1494 bp, Artificial Sequence
  Description of Artificial Sequence: Synthetic polynucleotide
gatattgcactgacacagcccgcctctgtgagcggctcccctggacagagcatcaccatctcctgcaccggcacaagctccgacatcggcggctacaactctgtgagctggtatcagcagcaccccggcaaggcccctaagctgatgatctacggcgtgaacaataggccatccggcgtgtctaaccgcttctccggctctaagagcggcaataccgcctctctgacaatcagcggcctgcaggcagaggacgaggcagattactattgctctagctacgatatcgagagcgccacccccgtgtttggaggaggaaccaagctgacagtgctgggcggcggcggctctggaggaggaggcagcggcggaggaggctcccaggtggagctggtgcagtccggagccgaggtgaagaagcctggcgagtccctgaagatctcttgtaagggcagcggctactccttcacatcttattggatcggatgggtgcggcaggccccaggcaagggcctggagtggatgggcatcatcgacccaggcgatagccggaccagatactccccctcttttcagggccaggtgaccatctccgccgacaagagcatctccacagcctatctgcagtggtcctctctgaaggccagcgatacagccatgtactattgcgccagaggccagctgtacggaggaacctatatggacggatggggacagggcaccctggtgacagtgagctccggaggaggaggctctgaggtgcagctgcagcagagcggaggagagctggccaagccaggggccagcgtgaagatgtcctgtaagtctagcggctacaccttcacagcctatgccatccactgggcaaagcaggccgccggggcagggctggagtggatcggatacatcgcccccgccgccggagccgccgcctataacgccgcctttaagggcaaggccaccctggccgccgataagtcctctagcacagcatacatggccgccgccgccctgaccagcgaggactccgccgtgtactattgcgcaagagccgccgccgccggagccgattattggggacagggcaccacactgacagtgtcctctgtggagggaggctctggaggcagcggaggctccggcggctctggcggcgtggacgatgtgctgatgacccaggccccactgacactgcccgtgagcctgggcgaccaggcctctatcagctgtaggagctcccaggccatcgtgcacgccaacggcaatacctacctggagtggtatctgcagaagcctggccagtccccagccctgctgatctacaaggtggccaatcggttctctggcgtgcctgacagattttccggctctggcagcggcaccgatttcacactgaagatctcccgcgtggaggcagaggatctgggcgtgtactattgttttcagggagcccacgccccctacaccttcggggggggcacaaaactggaaatcaag

SEQ ID NO: 209 - protein, 111 aa, Artificial Sequence; identical to SEQ ID NO: 92
  Description of Artificial Sequence: Synthetic polypeptide

SEQ ID NO: 210 - protein, 9 aa, Artificial Sequence
  Description of Artificial Sequence: Synthetic peptide
SSDIGGYNS

SEQ ID NO: 211 - protein, 11 aa, Artificial Sequence
  Description of Artificial Sequence: Synthetic peptide
SSYDIESATPV

SEQ ID NO: 212 - protein, 3 aa, Artificial Sequence
  Description of Artificial Sequence: Synthetic peptide
GVN

SEQ ID NO: 213 - protein, 120 aa, Artificial Sequence; identical to SEQ ID NO: 88
  Description of Artificial Sequence: Synthetic polypeptide

SEQ ID NO: 214 - protein, 8 aa, Artificial Sequence
  Description of Artificial Sequence: Synthetic peptide
GYSFTSYW

SEQ ID NO: 215 - protein, 13 aa, Artificial Sequence
  Description of Artificial Sequence: Synthetic peptide
ARGQLYGGTYMDG

SEQ ID NO: 216 - protein, 8 aa, Artificial Sequence
  Description of Artificial Sequence: Synthetic peptide
IDPGDSRT

SEQ ID NO: 217 - protein, 117 aa, Artificial Sequence; identical to SEQ ID NO: 30
  Description of Artificial Sequence: Synthetic polypeptide

SEQ ID NO: 218 - protein, 8 aa, Artificial Sequence
  Description of Artificial Sequence: Synthetic peptide
GYTFTAYA

SEQ ID NO: 219 - protein, 10 aa, Artificial Sequence
  Description of Artificial Sequence: Synthetic peptide
ARAAAAGADY

SEQ ID NO: 220 - protein, 8 aa, Artificial Sequence
  Description of Artificial Sequence: Synthetic peptide
IAPAAGAA

SEQ ID NO: 221 - protein, 112 aa, Artificial Sequence; identical to SEQ ID NO: 12
  Description of Artificial Sequence: Synthetic polypeptide

SEQ ID NO: 222 - protein, 11 aa, Artificial Sequence
  Description of Artificial Sequence: Synthetic peptide
QAIVHANGNTY

SEQ ID NO: 223 - protein, 9 aa, Artificial Sequence
  Description of Artificial Sequence: Synthetic peptide
FQGAHAPYT

SEQ ID NO: 224 - protein, 3 aa, Artificial Sequence
  Description of Artificial Sequence: Synthetic peptide
KVA

SEQ ID NO: 225 - protein, 491 aa, Artificial Sequence
  Description of Artificial Sequence: Synthetic polypeptide
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSVEGGSGGSGGSGGSGGVDDIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 226 - DNA, 1473 bp, Artificial Sequence
  Description of Artificial Sequence: Synthetic polynucleotide
gatattcagctgacccagtctcctagctccctgagcgcctccgtgggcgatagggtgaccatcacatgcaaggcctctcagagcgtggactacgagggcgattccttcctgaactggtatcagcagaagccaggcaaggcccccaagctgctgatctacgcagccagcaatctggagtccggagtgccatctcgcttctccggctctggcagcggaaccgactttaccctgacaatctctagcctgcagccagaggatttcgccacatactattgccagcagagcaacgaggaccccctgacctttggccagggcacaaaggtggagatcaagggaggaggaggctccggcggaggaggctctggcggcggcggcagcgaggtgcagctggtggagtccggcggcggcctggtgcagcccggcggcagcctgcggctgtcctgtgccgcctctggctacaccttttcctcttattggatcgagtgggtgagacaggcccccggcaagggcctggagtggatcggagagatcctgcctggaggaggcgataccaactacaatgagatcttcaagggaagggccaccttcagcgccgacacctccaagaacacagcctatctgcagatgaatagcctgagggccgaggataccgccgtgtactattgcacacggagagtgccaatcaggctggactactggggacagggcaccctggtgacagtgagctccggaggaggaggcagcgaggtgaagctggtggagtccggaggaggcctggtgcagcctggaggctctctgaagctgagctgtgccgcctccggcttcgatttttccaggtattggatgtcttgggtgcgccaggcccctggcaagggcctggaatggatcggcgagatcaacctggactctagcaccatcaattacacaccatctctgaaggacaagttcatcatcagccgggataacgccaagaataccctgtatctgcagatgtctaaggtgagaagcgaggatacagccctgtactattgcgccaggcgctacgacgccatggattattggggccagggcaccagcgtgacagtgtcctctgtggagggaggcagcggaggctccggaggctctggaggcagcggaggagtggacgatatcgtgctgacccagtccccagcctctctggccgtgtccctgggccagcgggccacaatctcttgtagagcctccgagtctgtggacgattacggcatctccttcatgaactggtttcagcagaagcccggccagccccctaagctgctgatctatgccgcccctaatcagggcagcggagtgccagccaggttcagcggctccggctctggaaccgacttttccctgaatatccaccctatggaggaggacgatacagccatgtacttttgtcagcagagcaaggacgtgaggtggagacatcaggcaggcgaccagacagga

SEQ ID NO: 227 - protein, 111 aa, Artificial Sequence [sequence omitted]

SEQ ID NO: 228 - protein, 10 aa, Artificial Sequence
  Description of Artificial Sequence: Synthetic peptide
QSVDYEGDSF

SEQ ID NO: 229 - protein, 9 aa, Artificial Sequence
  Description of Artificial Sequence: Synthetic peptide
QQSNEDPLT

SEQ ID NO: 230 - protein, 3 aa, Artificial Sequence
  Description of Artificial Sequence: Synthetic peptide
AAS

SEQ ID NO: 231 - protein, 117 aa, Artificial Sequence [sequence omitted]

SEQ ID NO: 232 - protein, 8 aa, Artificial Sequence
  Description of Artificial Sequence: Synthetic peptide
GYTFSSYW

SEQ ID NO: 233 - protein, 10 aa, Artificial Sequence
  Description of Artificial Sequence: Synthetic peptide
TRRVPIRLDY

SEQ ID NO: 234 - protein, 8 aa, Artificial Sequence
  Description of Artificial Sequence: Synthetic peptide
ILPGGGDT

SEQ ID NO: 235 - protein, 116 aa, Artificial Sequence; identical to SEQ ID NO: 102
  Description of Artificial Sequence: Synthetic polypeptide

SEQ ID NO: 236 - protein, 8 aa, Artificial Sequence
  Description of Artificial Sequence: Synthetic peptide
GFDFSRYW

SEQ ID NO: 237 - protein, 9 aa, Artificial Sequence
  Description of Artificial Sequence: Synthetic peptide
ARRYDAMDY

SEQ ID NO: 238 - protein, 8 aa, Artificial Sequence
  Description of Artificial Sequence: Synthetic peptide
INLDSSTI

SEQ ID NO: 239 - protein, 109 aa, Artificial Sequence; identical to SEQ ID NO: 19
  Description of Artificial Sequence: Synthetic polypeptide

SEQ ID NO: 240 - protein, 10 aa, Artificial Sequence
  Description of Artificial Sequence: Synthetic peptide
ESVDDYGISF

SEQ ID NO: 241 - protein, 12 aa, Artificial Sequence
  Description of Artificial Sequence: Synthetic peptide
QQSKDVRWRHQA

SEQ ID NO: 242 - protein, 3 aa, Artificial Sequence
  Description of Artificial Sequence: Synthetic peptide
AAP

SEQ ID NO: 243 - protein, 496 aa, Artificial Sequence
  Description of Artificial Sequence: Synthetic polypeptide
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGGGGSGGGGSGGGGSEVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSVEGGSGGSGGSGGSGGVDDIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 244 - DNA, 1488 bp, Artificial Sequence
  Description of Artificial Sequence: Synthetic polynucleotide
cagagcgtgctgacccagccacctagcgcctccggaaccccaggccagagggtgacaatctcttgcagcggcagctcctctaacatcggctccaacaccgtgaattggtaccagcagctgcctggcacagccccaaagctgctgatcttcaattatcaccagaggcccagcggagtgcctgaccgcttttccggctctaagagcggcagctccgcctccctggccatctctggcctgcagagcgaggacgaggccgattactattgcgccgcctgggacgattccctgaacggatgggtgttcggaggaggaaccaagctgacagtgctgggcggaggaggcagcggaggaggaggctccggcggcggcggctctgaggtgcagctggtggaatccggaggaggcctggtgaagccaggaggctccctgcgcctgtcttgtgccgccagcggcttcacctttggcgactacgccctgagctggttcaggcaggcccctggcaagggcctggagtgggtgggcgtgtcccgctctaaggcatacggaggcaccacagattatgccgcctccgtgaagggcaggtttaccatcagccgggacgatagcaagtccacagcctatctgcagatgaatagcctgaagaccgaggacacagccgtgtactattgcgcctctagcggctactcctctggctggaccccattcgattattggggccagggcaccctggtgacagtgagctccggaggaggaggctctgaggtgaagctggtggagagcggaggaggcctggtgcagccaggaggctccctgaagctgtcctgcgccgccagcggcttcgactttagccggtactggatgtcctgggtgagacaggcccctggcaagggcctggaatggatcggcgagatcaacctggattctagcaccatcaattacacaccaagcctgaaggacaagtttatcatctcccgggataacgccaagaataccctgtatctgcagatgtccaaggtgagatctgaggacacagccctgtactattgcgcccggagatacgacgccatggactactggggccagggcacctccgtgacagtgtcctctgtggagggaggctccggaggctctggaggcagcggcggctccggcggcgtggacgatatcgtgctgacccagtctcctgccagcctggccgtgtctctgggccagagggccacaatcagctgtagagcctctgagagcgtggacgattacggcatcagcttcatgaactggtttcagcagaagccaggccagccacccaagctgctgatctatgccgccccaaatcagggctccggagtgcccgcccggttctccggctctggcagcggcaccgatttttctctgaacatccaccctatggaggaggacgatacagccatgtacttttgtcagcagagcaaggacgtgcgctggagacatcaggcaggagaccagacagga

SEQ ID NO: 245 - protein, 110 aa, Artificial Sequence; identical to SEQ ID NO: 74
  Description of Artificial Sequence: Synthetic polypeptide

SEQ ID NO: 246 - protein, 8 aa, Artificial Sequence
  Description of Artificial Sequence: Synthetic peptide
SSNIGSNT

SEQ ID NO: 247 - protein, 11 aa, Artificial Sequence
  Description of Artificial Sequence: Synthetic peptide
AAWDDSLNGWV

SEQ ID NO: 248 - protein, 3 aa, Artificial Sequence
  Description of Artificial Sequence: Synthetic peptide
NYH

SEQ ID NO: 249 - protein, 123 aa, Artificial Sequence; identical to SEQ ID NO: 70
  Description of Artificial Sequence: Synthetic polypeptide

SEQ ID NO: 250 - protein, 8 aa, Artificial Sequence
  Description of Artificial Sequence: Synthetic peptide
GFTFGDYA

SEQ ID NO: 251 - protein, 14 aa, Artificial Sequence
  Description of Artificial Sequence: Synthetic peptide
ASSGYSSGWTPFDY

SEQ ID NO: 252 - protein, 10 aa, Artificial Sequence
  Description of Artificial Sequence: Synthetic peptide
SRSKAYGGTT

SEQ ID NO: 253 - protein, 116 aa, Artificial Sequence; identical to SEQ ID NO: 102
  Description of Artificial Sequence: Synthetic polypeptide

SEQ ID NO: 254 - protein, 8 aa, Artificial Sequence
  Description of Artificial Sequence: Synthetic peptide
GFDFSRYW

SEQ ID NO: 255 - protein, 9 aa, Artificial Sequence
  Description of Artificial Sequence: Synthetic peptide
ARRYDAMDY

SEQ ID NO: 256 - protein, 8 aa, Artificial Sequence
  Description of Artificial Sequence: Synthetic peptide
INLDSSTI

SEQ ID NO: 257 - protein, 109 aa, Artificial Sequence; identical to SEQ ID NO: 19
  Description of Artificial Sequence: Synthetic polypeptide

SEQ ID NO: 258 - protein, 10 aa, Artificial Sequence
  Description of Artificial Sequence: Synthetic peptide
ESVDDYGISF

SEQ ID NO: 259 - protein, 12 aa, Artificial Sequence
  Description of Artificial Sequence: Synthetic peptide
QQSKDVRWRHQA

SEQ ID NO: 260 - protein, 3 aa, Artificial Sequence
  Description of Artificial Sequence: Synthetic peptide
AAP

SEQ ID NO: 261 - protein, 494 aa, Artificial Sequence
  Description of Artificial Sequence: Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGGGGSGGGGSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGGGGSEVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSVEGGSGGSGGSGGSGGVDDIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 262 - DNA, 1482 bp, Artificial Sequence
  Description of Artificial Sequence: Synthetic polynucleotide
gacatcgcactgacccagcctgccagcgtgtccggctctccaggacagtccatcacaatctcttgcaccggcacaagctccgacatcggcggctacaacagcgtgtcctggtatcagcagcacccaggcaaggcccccaagctgatgatctacggcgtgaacaataggccttctggcgtgagcaaccgcttctctggcagcaagtccggcaataccgccagcctgacaatctccggcctgcaggcagaggacgaggcagattactattgctctagctatgatatcgagagcgccaccccagtgtttggaggaggaaccaagctgacagtgctgggcggaggaggcagcggaggaggaggctccggcggcggcggctctcaggtggagctggtgcagtccggagccgaggtgaagaagcccggcgagtctctgaagatcagctgtaagggctccggctactctttcaccagctattggatcggatgggtgcggcaggcccctggcaagggcctggagtggatgggcatcatcgacccaggcgattctaggacccgctactctcccagctttcagggccaggtgaccatctccgccgacaagtccatctctacagcctatctgcagtggtcctctctgaaggccagcgataccgccatgtactattgcgccagaggccagctgtacggcggcacatatatggacggatggggacagggcaccctggtgacagtgagctccggaggaggaggctctgaggtgaagctggtggagagcggaggaggcctggtgcagccaggaggctccctgaagctgtcttgtgccgccagcggcttcgactttagccggtactggatgtcctgggtgagacaggcccctggcaagggcctggaatggatcggcgagatcaacctggattctagcaccatcaattacacaccatccctgaaggacaagttcatcatctctagggataacgccaagaataccctgtatctgcagatgtccaaggtgcgctctgaggatacagccctgtactattgcgcccggagatacgacgccatggattattggggccagggcaccagcgtgacagtgtcctctgtggagggaggctccggaggctctggaggcagcggcggctccggcggcgtggacgatatcgtgctgacccagtctccagccagcctggccgtgagcctgggccagagggccacaatctcctgtagagccagcgagtccgtggacgattacggcatctccttcatgaactggtttcagcagaagcccggccagccccctaagctgctgatctatgccgcccctaatcagggcagcggagtgcctgcccggttctctggcagcggctccggcaccgacttttccctgaatatccaccctatggaggaggacgatacagccatgtacttttgtcagcagagcaaggacgtgcggtggaggcatcaggcaggggaccagacagga

SEQ ID NO: 263 - protein, 111 aa, Artificial Sequence; identical to SEQ ID NO: 92
  Description of Artificial Sequence: Synthetic polypeptide

SEQ ID NO: 264 - protein, 9 aa, Artificial Sequence
  Description of Artificial Sequence: Synthetic peptide
SSDIGGYNS

SEQ ID NO: 265 - protein, 11 aa, Artificial Sequence
  Description of Artificial Sequence: Synthetic peptide
SSYDIESATPV

SEQ ID NO: 266 - protein, 3 aa, Artificial Sequence
  Description of Artificial Sequence: Synthetic peptide
GVN

SEQ ID NO: 267 - protein, 120 aa, Artificial Sequence; identical to SEQ ID NO: 88
  Description of Artificial Sequence: Synthetic polypeptide

SEQ ID NO: 268 - protein, 8 aa, Artificial Sequence
  Description of Artificial Sequence: Synthetic peptide
GYSFTSYW

SEQ ID NO: 269 - protein, 13 aa, Artificial Sequence
  Description of Artificial Sequence: Synthetic peptide
ARGQLYGGTYMDG

SEQ ID NO: 270 - protein, 8 aa, Artificial Sequence
  Description of Artificial Sequence: Synthetic peptide
IDPGDSRT

SEQ ID NO: 271 - protein, 116 aa, Artificial Sequence; identical to SEQ ID NO: 102
  Description of Artificial Sequence: Synthetic polypeptide

SEQ ID NO: 272 - protein, 8 aa, Artificial Sequence
  Description of Artificial Sequence: Synthetic peptide
GFDFSRYW

SEQ ID NO: 273 - protein, 9 aa, Artificial Sequence
  Description of Artificial Sequence: Synthetic peptide
ARRYDAMDY

SEQ ID NO: 274 - protein, 8 aa, Artificial Sequence
  Description of Artificial Sequence: Synthetic peptide
INLDSSTI

SEQ ID NO: 275 - protein, 109 aa, Artificial Sequence; identical to SEQ ID NO: 19
  Description of Artificial Sequence: Synthetic polypeptide

SEQ ID NO: 276 - protein, 10 aa, Artificial Sequence
  Description of Artificial Sequence: Synthetic peptide
ESVDDYGISF

SEQ ID NO: 277 - protein, 12 aa, Artificial Sequence
  Description of Artificial Sequence: Synthetic peptide
QQSKDVRWRHQA

SEQ ID NO: 278 - protein, 3 aa, Artificial Sequence
  Description of Artificial Sequence: Synthetic peptide
AAP

SEQ ID NO: 279 - protein, 479 aa, Artificial Sequence
  Description of Artificial Sequence: Synthetic polypeptide
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSVEGGSGGSGGSGGSGGVDDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 280 - DNA, 1437 bp, Artificial Sequence
  Description of Artificial Sequence: Synthetic polynucleotide
gaggtccagctggtggagtctggaggaggcctggtgcagccaggaggctccctgcggctgtcttgcgcagccagcggatacaccttcagctcctattggatcgagtgggtgagacaggccccaggcaagggcctggagtggatcggagagatcctgccaggaggaggcgataccaactacaatgagatcttcaagggccgggccacattttccgccgacacctctaagaacacagcctatctgcagatgaatagcctgagggccgaggataccgccgtgtactattgcacacggagagtgccaatcaggctggactactggggacagggcaccctggtgacagtgtctagcgtggagggaggcagcggaggctccggaggctctggaggcagcggaggagtggacgatatccagctgacccagagcccttcctctctgtctgccagcgtgggcgatagggtgaccatcacctgtaaggcctcccagtctgtggactacgagggcgattcctttctgaactggtatcagcagaagcccggcaaggcccctaagctgctgatctatgcagccagcaatctggagtccggagtgccatctcgcttcagcggctccggctctggaaccgactttaccctgacaatcagctccctgcagcctgaggatttcgccacatactattgtcagcagtccaacgaggacccactgacctttggccagggcacaaaggtggaaatcaaagcagcagagccaaagtcatccgataagacccatacctgtcccccttgcccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 281 - protein, 117 aa, Artificial Sequence [sequence omitted]

SEQ ID NO: 282 - protein, 8 aa, Artificial Sequence
  Description of Artificial Sequence: Synthetic peptide
GYTFSSYW

SEQ ID NO: 283 - protein, 10 aa, Artificial Sequence
  Description of Artificial Sequence: Synthetic peptide
TRRVPIRLDY

SEQ ID NO: 284 - protein, 8 aa, Artificial Sequence
  Description of Artificial Sequence: Synthetic peptide
ILPGGGDT

SEQ ID NO: 285 - protein, 111 aa, Artificial Sequence [sequence omitted]

SEQ ID NO: 286 - protein, 10 aa, Artificial Sequence
  Description of Artificial Sequence: Synthetic peptide
QSVDYEGDSF

SEQ ID NO: 287 - protein, 9 aa, Artificial Sequence
  Description of Artificial Sequence: Synthetic peptide
QQSNEDPLT

SEQ ID NO: 288 - protein, 3 aa, Artificial Sequence
  Description of Artificial Sequence: Synthetic peptide
AAS

SEQ ID NO: 289 - protein, 110 aa, Artificial Sequence
  Description of Artificial Sequence: Synthetic polypeptide
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 290 - protein, 106 aa, Artificial Sequence
  Description of Artificial Sequence: Synthetic polypeptide
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 291 - protein, 484 aa, Artificial Sequence
  Description of Artificial Sequence: Synthetic polypeptide
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSVEGGSGGSGGSGGSGGVDQSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 292 - DNA, 1452 bp, Artificial Sequence
  Description of Artificial Sequence: Synthetic polynucleotide
gaggtccagctggtggagagcggaggaggcctggtgaagccaggaggctctctgaggctgagctgcgcagcctccggcttcacctttggcgactacgccctgtcctggttcaggcaggcccctggcaagggcctggagtgggtgggcgtgtctagaagcaaggcctacggcggcaccacagattatgccgcctctgtgaagggccggtttaccatcagcagagacgattccaagtctacagcctatctgcagatgaacagcctgaagaccgaggacacagccgtgtactattgcgccagctccggctactctagcggctggaccccattcgattattggggccagggcaccctggtgacagtgtcctctgtggagggaggctccggaggctctggaggcagcggcggctccggaggagtggaccagtccgtgctgacacagccacctagcgcctccggaaccccaggacagagagtgacaatctcttgtagcggcagctcctctaacatcggctccaacaccgtgaattggtaccagcagctgccaggcacagcccccaagctgctgatcttcaattatcaccagaggccttctggcgtgccagatcgcttttccggctctaagagcggcagctccgcctctctggccatcagcggcctgcagtccgaggacgaggcagattactattgtgccgcctgggacgatagcctgaatggctgggtgtttggcggcggcaccaagctgactgtcctggctgctgaaccaaaatcatccgataagacccacacttgcccaccctgcccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 293 - protein, 123 aa, Artificial Sequence; identical to SEQ ID NO: 70
  Description of Artificial Sequence: Synthetic polypeptide

SEQ ID NO: 294 - protein, 8 aa, Artificial Sequence
  Description of Artificial Sequence: Synthetic peptide
GFTFGDYA

SEQ ID NO: 295 - protein, 14 aa, Artificial Sequence
  Description of Artificial Sequence: Synthetic peptide
ASSGYSSGWTPFDY

SEQ ID NO: 296 - protein, 10 aa, Artificial Sequence
  Description of Artificial Sequence: Synthetic peptide
SRSKAYGGTT

SEQ ID NO: 297 - protein, 110 aa, Artificial Sequence; identical to SEQ ID NO: 74
  Description of Artificial Sequence: Synthetic polypeptide

SEQ ID NO: 298 - protein, 8 aa, Artificial Sequence
  Description of Artificial Sequence: Synthetic peptide
SSNIGSNT

SEQ ID NO: 299 - protein, 11 aa, Artificial Sequence
  Description of Artificial Sequence: Synthetic peptide
AAWDDSLNGWV

SEQ ID NO: 300 - protein, 3 aa, Artificial Sequence
  Description of Artificial Sequence: Synthetic peptide
NYH

SEQ ID NO: 301 - protein, 110 aa, Artificial Sequence; identical to SEQ ID NO: 289
  Description of Artificial Sequence: Synthetic polypeptide

SEQ ID NO: 302 - protein, 106 aa, Artificial Sequence; identical to SEQ ID NO: 290
  Description of Artificial Sequence: Synthetic polypeptide

SEQ ID NO: 303 - protein, 482 aa, Artificial Sequence
  Description of Artificial Sequence: Synthetic polypeptide
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSVEGGSGGSGGSGGSGGVDDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 304 - DNA, 1446 bp, Artificial Sequence
  Description of Artificial Sequence: Synthetic polynucleotide
caggtcgagctggtgcagtccggagccgaggtgaagaagcccggcgagtctctgaagatcagctgcaagggctctggctacagcttcacctcctattggatcggatgggtgcggcaggcccctggcaagggcctggagtggatgggcatcatcgaccctggcgattctcggaccagatactctccaagctttcagggccaggtgaccatcagcgccgacaagtccatctctacagcctatctgcagtggagctccctgaaggccagcgataccgccatgtactattgcgccaggggccagctgtacggaggaacatatatggacggatggggacagggcaccctggtgacagtgtctagcgtggagggaggctctggaggcagcggaggctccggaggctctggaggagtggacgatatcgccctgacccagccagccagcgtgtccggctctcccggccagtccatcacaatctcttgtaccggcacatcctctgatatcggcggctacaacagcgtgtcctggtatcagcagcaccccggcaaggcccctaagctgatgatctacggcgtgaacaataggccaagcggcgtgtccaaccgcttctctggcagcaagtccggcaataccgccagcctgacaatctccggcctgcaggcagaggacgaggcagattactattgtagctcctatgacatcgagtccgccacccccgtgtttggaggaggcacaaagctgacagtcctggctgctgaaccaaaatcatccgataagacccatacctgccccccctgcccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 305 - protein, 120 aa, Artificial Sequence; identical to SEQ ID NO: 88
  Description of Artificial Sequence: Synthetic polypeptide

SEQ ID NO: 306 - protein, 8 aa, Artificial Sequence
  Description of Artificial Sequence: Synthetic peptide
GYSFTSYW

SEQ ID NO: 307 - protein, 13 aa, Artificial Sequence
  Description of Artificial Sequence: Synthetic peptide
ARGQLYGGTYMDG

SEQ ID NO: 308 - protein, 8 aa, Artificial Sequence
  Description of Artificial Sequence: Synthetic peptide
IDPGDSRT

SEQ ID NO: 309 - protein, 111 aa, Artificial Sequence; identical to SEQ ID NO: 92
  Description of Artificial Sequence: Synthetic polypeptide

SEQ ID NO: 310 - protein, 9 aa, Artificial Sequence
  Description of Artificial Sequence: Synthetic peptide
SSDIGGYNS

SEQ ID NO: 311 - protein, 11 aa, Artificial Sequence
  Description of Artificial Sequence: Synthetic peptide
SSYDIESATPV

SEQ ID NO: 312 - protein, 3 aa, Artificial Sequence
  Description of Artificial Sequence: Synthetic peptide
GVN

SEQ ID NO: 313 - protein, 110 aa, Artificial Sequence; identical to SEQ ID NO: 289
  Description of Artificial Sequence: Synthetic polypeptide

SEQ ID NO: 314 - protein, 106 aa, Artificial Sequence; identical to SEQ ID NO: 290
  Description of Artificial Sequence: Synthetic polypeptide

SEQ ID NO: 315 - protein, 446 aa, Artificial Sequence
  Description of Artificial Sequence: Synthetic polypeptide
EVQLQQSGGELAKPGASVKMSCKSSGYTFTAYAIHWAKQAAGAGLEWIGYIAPAAGAAAYNAAFKGKATLAADKSSSTAYMAAAALTSEDSAVYYCARAAAAGADYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 316 - DNA, 1338 bp, Artificial Sequence
  Description of Artificial Sequence: Synthetic polynucleotide
gaggtccagctgcagcagtccggaggagagctggccaagccaggggccagcgtgaagatgtcttgcaagagctccggctacaccttcacagcctatgccatccactgggcaaagcaggccgccggagctggcctggagtggatcggatacatcgcacccgccgccggagccgccgcctataacgccgcctttaagggcaaggccaccctggccgccgacaagtctagctccacagcatacatggccgccgccgccctgaccagcgaggatagcgccgtgtactattgtgccagggcagcagcagcaggagccgactactgggggcaggggactactctgactgtgagctccgctagcaccaagggaccttccgtgttcccactggcaccaagctccaagtctacaagcggaggaaccgccgccctgggatgtctggtgaaggattacttcccagagcccgtgaccgtgtcttggaacagcggggccctgaccagcggagtgcacacctttcctgccgtgctgcagtctagcggcctgtattccctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggcagcaggaggaccaagcgtgttcctgtttccacccaagcctaaggacacactgatgatctccaggacaccagaggtgacctgcgtggtggtgtccgtgtctcacgaggaccccgaggtgaagttcaactggtacgtggatggcgtggaggtgcacaatgccaagaccaagcccagggaggagcagtataactctacataccgcgtggtgagcgtgctgaccgtgctgcaccaggattggctgaacggcaaggagtacaagtgcaaggtgagcaataaggccctgcccgcccctatcgagaagaccatctccaaggccaagggccagcctcgcgaaccacaggtgtacgtgtaccctccatctagagacgagctgacaaagaaccaggtgagcctgacctgtctggtgaagggcttttatcccagcgatatcgccgtggagtgggagtccaatggccagcctgagaacaattacaagacaaccccccctgtgctggactccgatggctctttcgccctggtgtccaagctgaccgtggacaagtctcggtggcagcagggcaacgtgttcagctgttccgtgatgcacgaggcactgcacaatcactacacccagaagtcactgtcactgtccccaggc

SEQ ID NO: 317 - protein, 117 aa, Artificial Sequence; identical to SEQ ID NO: 30
  Description of Artificial Sequence: Synthetic polypeptide

SEQ ID NO: 318 - protein, 8 aa, Artificial Sequence
  Description of Artificial Sequence: Synthetic peptide
GYTFTAYA

SEQ ID NO: 319 - protein, 10 aa, Artificial Sequence
  Description of Artificial Sequence: Synthetic peptide
ARAAAAGADY

SEQ ID NO: 320 - protein, 8 aa, Artificial Sequence
  Description of Artificial Sequence: Synthetic peptide
IAPAAGAA

SEQ ID NO: 321 - protein, 98 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 322 - protein, 110 aa, Artificial Sequence; identical to SEQ ID NO: 289
  Description of Artificial Sequence: Synthetic polypeptide

SEQ ID NO: 323 - protein, 106 aa, Artificial Sequence
  Description of Artificial Sequence: Synthetic polypeptide
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 324 - protein, 445 aa, Artificial Sequence
  Description of Artificial Sequence: Synthetic polypeptide
EVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 325 - DNA, 1335 bp, Artificial Sequence
  Description of Artificial Sequence: Synthetic polynucleotide
gaggtcaagctggtggagtctggaggaggcctggtgcagccaggaggctctctgaagctgagctgcgccgcctccggcttcgacttttcccggtactggatgtcttgggtgagacaggcccccggcaagggcctggagtggatcggcgagatcaacctggatagctccaccatcaattacacacctagcctgaaggacaagttcatcatctccagggataacgccaagaataccctgtatctgcagatgtctaaggtgcggagcgaggacacagccctgtactattgtgcacgcagatacgatgctatggattattgggggcagggaacctcagtcaccgtctcttctgctagcaccaagggaccttccgtgttcccactggcaccaagctccaagtctacaagcggaggaaccgccgccctgggatgtctggtgaaggattacttcccagagcccgtgaccgtgtcttggaacagcggggccctgaccagcggagtgcacacctttcctgccgtgctgcagtctagcggcctgtattccctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggcagcaggaggaccaagcgtgttcctgtttccacccaagcctaaggacacactgatgatctccaggacaccagaggtgacctgcgtggtggtgtccgtgtctcacgaggaccccgaggtgaagttcaactggtacgtggatggcgtggaggtgcacaatgccaagaccaagcccagggaggagcagtataactctacataccgcgtggtgagcgtgctgaccgtgctgcaccaggattggctgaacggcaaggagtacaagtgcaaggtgagcaataaggccctgcccgcccctatcgagaagaccatctccaaggccaagggccagcctcgcgaaccacaggtgtacgtgtaccctccatctagagacgagctgacaaagaaccaggtgagcctgacctgtctggtgaagggcttttatcccagcgatatcgccgtggagtgggagtccaatggccagcctgagaacaattacaagacaaccccccctgtgctggactccgatggctctttcgccctggtgtccaagctgaccgtggacaagtctcggtggcagcagggcaacgtgttcagctgttccgtgatgcacgaggcactgcacaatcactacacccagaagtcactgtcactgtccccaggc

SEQ ID NO: 326 - protein, 116 aa, Artificial Sequence; identical to SEQ ID NO: 102
  Description of Artificial Sequence: Synthetic polypeptide

SEQ ID NO: 327 - protein, 8 aa, Artificial Sequence
  Description of Artificial Sequence: Synthetic peptide
GFDFSRYW

SEQ ID NO: 328 - protein, 9 aa, Artificial Sequence
  Description of Artificial Sequence: Synthetic peptide
ARRYDAMDY

SEQ ID NO: 329 - protein, 8 aa, Artificial Sequence
  Description of Artificial Sequence: Synthetic peptide
INLDSSTI

SEQ ID NO: 330 - protein, 98 aa, Artificial Sequence; identical to SEQ ID NO: 321
  Description of Artificial Sequence: Synthetic polypeptide

SEQ ID NO: 331 - protein, 110 aa, Artificial Sequence; identical to SEQ ID NO: 289
  Description of Artificial Sequence: Synthetic polypeptide

SEQ ID NO: 332 - protein, 106 aa, Artificial Sequence; identical to SEQ ID NO: 323
  Description of Artificial Sequence: Synthetic polypeptide

SEQ ID NO: 333 - protein, 478 aa, Artificial Sequence
  Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVEGGSGGSGGSGGSGGVDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 334 - DNA, 1434 bp, Artificial Sequence
  Description of Artificial Sequence: Synthetic polynucleotide
gaggtcaagctgcaggagagcggaccaggcctggtggccccctcccagtctctgagcgtgacctgcacagtgtctggcgtgagcctgcccgactacggcgtgtcttggatcagacagccccctagaaagggcctggagtggctgggcgtgatctggggctccgagacaacatactataactctgccctgaagagcagactgaccatcatcaaggacaactccaagtctcaggtgttcctgaagatgaacagcctgcagaccgacgatacagccatctactattgtgccaagcactactattacggcggcagctatgccatggattactggggccagggcacctccgtgacagtgagctccgtggagggaggctccggaggctctggaggcagcggcggctccggcggcgtggacgatatccagatgacccagaccacatctagcctgagcgcctccctgggcgacagggtgacaatctcctgccgcgcctctcaggatatcagcaagtatctgaattggtaccagcagaagcctgatggcaccgtgaagctgctgatctatcacacatcccggctgcactctggcgtgccaagcaggttttctggcagcggctccggaaccgactactccctgacaatctctaacctggagcaggaggatatcgccacctatttctgtcagcagggcaataccctgccttacacatttggcggcggcacaaagctggaaatcaccgcagcagaaccaaaatcctccgataaaactcacacttgccccccttgcccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 335 - protein, 120 aa, Artificial Sequence; identical to SEQ ID NO: 34
  Description of Artificial Sequence: Synthetic polypeptide

SEQ ID NO: 336 - protein, 8 aa, Artificial Sequence
  Description of Artificial Sequence: Synthetic peptide
GVSLPDYG

SEQ ID NO: 337 - protein, 14 aa, Artificial Sequence
  Description of Artificial Sequence: Synthetic peptide
AKHYYYGGSYAMDY

SEQ ID NO: 338 - protein, 7 aa, Artificial Sequence
  Description of Artificial Sequence: Synthetic peptide
IWGSETT

SEQ ID NO: 339 - protein, 107 aa, Artificial Sequence; identical to SEQ ID NO: 38
  Description of Artificial Sequence: Synthetic polypeptide

SEQ ID NO: 340 - protein, 6 aa, Artificial Sequence
  Description of Artificial Sequence: Synthetic peptide
QDISKY

SEQ ID NO: 341 - protein, 9 aa, Artificial Sequence
  Description of Artificial Sequence: Synthetic peptide
QQGNTLPYT

SEQ ID NO: 342 - protein, 3 aa, Artificial Sequence
  Description of Artificial Sequence: Synthetic peptide
HTS

SEQ ID NO: 343 - protein, 110 aa, Artificial Sequence; identical to SEQ ID NO: 289
  Description of Artificial Sequence: Synthetic polypeptide

SEQ ID NO: 344 - protein, 106 aa, Artificial Sequence; identical to SEQ ID NO: 290
  Description of Artificial Sequence: Synthetic polypeptide

SEQ ID NO: 345 - protein, 5 aa, Artificial Sequence
  Description of Artificial Sequence: Synthetic peptide
GSGGS

SEQ ID NO: 346 - protein, 4 aa, Artificial Sequence
  Description of Artificial Sequence: Synthetic peptide
GGGS

SEQ ID NO: 347 - protein, 5 aa, Artificial Sequence
  Description of Artificial Sequence: Synthetic peptide
GGGGS

SEQ ID NO: 348 - protein, 6 aa, Artificial Sequence
  Description of Artificial Sequence: Synthetic 6xHis tag
HHHHHH

SEQ ID NO: 349 - protein, 8 aa, Artificial Sequence
  Description of Artificial Sequence: Synthetic peptide
DYKDDDDK

SEQ ID NO: 350 - protein, 25 aa, Artificial Sequence
  Description of Artificial Sequence: Synthetic peptide
GGGGSGGGGSGGGGSGGGGSGGGGS

(19) United States
(12) Patent Application Publication
(10) Pub. No.: US 2019/0111079 A1
(43) Pub. Date: Apr. 18, 2019

(54) MULTI-SPECIFIC ANTIGEN-BINDING CONSTRUCTS TARGETING IMMUNOTHERAPEUTICS

(71) Applicant: Zymeworks Inc., Vancouver (CA)

(72) Inventor: David M. Mills, Seattle, WA (US)

(21) Appl. No.: 16/088,760

(22) PCT Filed: Apr. 13, 2017
(86) PCT No.: PCT/CA2017/050463

§ 371 Date: Sep. 26, 2018

Related U.S. Application Data
(60) Provisional application No. 62/323,432, filed on Apr. 15, 2016.

(51) Int. Cl. A61K 35/17; C07K 16/28; A61P 35/00; C12N 5/0783; C07K 16/30

(52) U.S. Cl. CPC A61K 35/17; C07K 16/2803; C07K 16/30; A61P 35/00; C12N 5/0636; C07K 16/2878

(57) ABSTRACT

Multi-specific antigen-binding constructs that target immunotherapeutics are described. The multi-specific antigen-binding constructs comprise a first antigen-binding polypeptide construct that binds to an immunotherapeutic (such as a CAR-T cell or a bispecific T-cell engager), and a second antigen binding polypeptide construct that binds to a tumour-associated antigen. Also described are methods of using the multi-specific antigen-binding constructs to re-direct or enhance the binding of the immunotherapeutic to a tumour cell, and methods of treating patients who have relapsed from or failed treatment with the immunotherapeutic.

### BACKGROUND

Compared to conventional anti-cancer chemotherapeutics, immunotherapeutics display enhanced ability to overcome tumour genetic resistance mechanisms and reduced healthy tissue toxicity profiles. In particular, directing immune-mediated tumour cytolysis toward tumour-associated antigens (TAAs) has revolutionized hematopoietic and solid tissue neoplasm treatment protocols, providing long-lasting remission in many patients. However, antigen-directed immunotherapy resistance mechanisms have emerged, including TAA downregulation, necessitating development of refined treatment options.

Autologous adoptive cell therapy with T lymphocytes expressing engineered, TAA-specific, chimeric antigen receptors (CARs) is a particularly effective treatment modality in relapsed/refractory B cell acute lymphoblastic leukemia (B-ALL) patients, and is now being pursued for numerous oncologic indications. Similarly, bispecific T-cell engager (BiTE) biologics promote targeted cytotoxic responses by co-engaging TCR CD3 signaling subunits with TAAs, and are approved for B-ALL treatment. Although these approaches can harness adaptive immune potential for antigen-specific cytotoxicity and long-lived immunologic memory, a sizeable percentage of BiTE and CAR-T therapy patients relapse due to TAA-negative tumour variant outgrowth.

### SUMMARY

Described herein are multi-specific antigen-binding constructs targeting immunotherapeutics and methods of using same. Certain aspects of the disclosure relate to a method of re-directing tumour cell binding by an immunotherapeutic, the method comprising contacting the immunotherapeutic with a multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a method of extending the therapeutic effect of an immunotherapeutic in a patient who is undergoing or has undergone treatment with the immunotherapeutic, the method comprising administering to the patient an effective amount of a multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a method of treating cancer in a patient who is undergoing or has undergone treatment with an immunotherapeutic, the method comprising administering an effective amount of a multi-specific antigen-binding construct to the patient, the multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a method of activating a T-cell or NK cell comprising contacting a T-cell or NK cell engineered to express a chimeric antigen receptor (CAR) or a T-cell receptor (TCR) with a multi-specific antigen-binding construct comprising a first antigen-binding polypeptide construct that binds to the CAR or TCR and a second antigen-binding polypeptide construct that binds to a first tumour-associated antigen epitope, wherein the CAR or TCR comprises an antigen-binding domain that binds to a second tumour-associated antigen epitope.

Some aspects of the present disclosure relate to a multi-specific antigen-binding construct comprising: a first antigen-binding polypeptide construct that binds to an immunotherapeutic, and a second antigen binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to nucleic acid encoding a multi-specific antigen-binding construct as described herein. Some aspects relate to a host cell comprising nucleic acid encoding a multi-specific antigen-binding construct as described herein.

Certain aspects of the disclosure relate to a use of a multi-specific antigen-binding construct to re-direct tumour cell binding by an immunotherapeutic, the multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a use of a multi-specific antigen-binding construct to extend the therapeutic effect of an immunotherapeutic in a patient who is undergoing or has undergone treatment with the immunotherapeutic, the multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a use of a multi-specific antigen-binding construct to treat cancer in a patient who is undergoing or has undergone treatment with an immunotherapeutic, the multi-specific antigen-binding construct comprising a first antigen-binding polypeptide construct that binds to the immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a use of a multi-specific antigen-binding construct to activate a T-cell or NK cell that is engineered to express a chimeric antigen receptor (CAR) or a T-cell receptor (TCR), the multi-specific antigen-binding construct comprising a first antigen-binding polypeptide construct that binds to the CAR or TCR and a second antigen-binding polypeptide construct that binds to a first tumour-associated antigen epitope, wherein the CAR or TCR comprises an antigen-binding domain that binds to a second tumour-associated antigen epitope.

Some aspects of the present disclosure relate to a pharmaceutical composition comprising a multi-specific antigen-binding construct and a pharmaceutically acceptable carrier, the multi-specific antigen-binding construct comprising a first antigen-binding polypeptide construct that binds to an immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

Some aspects of the present disclosure relate to a use of a multi-specific antigen-binding construct in the manufacture of a medicament, the multi-specific antigen-binding construct comprising a first antigen-binding polypeptide construct that binds to an immunotherapeutic and a second antigen-binding polypeptide construct that binds to a first tumour-associated antigen epitope, wherein the immunotherapeutic is: i) a T-cell or NK cell engineered to express an antigen-binding domain that binds to a second tumour-associated antigen epitope, or ii) a therapeutic agent capable of binding to a T-cell and to a second tumour-associated antigen epitope, and wherein the first and second tumour-associated antigen epitopes are different.

### BRIEF DESCRIPTION OF THE DRAWINGS

FIG. 1 depicts (A) a schematic diagram of one embodiment of a multi-specific antigen-binding construct which targets an anti-CD19 CAR-T and CD79b as the tumour-associated antigen, and (B) some exemplary formats for the described multi-specific antigen-binding constructs.

FIG. 2 depicts binding of an anti-FLAG×anti-mesothelin (MSLN) bispecific antibody and an anti-FMC63id×anti-MSLN bispecific antibody to MSLN+A1847 cells, but not control RPMI8226 cells (A), and binding of an anti-FLAG×anti-BCMA bispecific antibody and an anti-FMC63id×anti-BCMA bispecific antibody to BCMA+RPMI8226 cells, but not control A1847 cells (B).

FIG. 3 depicts selective binding of anti-FMC63id×anti-mesothelin and anti-FMC63id×anti-BCMA bispecific antibodies to anti-CD19 CAR constructs containing FMC63 that are stably expressed on either HEK293 (A) or primary CAR-T cells (B).

FIG. 4 shows (A) CD19-CAR-T cells are robustly activated upon co-culture with CD19+Raji cells, but not CD19-negative SKOV3 cells, and (B) an anti-FMC63id×anti-mesothelin bispecific antibody re-directed CAR-T cells and potentiated activation in the presence of MSLN+SKOV3 cells, and an anti-FMC63id×anti-BCMA bispecific antibody re-directed CAR-T cells and potentiated activation in the presence of BCMA+RPMI8226 cells.

### DETAILED DESCRIPTION

Described herein are multi-specific antigen-binding constructs that target immunotherapeutics. Specifically, the multi-specific antigen-binding constructs are capable of binding to an immunotherapeutic and to at least one tumour-associated antigen. In certain embodiments, the multi-specific antigen-binding constructs comprise a first antigen-binding polypeptide construct that binds to an immunotherapeutic, and a second antigen-binding polypeptide construct that binds to a tumour-associated antigen. In some embodiments, the immunotherapeutic may be an effector cell, such as a T-cell or an NK cell, that is engineered to express an antigen-binding domain that binds to a tumour-associated antigen. In some embodiments, the immunotherapeutic may be a therapeutic agent that is capable of binding to a T-cell and to a tumour-associated antigen. In some embodiments, the tumour-associated antigen that is targeted by the multi-specific antigen-binding construct is different to the tumour-associated antigen that is targeted by the immunotherapeutic. In some embodiments, the tumour-associated antigen that is targeted by the multi-specific antigen-binding construct is the same as the tumour-associated antigen targeted by the immunotherapeutic, but the multi-specific antigen-binding construct and the immunotherapeutic bind to different epitopes on the tumour-associated antigen.

Also described herein are methods of using the multi-specific antigen-binding constructs to re-direct or enhance the binding of the immunotherapeutic to a tumour cell. In accordance with these methods, the multi-specific antigen-binding construct binds to the immunotherapeutic through a first antigen-binding polypeptide construct, and binds to a tumour-associated antigen on a tumour cell through a second antigen-binding polypeptide. The second antigen-binding polypeptide either binds to a different tumour-associated antigen to that targeted by the immunotherapeutic, or binds to a different epitope on the tumour-associated antigen to that targeted by the immunotherapeutic. Thus, in some embodiments, the multi-specific antigen-binding construct re-directs the binding of the immunotherapeutic from its cognate tumour-associated antigen or epitope to the tumour-associated antigen or epitope targeted by the second antigen-binding polypeptide construct. In some embodiments, the immunotherapeutic retains binding to its cognate tumour-associated antigen or epitope on a tumour cell, and also binds the tumour cell via the multi-specific antigen-binding construct and its cognate tumour-associated antigen or epitope. In this embodiment, binding of the tumour cell by the immunotherapeutic may thus be enhanced. In certain embodiments, the multi-specific antigen-binding constructs may find use as a follow-on or adjunctive therapy. For example, for patients who are undergoing, or have previously undergone, treatment with an immunotherapeutic and in whom there is a risk of loss, or a decrease in expression, of the immunotherapeutic target tumour-associated antigen, for patients who may become unresponsive via alternative mechanisms to immunotherapeutic-directed cytolysis, or for patients who display significant heterogeneity in expression of the immunotherapeutic target tumour-associated antigen.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

Where a range of values is provided, it is understood that each intervening value between the upper and lower limit of that range, to the tenth of the unit of the lower limit unless the context clearly dictates otherwise, is encompassed within the range and that each of these intervening values form embodiments of the present disclosure. These intervening values may also represent the upper and lower limits of smaller ranges included within the stated range and each of such smaller ranges also form embodiments of the present disclosure, subject to any specifically excluded limits in the stated range.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method, use or composition disclosed herein, and vice versa.

### Multi-Specific Antigen-Binding Constructs

Described herein are multi-specific antigen-binding constructs capable of binding to an immunotherapeutic and at least one tumour-associated antigen. In certain embodiments, the multi-specific antigen-binding constructs comprise a first antigen-binding polypeptide construct that binds to an immunotherapeutic, and a second antigen-binding polypeptide construct that binds to a tumour-associated antigen. In some embodiments, the multi-specific antigen-binding constructs may comprise one or more additional antigen-binding polypeptide constructs each of which binds to a tumour-associated antigen. In certain embodiments, each antigen-binding polypeptide construct comprised by the multi-specific antigen-binding construct specifically binds to its target antigen.

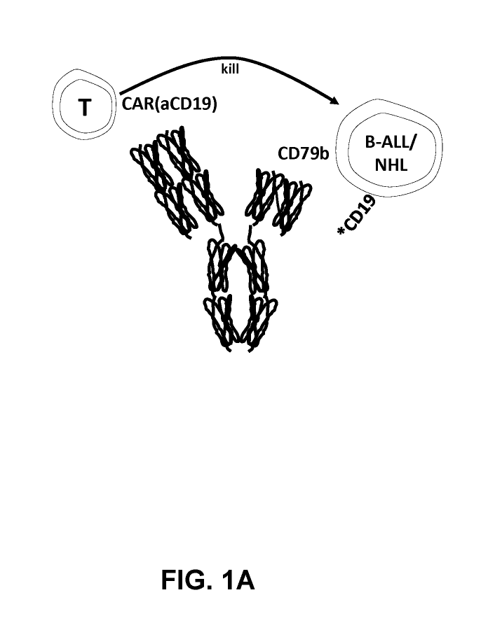

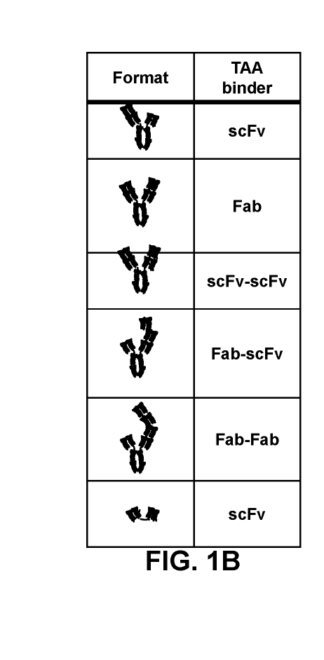

The term “antigen-binding construct” refers to an agent, e.g. polypeptide or polypeptide complex, capable of binding to an antigen. In some aspects, an antigen-binding construct may be a polypeptide that specifically binds to a target antigen of interest. An antigen-binding construct may be a monomer, dimer, multimer, a protein, a peptide, a protein or peptide complex, an antibody, an antibody fragment, a Fab, an scFv, a single domain antibody (sdAb), a VHH, or the like. In some embodiments, a multi-specific antigen-binding construct may include one or more antigen-binding moieties (e.g. Fabs, scFvs, VHHs or sdAbs) linked to a scaffold. Examples of multi-specific antigen-binding constructs are described below and provided in the Examples section. Some exemplary, non-limiting, formats of multi-specific antigen-binding constructs are shown in FIG. 1B.

In the present context, the antigen-binding construct is a multi-specific antigen-binding construct. The term “multi-specific antigen-binding construct,” as used herein, is an antigen-binding construct which has two or more antigen-binding moieties (e.g. antigen-binding polypeptide constructs), each with a unique binding specificity. In certain embodiments, the multi-specific antigen-binding construct comprises two antigen-binding moieties (i.e. is bispecific). In some embodiments, the multi-specific antigen-binding construct comprises three antigen-binding moieties (i.e. is trispecific). In some embodiments, the multi-specific antigen-binding construct comprises more than three antigen-binding moieties, for example, four antigen-binding moieties.

Certain embodiments of the present disclosure relate to bispecific antigen-binding constructs. The term “bispecific antigen-binding construct” refers to an antigen-binding construct that has two antigen-binding moieties (e.g. antigen-binding polypeptide constructs), each with a unique binding specificity. For example, the bispecific antigen-binding construct may comprise a first antigen-binding moiety that binds to an epitope on a first antigen and a second antigen-binding moiety that binds to an epitope on a second antigen, or the bispecific antigen-binding construct may comprise a first antigen-binding moiety that binds to an epitope on a first antigen and a second antigen-binding moiety that binds to a different epitope on the first antigen. The term “biparatopic” may be used to refer to a bispecific antigen-binding construct in which the first antigen-binding moiety and the second antigen-binding moiety bind to different epitopes on the same antigen. The biparatopic antigen-binding construct may bind to a single antigen molecule through the two epitopes, or it may bind to two separate antigen molecules, each through a different epitope.

In some embodiments, the antigen-binding construct comprises two or more antigen-binding moieties that are antigen-binding polypeptide constructs, each of the antigen-binding polypeptide constructs being independently a Fab, an scFv or an sdAb, optionally of camelid origin (VHH).

In some embodiments, the multi-specific antigen-binding construct further comprises a scaffold and the antigen-binding polypeptide constructs are operably linked to the scaffold. The term “operably linked,” as used herein, means that the components described are in a relationship permitting them to function in their intended manner.

In certain embodiments, the multi-specific antigen-binding construct may be an antibody or antigen-binding antibody fragment. The terms “antibody” and “immunoglobulin” are used interchangeably herein to refer to a polypeptide encoded by an immunoglobulin gene or genes, or a modified version of an immunoglobulin gene, which polypeptide specifically binds and recognizes an analyte (e.g. antigen). The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG and IgM, and several of these may be further divided into subclasses (isotypes), e.g. IgG₁, IgG₂, IgG₃, IgG₄, IgA₁ and IgA₂. The heavy chain constant domains that correspond to the different classes of immunoglobulins are called α, δ, ε, γ and μ, respectively.

An exemplary immunoglobulin (antibody) structural unit is composed of two pairs of polypeptide chains, each pair having one “light” chain (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises the VH, CH1, CH2 and CH3 domains, respectively, from N- to C-terminus. The light chain comprises the VL and CL domains from N- to C-terminus. The IgG1 heavy chain comprises a hinge between the CH1 and CH2 domains. In certain embodiments, the multi-specific antigen-binding constructs comprise at least one immunoglobulin domain from IgG, IgM, IgA, IgD or IgE. In some embodiments, the multi-specific antigen-binding construct comprises one or more immunoglobulin domains from or derived from an immunoglobulin-based construct such as a diabody or a nanobody. In certain embodiments, the multi-specific antigen-binding construct comprises at least one immunoglobulin domain from a heavy chain antibody such as a camelid antibody. In certain embodiments, the multi-specific antigen-binding construct comprises at least one immunoglobulin domain from a mammalian antibody such as a bovine antibody, a human antibody, a camelid antibody, a mouse antibody or any chimeric antibody.

The term “hypervariable region” (HVR) as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. The terms hypervariable regions (HVRs) and complementarity determining regions (CDRs) are used herein interchangeably in reference to the portions of the variable region that form the antigen-binding regions. This particular region has been described by Kabat et al., U.S. Dept. of Health and Human Services, Sequences of Proteins of Immunological Interest (1983) and by Chothia et al., J Mol Biol, 196:901-917 (1987), where the definitions include overlapping or subsets of amino acid residues when compared against each other. Nevertheless, application of either definition to refer to a CDR is intended to be within the scope of the term as defined and used herein.

### Antigen-Binding Polypeptide Constructs

The multi-specific antigen-binding constructs described herein comprise two or more antigen-binding polypeptide constructs, one of which binds (e.g. specifically binds) to an immunotherapeutic, and one or more of which each independently bind (e.g. specifically bind) to a tumour-associated antigen. In some embodiments, one or more of the antigen-binding polypeptide constructs are immunoglobulin-based constructs, for example, antibody fragments. In some embodiments, one or more of the antigen-binding polypeptide constructs may be a non-immunoglobulin based antibody mimetic format, including, but not limited to, an anticalin, a fynomer, an affimer, an alphabody, a DARPin or an avimer.

In certain embodiments, the antigen-binding polypeptide constructs may each independently be a Fab, an scFv or a sdAb, depending on the intended application of the multi-specific antigen-binding construct.

In certain embodiments, at least one of the antigen-binding polypeptide constructs comprised by the multi-specific antigen-binding construct may be a Fab fragment. A “Fab fragment” (also referred to as fragment antigen-binding) contains the constant domain (CL) of the light chain and the first constant domain (CH1) of the heavy chain along with the variable domains VL and VH on the light and heavy chains, respectively. The variable domains comprise the CDRs, which are involved in antigen-binding. Fab′ fragments differ from Fab fragments by the addition of a few amino acid residues at the C-terminus of the heavy chain CH1 domain, including one or more cysteines from the antibody hinge region. In some embodiments, one of the antigen-binding polypeptide constructs comprised by the multi-specific antigen-binding construct may be a Fab′ fragment.

As used herein, the term “single-chain” refers to a molecule comprising amino acid monomers linearly linked by peptide bonds. In certain embodiments, one or more of the antigen-binding polypeptide constructs comprised by the multi-specific antigen-binding construct may be a single-chain Fab molecule, i.e. a Fab molecule in which the Fab light chain and the Fab heavy chain are connected by a peptide linker to form a single peptide chain. For example, in some embodiments in which an antigen-binding polypeptide construct comprised by the multi-specific antigen-binding construct is a single-chain Fab molecule, the C-terminus of the Fab light chain may be connected to the N-terminus of the Fab heavy chain in the single-chain Fab molecule.

In certain embodiments, at least one of the antigen-binding polypeptide constructs comprised by the multi-specific antigen-binding construct may be a single-chain Fv (scFv). An “scFv” includes a heavy chain variable domain (VH) and a light chain variable domain (VL) of an antibody in a single polypeptide chain. The scFv may optionally further comprise a polypeptide linker between the VH and VL domains which enables the scFv to form a desired structure for antigen binding. In some embodiments, an scFv may include a VL connected from its C-terminus to the N-terminus of a VH by a polypeptide linker. Alternately, an scFv may comprise a VH connected through its C-terminus to the N-terminus of a VL by a polypeptide chain or linker. For a review of scFvs see Pluckthun in The Pharmacology of Monoclonal Antibodies, vol. 113, Rosenburg and Moore eds., Springer-Verlag, New York, pp. 269-315 (1994).

In certain embodiments, at least one of the antigen-binding polypeptide constructs comprised by the multi-specific antigen-binding construct may be in a single domain antibody (sdAb) format. An sdAb format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. SdAbs are fairly stable and easy to express, for example, as a fusion with the Fc chain of an antibody (see, for example, Harmsen & De Haard, Appl. Microbiol Biotechnol. 77(1): 13-22 (2007)).

In certain embodiments, at least one of the antigen-binding polypeptide constructs comprised by the multi-specific antigen-binding construct that binds a tumour-associated antigen may be a natural ligand for a tumour-associated antigen, or a functional fragment of such a ligand. Examples include, but are not limited to, folate (ligand for FRalpha), recombinant EGF (ligand for EGFR) or Wnt5a (ligand for ROR1).

### Formats

The multi-specific antigen-binding constructs described herein may be considered to have a modular architecture that includes two or more antigen-binding polypeptide construct modules and an optional scaffold module. One skilled in the art will understand that these modules may be combined in various ways to provide multi-specific antigen-binding constructs having different formats. These formats are based generally on art-known antibody formats (see, for example, review by Brinkmann & Kontermann, MABS, 9(2):182-212 (2017), and Müller & Kontermann, “Bispecific Antibodies” in Handbook of Therapeutic Antibodies, Wiley-VCH Verlag GmbH & Co. (2014)), and include those described above and the exemplary, non-limiting, formats of multi-specific antigen-binding constructs shown in FIG. 1B.

Multi-specific antigen-binding constructs that lack a scaffold typically comprise two or more antigen-binding polypeptide constructs operably linked by one or more linkers. The antigen-binding polypeptide constructs may be in the form of scFvs, Fabs, sdAbs, or a combination thereof. For example, using scFvs as the antigen-binding polypeptide constructs, formats such as a tandem scFv ((scFv)₂ or taFv) or a triplebody (3 scFvs) may be constructed, in which the scFvs are connected together by a flexible linker. scFvs may also be used to construct diabody, triabody and tetrabody (tandem diabodies or TandAbs) formats, which comprise 2, 3 and 4 scFvs, respectively, connected by a short linker (usually about 5 amino acids in length). The restricted length of the linker results in dimerization of the scFvs in a head-to-tail manner. In any of the preceding formats, the scFvs may be further stabilized by inclusion of an interdomain disulfide bond. For example, a disulfide bond may be introduced between VL and VH through introduction of an additional cysteine residue in each chain (for example, at position 44 in VH and 100 in VL) (see, for example, Fitzgerald et al., Protein Engineering, 10:1221-1225 (1997)), or a disulfide bond may be introduced between two VHs to provide construct having a DART format (see, for example, Johnson et al., J Mol. Biol., 399:436-449 (2010)).

Similarly, formats comprising two or more sdAbs, such as VHs or VHHs, connected together through a suitable linker may be used for the multi-specific antigen-binding construct.

Other examples of multi-specific antigen-binding construct formats that lack a scaffold include those based on Fab fragments, for example, Fab₂, F(ab′)₂ and F(ab′)₃ formats, in which the Fab fragments are connected through a linker or an IgG hinge region.

Combinations of antigen-binding polypeptide constructs in different forms may also be employed to generate alternative scaffold-less formats. For example, an scFv or a sdAb may be fused to the C-terminus of either or both of the light and heavy chain of a Fab fragment resulting in a bivalent (Fab-scFV/sdAb) or trivalent (Fab-(scFv)₂ or Fab-(sdAb)₂) construct. Similarly, one or two scFvs or sdAbs may be fused at the hinge region of a F(ab′) fragment to produce a tri- or tetravalent F(ab′)₂-scFv/sdAb construct.

In certain embodiments, the multi-specific antigen-binding construct comprises two or more antigen-binding polypeptide constructs and one or more linkers, and does not include a scaffold. In some embodiments, the multi-specific antigen-binding construct comprises two or more antigen-binding polypeptide constructs and one or more linkers, in which the antigen-binding polypeptide constructs are scFvs, Fabs, sdAbs, or a combination thereof. In some embodiments, the multi-specific antigen-binding construct comprises two or more antigen-binding polypeptide constructs and one or more linkers, in which the antigen-binding polypeptide constructs are scFvs.

Multi-specific antigen-binding constructs comprising a scaffold may be constructed by linking two or more antigen-binding polypeptide constructs to a suitable scaffold. The antigen-binding polypeptide constructs may be in one or a combination of the forms described above (e.g. scFvs, Fabs and/or sdAbs). Examples of suitable scaffolds are described in more detail below and include, but are not limited to, immunoglobulin Fc regions, albumin, albumin analogs and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include multi-specific antigen-binding constructs based on the DOCK-AND-LOCK™ (DNL™) technology developed by IBC Pharmaceuticals, Inc. and Immunomedics (see, for example, Chang, et al., Clin Cancer Res 13:5586s-5591s (2007)).

In certain embodiments, the multi-specific antigen-binding construct comprises two or more antigen-binding polypeptide constructs and a scaffold. In some embodiments, the multi-specific antigen-binding construct comprises two or more antigen-binding polypeptide constructs and a scaffold which is based on an IgG Fc region, an albumin or an albumin analog or derivative. In some embodiments, the multi-specific antigen-binding construct comprises a scaffold that is based on an Fc, which may be a dimeric or a heterodimeric Fc, comprising a first Fc polypeptide and a second Fc polypeptide, each comprising a CH3 sequence, and optionally a CH2 sequence.

In some embodiments, the multi-specific antigen-binding construct comprises an Fc which comprises first and second Fc polypeptides, and a first antigen-binding polypeptide construct is operably linked to the first Fc polypeptide and a second antigen-binding polypeptide construct is operably linked to the second Fc polypeptide. In some embodiments, the multi-specific antigen-binding construct comprises an Fc which comprises first and second Fc polypeptides, and a first antigen-binding polypeptide construct is operably linked to the C-terminus of the first Fc polypeptide or the second Fc polypeptide, with or without a linker. In some embodiments, the multi-specific antigen-binding construct comprises a heavy chain polypeptide comprising a CH1 and a VH and light chain polypeptide comprising a CL and a VL, in which a first antigen-binding polypeptide construct is operably linked to the N-terminus of the VL, the C-terminus of the CL, or the N-terminus of the VH, with or without a linker.

Also contemplated herein are multi-specific antigen-binding constructs that comprise three or more antigen-binding polypeptide constructs, including multi-specific antigen-binding constructs in an “Octopus antibody” or “dual-variable domain immunoglobulin” (DVD) format (see, e.g. U.S. Patent Application Publication No. US2006/0025576, and Wu et al., Nature Biotechnology 25:1290-1297 (2007)).

Certain embodiments contemplate that the multi-specific antigen-binding construct may also include a “Dual Acting FAb” or “DAF” comprising an antigen-binding polypeptide construct that binds to an immunotherapeutic as well as to the target tumour-associated antigen (see, U.S. Patent Application Publication No. US2008/0069820, for example).

### Scaffolds

In some embodiments, the multi-specific antigen-binding constructs described herein comprise a scaffold. A scaffold may be a peptide, polypeptide, polymer, nanoparticle or other chemical entity. Where the scaffold is a polypeptide, each antigen-binding polypeptide construct of the multi-specific antigen-binding construct may be linked to either the N- or C-terminus of the polypeptide scaffold. Multi-specific antigen-binding constructs comprising a polypeptide scaffold in which one or more of the antigen-binding polypeptide constructs are linked to a region other than the N- or C-terminus, for example, via the side chain of an amino acid with or without a linker, are also contemplated in certain embodiments.

In embodiments where the scaffold is a peptide or polypeptide, the antigen-binding construct may be linked to the scaffold by genetic fusion or chemical conjugation. In some embodiments, where the scaffold is a polymer or nanoparticle, the antigen-binding construct may be linked to the scaffold by chemical conjugation.

A number of protein domains are known in the art that comprise selective pairs of two different antigen-binding polypeptides and may be used to form a scaffold. An example is leucine zipper domains such as Fos and Jun that selectively pair together (Kostelny, et al., J Immunol, 148:1547-53 (1992); Wranik, et al., J. Biol. Chem., 287: 43331-43339 (2012)). Other selectively pairing molecular pairs include, for example, the barnase barstar pair (Deyev, et al., Nat Biotechnol, 21:1486-1492 (2003)), DNA strand pairs (Chaudri, et al., FEBS Letters, 450 (1-2):23-26 (1999)) and split fluorescent protein pairs (International Patent Publication No. WO 2011/13504).

Other examples of protein scaffolds include immunoglobulin Fc regions, albumin, albumin analogs and derivatives, toxins, cytokines, chemokines and growth factors. The use of protein scaffolds in combination with antigen-binding moieties has been described, for example, in Midler et al., J Biol Chem, 282:12650-12660 (2007); McDonaugh et al., Mol Cancer Ther, 11:582-593 (2012); Vallera et al., Clin Cancer Res, 11:3879-3888 (2005); Song et al., Biotech Appl Biochem, 45:147-154 (2006), and U.S. Patent Application Publication No. US2009/0285816.

For example, fusing antigen-binding moieties such as scFvs, diabodies or single chain diabodies to albumin has been shown to improve the serum half-life of the antigen-binding moieties (Müller et al., ibid.). Antigen-binding moieties may be fused at the N- and/or C-termini of albumin, optionally via a linker.

Derivatives of albumin in the form of heteromultimers that comprise two transporter polypeptides obtained by segmentation of an albumin protein such that the transporter polypeptides self-assemble to form quasi-native albumin have been described (see International Patent Publication Nos. WO 2012/116453 and WO 2014/012082). As a result of the segmentation of albumin, the heteromultimer includes four termini and thus can be fused to up to four different antigen-binding moieties, optionally via linkers.

In certain embodiments, the multi-specific antigen-binding construct comprises a protein scaffold. In some embodiments, the multi-specific antigen-binding construct comprises a protein scaffold that is based on an Fc region (as described below), an albumin or an albumin analog or derivative. In some embodiments, the multi-specific antigen-binding construct comprises a protein scaffold that is based on an albumin, for example human serum albumin (HSA), or an albumin analog or derivative. In some embodiments, the multi-specific antigen-binding construct comprises a protein scaffold that is based on an albumin derivative as described in International Patent Publication No. WO 2012/116453 or WO 2014/012082. In some embodiments, the multi-specific antigen-binding construct comprises two or more antigen-binding polypeptide constructs that are in the form of scFvs and a protein scaffold that is based on an albumin derivative as described in International Patent Publication No. WO 2012/116453 or WO 2014/012082.

### Fc Regions

In certain embodiments, the multi-specific antigen-binding constructs described herein comprise a scaffold that is based on a Fc region. The terms “Fc region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md. (1991). An “Fc polypeptide” of a dimeric Fc refers to one of the two polypeptides forming the dimeric Fc domain, i.e. a polypeptide comprising C-terminal constant regions of an immunoglobulin heavy chain that is capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some embodiments, the multi-specific antigen-binding construct comprises an Fc comprising one or two CH3 sequences. In some embodiments, the Fc is coupled, with or without one or more linkers, to a first antigen-binding polypeptide construct and a second antigen-binding polypeptide construct. In some embodiments, the Fc is based on a human Fc. In some embodiments, the Fc is based on a human IgG Fc, for example a human IgG1 Fc. In some embodiments, the Fc is a heterodimeric Fc. In some embodiments, the Fc comprises one or two CH2 sequences.

In some embodiments, the Fc comprises one or two CH3 sequences at least one of which comprises one or more amino acid modifications. In some embodiments, the Fc comprises one or two CH2 sequences, at least one of which comprises one or more amino acid modifications. In some embodiments, the Fc may be composed of a single polypeptide. In some embodiments, the Fc may be composed of multiple peptides, e.g. two polypeptides.

In some embodiments, the multi-specific antigen-binding construct comprises an Fc as described in International Patent Publication No. WO 2012/058768 or International Patent Publication No. WO 2013/063702.

### Modified CH3 Domains

In some embodiments, the multi-specific antigen-binding construct described herein comprises a heterodimeric Fc comprising a modified CH3 domain, wherein the modified CH3 domain is an asymmetrically modified CH3 domain. The heterodimeric Fc may comprise two heavy chain constant domain polypeptides: a first Fc polypeptide and a second Fc polypeptide, which can be used interchangeably provided that the Fc comprises one first Fc polypeptide and one second Fc polypeptide. Generally, the first Fc polypeptide comprises a first CH3 sequence and the second Fc polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to a modification where an amino acid at a specific position on a first CH3 sequence is different to the amino acid on a second CH3 sequence at the same position. For CH3 sequences comprising asymmetric amino acid modifications, the first and second CH3 sequence will typically preferentially pair to form a heterodimer, rather than a homodimer. These asymmetric amino acid modifications can be a result of modification of only one of the two amino acids at the same respective amino acid position on each sequence, or different modifications of both amino acids on each sequence at the same respective position on each of the first and second CH3 sequences. The first and second CH3 sequence of a heterodimeric Fc can comprise one or more than one asymmetric amino acid modification.

Table A provides the amino acid sequence of the human IgG1 Fc sequence, corresponding to amino acids 231 to 447 of the full-length human IgG1 heavy chain. The CH3 sequence comprises amino acids 341-447 of the full-length human IgG1 heavy chain.

Typically, an Fc includes two heavy chain polypeptide sequences (A and B) that are capable of dimerizing. In some embodiments, one or both polypeptide sequences of an Fc may include modifications at one or more of the following positions: L351, F405, Y407, T366, K392, T394, T350, 5400 and/or N390, using EU numbering.

In certain embodiments, the multi-specific antigen-binding construct comprises a heterodimeric Fc comprising a modified CH3 domain having a first polypeptide sequence that comprises amino acid modifications at positions F405 and Y407, and optionally further comprises an amino acid modification at position L351, and a second polypeptide sequence that comprises amino acid modifications at positions T366 and T394, and optionally further comprises an amino acid modification at position K392. In some embodiments, a first polypeptide sequence of the modified CH3 domain comprises amino acid modifications at positions F405 and Y407, and optionally further comprises an amino acid modification at position L351, and a second polypeptide sequence of the modified CH3 domain comprises amino acid modifications at positions T366 and T394, and optionally further comprises an amino acid modification at position K392, and the amino acid modification at position F405 is F405A, F4051, F405M, F405S, F405T or F405V; the amino acid modification at position Y407 is Y4071 or Y407V; the amino acid modification at position T366 is T366I, T366L or T366M; the amino acid modification at position T394 is T394W; the amino acid modification at position L351 is L351Y, and the amino acid modification at position K392 is K392F, K392L or K392M.

In some embodiments, a first polypeptide sequence of the Fc comprises amino acid modifications at positions F405 and Y407, and optionally further comprises an amino acid modification at position L351, and a second polypeptide sequence of the Fc comprises amino acid modifications at positions T366 and T394, and optionally further comprises an amino acid modification at position K392, and the amino acid modification at position F405 is F405A, F4051, F405M, F405S, F405T or F405V; the amino acid modification at position Y407 is Y4071 or Y407V; the amino acid modification at position T366 is T366I, T366L or T366M; the amino acid modification at position T394 is T394W; the amino acid modification at position L351 is L351Y, and the amino acid modification at position K392 is K392F, K392L or K392M, and one or both of the first and second polypeptide sequences of the Fc further comprises the amino acid modification T350V.

In certain embodiments, the multi-specific antigen-binding construct comprises a heterodimeric Fc comprising a modified CH3 domain having a first polypeptide sequence that comprises amino acid modifications at positions F405 and Y407, and optionally further comprises an amino acid modification at position L351, and a second polypeptide sequence that comprises amino acid modifications at positions T366 and T394, and optionally further comprises an amino acid modification at position K392, and the first polypeptide sequence further comprises an amino acid modification at one or both of positions S400 or Q347 and/or the second polypeptide sequence further comprises an amino acid modification at one or both of positions K360 or N390, where the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D.

In some embodiments, the multi-specific antigen-binding construct comprises a heterodimeric Fc comprising a modified CH3 domain comprising the modifications of any one of Variant 1, Variant 2, Variant 3, Variant 4 or Variant 5, as shown in Table A.

TABLE A
IgG1 Fc sequences

| Human IgG1 | APELLGGPSVFLFPPKPKDTLMISRTPEVTCVVVDVSH | |
|---|---|---|
| Fc sequence | EDPEVKFNWYVDGVEVHNAKTKPREEQYNSTYRVVS | |
| 231-447 | VLTVLHQDWLNGKEYKCKVSNKALPAPIEKTISKAKG | |
| (EU- | QPREPQVYTLPPSRDELTKNQVSLTCLVKGFYPSDIAV | |
| numbering) | EWESNGQPENNYKTTPPVLDSDGSFFLYSKLTVDKSR | |
| | WQQGNVFSCSVMHEALHNHYTQKSLSLSPGK | |
| | (SEQ ID NO: 9) | |
| Variant | | |
| IgG1 Fc | | |
| sequence | | |
| (231-447) | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

In some embodiments, the multi-specific antigen-binding construct comprises a heterodimeric Fc comprising a modified CH3 domain having a first CH3 sequence having amino acid modifications at positions F405 and Y407, and a second CH3 sequence having amino acid modifications at position T394. In some embodiments, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In some embodiments, the multi-specific antigen-binding construct comprises a heterodimeric Fc comprising a modified CH3 domain having a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In some embodiments, the heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In some embodiments, the multi-specific antigen-binding construct comprises a heterodimeric Fc comprising a modified CH3 domain having a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In some embodiments, the heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In some embodiments, the multi-specific antigen-binding construct comprises a heterodimeric Fc comprising a modified CH3 domain having a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In certain embodiments, the multi-specific antigen-binding construct comprises a heterodimeric Fc comprising a modified CH3 domain having a first polypeptide sequence that comprises an amino acid modification at position Y407, and a second polypeptide sequence that comprises amino acid modifications at positions T366 and K409. In some embodiments, a first polypeptide sequence of the modified CH3 domain comprises an amino acid modification at position Y407, and a second polypeptide sequence of the modified CH3 domain comprises amino acid modifications at positions T366 and K409, and the amino acid modification at position Y407 is Y407A, Y4071, Y407L or Y407V; the amino acid modification at position T366 is T366A, T366I, T366L, T366M or T366V, and the amino acid modification at position K409 is K409F, K4091, K409S or K409W.

In certain embodiments, the one or more asymmetric amino acid modifications comprised by the Fc can promote the formation of a heterodimeric Fc in which the heterodimeric CH3 domain has a stability that is comparable to a wild-type homodimeric CH3 domain. In some embodiments, the one or more asymmetric amino acid modifications promote the formation of a heterodimeric Fc domain in which the heterodimeric Fc domain has a stability that is comparable to a wild-type homodimeric Fc domain.

In some embodiments, the stability of the CH3 domain can be assessed by measuring the melting temperature (Tm) of the CH3 domain, for example by differential scanning calorimetry (DSC). In some embodiments, the one or more asymmetric amino acid modifications promote the formation of a heterodimeric Fc domain in which the CH3 domain has a stability as observed via the melting temperature (Tm) in a differential scanning calorimetry study that is within about 8° C., for example, within about 7° C., about 6° C., about 5° C., or about 4° C., of that observed for the corresponding symmetric wild-type homodimeric CH3 domain.

In some embodiments, the CH3 domain of the heterodimeric Fc may have a melting temperature (Tm) of about 68° C. or higher, about 70° C. or higher, about 72° C. or higher, 73° C. or higher, about 75° C. or higher, about 78° C. or higher, about 80° C. or higher, about 82° C. or higher, or about 84° C. or higher.

In some embodiments, a heterodimeric Fc comprising modified CH3 sequences can be formed with a purity of at least about 75% as compared to homodimeric Fc in the expressed product. In some embodiments, the heterodimeric Fc is formed with a purity greater than about 80%, greater than about 85%, greater than about 90%, greater than about 95% or greater than about 97%. In some embodiments, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98 or 99% when expressed.

Additional methods for modifying monomeric Fc polypeptides to promote heterodimeric Fc formation are known in the art and include, for example, those described in International Patent Publication No. WO 96/027011 (knobs into holes), in Gunasekaran et al. J Biol Chem, 285, 19637-46 (2010) (electrostatic design to achieve selective heterodimerization), in Davis et al., Prot Eng Des Sel, 23(4):195-202 (2010) (strand exchange engineered domain (SEED) technology), and in Labrijn et al., Proc Natl Acad Sci USA, 110(13):5145-50 (2013) (Fab-arm exchange).

### CH2 Domains

In some embodiments, the multi-specific antigen-binding construct comprises an Fc comprising a CH2 domain. One example of a CH2 domain of an Fc is amino acids 231-340 of the sequence shown in Table A. Several effector functions are mediated by Fc receptors (FcRs), which bind to the Fc of an antibody.

The term “Fc receptor” (“FcR”) is used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). The term “FcR” also includes in certain embodiments the neonatal receptor, FcRn, which is responsible for the transfer of maternal IgGs to the fetus (Guyer et al., J. Immunol. 117:587 (1976) and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some embodiments, the Fc comprised by the multi-specific antigen-binding construct may comprise one or more modifications to promote selective binding of Fc-gamma receptors.

Non-limiting examples of modifications that alter the binding of the Fc by FcRs include: S298A/E333A/K334A and S298A/E333A/K334A/K326A (Lu, et al., J Immunol Methods, 365(1-2): 132-41 (2011)); F243L/R292P/Y300LN305I/P396L and F243L/R292P/Y300L/L235V/P396L (Stavenhagen, et al., Cancer Res, 67(18):8882-90 (2007) and Nordstrom J L, et al., Breast Cancer Res, 13(6):R123 (2011)); F243L (Stewart, et al., Protein Eng Des Sel. 24(9):671-8 (2011)); S298A/E333A/K334A (Shields, et al., J Biol Chem, 276(9):6591-604 (2001)); S239D/I332E/A330L and S239D/I332E (Lazar, et al., Proc Natl Acad Sci USA, 103(11):4005-10 (2006)); S239D/S267E and S267E/L328F (Chu, et al., Mol Immunol, 45(15):3926-33 (2008)). Other examples include S239D/D265S/S298A/I332E; S239E/S298A/K326A/A327H; G237F/S298A/A330L/I332; S239D/I332E/S298A; S239D/K326E/A330L/I332E/S298A; G236A/S239D/D270L/I332E; S239E/S267E/H268D; L234F/S267E/N325L; G237FN266L/S267D, and other mutations described in International Patent Publication No. WO 2011/120134.

Additional modifications that affect Fc binding by FcRs are described in Therapeutic Antibody Engineering (Strohl & Strohl, Woodhead Publishing series in Biomedicine No 11, ISBN 1 907568 37 9, October 2012, page 283).

Fc regions that comprise asymmetric modifications that affect binding by FcRs are described in International Patent Publication No. WO 2014/190441. In some embodiments, the multi-specific antigen-binding construct comprises an Fc including a CH2 domain comprising one or more asymmetric amino acid modifications. In some embodiments, the multi-specific antigen-binding construct comprises an Fc including a CH2 domain comprising asymmetric modifications that provide superior biophysical properties, for example stability and/or ease of manufacture, relative to an antigen-binding construct which does not include the asymmetric modifications.

### Additional Modifications

In some embodiments, a multi-specific antigen-binding construct comprising an Fc region may include modifications to improve its ability to mediate effector function. Such modifications are known in the art and include afucosylation, or engineering of the affinity of the Fc towards an activating receptor, mainly FcγRIIIa for ADCC, and towards C1q for CDC.

Methods of producing antibodies with little or no fucose on the Fc glycosylation site (Asn 297, EU numbering) without altering the amino acid sequence are well known in the art. For example, the GlymaX® technology (ProBioGen AG) (see von Horsten et al., Glycobiology, 20(12):1607-18 (2010)) and U.S. Pat. No. 8,409,572. In certain embodiments, the multi-specific antigen-binding constructs may be aglycosylated. In this context, the multi-specific antigen-binding constructs may be fully afucosylated (i.e. they contain no detectable fucose) or they may be partially afucosylated such that the multi-specific antigen-binding construct contains less than 95%, less than 85%, less than 75%, less than 65%, less than 55%, less than 45%, less than 35%, less than 25%, less than 15% or less than 5% of the amount of fucose normally detected for a similar construct produced by a mammalian expression system.

Fc modifications reducing FcγR and/or complement binding and/or effector function are known in the art and include those described above. Various publications describe strategies that have been used to engineer antibodies with reduced or silenced effector activity (see, for example, Strohl, Curr Opin Biotech 20:685-691 (2009), and Strohl & Strohl, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc (see also, U.S. Patent Publication No. 2011/0212087, International Patent Publication No. WO 2006/105338, U.S. Patent Publication No. 2012/0225058, U.S. Patent Publication No. 2012/0251531 and Strop et al., J. Mol. Biol. 420: 204-219 (2012)).

Specific, non-limiting examples of known amino acid modifications to reduce FcγR or complement binding to the Fc include those identified in Table B.

TABLE B
Modifications to reduce FcγR or complement binding to the Fc

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Alexion | IgG2/IgG4combo |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glycosylated |
| Medimmune | L234F/L235E/P331S |
| Trubion | Hinge mutant, possibly C226S/P230S |

In some embodiments, the multi-specific antigen-binding construct comprises an Fc that comprises at least one amino acid modification identified in Table B. In some embodiments, the multi-specific antigen-binding construct comprises an Fc that comprises amino acid modification of at least one of L234, L235, or D265. In some embodiments, the multi-specific antigen-binding construct comprises an Fc that comprises amino acid modifications at L234, L235 and D265. In some embodiments, the multi-specific antigen-binding construct comprises an Fc that comprises the amino acid modifications L234A, L235A and D265S.

### Linkers

In some embodiments, the multi-specific antigen-binding constructs described herein include two or more antigen-binding polypeptide constructs and one or more linkers. The linkers may, for example, function to join two domains of an antigen-binding polypeptide construct (such as the VH and VL of an scFv or diabody), or they may function to join two antigen-binding polypeptide constructs together (such as two or more Fabs or sdAbs), or they may function to join an antigen-binding polypeptide construct to a scaffold. In some embodiments, the multi-specific antigen-binding constructs may comprise multiple linkers (i.e. two or more), for example, a multi-specific antigen-binding construct one or more scFvs linked to a scaffold may comprise a linker joining the VH and VL of the scFv and a linker joining the scFv to the scaffold. Appropriate linkers are known in the art and can be readily selected by the skilled artisan based on the intended use of the linker (see, for example, Müller & Kontermann, “Bispecific Antibodies” in Handbook of Therapeutic Antibodies, Wiley-VCH Verlag GmbH & Co. (2014)).

Useful linkers include glycine-serine (GlySer) linkers, which are well-known in the art and comprise glycine and serine units combined in various orders. Examples include, but are not limited to, (GS)ₙ, (GSGGS)ₙ, (GGGS)ₙ and (GGGGS)ₙ, where n is an integer of at least one, typically an integer between 1 and about 10, for example, between 1 and about 8, between 1 and about 6, or between 1 and about 5.

Other useful linkers include sequences derived from immunoglobulin hinge sequences. The linker may comprise all or part of a hinge sequence from any one of the four IgG classes and may optionally include additional sequences. For example, the linker may include a portion of an immunoglobulin hinge sequence and a glycine-serine sequence. A non-limiting example is a linker that includes approximately the first 15 residues of the IgG1 hinge followed by a GlySer linker sequence, such as those described above, that is about 10 amino acids in length.

The length of the linker will vary depending on its application. Appropriate linker lengths can be readily selected by the skilled person. For example, when the linker is to connect the VH and VL domains of an scFv, the linker is typically between about 5 and about 20 amino acids in length, for example, between about 10 and about 20 amino acid in length, or between about 15 and about 20 amino acids in length. When the linker is to connect the VH and VL domains of a diabody, the linker should be short enough to prevent association of these two domains within the same chain. For example, the linker may be between about 2 and about 12 amino acids in length, such as, between about 3 and about 10 amino acids in length, or about 5 amino acids in length.

In some embodiments, when the linker is to connect two Fab fragments, the linker may be selected such that it maintains the relative spatial conformation of the paratopes of a F(ab′) fragment, and is capable of forming a covalent bond equivalent to the disulphide bond in the core hinge of IgG. In this context, suitable linkers include IgG hinge regions such as, for example those from IgG1, IgG2 or IgG4. Modified versions of these exemplary linkers can also be used. For example, modifications to improve the stability of the IgG4 hinge are known in the art (see for example, Labrijn et al., Nature Biotechnology, 27:767-771 (2009)).

In some embodiments, the multi-specific antigen-binding construct comprises a linker operably linking an antigen-binding polypeptide construct to a scaffold as described herein. In some aspects, the multi-specific antigen-binding construct comprises an Fc coupled to the one or more antigen-binding polypeptide constructs with one or more linkers. In some aspects, the multi-specific antigen-binding construct comprises an Fc coupled to the heavy chain of each antigen-binding polypeptide construct by a linker.

### Immunotherapeutics

The multi-specific antigen-binding constructs described herein comprise an antigen-binding polypeptide construct that binds to an immunotherapeutic. The immunotherapeutic may be an effector cell, such as a T-cell or a NK cell, engineered to express an antigen-binding domain, or the immunotherapeutic may be a therapeutic agent, such as an antibody or antibody fragment, capable of binding to a T-cell and to a tumour-associated antigen.

In certain embodiments, the immunotherapeutic is an engineered T-cell or NK cell. Typically, the antigen-binding domain comprised by the T-cell or NK cell is part of an engineered receptor. In some embodiments, the antigen-binding domain comprised by the engineered T-cell or NK cell may be, for example, part of a chimeric antigen receptor (CAR) or a T-cell receptor (TCR), such as a transgenic or recombinant TCR. In accordance with these embodiments, the multi-specific antigen-binding construct binds to an extracellular portion of the CAR or TCR. The multi-specific antigen-binding construct may bind to the antigen-binding domain of the CAR or TCR, or it may bind to an extracellular region of the CAR or TCR that is not involved in antigen binding.

As is known in the art, CAR and TCR constructs may be designed to include a “tag,” which is typically a short amino acid sequence that is specifically recognized by an antibody. In some embodiments, the immunotherapeutic is a T-cell or a NK cell engineered to express a CAR or TCR which includes a tag. In the context of such embodiments, the multi-specific antigen-binding construct may bind to the tag or it may bind to a region of the CAR or TCR other than the tag. In some embodiments in which the immunotherapeutic is a T-cell or a NK cell engineered to express a CAR or TCR which includes a tag, the multi-specific antigen-binding construct binds to a region of the CAR or TCR other than the tag.

In some embodiments, the immunotherapeutic is a T-cell or a NK cell engineered to express a CAR or TCR which does not include a tag. In some embodiments, the immunotherapeutic is a T-cell or a NK cell engineered to express a CAR or TCR which does not include a tag or any heterologous tumour-associated antigens or fragments of tumour-associated antigens.

In certain embodiments, the immunotherapeutic is a T-cell or a NK cell engineered to express a CAR and the multi-specific antigen-binding construct binds to an extracellular part of the CAR. As is known in the art, a CAR is a cell-surface receptor comprising an extracellular domain, a transmembrane domain and a cytoplasmic domain in a combination that is not naturally found in a single protein. The extracellular domain comprises an antigen-binding domain, which may be an antibody or antibody fragment. The antibody or antibody fragment may be a human antibody or fragment, humanized antibody or fragment or a non-human antibody or fragment. Typically, the antigen-binding domain is an antibody fragment, such as a Fab or scFv. Most typically, the antigen-binding domain is an scFv. The extracellular domain also typically comprises a spacer (or hinge) region linking the antigen-binding domain to the transmembrane domain. The spacer region may be derived from an immunoglobulin, such as IgG1 or IgG4, or it may be derived from alternative cell-surface proteins, including, but not limited to, CD4, CD8, or CD28.

The transmembrane domain of the CAR links the extracellular domain to the cytoplasmic domain. Typically, the transmembrane domain is derived from a type I membrane protein, such as CD3 zeta, CD4, CD8 or CD28. In some instances, the transmembrane domain may be modified by amino acid substitution to avoid binding of such domains to the transmembrane domains of the same or different surface membrane proteins to minimize interactions with other members of the receptor complex. Other examples of transmembrane domains include those derived from the alpha, beta or zeta chain of the T-cell receptor, CD3 epsilon, CD45, CD5, CD9, CD16, CD22, CD33, CD37, CD64, CD80, CD86, CD134, CD137, CD154 or ICOS.

The cytoplasmic domain of the CAR comprises at least one intracellular signalling domain and is responsible for activation of at least one of the normal effector functions of the immune cell into which the CAR has been placed. The term “effector function” refers to a specialized function of a cell. Effector function of a T-cell, for example, may be cytolytic activity or helper activity including the secretion of cytokines. Thus, the term “intracellular signalling domain” refers to the portion of a protein that transduces the effector function signal and directs the cell to perform a specialized function. Examples of intracellular signalling domains frequently used in CARs include the cytoplasmic sequences of the TCR and co-receptors that act in concert to initiate signal transduction following antigen receptor engagement, as well as derivatives or variants of these sequences having the same functional capability.

It is known that signals generated through the TCR alone are insufficient for full activation of the T-cell and that a secondary or co-stimulatory signal is also required. Thus, T-cell activation can be said to be mediated by two distinct classes of cytoplasmic signalling sequence: those that initiate antigen-dependent primary activation through the TCR (primary cytoplasmic signalling sequences) and those that act in an antigen-independent manner to provide a secondary or co-stimulatory signal (secondary cytoplasmic signalling sequences).

Primary cytoplasmic signalling sequences regulate primary activation of the TCR complex either in a stimulatory way, or in an inhibitory way. Primary cytoplasmic signalling sequences that act in a stimulatory manner may contain signalling motifs which are known as immunoreceptor tyrosine-based activation motifs or ITAMs.

Examples of ITAM containing primary cytoplasmic signalling sequences that may be used in CARs include those derived from TCR zeta, FcR gamma, FcR beta, CD3 gamma, CD3 delta, CD3 epsilon, CD3 zeta, CD5, CD22, CD79a, CD79b and CD66d. Typically, the cytoplasmic domain in a CAR will comprise a cytoplasmic signalling sequence derived from CD3 zeta.

The cytoplasmic domain of the CAR may comprise an ITAM containing primary cytoplasmic signalling sequence by itself or combined with one or more co-stimulatory domains. A co-stimulatory domain is derived from the intracellular domain of a co-stimulatory molecule. A co-stimulatory molecule is a cell surface molecule other than an antigen receptor that is required for an efficient response of lymphocytes to an antigen. Examples of such molecules include CD27, CD28, 4-1BB (CD 137), OX40, CD30, CD40, PD-1, ICOS, lymphocyte function-associated antigen-1 (LFA-1), CD2, CD7, LIGHT, NKG2C and B7-H3. Typically, CARs comprise one or more co-stimulatory domains derived from 4-1BB, CD28 or OX40. First generation CARs, for example, include only a CD3 zeta-derived intracellular signalling domain, whereas second generation CARs include a CD3 zeta-derived intracellular signalling domain, together with a co-stimulatory domain derived from either 4-1BB or CD28. Third generation CARs include a CD3 zeta-derived intracellular signalling domain, together with two co-stimulatory domains, the first co-stimulatory domain derived from either 4-1BB or CD28, and the second co-stimulatory domain derived from 4-1BB, CD28 or OX40.

Examples of CAR constructs currently in development, and their component domains are provided in Table 1.

TABLE 1
Examples of CAR constructs

| | | Hinge/Trans- | |
|---|---|---|---|
| | | membrane | Cytoplasmic |
| Institute | scFv | Domain | Domain |
| NCI | FMC63 (anti- | CD28 | CD28, CD3 zeta |
| | CD 19) | | |
| Baylor | FMC63 (anti- | IgG-CD28 | CD28, CD3 zeta |
| | CD19) | | |
| City of Hope | FMC63 (anti- | IgG4-Fc | CD28, CD3 zeta |
| | CD19) | | |
| M D Anderson | FMC63 (anti- | IgG4-Fc | CD28, CD3 zeta |
| Cancer Center | CD19) | | |
| Fred Hutchinson | FMC63 (anti- | IgG1-CD4 | CD28, CD3 zeta |
| | CD19) | | |
| Memorial Sloan | SJ25C1 (anti- | CD28 | CD28, CD3 zeta |
| Kettering Cancer | CD19) | | |
| Center | | | |
| University of | FMC63 (anti- | CD8 | 4-1BB, CD3 zeta |
| Pennsylvania | CD19) | | |
| Fred Hutchinson | FMC63 (anti- | IgG1-CD4 | 4-1BB, CD3 zeta |
| | CD19) | | |

* Adapted from Batlevi et al., Nature Reviews Clinical Oncology, 13: 25-40 (2016)

In certain embodiments, the immunotherapeutic targeted by the multi-specific antigen-binding construct is a T-cell engineered to express a CAR (CAR-T). In some embodiments, the immunotherapeutic is a CAR-T and an antigen-binding polypeptide construct of the multi-specific antigen-binding construct binds to the antigen-binding domain of the CAR. In accordance with such embodiments, the antigen-binding polypeptide construct may comprise an anti-idiotype antibody or antigen-binding fragment thereof. Antigens targeted by CARs are typically cell surface tumour-associated antigens.

As used herein “tumour-associated antigen” refers to an antigen that is expressed by cancer cells. A tumour-associated antigen may or may not be expressed by normal cells. When a tumour-associated antigen is not expressed by normal cells (i.e. when it is unique to tumour cells) it may also be referred to as a “tumour-specific antigen.” When a tumour-associated antigen is not unique to a tumour cell, it is also expressed on a normal cell under conditions that fail to induce a state of immunologic tolerance to the antigen. The expression of the antigen on the tumour may occur under conditions that enable the immune system to respond to the antigen. Tumour-associated antigens may be antigens that are expressed on normal cells during fetal development when the immune system is immature and unable to respond, or they may be antigens that are normally present at low levels on normal cells but which are expressed at much higher levels on tumour cells. Those tumour-associated antigens of greatest clinical interest are differentially expressed compared to the corresponding normal tissue and allow for a preferential recognition of tumour cells by specific T-cells or immunoglobulins.

Examples of tumour-associated antigens targeted by CARs or engineered TCRs currently in clinical development include NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), myelin, NKG2D (Natural Killer Group 2D) ligands, PSMA (prostate specific membrane antigen), and ROR1 (type I receptor tyrosine kinase-like orphan receptor).

Accordingly, in certain embodiments, the multi-specific antigen-binding construct comprises an antigen-binding polypeptide construct derived from an anti-idiotype antibody or antigen-binding fragment thereof, wherein the anti-idiotype antibody is an anti-idiotype antibody of NY-ESO-1, MART-1, HPV E6, BCMA, CD123, CD133, CD171, CD19, CD20, CD22, CD30, CD33, CEA, EGFR, EGFRvIII, EpCAM, EphA2, disialoganglioside GD2, GPC3, HER2, IL13Ralpha2, LeY, MAGE-A3, melanoma glycoprotein, mesothelin, MUC1, myelin, NKG2D ligands, PSMA or ROR1. In some embodiments, the multi-specific antigen-binding construct comprises an antigen-binding polypeptide construct derived from an anti-idiotype antibody specific for an anti-CD19 antibody, or antigen-binding fragment of the anti-idiotype antibody. In some embodiments, the multi-specific antigen-binding construct comprises an antigen-binding polypeptide construct derived from an anti-idiotype antibody specific for an anti-mesothelin antibody, or antigen-binding fragment of the anti-idiotype antibody.

A number of anti-idiotype antibodies are known in the art. For example, International Patent Application Publication No. WO 2014/190273 and Jena et al. PLOS One, 8:3 e57838 (2013), describe an anti-idiotype antibody (mAb clone no. 136.20.1) that recognizes the anti-CD19 scFv FMC63, which is used in a number of CAR constructs in current development. The sequence of the VH and VL of mAb clone no. 136.20.1 are provided in Table 5 (SEQ ID NOs: 1 and 2, respectively).

In certain embodiments, the multi-specific antigen-binding construct comprises an antigen-binding polypeptide construct derived from an anti-idiotype antibody specific for an anti-CD19 antibody, or antigen-binding fragment of the anti-idiotype antibody, that may have one or more of the same CDRs (i.e. one or more of, or all of, VH CDR1, VH CDR2, CH CDR3, VL CDR1, VL CDR2, and VL CDR3, using the Kabat definition, the Chothia definition, or a combination of the Kabat and Chothia definitions) as mAb clone no. 136.20.1. In some embodiments, the multi-specific antigen-binding construct comprises an antigen-binding polypeptide construct derived from an anti-idiotype antibody specific for an anti-CD19 antibody, or antigen-binding fragment of the anti-idiotype antibody, that may have one or more (for example, two) variable regions from mAb clone no. 136.20.1. In some embodiments, the multi-specific antigen-binding construct comprises an antigen-binding polypeptide construct derived from an anti-idiotype antibody specific for an anti-CD19 antibody, or antigen-binding fragment of the anti-idiotype antibody, that binds to the same epitope as mAb clone no. 136.20.1.

Other examples of anti-idiotype antibodies include those that are commercially available from AbD Serotec®, an anti-idiotype antibody specific for an anti-CD22 antibody described in International Patent Publication No. WO 2013/188864, an anti-idiotype antibody specific for an anti-CEA antibody described in International Patent Publication No. WO 97/34636, an anti-idiotype antibody specific for an anti-GD2 antibody described in U.S. Pat. No. 5,935,821, and an anti-idiotype antibody specific for an anti-NY-ESO-1 antibody described in Jakka et al., Anticancer Research, 33:10, 4189-420 (2013). Custom anti-idiotype antibodies may also be obtained from AbD Serotec®.

Alternatively, anti-idiotype antibodies to CARs targeting CD19 or other tumour-associated antigens may be made according to the method described in Jena et al., PLOS One, 8:3 e57838 (2013), and used for the construction of an anti-idiotype antigen-binding polypeptide construct.

In some embodiments, the multi-specific antigen-binding construct comprises an antigen-binding polypeptide construct that binds to an extracellular region of a CAR that is not involved in antigen binding. For example, in certain embodiments, the antigen-binding polypeptide construct may bind to a hinge region of the CAR. In some embodiments, the hinge region may be an scFv-CD28 or scFv-CD8 junction, which comprises neo-epitopes that may be targeted by the antigen-binding polypeptide constructs. In some embodiments, the hinge region may comprise mutated (Fc-binding null) IgG CH2/3 that may be targeted by the antigen-binding polypeptide constructs. In some embodiments, the hinge region may comprise a spacer such as a Strep-tag II as described by Liu et al. (Nature Biotechnology, 34, 430-434 (2016)) that may be targeted by the antigen-binding polypeptide constructs.

An example of an anti-CAR antibody that binds to a hinge region of the CAR molecule is the 2D3 antibody described in International Patent Application Publication No. WO 2014/190273, which binds to an IgG4 CH2-CH3 hinge region. In some embodiments, the multi-specific antigen-binding construct comprises an antigen-binding polypeptide construct that binds to an IgG4 CH2-CH3 hinge region. In some embodiments, the multi-specific antigen-binding construct comprises an antigen-binding polypeptide construct that binds to an IgG4 CH2-CH3 hinge region and has one or more of the same CDRs (i.e. one or more of, or all of, VH CDR1, VH CDR2, CH CDR3, VL CDR1, VL CDR2 and VL CDR3) as 2D3, or has one or more (for example, two) variable regions of 2D3 as described in WO 2014/190273. In some embodiments, the multi-specific antigen-binding construct comprises an antigen-binding polypeptide construct that binds to an IgG4 CH2-CH3 hinge region and binds to the same epitope as 2D3 as described in WO 2014/190273.

In certain embodiments, the immunotherapeutic is an engineered T-cell or NK cell that expresses an engineered TCR and the multi-specific antigen-binding construct binds an extracellular part of the TCR.

Native TCRs comprise two different protein chains, an alpha and beta chain. The TCRalpha/beta pair is expressed on the T-cell surface in a complex with CD3 epsilon, CD3 gamma, CD3 delta and CD3 epsilon. In an engineered TCR, the native alpha and beta chains of a TCR are modified to introduce an improved or new specificity for a tumour-associated antigen. As the engineered TCR retains most of the native sequence of the alpha and beta chains, when a multi-specific antigen-binding construct as described herein comprises a antigen-binding polypeptide construct targeting an engineered TCR immunotherapeutic, the antigen-binding polypeptide construct will typically target the antigen-binding domain of the TCR. For example, in certain embodiments in which the immunotherapeutic is a T-cell or NK cell comprising an engineered TCR, the antigen-binding polypeptide construct of the multi-specific antigen-binding construct may be derived from an anti-idiotype antibody or fragment thereof, as described above.

Antigen-binding polypeptide constructs that bind to a non-antigen binding region of an engineered TCR are also contemplated in some embodiments, for example, where the engineered TCR includes one or more non-native sequences in the non-antigen binding domains to which the antigen-binding polypeptide construct could be targeted. In some embodiments, the antigen-binding polypeptide construct is targeted to the engineered TCR Valpha or Vbeta region. In such embodiments, the antigen-binding polypeptide construct may also bind to native TCRs as engineered TCR V region domains would also be present in the endogenous TCR repertoire, but at very low frequencies.

As TCRs bind to antigens presented in the context of an MHC, engineered TCRs may be targeted to intracellular tumour-associated antigens. Examples of intracellular tumour-associated antigens include, but are not limited to, peptides derived from NY-ESO-1, MART-1, WT-1, HPV E6 or HPV E7. Accordingly, in certain embodiments, the multi-specific antigen-binding construct comprises an antigen-binding polypeptide construct that is derived from an anti-TCR idiotype antibody, wherein the TCR specifically binds MHC complexes containing peptides derived from, for example, NY-ESO, MART-1, WT-1, HPV-E6 or HPV-E7, or an antigen-binding fragment of such an anti-TCR idiotype antibody. In some embodiments, the multi-specific antigen-binding construct comprises an antigen-binding polypeptide construct derived from an anti-TCR idiotype (or clonotype) antibody, wherein the TCR specifically binds MHC complexes containing peptides derived from NY-ESO, MART-1 or HPV-E6, or an antigen-binding fragment of such an anti-TCR idiotype/clonotype antibody. Anti-TCR idiotype/clonotype antibodies are well-known in the art and include, but are not limited to, 6B11 (Montoya, et al., Immunology, 122(1):1-14 (2007)) and KJI-26 (Haskins, et al., J Exp Med, 157(4):1149-69 (1983)).

In certain embodiments, the immunotherapeutic may be a therapeutic agent, such as an antibody or antibody fragment, capable of binding to a T-cell and to a tumour-associated antigen. In accordance with these embodiments, the therapeutic agent typically comprises at least two antigen-binding domains, one of which binds to an extracellular portion of the T-cell and the other binds to the tumour-associated antigen. Examples of such therapeutic agents include, for example, bispecific T-cell engagers (BiTEs), such as blinotumumab, which targets CD3 and CD19, and solitomab, which targets CD3 and EpCAM, and other “T-cell engaging” antibodies or antibody fragments. In accordance with these embodiments, the antigen-binding polypeptide construct of the multi-specific antigen-binding construct typically binds to the antigen-binding domain of the therapeutic agent. For example, in some embodiments, the antigen-binding polypeptide construct of the multi-specific antigen-binding construct may be derived from an anti-idiotype antibody or fragment thereof, as described above. In some embodiments, the antigen-binding polypeptide construct is derived from an anti-idiotype antibody specific for an anti-CD19 antibody or an anti-EpCAM antibody, or an antigen-binding fragment of the anti-idiotype antibody. Examples of such anti-idiotype antibodies include those described above.

The immunotherapeutic targeted antigen-binding polypeptide construct comprised by the multi-specific antigen-binding constructs described herein may be in any one of various known formats, including for example, a Fab format, scFv format or sdAb format. In certain embodiments, the immunotherapeutic targeted antigen-binding polypeptide construct may be in a Fab or scFv format. In some embodiments, the immunotherapeutic targeted antigen-binding polypeptide construct may be in a non-immunoglobulin based antibody mimetic format as described above.

### Tumour-Associated Antigens

The multi-specific antigen-binding constructs described herein comprise at least one antigen-binding polypeptide construct that binds to a tumour-associated antigen (TAA). In certain embodiments, the multi-specific antigen-binding constructs comprise two or more TAA-binding polypeptide constructs. When the multi-specific antigen-binding constructs comprise two or more TAA-binding polypeptide constructs, each of the TAA-binding polypeptide constructs may bind a different TAA, or two or more of the TAA-binding polypeptide constructs may bind different epitopes on the same TAA. TAAs are defined above and include antigens that are expressed only by tumour cells (tumour-specific antigens), as well as antigens that are expressed on both tumour cells and normal cells, but typically at a lower level on normal cells.

Selection of a TAA as a target for the multi-specific antigen-binding constructs described herein will be dependent on the intended use of the multi-specific antigen-binding construct. As described above, the multi-specific antigen-binding construct binds to an immunotherapeutic that targets a TAA, and also itself binds to a TAA. The TAA epitope bound by the multi-specific antigen-binding construct is different to the TAA epitope bound by the immunotherapeutic. Thus, the multi-specific antigen-binding construct and the immunotherapeutic may both target the same TAA but bind to different epitopes on the antigen molecule, or they may target different TAAs. In certain embodiments, the multi-specific antigen-binding construct and the immunotherapeutic target different TAAs. When the TAAs targeted by the multi-specific antigen-binding construct and the immunotherapeutic are different, the different antigens will typically both be associated with the same type of cancer. However, targeting TAAs that are associated with different types of cancer is also contemplated in certain embodiments.

Examples of TAAs that may be targeted by the multi-specific antigen-binding construct include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, EGFR, EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Le^{γ}, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumour necrosis antigens, TRAG-3, TRAIL receptors, VEGF, VEGFR and WT-1 (see, e.g., Sensi et al., Clin Cancer Res, 12:5023-32 (2006); Parmiani et al., J Immunol, 178:1975-79 (2007); Novellino et al., Cancer Immunol Immunother, 54:187-207 (2005)).

In certain embodiments, the TAA targeted by the multi-specific antigen-binding construct is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor and VEGF. In some embodiments, the TAA is an antigen expressed by malignant B cells, such as CD19, CD20, CD22, CD25, CD38, CD40, CD45, CD74, CD80, CTLA-4, IGF-R1, IL6, PD-1, TRAILR2 or VEGF.

In some embodiments, the TAA targeted by the multi-specific antigen-binding construct is an antigen associated with a solid tumour. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{γ}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

The TAA-binding polypeptide construct(s) comprised by the multi-specific antigen-binding constructs may be in any one of various known formats, including for example, a Fab format, scFv format or sdAb format. In some embodiments, the TAA-binding polypeptide construct comprised by the multi-specific antigen-binding construct may be a natural ligand for the TAA, or a functional fragment of the natural ligand. In certain embodiments, the multi-specific antigen-binding construct comprises more than one TAA-binding polypeptide construct. In such embodiments, the TAA-binding polypeptide constructs may be linked together, for example, as a Fab-Fab, an scFv-scFv or a Fab-scFv, as shown in FIG. 1B. Other formats are also contemplated including, for example, multi-specific antigen binding constructs comprising an Fc and two or more antigen binding polypeptide constructs each targeting a TAA in which the antigen binding polypeptide constructs are linked to different parts of the Fc. In certain embodiments, the one or more TAA-binding polypeptide constructs are in a Fab or scFv format, or a combination thereof.

In certain embodiments, the antigen-binding polypeptide constructs can be derived from known antibodies directed against a TAA or their binding domains or fragments of the antibodies. Examples of types of binding domains include Fab fragments, scFvs, and sdAbs. Furthermore, if the antigen-binding moieties of a known anti-TAA antibody or binding domain is a Fab, the Fab can be converted to an scFv. Likewise, if the antigen-binding moiety of a known anti-TAA antibody or binding domain is an scFv, the scFv can be converted to a Fab. Methods of converting between types of antigen-binding domains are known in the art (see, for example, methods for converting an scFv to a Fab format described in Zhou et al., Mol Cancer Ther, 11:1167-1476 (2012)).

Known antibodies directed against TAAs may be commercially obtained from a number of known sources. For example, a variety of antibody secreting hybridoma lines are available from the American Type Culture Collection (ATCC, Manassas, Va.). A number of antibodies against various TAAs have been deposited at the ATCC and/or have published variable region sequences and may be used to prepare the multi-specific antigen-binding constructs in certain embodiments. The skilled artisan will appreciate that antibody sequences or antibody-secreting hybridomas against various TAAs may be obtained by a simple search of the ATCC, NCBI and/or USPTO databases.

Particular TAA-targeted antibodies that may be of use in preparing the multi-specific antigen-binding constructs described herein include, but are not limited to, LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), trastuzumab (anti-HER2), pertuzumab (anti-HER2), polatuzumab (anti-CD79b) and anetumab (anti-mesothelin).

In certain embodiments, the TAA-binding polypeptide construct comprised by the multi-specific antigen binding construct is derived from a humanized, or chimeric version of a known antibody.

“Humanized” forms of non-human (e.g. rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody may optionally also comprise at least a portion of an immunoglobulin constant region (Fc), typically that of a human immunoglobulin. For further details, see Jones et al., Nature, 321:522-525 (1986); Riechmann et al., Nature, 332:323-329 (1988); and Presta, Curr. Op. Struct. Biol., 2:593-596 (1992).

Alternatively, antibodies to a specific target TAA of interest may be generated by standard techniques and used as a basis for the preparation of the TAA-binding polypeptide construct(s) of the multi-specific antigen-binding construct.

### Methods of Preparing the Multi-Specific Antigen-Binding Constructs

The multi-specific antigen-binding constructs described herein may be produced using standard recombinant methods known in the art (see, e.g., U.S. Pat. No. 4,816,567 and “Antibodies: A Laboratory Manual,” 2^{nd} Edition, Ed. Greenfield, Cold Spring Harbor Laboratory Press, New York, 2014).

Typically, for recombinant production of a multi-specific antigen-binding construct, nucleic acid encoding the multi-specific antigen-binding construct is isolated and inserted into one or more vectors for further cloning and/or expression in a host cell. Such nucleic acid may be readily isolated and sequenced using conventional procedures (e.g. by using oligonucleotide probes that are capable of binding specifically to genes encoding the heavy and light chains of the multi-specific antigen-binding construct).

Suitable host cells for cloning or expression of antigen-binding construct-encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles and birds), ciliates, plants (including but not limited to, monocots, dicots and algae), fungi, yeasts, flagellates, microsporidia, protists, and the like.

As used herein, the term “prokaryote” refers to prokaryotic organisms. For example, a non-eukaryotic organism can belong to the Eubacteria (including but not limited to, Escherichia coli, Therms thermophilus, Bacillus stearothermophilus, Pseudomonas fluorescens, Pseudomonas aeruginosa, Pseudomonas putida, and the like) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, and the like) phylogenetic domain.

For example, a multi-specific antigen-binding construct may be produced in bacteria, in particular when glycosylation and Fc effector function are not needed. For expression of antigen-binding construct fragments and polypeptides in bacteria, see, for example, U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antigen-binding construct may be isolated from the bacterial cell paste in a soluble fraction and can be further purified.

In addition to prokaryotes, eukaryotic microbes such as filamentous fungi or yeast are suitable cloning or expression hosts for multi-specific antigen-binding construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antigen-binding constructs are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol., 36:59 (1977)); baby hamster kidney cells (BHK); mouse sertoli cells (TM4 cells as described, e.g., in Mather, Biol Reprod, 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumour (MMT 060562); TRI cells, as described, e.g., in Mather et al., Annals N.Y. Acad Sci, 383:44-68 (1982); MRC 5 cells; and FS4 cells. Other useful mammalian host cell lines include Chinese hamster ovary (CHO) cells, including DHFR⁻CHO cells (Urlaub et al., Proc Natl Acad Sci USA, 77:4216 (1980)); and myeloma cell lines such as Y0, NS0 and Sp2/0. For a review of certain mammalian host cell lines suitable for antigen-binding construct production, see, e.g., Yazaki & Wu, Methods in Molecular Biology, Vol. 248 (B. K. C. Lo, ed., Humana Press, Totowa, N.J.), pp. 255-268 (2003).

In some embodiments, the multi-specific antigen-binding constructs described herein are produced in stable mammalian cells by a method comprising transfecting at least one stable mammalian cell with nucleic acid encoding the multi-specific antigen-binding construct, in a predetermined ratio, and expressing the nucleic acid in the at least one mammalian cell. In some embodiments, the predetermined ratio of nucleic acid is determined in transient transfection experiments to determine the relative ratio of input nucleic acids that results in the highest percentage of the multi-specific antigen-binding construct in the expressed product.

In some embodiments, in the method of producing a multi-specific antigen-binding construct in stable mammalian cells, the expression product of the stable mammalian cell comprises a larger percentage of the desired multi-specific antigen-binding construct as compared to the monomeric heavy or light chain polypeptides, or other antibodies. In certain embodiments, the multi-specific antigen-binding construct is glycosylated.

In some embodiments, in the method of producing a multi-specific antigen-binding construct in stable mammalian cells, the method further comprises identifying and purifying the desired multi-specific antigen-binding construct. In some embodiments, identification is by one or both of liquid chromatography and mass spectrometry.

If required, the multi-specific antigen-binding constructs can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can be used for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed, or immobilized anti-flag antibody if a flag-tag is used. For general guidance in suitable purification techniques, see, e.g., Protein Purification: Principles and Practice, 3^{rd} Ed., Scopes, Springer-Verlag, NY (1994). The degree of purification necessary will vary depending on the use of the antigen-binding constructs. In some instances, no purification may be necessary.

In certain embodiments, the multi-specific antigen-binding constructs may be purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q or DEAE columns, or their equivalents or comparables.

In some embodiments, the multi-specific antigen-binding constructs may be purified using Cation Exchange Chromatography including, but not limited to, chromatography on SP-sepharose, CM sepharose, poros HS, poros CM, Toyopearl SP, Toyopearl CM, Resource/Source S or CM, or Fractogel S or CM columns, or their equivalents or comparables.

In certain embodiments, the multi-specific antigen-binding constructs are substantially pure. The term “substantially pure” (or “substantially purified”) refers to a construct described herein, or variant thereof, that may be substantially or essentially free of components that normally accompany or interact with the protein as found in its naturally occurring environment, i.e. a native cell, or host cell in the case of recombinantly produced construct. In certain embodiments, a construct that is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the construct is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the construct is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less.

In certain embodiments, the term “substantially purified” as applied to a multi-specific antigen-binding construct comprising a heterodimeric Fc as described herein means that the heterodimeric Fc has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, size-exclusion chromotagraphy (SEC) and capillary electrophoresis.

The multi-specific antigen-binding constructs may also be chemically synthesized using techniques known in the art (see, e.g., Creighton, Proteins: Structures and Molecular Principles, W. H. Freeman & Co., N.Y (1983), and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, alpha-amino isobutyric acid, 4 aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, Cα-methyl amino acids, Nα-methyl amino acids, and amino acid analogs in general.

Certain embodiments of the present disclosure relate to isolated nucleic acid encoding a multi-specific antigen-binding construct described herein. Such nucleic acid may encode an amino acid sequence comprising the VL and/or an amino acid sequence comprising the VH of the multi-specific antigen-binding construct (e.g. the light and/or heavy chains of the antigen-binding construct).

Certain embodiments relate to vectors (e.g. expression vectors) comprising nucleic acid encoding a multi-specific antigen-binding construct described herein. The nucleic acid may be comprised by a single vector or it may be comprised by more than one vector. In some embodiments, the nucleic acid is comprised by a multicistronic vector.

Certain embodiments relate to host cells comprising such nucleic acid or one or more vectors comprising the nucleic acid. In some embodiments, a host cell comprises (e.g. has been transformed with) a vector comprising a nucleic acid that encodes an amino acid sequence comprising the VL of the antigen-binding polypeptide construct and an amino acid sequence comprising the VH of the antigen-binding polypeptide construct. In some embodiments, a host cell comprises (e.g. has been transformed with) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In some embodiments, the host cell is eukaryotic, e.g. a Chinese Hamster Ovary (CHO) cell, or human embryonic kidney (HEK) cell, or lymphoid cell (e.g. Y0, NS0, Sp20 cell).

Certain embodiments relate to a method of making a multi-specific antigen-binding construct culturing a host cell into which nucleic acid encoding the multi-specific antigen-binding construct has been introduced, under conditions suitable for expression of the multi-specific antigen-binding construct, and optionally recovering the multi-specific antigen-binding construct from the host cell (or host cell culture medium).

Certain embodiments of the present disclosure relate to the co-expression of a multi-specific antigen-binding construct as described herein and a CAR or engineered TCR in a T-cell or NK-cell. Methods of co-expression of a CAR and an antibody in T-cells are known in the art (see, for example, International Patent Publication No. WO 2014/011988).

Accordingly, some embodiments relate to an engineered T-cell or NK-cell comprising nucleic acid encoding a CAR or engineered TCR, and nucleic acid encoding a multi-specific antigen-binding construct. Some embodiments relate to a method of co-expressing a multi-specific antigen-binding construct as described herein and a CAR or engineered TCR in a T-cell or NK-cell, which comprises introducing nucleic acid encoding the CAR or engineered TCR and nucleic acid encoding the multi-specific antigen-binding construct into the cell, and culturing the cell under conditions suitable for expression of the CAR or engineered TCR and the multi-specific antigen-binding construct. In certain embodiments, the nucleic acid encoding the CAR or engineered TCR, and the nucleic acid encoding the multi-specific antigen-binding construct are each in the form of a vector.

### Post-Translational Modifications

In certain embodiments, the multi-specific antigen-binding constructs described herein may be differentially modified during or after translation.

The term “modified,” as used herein, refers to any changes made to a given polypeptide, such as changes to the length of the polypeptide, the amino acid sequence, chemical structure, co-translational modification, or post-translational modification of a polypeptide.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the multi-specific antigen-binding constructs may comprise a modification such as glycosylation, acetylation, phosphorylation, amidation, derivatization by known protecting/blocking groups, proteolytic cleavage or linkage to an antibody molecule or antigen-binding construct or other cellular ligand, or a combination of these modifications. In some embodiments, the multi-specific antigen-binding construct may be chemically modified by known techniques including, but not limited to, specific chemical cleavage by cyanogen bromide, trypsin, chymotrypsin, papain, V8 protease or NaBH₄; acetylation; formylation; oxidation; reduction or metabolic synthesis in the presence of tunicamycin.

Additional optional post-translational modifications of antigen-binding constructs include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. The multi-specific antigen-binding constructs described herein may optionally be modified with a detectable label, such as an enzymatic, fluorescent, isotopic or affinity label to allow for detection and isolation of the protein. Examples of suitable enzyme labels include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin or aequorin; and examples of suitable radioactive materials include iodine, carbon, sulfur, tritium, indium, technetium, thallium, gallium, palladium, molybdenum, xenon or fluorine.

In some embodiments, the multi-specific antigen-binding constructs described herein may be attached to macrocyclic chelators that associate with radiometal ions.

In those embodiments in which the multi-specific antigen-binding constructs are modified, either by natural processes, such as post-translational processing, or by chemical modification techniques, the same type of modification may optionally be present in the same or varying degrees at several sites in a given polypeptide. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Proteins-Structure and Molecular Properties, 2nd Ed., T. E. Creighton, W. H. Freeman and Company, New York (1993); Post-Translational Covalent Modification of Proteins, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, the multi-specific antigen-binding constructs may be attached to a solid support, which may be particularly useful for immunoassays or purification of polypeptides that are bound by, or bind to, or associate with proteins described herein. Such solid supports include, but are not limited to, glass, cellulose, polyacrylamide, nylon, polystyrene, polyvinyl chloride or polypropylene.

### Testing the Multi-Specific Antigen-Binding Constructs

The multi-specific antigen binding constructs may be tested for their ability to bind to the target immunotherapeutic and tumour-associated antigen(s) using standard assays and protocols known in the art. Such assays and protocols include, for example, ELISA-based assays and surface-plasmon resonance (SPR) techniques. Cells expressing a target CAR or recombinant TCR may be purchased commercially (for example, from ProMab Biotechnologies Inc., Richmond, Calif., or from Creative Biolabs, Shirley, N.Y.) or may be prepared by standard techniques (see, for example, Yam et al., Mol. Ther. 5:479 (2002); and International Patent Publication No. WO 2015/095895). Cell lines expressing various target tumour-associated antigens are also available commercially.

The multi-specific antigen-binding constructs may additionally be tested for their ability to re-direct the target immunotherapeutic to a tumour cell expressing the target tumour-associated antigen. For example, where the immunotherapeutic comprises an engineered T-cell or NK cell, functional responses of the T-cell or NK cell after being contacted by the multi-specific antigen-binding construct may be assessed in vitro using standard assays known in the art. Some exemplary assays are provided in the Examples and described below.

For example, cytokine release from the engineered T-cells or NK cells may be assessed following incubation of the engineered cells with tumour-associated antigen-expressing and control cells in the presence or absence of the multi-specific antigen-binding construct. After incubation of the co-cultured cells for an appropriate time, supernatants can be collected and levels of IFN-γ, TNF-alpha and/or IL-2 may be determined, for example by multiplex cytokine immunoassay (Luminex®) or ELISA. Cytokine release by T-cells or NK cells is an indicator of cell activation and is known in the art to correlate with cytotoxity (see, for example, Kochenderfer, et al., J Immunother, 32(7):689-702 (2009); Lanitis, et al., Molec Ther, 20(3):633-643 (2012) and Mardiros, et al., Blood, 122(18):3138-3148 (2013)).

Cytolytic activity of the T-cell or NK cell may also optionally be assessed, for example, by incubating the engineered T-cells or NK cells and the target tumour cells in the presence and absence of varying concentrations of the multi-specific antigen-binding construct. Following incubation, lysis of the target tumour cells may be monitored by various techniques, such as flow cytometry, ⁵¹Cr release, fluorimetry, or a kinetic viability platform (such as Xcelligence (Acea)).

Proliferation of the engineered T-cells or NK cells may also be assessed following incubation with both cells expressing the target tumour-associated antigen and the multi-specific antigen-binding construct. For example, the engineered T-cells or NK cells can be labelled with an appropriate label, such as carboxyfluorescein succinmidyl ester (CFSE), and proliferation of the T-cells or NK cells may be assessed by flow cytometry.

In vivo effects of the multi-specific antigen-binding constructs may also be evaluated by standard techniques. For example, by monitoring tumours following adoptive transfer of engineered cells and administration of the multi-specific antigen-binding construct to patient-derived xenograft (PDX) tumour model animal subjects. Various PDX tumour models are available commercially and an appropriate model can be readily selected by the skilled person based on the target tumour-associated antigen being employed. The engineered T-cells or NK cells may be administered to the animals after tumour engraftment and then the multi-specific antigen-binding construct may be administered after an appropriate time period. The multi-specific antigen-binding construct may be administered intravenously (i.v.), intraperitoneally (i.p.) or subcutaneously (s.c.). Dosing schedules and amounts vary, but can be readily determined by the skilled person. An exemplary dosage would be 10 mg/kg once weekly. Tumour growth can be monitored by standard procedures. For example, when labelled tumour cells have been used, tumour growth may be monitored by appropriate imaging techniques. For solid tumours, tumour size may also be measured by caliper.

The ability of the multi-specific antigen-binding constructs to re-direct immunotherapeutics that are therapeutic agents capable of binding to a T-cell and a tumour-associated antigen, such as bispecific T-cell engagers (BiTEs), may be tested by first pre-treating T-cells with the therapeutic agent to allow the agent to engage the T-cell, then contacting the cells with the multi-specific antigen-binding construct. Cytotoxicity, cytokine release and proliferation of the T-cells may then be assayed using the same methods as described above.

### Pharmaceutical Compositions

Certain embodiments relate to pharmaceutical compositions comprising a multi-specific antigen-binding construct described herein and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, vehicle, or combination thereof, with which the construct is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

The pharmaceutical compositions may be in the form of solutions, suspensions, emulsion, tablets, pills, capsules, powders, sustained-release formulations and the like. The composition may be formulated as a suppository, with traditional binders and carriers such as triglycerides. Oral formulations may include standard carriers such as pharmaceutical grades of mannitol, lactose, starch, magnesium stearate, sodium saccharine, cellulose, magnesium carbonate, and the like.

Pharmaceutical compositions will contain a therapeutically effective amount of the multi-specific antigen-binding construct, together with a suitable amount of carrier so as to provide the form for proper administration to a patient. The formulation should suit the mode of administration.

In certain embodiments, the composition comprising the multi-specific antigen-binding construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, and the like.

### Methods of Using the Multi-Specific Antigen-Binding Constructs

The multi-specific antigen-binding constructs described herein may be used to re-direct a target immunotherapeutic such that it binds to a tumour cell antigen or epitope that is different from its cognate antigen or epitope. In this context, the tumour-associated antigen targeted antigen-binding domain comprised by the multi-specific antigen-binding construct provides an alternate antigen-binding domain to the antigen-binding domain comprised by the immunotherapeutic. In some embodiments, the target tumour cell may have lost, mutated, post-translationally modified or down-regulated expression of the tumour-associated antigen targeted by the immunotherapeutic, and the multi-specific antigen-binding construct thus provides an alternate antigen-binding domain through which the immunotherapeutic may bind to the tumour cell. The alternate antigen-binding domain may bind to a different tumour-associated antigen on the target tumour cell, or it may bind to the same tumour-associated antigen at a different epitope.

Certain embodiments, therefore, relate to methods for re-directing tumour-associated antigen specific immunotherapeutics toward alternative tumour antigens. In some embodiments, such re-direction may help to overcome common treatment resistance mechanisms in tumour cells involving antigen downregulation and/or neoplastic cell heterogeneity.

In some embodiments, the multi-specific antigen-binding construct may be used to increase the ability of the target immunotherapeutic to bind a tumour cell. In this context, the multi-specific antigen-binding construct provides an additional antigen-binding domain that binds a tumour-associated antigen on the target tumour cell. The additional antigen-binding domain may bind to a different tumour-associated antigen on the target tumour cell, or it may bind to the same tumour-associated antigen at a different epitope.

Certain embodiments relate to methods of using the multi-specific antigen-binding construct to extend the therapeutic effect of an immunotherapeutic. Certain embodiments relate to methods of using the multi-specific antigen-binding construct to improve the therapeutic effect of an immunotherapeutic. For example, in some embodiments, the multi-specific antigen-binding construct may be administered to a patient currently undergoing treatment with the immunotherapeutic in order to increase the likelihood of the immunotherapeutic treatment being effective. Patients that would benefit from such treatment would include, for example, patients displaying low levels of the immunotherapeutic target tumour-associated antigen, or in whom there is a risk of loss, modification or a decrease in expression, of the immunotherapeutic target tumour-associated antigen, or who display significant heterogeneity in expression of the immunotherapeutic target tumour-associated antigen. In this context, the multi-specific antigen-binding construct may be administered concurrently with the immunotherapeutic or it may be administered subsequently to administration of the immunotherapeutic. Such subsequent administration of the multi-specific antigen-binding construct means that administration of the immunotherapeutic and the multi-specific antigen-binding construct are separated by a defined time period, which may be short (for example in the order of minutes or hours) or extended (for example in the order of days or weeks).

In some embodiments, the multi-specific antigen-binding construct may be administered to a patient who has previously undergone treatment with the immunotherapeutic and who has relapsed or failed to respond to treatment, for example due to low levels or loss of expression of the immunotherapeutic target tumour-associated antigen. In such embodiments, re-direction of the immunotherapeutic by administration of the multi-specific antigen-binding construct is expected to initiate or re-initiate the therapeutic effect of the immunotherapeutic.

Certain embodiments relate to methods of treating cancer in a patient who is undergoing or has undergone treatment with an immunotherapeutic, comprising administering the multi-specific antigen-binding construct to the patient. In some embodiments, the patient has undergone prior treatment with the immunotherapeutic. In such embodiments, the patient may have relapsed from or failed the prior treatment with the immunotherapeutic.

In some embodiments, patients most likely to respond to treatment with the multi-specific antigen-binding construct may be identified by assessing expression of the tumour-associated antigen targeted by the immunotherapeutic and/or assessing the presence of an appropriate biomarker indicative of persistence of the prior immunotherapy. Assessment of the appropriate biomarker may comprise, for example, direct detection of a CAR or transgenic TCR on T-cells or NK cells, detection of increased activated memory T-cells, or detection of a pharmacodynamic marker such as low healthy B cell numbers in B cell-targeted immunotherapies. Patients having reduced neoplastic cell expression of the tumour-associated antigen targeted by the immunotherapeutic and evidence of prior immunotherapy persistence are more likely to respond to treatment with the multi-specific antigen-binding construct.

Many current immunotherapies are used in the treatment of hematological cancers. Accordingly, in certain embodiments, the multi-specific antigen-binding construct may be used in methods of treating a hematological cancer. Examples of hematological cancers include, but are not limited to, acute leukemia, for example, B-cell acute lymphoid leukemia (BALL), T-cell acute lymphoid leukemia (TALL), small lymphocytic leukemia (SLL), acute lymphoid leukemia (ALL) or acute myelogenous leukemia (AML); chronic leukemia, for example, chronic myelogenous leukemia (CML) or chronic lymphocytic leukemia (CLL); mantle cell lymphoma (MCL), B cell prolymphocytic leukemia, blastic plasmacytoid dendritic cell neoplasm, Burkitt's lymphoma, diffuse large B cell lymphoma (DLBCL) (e.g. T-cell/histiocyte rich large B-cell lymphoma, primary DLCBL of the CNS, primary cutaneous DLBCL leg type, or EBV+DLBCL of the elderly), DLBCL associated with chronic inflammation, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, splenic marginal zone lymphoma, splenic lymphoma/leukemia (e.g. unclassifiable), splenic diffuse red pulp small B-cell lymphoma, hairy cell leukemia-variant, lymphoplasmacytic lymphoma, a heavy chain disease (e.g. alpha heavy chain disease, gamma heavy chain disease, or mu heavy chain disease), plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, or an unclassifiable haematological cancer (e.g., with features intermediate between DLBCL and Burkitt lymphoma or intermediate between DLBCL and classical Hodgkin lymphoma).

Immunotherapies are also finding increasing use in the treatment of solid tumours. Accordingly, in some embodiments, the multi-specific antigen-binding construct may be used in methods of treating a solid tumour. Examples of commonly occurring solid tumours include, but are not limited to, cancer of the brain, breast, cervix, colon, head and neck, kidney, lung, ovary, pancreas, prostate, stomach and uterus, as well as non-small cell lung cancer and colorectal cancer. Various forms of lymphoma also may result in the formation of a solid tumour and, therefore, are also often considered to be solid tumours.

Certain embodiments relate to methods of using multi-specific antigen-binding constructs that bind to a CAR or TCR and a tumour-associated antigen to activate a T-cell or NK cell engineered to express the CAR or TCR. Activation of the T-cell or NK cell may result in release of cytokines, such as IFN-γ, TNF-alpha and/or IL-2, and/or cytotoxicity towards cells expressing the tumour-associated antigen. The method may be conducted in vitro, ex vivo or in vivo.

### Administration

Various modes of administration are suitable for administering the multi-specific antigen-binding constructs to a patient, for example, aerosol inhalation, injection, ingestion, transfusion, implantation or transplantation. An appropriate mode and route of administration of the multi-specific antigen-binding construct can be determined by the skilled practitioner taking account of the condition and patient to be treated. In certain embodiments, the multi-specific antigen-binding constructs may be administered to a patient subcutaneously, intradermally, intratumorally, intranodally, intramedullary, intramuscularly, intravenously (i.v.) or intraperitoneally. Typically, in the treatment of cancer, therapeutic compounds are administered systemically to patients, for example, by bolus injection or continuous infusion into a patient's bloodstream.

In certain embodiments in which the multi-specific antigen-binding construct is to be co-expressed in T-cells or NK cells with a CAR or engineered TCR, at least one of the following occurs in vitro prior to administering the cells to a patient: i) expansion of the cells, ii) introducing nucleic acid encoding the CAR or TCR and nucleic acid encoding the multi-specific antigen-binding construct into the cells, and/or iii) cryopreservation of the cells. Such ex vivo procedures are well known in the art. Briefly, isolated T-cells or NK cells are genetically modified by standard in vitro transduction or transfection techniques to introduce vectors expressing the CAR or TCR and the multi-specific antigen-binding construct. Typically, the cells are isolated from the patient to be treated (i.e. the cells are autologous). However, certain embodiments contemplate the use of cells that are allogeneic, syngeneic or xenogeneic with respect to the patient.

The modified cells are expanded ex vivo using standard methods are known in the art (see, for example, the procedure for expansion of hematopoietic stem and progenitor cells described in U.S. Pat. No. 5,199,942). Typically, ex vivo culture and expansion of T-cells comprises collecting PBMCs and, optionally, purifying T-cells from a subject. T-cells are expanded using a combination of mitogenic and, optionally, differentiative stimuli, for example anti-CD3/CD28 beads with exogenous cytokines such as IL-2, IL-7, IL-15 and/or IL-21 (Singh, et al., Cancer Res, 71(10):3516-27 (2011)). In some cases, CD34+ hematopoietic stem and progenitor cells are isolated from a mammal from peripheral blood harvest or bone marrow explants, and such cells are expanded ex vivo in media comprising appropriate cellular growth factors, as described in U.S. Pat. No. 5,199,942. Other factors such as Flt3-L, IL-1, IL-3 and c-kit ligand, may optionally be used for culturing and expansion of the cells.

The modified and expanded cells are then administered to the patient by a suitable route, for example, by intradermal injection, subcutaneous injection, i.v. injection, or direct injection into a tumour or lymph node.

The dosage of the above treatments to be administered to a patient will vary with the precise nature of the condition and patient being treated. The scaling of dosages for human administration can be performed according to art-accepted practices.

### Kits and Articles of Manufacture

Also encompassed herein are kits comprising one or more multi-specific antigen-binding constructs and kits comprising one or more polynucleotides encoding a multi-specific antigen-binding construct. In certain embodiments in which the kit comprises one or more polynucleotides, the polynucleotides may be provided in the form of a vector that may be used to transform host cells.

Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the multi-specific antigen-binding construct or polynucleotide.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition comprising the multi-specific antigen-binding construct which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises a multi-specific antigen-binding construct described herein; and (b) a second container with a composition contained therein, wherein the composition in the second container comprises a further cytotoxic or otherwise therapeutic agent. In such embodiments, the article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. The article of manufacture may optionally further include other materials desirable from a commercial and user standpoint, including other buffers, diluents, filters, needles, and syringes.

### Polypeptides and Polynucleotides

As described herein, the multi-specific antigen-binding constructs comprise at least one polypeptide. Certain embodiments relate to polynucleotides encoding such polypeptides described herein.

The multi-specific antigen-binding constructs, polypeptides and polynucleotides described herein are typically isolated. As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the antigen-binding construct, and may include enzymes, hormones, and other proteinaceous or non-proteinaceous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full-length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs are compounds that have the same basic chemical structure as a naturally occurring amino acid, i.e., a carbon that is bound to a hydrogen, a carboxyl group, an amino group, and an “R” group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, and the like, and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the antigen-binding constructs described herein may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. D-peptides, for example, are typically resistant to endogenous peptidases and proteases, thereby providing improved bioavailability of the molecule, and prolonged lifetimes in vivo when such properties are desirable. Additionally, D-peptides cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also included herein are polynucleotides encoding polypeptides of the multi-specific antigen-binding constructs. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof, and may include deoxyribonucleotides, deoxyribonucleosides, ribonucleosides, or ribonucleotides and polymers thereof in either single- or double-stranded form. Unless specifically limited, the term encompasses polynucleotides containing known analogs of natural nucleotides that have similar binding properties to the reference polynucleotide and are metabolized in a manner similar to naturally occurring nucleotides. Unless specifically limited otherwise, the term also refers to oligonucleotide analogs including PNA (peptidonucleic acid) and analogs of DNA used in antisense technology (phosphorothioates, phosphoroamidates, and the like). Unless otherwise indicated, a particular nucleotide sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” applies to both amino acid and nucleotide sequences. With respect to particular nucleotide sequences, “conservatively modified variants” refers to those nucleotide sequences which encode identical or essentially identical amino acid sequences, or where the nucleotide sequence does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. One of ordinary skill in the art will recognize that each codon in a nucleotide sequence (except AUG, which is ordinarily the only codon for methionine, and TGG, which is ordinarily the only codon for tryptophan) can be modified to yield a functionally identical molecule. Accordingly, each silent variation of a nucleotide sequence that encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M) (see, e.g., Creighton, Proteins: Structures and Molecular Properties (W H Freeman & Co.; 2^{nd} edition (December 1993)).

The term “identical” in the context of two or more nucleic acids or polypeptide sequences, refers to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide described herein, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (for example, GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al., Nuc. Acids Res. 25:3389-3402 (1997), and Altschul et al., J. Mol. Biol. 215:403-410 (1990), respectively. Software for performing BLAST analyses is publicly available through the website for the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1992)) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

In some aspects, a multi-specific antigen-binding construct comprises an amino acid sequence that is at least 80, 85, 90, 91, 92, 93, 94, 95, 96, 97, 98, 99, or 100% identical to a relevant amino acid sequence or fragment thereof set forth in the Tables or accession numbers disclosed herein. In some aspects, an isolated multi-specific antigen-binding construct comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in Tables or accession numbers disclosed herein.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Bispecific Antibody Variants

Bispecific antigen-binding constructs were prepared in the following formats:
a) A hybrid antibody format in which one antigen-binding domain is an scFv and the other is a Fab. These bispecific antigen-binding constructs further comprise a IgG1 heterodimeric Fc having CH3 domain amino acid substitutions that drive heterodimeric association of the two component Fc polypeptides, HetFcA and HetFcB.
HetFcA comprises the amino acid substitutions: T350V/L351Y/F405A/Y407V HetFcB comprises the amino acid substitutions: T350V/T366L/K392L/T394W The amino acid residues in the Fc region are identified according to the EU index as in Kabat referring to the numbering of the EU antibody (Edelman et al., Proc Natl Acad Sci USA, 63:78-85 (1969)). The hybrid antibody format constructs include 3 polypeptide chains: a first Fc polypeptide fused to an scFv that binds the first target, a second Fc polypeptide fused to VH-CH1 domains, and a light chain, where the VH-CH1 domains and the light chain form a Fab region that binds to the second target.
b) A tandem scFv format in which a first VL-VH sequence binding to the first target is connected by a GlySer based spacer to a second VL-VH sequence binding to the second target. The tandem ScFv constructs also contained a 6×His-tag.

The bispecific antigen-binding constructs prepared in this are described in Table C. “anti-FMC63id” is an anti-CD19 scFv (see, Immunology and Cell Biology (1991) 69:411-422, and International Patent Publication No. WO 2014/190273). “FLAG” is a well-known amino acid motif “DYKDDDDK” (Hopp, et al., Bio/Technology, 6 (10):1204-10 (1988)) used as a negative control arm in some exemplary constructs described herein. BCMA and mesothelin are tumour-associated antigens (TAAs). The scFv and Fab sequences were generated from the sequences of known antibodies, identified in Table 4 (see Example 7). Amino acid and nucleotide sequences for each of the variants listed in Table C are provided in Table 6. Tandem scFv sequences are provided without the 6×His tag.

TABLE C
Bispecific Antigen-Binding Constructs

| Variant | | | | | |
|---|---|---|---|---|---|
| # | Format | Specificity | Chain A | Chain B | Chain C |
| 16442 | hybrid | FLAG- | anti-FLAGVH- | anti-CD19scFv- | anti-FLAGVL- |
| | | CD19 | CH-HetFcA | HetFcB | IgKC |
| 16443 | hybrid | FLAG- | anti-FLAGVH- | anti- | anti-FLAGVL- |
| | | Mesothelin | CH-HetFcA | mesothelinscFv- | IgKC |
| | | | | HetFcB | |
| 16444 | hybrid | FMC63id- | anti- | anti-CD79bscFv- | anti- |
| | | CD79b | FMC63idVH- | HetFcB | FMC63idVL- |
| | | | CH-HetFcA | | IgKC |
| 16445 | hybrid | FMC63id- | anti- | anti- | anti- |
| | | BCMA | FMC63idVH- | BCMAscFv- | FMC63idVL- |
| | | | CH-HetFcA | HetFcB | IgKC |
| 16446 | hybrid | FMC63id- | anti- | anti- | Anti- |
| | | Mesothelin | FMC63idVH- | mesothelinscFv- | FMC63idVL- |
| | | | CH-HetFcA | HetFcB | IgKC |
| 16447 | hybrid | FLAG- | anti-FLAGVH- | anti-CD79bscFv- | anti-FLAGVL- |
| | | CD79b | CH-HetFcA | HetFcB | IgKC |
| 16448 | hybrid | FLAG- | anti-FLAGVH- | anti- | anti-FLAGVL- |
| | | BCMA | CH-HetFcA | BCMAscFv- | IgKC |
| | | | | HetFcB | |
| 16449 | tandem | Mesothelin- | anti- | — | — |
| | scFv | FLAG | mesothelinVL- | | |
| | | | VH-anti- | | |
| | | | FLAGVH-VL | | |
| 16450 | tandem | FMC63id- | anti- | — | — |
| | scFv | CD79b | FMC63idVL- | | |
| | | | VH-anti- | | |
| | | | CD79bVH-VL | | |
| 16451 | tandem | FMC63id- | anti- | — | — |
| | scFv | BCMA | FMC63idVL- | | |
| | | | VH-anti- | | |
| | | | BCMAVH-VL | | |
| 16452 | tandem | FMC63id- | anti- | — | — |
| | scFv | Mesothelin | FMC63idVL- | | |
| | | | VH-anti- | | |
| | | | mesothelinVH- | | |
| | | | VL | | |
| 16453 | tandem | CD19- | anti-CD19VL- | — | — |
| | scFv | FLAG | VH-anti- | | |
| | | | FLAGVH-VL | | |
| 16454 | tandem | CD79b- | anti-CD79bVL- | — | — |
| | scFv | FMC63id | VH-anti- | | |
| | | | FMC63idVH- | | |
| | | | VL | | |
| 16455 | tandem | BCMA- | anti-BCMAVL- | — | — |
| | scFv | FMC63id | VH-anti- | | |
| | | | FMC63idVH- | | |
| | | | VL | | |
| 16456 | tandem | Mesothelin- | anti- | — | — |
| | scFv | FMC63id | mesothelinVL- | | |
| | | | VH-anti- | | |
| | | | FMC63idVH- | | |
| | | | VL | | |
| 16457 | tandem | FLAG- | anti-FLAGVL- | — | — |
| | scFv | CD19 | VH-anti- | | |
| | | | CD19VH-VL | | |
| 16458 | tandem | FLAG- | anti-FLAGVL- | — | — |
| | scFv | CD79b | VH-anti- | | |
| | | | CD79bVH-VL | | |
| 16459 | tandem | FLAG- | anti-FLAGVL- | — | — |
| | scFv | BCMA | VH-anti- | | |
| | | | BCMAVH-VL | | |
| 16460 | tandem | FLAG- | anti-FLAGVL- | — | — |
| | scFv | Mesothelin | VH-anti- | | |
| | | | mesothelinVH- | | |
| | | | VL | | |
| 16461 | tandem | CD79b- | anti-CD79bVL- | — | — |
| | scFv | FLAG | VH-anti- | | |
| | | | FLAGVH-VL | | |
| 16462 | tandem | BCMA- | anti-BCMAVL- | — | — |
| | scFv | FLAG | VH-anti- | | |
| | | | FLAGVH-VL | | |

### Example 2: Bispecific Antibody Production

The bispecific antigen-binding constructs designated as Variants #16443 (FLAG-Mesothelin), 16445 (FMC63id-BCMA), 16446 (FMC63id-Mesothelin) and 16448 (FLAG-BCMA) described in Example 1 were prepared as follows.

The genes encoding the antibody heavy and light chains were constructed via gene synthesis using codons optimized for human/mammalian expression. The bispecific antibodies were cloned and expressed following the general procedure outlined in Example 7. Heterodimeric species were isolated to >90% purity via Protein A affinity chromatography followed by size-exclusion chromatography. All preparations had <5% multimeric species as verified by non-reducing SDS-PAGE and SEC.

### Example 3: Binding of Bispecific Antibodies to Tumour Cells

### Methods

Raji cells (ATCC CCL-86) and RPMI8226 cells (ATCC CCL-155) were cultured in RPMI-1640 medium containing 10% FBS. A1847 cells were cultured in DMEM containing 10% FBS. Each of the three cell lines was centrifuged and suspended at 5 million cells/ml in cold FACS buffer (PBS+2 mM EDTA pH 7.4+0.5% BSA). Test antibodies were diluted with PBS to 0.3 mg/ml. The antibodies were then serially diluted with PBS to 0.1 mg/ml, 30 ug/ml, 10 ug/ml, 3 ug/ml, 1 ug/ml and 0.3 ug/ml. Ten microliters of diluted antibody was mixed with 90 ul of cells in 96-well plates on ice, and the plates were incubated on ice for 30 min. The plates were then centrifuged, the supernatants were removed by decanting, and the cell pellets were suspended in 200 ul of cold FACS buffer. The plates were centrifuged again, the supernatants were removed by decanting, and the cells were suspended in 100 ul of cold FACS buffer containing 1 ug of Alexa Fluor 488-conjugated goat anti-human IgG (Jackson ImmunoResearch, West Grove, Pa.) and 0.1 ug of 7-aminoactinomycin D (7-AAD). The plates were incubated on ice for 30 min, then rinsed as above and cells were suspended in 200 ul of cold FACS buffer containing 1% paraformaldehyde. The plates were incubated at 4° C. overnight and the cells were acquired the following day on a BD LSR Fortessa X20 flow cytometer. The data were analyzed with FlowJo software (FlowJo, LLC, Ashland, Oreg.). The cells were first plotted by forward light scatter versus 7-AAD staining, then the live cells (7-AAD-negative) were gated and plotted as a histogram for Alexa Fluor 488 staining. The mean fluorescence was then recorded and pasted into Prism software (GraphPad Software, Inc., La Jolla, Calif.), with which mean fluorescence was plotted versus antibody concentration.

### Results

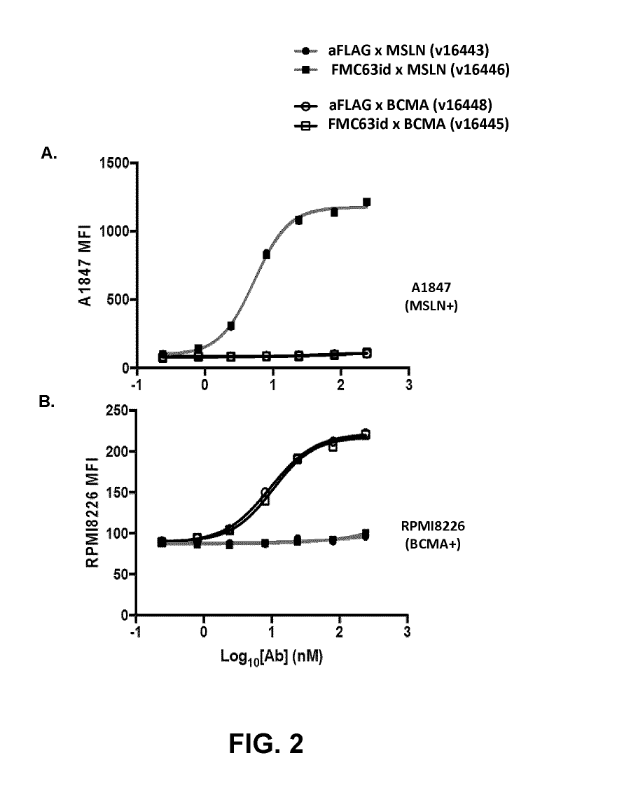

As shown in FIG. 2, the bispecific mesothelin (MSLN)-directed constructs (v16443 and v16446) bound to MSLN+A1847 cells, but not control RPMI8226 cells. Analogously, the bispecific BCMA-directed constructs (v16448 and v16445) bound to BCMA+RPMI8226 cells, but not control A1847 cells.

### Example 4: Binding of Bispecific Antibodies to Car-Expressing T-Cells

### Methods

Human T-cells were engineered to express FLAG-tagged second-generation CARs specific for CD19 (containing extracellular anti-CD19 (FMC63) scFv, FLAG, CD28 “hinge” and transmembrane, followed by intracellular CD28 and CD3-zeta signaling domains) were produced by ProMab Biotechnologies, Inc., Richmond, Calif. Briefly, PBMC were isolated from the peripheral blood of a healthy individual using density sedimentation over Ficoll, and the PBMC were cryopreserved. Lentivirus particles containing the CAR sequences were produced by co-transfection of HEK293 cells with a CAR-encoding vector and third-generation packaging constructs. The lentivirus particles were collected from the culture medium by ultracentrifugation, titered by qRT-PCR and frozen. The PBMC were thawed and cultured overnight in AIM-V® medium containing 5% human AB serum, CD3/CD28 antibody-coated magnetic beads and IL-2. The cells were transduced with the lentivirus preparations the next day at a multiplicity of infection of 5:1 in the presence of 5 ug/ml DEAE-dextran. Over the next two weeks of culture, the cells were counted every 2-3 days and additional medium was added to keep the cells at a density between 0.5 and 3 million per ml. CAR expression was evaluated by flow cytometry on day 9 of culture, using an antibody specific for FLAG.

To measure antibody binding to the CAR-T cells, either CAR-T cell preparations or HEK293 cells stably expressing the CD19 CAR were centrifuged and suspended in cold FACS buffer at 2.5 million cells per ml. Test antibodies were diluted in PBS to 0.4 mg/ml, and then serially diluted in PBS to 120 ug/ml and 40 ug/ml. Twenty-five microliters of antibody was mixed in triplicate with 75 ul of cells in 96-well plates on ice, and the plates were incubated on ice for 30 min. The plates were then centrifuged, the supernatants were removed by decanting, and the cell pellets were suspended in 200 ul of cold FACS buffer. The plates were centrifuged again, the supernatants were removed by decanting, and the cells were suspended in 100 ul of cold FACS buffer containing 1 ug of Alexa Fluor 488-conjugated goat anti-human IgG (Jackson ImmunoResearch, West Grove, Pa.) and 0.1 ug of 7-AAD. The plates were incubated on ice for 30 min, then rinsed as above and suspended in 200 ul of cold FACS buffer containing 1% paraformaldehyde. The plates were incubated at 4° C. overnight and the cells were acquired the following day on a BD FACSCalibur™ flow cytometer (BD Biosciences, San Jose, Calif.). The data were analyzed with FlowJo software (FlowJo, LLC, Ashland, Oreg.). The cells were first plotted by forward light scatter versus 7-AAD staining, then the live cells (7-AAD-negative) were gated and plotted by Alexa Fluor 488 staining versus a dummy channel.

### Results

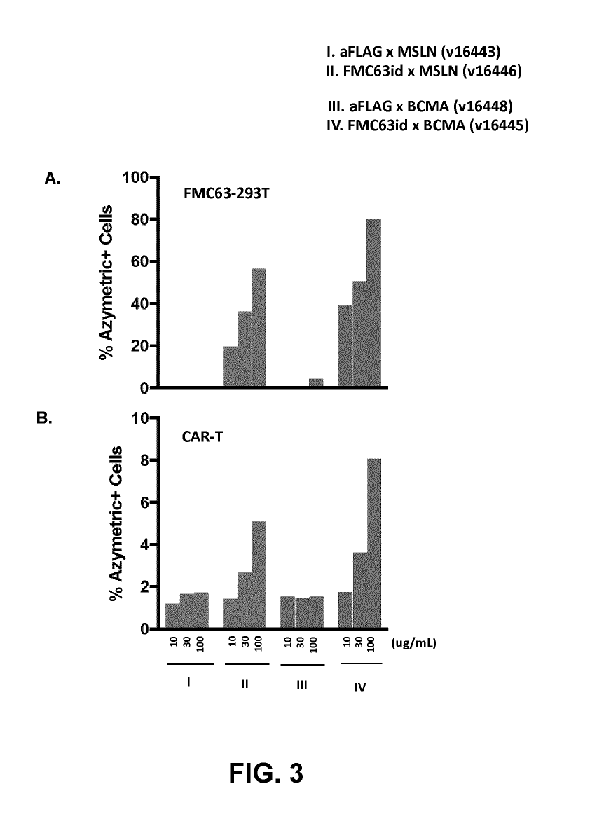

As shown in FIG. 3, anti-FMC63 idiotype-containing bispecific constructs (v16446 and v16445) bound selectively to anti-CD19 CAR constructs containing FMC63 stably expressed on either HEK293 or primary CAR-T cells.

Although the CAR constructs used in this Example contained extracellular FLAG sequences, no FLAG binding by the variants including an anti-FLAG domain was observed. This is likely due to conformational restrictions as the FLAG tag is located between the scFv and CD28 hinge of the CAR construct. This lack of binding allowed the anti-FLAG domain of these variants to be used as a negative control binding domain.

### Example 5: Modulation of CAR-T Cell Function by Bispecific Antibodies

### Methods

Antibodies were diluted in PBS to 0.4 mg/ml, then serially diluted in RPMI-1640 medium to 120 ug/ml and 40 ug/ml. CD19 CAR-T cells (see Example 4) were centrifuged and suspended in RPMI-1640 medium at 2 million cells per ml. Raji, RPMI8226 and SKOV3 target cells were centrifuged and suspended in RPMI-1640 medium at 0.2 million cells per ml. Fifty microliters of target cells were mixed in triplicate with 50 ul of CAR-T cells and 100 ul of antibody in 96-well plates. The plates were cultured 6 or 18 hours, and cells pelleted via centrifugation. The supernatants were transferred to fresh 96-well plates and frozen. Supernatant IFN-γ levels were quantified by sandwich ELISA.

### Results

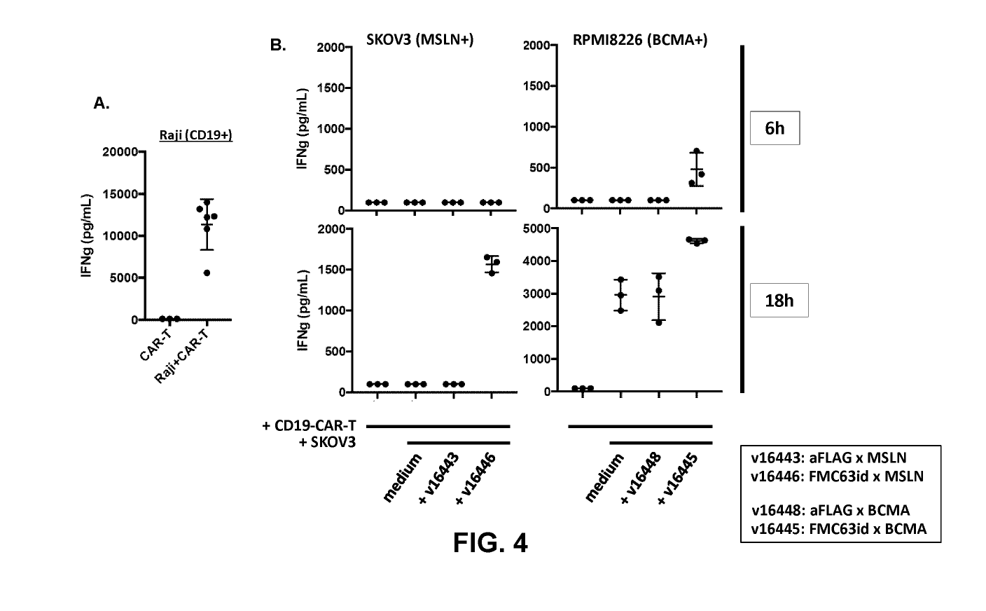

As shown in FIG. 4, CD19-CAR-T cells were robustly activated upon co-culture with CD19+Raji cells, but not CD19-negative SKOV3 cells. However, the anti-FMC63id×MSLN construct (v16446) re-directed CAR-T cells and potentiated robust activation in the presence of MSLN+SKOV3 cells. Similarly, CD19-CAR-T cell responses were re-directed to BCMA-expressing RPMI8226 target cells in the presence of the anti-FMC63id×BCMA construct (v16445) at 6 hours following co-culture initiation. At 18 hours post-co-culture initiation, RPMI8226 cells alone induced moderate CD19-CAR-T cell activation, consistent with low-level CD19 expression on a subset of RPMI8226 cells (see, Matsui, et al., Blood, 103(6):2332-2336 (2004)), which was further enhanced by addition of the anti-FMC63id×BCMA, but not control, construct.

The findings described in Examples 3-5, suggest that, while kinetics may vary between targets and/or cell types, CAR-engaging multi-specific antigen-binding constructs can be used to re-direct TAA-specific engineered cells toward alternative antigens, and enhance moderate cell activation induced by low-level cognate target expression. CAR constructs are designed to mimic natural TCR/CD3 signals (but with added co-stimulatory potential). As such, these findings support the use of multi-specific antigen-binding constructs directed to TCRs (using anti-TCR idiotype, V-region, or other similar binding domains) and TAAs to re-direct engineered or endogenous TCR-mediated T-cell responses toward alternative TAA targets.

While the multi-specific antigen-binding constructs used in these Examples are in a bispecific antibody format, T-cell engagement via CD3×TAA binding is well established in the art using a wide variety of biologics platforms, and thus these findings support the use of multi-specific antigen-binding constructs of alternative scaffold formats (BiTE, DART, and the like, as described herein) for re-directing T-cells toward alternative TAAs.

### Example 6: Description of Bispecific Antibody Variants

Bispecific antigen-binding constructs are prepared in the following exemplary formats:
a) A hybrid antibody format as described in Example 1 a).
b) A full-size antibody (FSA) format in which both antigen-binding domains are Fabs. These bispecific antigen-binding constructs also comprise the heterodimeric Fc described in Example 1. The full-size antibody format constructs include 4 polypeptide chains: a first Fc polypeptide fused to first VH-CH1 domains, and a first light chain, where the first VH-CH1 domains and the first light chain form a Fab region that binds to the first target; and a second Fc polypeptide fused to second VH-CH1 domains, and a second light chain, where the second VH-CH1 domains and the light chain form a Fab region that binds to the second target.
c) A tandem scFv format in which one VL-VH sequence binding to one target is connected by a (GGGGS)₅ spacer to a second VL-VH sequence binding to a second target.

A description of bispecific antigen-binding constructs to be prepared in the hybrid and FSA formats described above is provided in Table 2. A description of tandem scFv constructs to be prepared is provided in Table 3. “FMC63” is an anti-CD19 scFv (see Example 1, “FMC63id”).

TABLE 2
Bispecific antibodies in hybrid and FSA formats

| | FCA | | FcB | | |
|---|---|---|---|---|---|
| | | Paratope | | Paratope | |
| Variant | Target | format | Target | format | Ab format |
| 1 | FMC63 | Fab | CD79b | scFv | Hybrid |
| 2 | FMC63 | Fab | BCMA | scFv | Hybrid |
| 3 | FMC63 | Fab | Mesothelin | scFv | Hybrid |
| 4 | FMC63 | Fab | CD79b | Fab | Full size |
| 5 | FMC63 | Fab | BCMA | Fab | Full size |
| 6 | FMC63 | Fab | Mesothelin | Fab | Full size |

TABLE 3
Bispecific Tandem scFv constructs

| Variant | Target 1 | Target 2 |
|---|---|---|
| 7 | FMC63 | CD79b |
| 8 | FMC63 | BCMA |
| 9 | FMC63 | Mesothelin |

### Example 7: Bispecific Antibody Production

The bispecific antigen-binding constructs described in Example 6 are prepared as follows.

The genes encoding the antibody heavy and light chains are constructed via gene synthesis using codons optimized for human/mammalian expression. The scFv and Fab sequences are generated from the sequences of known antibodies, identified in Table 4. Sequences are provided in Table 5.

TABLE 4
References for Antibody Sequences

| Target | Antibody | Reference | Sequences |
|---|---|---|---|
| FMC63 | U. Texas anti-FMC63 | WO 2014/190273 | VH (SEQ ID NO: 1) |
| | (anti-CD19) idiotype clone | | VL (SEQ ID NO: 2) |
| | 136.20.1 | | |
| CD79b | Polatuzumab (humanized | IMGT/mAb-DB ID 458 | heavy chain (SEQ ID |
| | anti-CD79b) | | NO: 3) |
| | | | light chain (SEQ ID |
| | | | NO: 4) |
| BCMA | anti-BCMA (ADC, human | WO 2014/089335 | heavy chain (SEQ ID |
| | Ab); 2A1(Ab-1) | | NO: 7) |
| | | | light chain (SEQ ID |
| | | | NO: 8) |
| Mesothelin | Anetumab (anti- | IMGT/mAb-DB ID 471 | heavy chain (SEQ ID |
| | mesothelin) | | NO: 5) |
| | | | light chain (SEQ ID |
| | | | NO: 6) |

For constructs including scFvs, a disulphide link between the VH and VL of the scFv is introduced at positions VH 44 and VL 100, according to the Kabat numbering system (see Reiter et al, Nat Biotechnol, 14:1239-1245 (1996)).

The final gene products are sub-cloned into a mammalian expression vector and expressed in CHO cells (or a functional equivalent) (Durocher, et al., Nucl Acids Res, 30:E9 (2002)).

The CHO cells are transfected in exponential growth phase. In order to determine the optimal concentration range for forming heterodimers, the DNA may be transfected in various DNA ratios of the FcA, light chain (LC), and FcB that allow for heterodimer formation. Transfected cell culture medium is collected after several days, centrifuged at 4000 rpm and clarified using a 0.45 micron filter.

Bispecific antigen-binding constructs are purified from the culture medium via established methods. For example, the clarified culture medium is loaded onto a MabSelect SuRe (GEHealthcare) protein-A column and washed with PBS buffer at pH 7.2, eluted with citrate buffer at pH 3.6, and pooled fractions neutralized with TRIS at pH 11. The protein is finally desalted using an Econo-Pac 10DG column (Bio-Rad). In some cases, the protein is further purified by protein L chromatography or gel filtration.

### Example 8: Ability of Bispecific Antigen-Binding Constructs to Mediate Selective Lysis of Target Cells by CD19-Specific CAR-T Cells In Vitro

The ability of the bispecific antigen-binding constructs described in Example 6 to mediate lysis of target cells by CD19-specific CAR-T cells is assessed as outlined below. Genetically engineered human T cells expressing various CARs are commercially available. For example, CD19-specific CAR-T cells that comprise the scFv FMC63 are available from ProMab Biotechnologies Inc., Richmond, Calif.

CD19-specific CAR-expressing T cells and target cells are incubated in triplicate at multiple ratios (optimally approximately 20:1), in the presence or absence of varying concentrations of the bispecific antibodies described in Example 6. Target cells include: parental or control HeLa cells, and HeLa cells engineered via well-known methods to stably express CD19, CD79b, BCMA or mesothelin. Target cells may also include cell lines with endogenous CD19, CD79b, BCMA and/or mesothelin expression (such as Raji, Ramos, RPMI8226, and A1847), or primary tumour samples. Following incubation, lysis of target cells is monitored via flow cytometry, ⁵¹Cr release, fluorimetry, or a kinetic viability platform (such as Xcelligence (Acea)).

Target cell lysis values (Experimental lysis value) from different assay platforms are events/time period (flow cytometry), ⁵¹Cr release counts, relative luminescence units or relative fluorescence units. To measure spontaneous lysis, target cells are incubated without effector cells (CAR-T cells), and maximum lysis is determined following incubation of target cells with cytotoxic detergent.

The percent specific lysis is calculated as:

[(Experimental lysis value−Spontaneous lysis value)/(Maximum lysis value−Spontaneous lysis value)]×100.

### Results

T cells expressing CD19-specific CARs are expected to be able to efficiently lyse CD19-expressing target cells (HeLa-CD19 or Raji), but not CD19-negative target cell types (HeLa, HeLa-CD79b, HeLa-BCMA, RPMI8226 (CD19-low/negative), HeLa-mesothelin, or A1847). Analogously, mesothelin-specific CARs are able to lyse mesothelin-expressing target cells (Hela-mesothelin or A1847), but do not lyse mesothelin-negative target cell types (HeLa or HeLa-CD19). These results define cognate CAR-driven selectivity profiles.

Cognate CAR-driven selectivity profiles are altered upon incubation of CAR-T cells with multi-specific binding molecules that interact with CAR epitopes and alternative TAAs. Incubation of T cells expressing CD19-specific CARs with bispecific antibodies targeting the CAR scFv idiotype and a TAA can re-direct cytotoxic responses to alternative TAAs. For example:

a) CD19-specific CAR-T populations lyse HeLa-mesothelin or A1847 target cells in the presence of Variants 3, 6 or 9 (anti-CD19scFv idiotype/mesothelin);

b) CD19-specific CAR-T populations lyse HeLa-CD79b target cells in the presence of Variants 1, 4 or 7 (anti-CD19scFv idiotype/CD79b);

c) CD19-specific CAR-T populations lyse HeLa-BCMA or RPMI8226 target cells with increased efficacy in the presence of Variants 2, 5 or 8 (anti-CD19scFv idiotype/BCMA).

### Example 9: Ability of Bispecific Antigen-Binding Constructs to Stimulate Cytokine Production in Co-Culture of Target Cells and CD19-Specific CAR-T Cells In Vitro

Cytokine release is assessed following incubation of the CAR-expressing cells with antigen-expressing or control target cells in the presence or absence of bispecific antigen binding molecules. The target cells are the same as those described in Example 7. CD19-specific CAR-T cells are co-cultured with target cells at an optimal effector to target (E:T) ratio (approximately 2:1). The co-cultured cells are incubated for about 24 hours, and supernatants collected for measurement of IFN-γ, TNF-α, or IL-2 using a multiplex cytokine immunoassay (Luminex®) or ELISA.

### Results

Incubation of T-cells expressing CD19-specific CARs with bispecific antibodies targeting the CAR scFv idiotype and a TAA are expected to re-direct cytokine production responses to alternative TAAs. For example:

a) CD19-specific CAR-T populations produce IFN-γ, TNF-α and IL-2 in response to HeLa-mesothelin or A1847 target cells in the presence of Variants 3, 6 or 9 (anti-CD19scFv idiotype/mesothelin);

b) CD19-specific CAR-T populations produce IFN-γ, TNF-α and IL-2 in response to HeLa-CD79b target cells in the presence of Variants 1, 4 or 7 (anti-CD19scFv idiotype/CD79b);

c) CD19-specific CAR-T populations more efficiently produce IFN-γ, TNF-α and IL-2 in response to HeLa-BCMA or RPMI8226 target cells in the presence of Variants 2, 5 or 8 (anti-CD19scFv idiotype/BCMA).

### Example 10: Ability of Bispecific Antigen-Binding Constructs to Stimulate Proliferation of CD19-Specific CAR-T Cells in the Presence of Target Cells

Proliferation of CD19-specific CAR-T cells following incubation with CD19-expressing target cells is assessed by flow cytometry. CD19-specific CAR-T cells are labeled with carboxyfluorescein succinmidyl ester (CFSE), washed and incubated for 72 hours with target cells in serum-containing medium without exogenous cytokines. The target cells are the same as those described in Example 7. Division of live T-cells is indicated by CFSE dilution, as assessed by flow cytometry.

### Results

Incubation of T-cells expressing CD19-specific CARs with bispecific antibodies targeting the CAR scFv idiotype and a TAA is expected to re-direct proliferation responses to alternative TAAs. For example:

a) CD19-specific CAR-T populations proliferate in response to HeLa-mesothelin or A1847 target cells in the presence of Variants 3, 6 or 9 (anti-CD19scFv idiotype/mesothelin);

b) CD19-specific CAR-T populations proliferate in response to HeLa-CD79b target cells in the presence of Variants 1, 4 or 7 (anti-CD19scFv idiotype/CD79b);

c) CD19-specific CAR-T populations efficiently proliferate in response to HeLa-BCMA or RPMI8226 target cells in the presence of Variants 2, 5 or 8 (anti-CD19scFv idiotype/BCMA).

### Example 11: Ability of Bispecific Antigen-Binding Constructs to Re-Direct Cd19-Specific CAR-T Cells to Alternate TAAs In Vivo

The ability of the bispecific antigen-binding constructs to re-direct the CD19-specific CAR-T cells towards alternative TAAs in vivo is assessed in a patient-derived xenograft (PDX) tumour model by monitoring tumour growth following adoptive transfer of CAR-T cells and administration of the bispecific antigen-binding constructs as described below. To facilitate these studies, CD19-negative Raji variants (19negRaji) are generated via CRISPR/Cas9-mediated gene editing (for example, using services available from GenScript, Piscataway, N.J.), or repeated cycles of flow-cytometric CD19-low population sorting, limiting dilution, and daughter line expansion.

Groups of six- to eight-week old female NOD.Cg.Prkdc^{scid}IL2rg^{tm/Wi/}/SzJ (NSG) mice are injected intravenously (i.v.) with one of the following:

a) Raji lymphoma tumour cells transfected with firefly luciferase;

b) CD19-negative Raji (19negRaji) lymphoma tumour cells transfected with firefly luciferase;

c) RPMI-8226 multiple myeloma cell (CD19-negative/low, BCMA-positive) tumour cells transfected with firefly luciferase.

A suitable number of cells for administration to the mice is, for example, 0.5×10⁶ cells. Tumour engraftment is allowed to occur for about 6 days and verified using bioluminescence imaging.

On day 7, mice receive a single intravenous (i.v.) injection of a sub-optimal dose (an exemplary dose is 1×10⁶) of CD19-specific CAR-T cells.

On various days after CAR-T cell engraftment (commonly day 7), the bispecific antibodies described in Example 1 are administered i.v., intraperitoneally or subcutaneously. Dosing schedules and amounts vary, but exemplary studies administer 10 mg/kg once weekly.

Tumour growth in the mice is monitored by bioluminescence imaging at various time points after tumour cell engraftment, commonly days 4, 7, 14, 21, 27, 34 and 41.

For bioluminescence imaging, mice receive intraperitoneal (i.p.) injections of luciferin substrate (CaliperLife Sciences, Hopkinton, Mass.) in PBS (an exemplary dose is about 15 μg/g body weight). Mice are anesthetized and imaged essentially as described in Example 7 of International Patent Publication No. WO 2015/095895 and the average radiance (p/s/cm/sr) is determined.

### Results

Control mouse tumours are expected to continue to grow over the course of the study following adoptive transfer of non-target cell directed CAR-T cells, while CD19-specific CAR-T cells are expected to reduce CD19+ tumour growth compared to expanded, non-transduced T-cell populations. Specifically:

19negRaji and RPMI-8226 multiple myeloma tumours are expected to grow normally in mice following administration of CD19-specific CAR-T cells

administration of CD19-specific CAR-T cell is expected to reduce Raji tumour growth

Analogous to in vitro results, CD19-specific CAR-T cells are expected to reduce CD19-negative tumour growth in mice upon administration of bispecific antigen-binding constructs that bind CAR epitopes and alternative TAAs. Specifically:

Administration of Variants 1, 4 or 7 (anti-CAR/CD79b) is expected to enable CD19-specific CAR-T cell control of 19negRaji and RPMI-8226 tumours;

RPMI-8226 tumour growth is also expected to be reduced by CD19-specific CAR-T populations in the presence of Variants 2, 5 or 8 (anti-CAR/BCMA).

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

TABLE 5
Sequences

| SEQ | | |
|---|---|---|
| ID | | |
| NO: | Description | Sequence |
| 1 | University | LKPREVKLVESGGGLVQPGGSLKLSCAAS |
| | of Texas | GFDFSRYWMSWVRQAPGKGLEWIGEINLD |
| | anti-FMC63 | SSTINYTPSLKDKFIISRDNAKNTLYLQM |
| | (anti-CD19) | SKVRSEDTALYYCARRYDAMDYWGQGTSV |
| | idiotype | TVSSAKTTAPSVYPLAPVCGDTTGSSVTL |
| | clone | GCLVKASQ |
| | 136.20.1; | |
| | VH domain | |
| 2 | University | ASDIVLTQSPASLAVSLGQRATISCRASE |
| | of Texas | SVDDYGISFMNWFQQKPGQPPKLLIYAAP |
| | anti-FMC63 | NQGSGVPARFSGSGSGTDFSLNIHPMEED |
| | (anti-CD19) | DTAMYFCQQSKDVRWRHQAGDQTG |
| | idiotype | |
| | clone | |
| | 136.20.1; | |
| | VL domain | |
| 3 | Polatuzumab | EVQLVESGGGLVQPGGSLRLSCAASGYTF |
| | (humanized | SSYWIEWVRQAPGKGLEWIGEILPGGGDT |
| | anti-CD79b); | NYNEIFKGRATFSADTSKNTAYLQMNSLR |
| | heavy chain; | AEDTAVYYCTRRVPIRLDYWGQGTLVTVS |
| | (VH = resi- | SASTKGPSVFPLAPSSKSTSGGTAALGCL |
| | dues 1-117, | VKDYFPEPVTVSWNSGALTSGVHTFPAVL |
| | CH1 = resi- | QSSGLYSLSSVVTVPSSSLGTQTYICNVN |
| | dues 118-215, | HKPSNTKVDKKVEPKSCDKTHTCPPCPAP |
| | CH2 = resi- | ELLGGPSVFLFPPKPKDTLMISRTPEVTC |
| | dues 231-340, | VVVDVSHEDPEVKFNWYVDGVEVHNAKTK |
| | CH3 = resi- | PREEQYNSTYRVVSVLTVLHQDWLNGKEY |
| | dues 341-445) | KCKVSNKALPAPIEKTISKAKGQPREPQV |
| | | YTLPPSREEMTKNQVSLTCLVKGFYPSDI |
| | | AVEWESNGQPENNYKTTPPVLDSDGSFFL |
| | | YSKLTVDKSRWQQGNVFSCSVMHEALHNH |
| | | YTQKSLSLSPGK |
| 4 | Polatuzumab | DIQLTQSPSSLSASVGDRVTITCKASQSV |
| | (humanized | DYEGDSFLNWYQQKPGKAPKLLIYAASNL |
| | anti-CD79b); | ESGVPSRFSGSGSGTDFTLTISSLQPEDF |
| | light chain; | ATYYCQQSNEDPLTFGQGTKVEIKRTVAA |
| | (VL = resi- | PSVFIFPPSDEQLKSGTASVVCLLNNFYP |
| | dues 1-111, | REAKVQWKVDNALQSGNSQESVTEQDSKD |
| | CL = resi- | STYSLSSTLTLSKADYEKHKVYACEVTHQ |
| | dues 112-218) | GLSSPVTKSFNRGEC |
| 5 | Anetumab | QVELVQSGAEVKKPGESLKISCKGSGYSF |
| | (anti- | TSYWIGWVRQAPGKGLEWMGIIDPGDSRT |
| | Mesothelin); | RYSPSFQGQVTISADKSISTAYLQWSSLK |
| | heavy chain; | ASDTAMYYCARGQLYGGTYMDGWGQGTLV |
| | (VH = resi- | TVSSASTKGPSVFPLAPSSKSTSGGTAAL |
| | dues 1-120, | GCLVKDYFPEPVTVSWNSGALTSGVHTFP |
| | CH1 = resi- | AVLQSSGLYSLSSVVTVPSSSLGTQTYIC |
| | dues 121-218, | NVNHKPSNTKVDKKVEPKSCDKTHTCPPC |
| | CH2 = resi- | PAPELLGGPSVFLFPPKPKDTLMISRTPE |
| | dues 234-343, | VTCVVVDVSHEDPEVKFNWYVDGVEVHNA |
| | CH3 = resi- | KTKPREEQYNSTYRVVSVLTVLHQDWLNG |
| | dues 344-448) | KEYKCKVSNKALPAPIEKTISKAKGQPRE |
| | | PQVYTLPPSRDELTKNQVSLTCLVKGFYP |
| | | SDIAVEWESNGQPENNYKTTPPVLDSDGS |
| | | FFLYSKLTVDKSRWQQGNVFSCSVMHEAL |
| | | HNHYTQKSLSLSPGK |
| 6 | Anetumab | DIALTQPASVSGSPGQSITISCTGTSSDI |
| | (anti- | GGYNSVSWYQQHPGKAPKLMIYGVNNRPS |
| | Mesothelin); | GVSNRFSGSKSGNTASLTISGLQAEDEAD |
| | light chain; | YYCSSYDIESATPVFGGGTKLTVLGQPKA |
| | (VL = resi- | APSVTLFPPSSEELQANKATLVCLISDFY |
| | dues 3-111, | PGAVTVAWKGDSSPVKAGVETTTPSKQSN |
| | CL = resi- | NKYAASSYLSLTPEQWKSHRSYSCQVTHE |
| | dues 112-217) | GSTVEKTVAPTECS |
| 7 | Anti-BCMA | EVQLVESGGGLVKPGGSLRLSCAASGFTF |
| | (ADC, human | GDYALSWFRQAPGKGLEWVGVSRSKAYGG |
| | Ab) 2A1 | TTDYAASVKGRFTISRDDSKSTAYLQMNS |
| | (Ab-1); | LKTEDTAVYYCASSGYSSGWTPFDYWGQG |
| | heavy chain | TLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQT |
| | | YICNVNHKPSNTKVDKKVEPKSCDKTHTC |
| | | PPCPAPELLGGPSVFLFPPKPKDTLMISR |
| | | TPEVTCVVVDVSHEDPEVKFNWYVDGVEV |
| | | HNAKTKPREEQYNSTYRVVSVLTVLHQDW |
| | | LNGKEYKCKVSNKALPAPIEKTISKAKGQ |
| | | PREPQVYTLPPSREEMTKNQVSLTCLVKG |
| | | FYPSDIAVEWESNGQPENNYKTTPPVLDS |
| | | DGSFFLYSKLTVDKSRWQQGNVFSCSVMH |
| | | EALHNHYTQKSLSLSPGK |
| 8 | Anti-BCMA | QSVLTQPPSASGTPGQRVTISCSGSSSNI |
| | (ADC, human | GSNTVNWYQQLPGTAPKLLIFNYHQRPSG |
| | Ab) 2A1 | VPDRFSGSKSGSSASLAISGLQSEDEADY |
| | (Ab-1); | YCAAWDDSLNGWVFGGGTKLTVLGQPKAA |
| | light chain | PSVTLFPPSSEELQANKATLVCLISDFYP |
| | | GAVTVAWKADSSPVKAGVETTTPSKQSNN |
| | | KYAASSYLSLTPEQWKSHRSYSCQVTHEG |
| | | STVEKTVAPTECS |

TABLE 6
Sequences

| SEQ | | Portion of | |
|---|---|---|---|
| ID | | Sequence | |
| NO. | Description | (Location) | Sequence |
| 10 | Anti- | Full | DVLMTQAPLTLPVSLGDQASISCRSSQAIVHANGNTYLEWYL |
| | FLAGVL- | | QKPGQSPALLIYKVANRFSGVPDRFSGSGSGTDFTLKISRVEA |
| | IgK C | | EDLGVYYCFQGAHAPYTFGGGTKLEIKRTVAAPSVFIFPPSDE |
| | | | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 11 | Anti- | Full | GATGTGCTGATGACCCAGGCCCCCCTGACACTGCCTGTGA |
| | FLAGVL- | | GCCTGGGCGACCAGGCCTCTATCAGCTGCAGGAGCTCCCA |
| | IgK C | | GGCCATCGTGCACGCCAACGGCAATACCTACCTGGAGTGG |
| | | | TATCTGCAGAAGCCAGGACAGTCCCCCGCCCTGCTGATCT |
| | | | ACAAGGTGGCCAACCGGTTCTCTGGCGTGCCCGACAGATT |
| | | | TTCCGGCTCTGGCAGCGGCACCGATTTCACACTGAAGATCT |
| | | | CCCGGGTGGAGGCAGAGGATCTGGGCGTGTACTATTGTTT |
| | | | TCAGGGAGCACACGCACCATACACCTTCGGGGGAGGAACT |
| | | | AAACTGGAAATCAAGAGGACCGTCGCGGCGCCCAGTGTCT |
| | | | TCATTTTTCCCCCTAGCGACGAACAGCTGAAGTCTGGGACA |
| | | | GCCAGTGTGGTCTGTCTGCTGAACAACTTCTACCCTAGAGA |
| | | | GGCTAAAGTGCAGTGGAAGGTCGATAACGCACTGCAGTCC |
| | | | GGAAATTCTCAGGAGAGTGTGACTGAACAGGACTCAAAAG |
| | | | ATAGCACCTATTCCCTGTCAAGCACACTGACTCTGAGCAA |
| | | | GGCCGACTACGAGAAGCATAAAGTGTATGCTTGTGAAGTC |
| | | | ACCCACCAGGGGCTGAGTTCACCAGTCACAAAATCATTCA |
| | | | ACAGAGGGGAGTGC |
| 12 | Anti- | VL | DVLMTQAPLTLPVSLGDQASISCRSSQAIVHANGNTYLEWYL |
| | FLAGVL- | (D1-K112) | QKPGQSPALLIYKVANRFSGVPDRFSGSGSGTDFTLKISRVEA |
| | IgK C | | EDLGVYYCFQGAHAPYTFGGGTKLEIK |
| 13 | Anti- | L1 | QAIVHANGNTY |
| | FLAGVL- | (Q27-Y37) | |
| | IgK C | | |
| 14 | Anti- | L3 | FQGAHAPYT |
| | FLAGVL- | (F94- | |
| | IgK C | T102) | |
| 15 | Anti- | L2 | KVA |
| | FLAGVL- | (K55-A57) | |
| | IgK C | | |
| 16 | Anti- | CL | RTVAAPSVFIFPPSDEQLKSGTASVVCLLNNFYPREAKVQWK |
| | FLAGVL- | (R113- | VDNALQSGNSQESVTEQDSKDSTYSLSSTLTLSKADYEKHKV |
| | IgK C | C219) | YACEVTHQGLSSPVTKSFNRGEC |
| 17 | Anti- | Full | DIVLTQSPASLAVSLGQRATISCRASESVDDYGISFMNWFQQK |
| | FMC63id | | PGQPPKLLIYAAPNQGSGVPARFSGSGSGTDFSLNIHPMEEDD |
| | VL-IgK C | | TAMYFCQQSKDVRWRHQAGDQTGRTVAAPSVFIFPPSDEQL |
| | | | KSGTASVVCLLNNFYPREAKVQWKVDNALQSGNSQESVTEQ |
| | | | DSKDSTYSLSSTLTLSKADYEKHKVYACEVTHQGLSSPVTKS |
| | | | FNRGEC |
| 18 | Anti- | Full | GATATTGTGCTGACCCAGTCTCCTGCCAGCCTGGCCGTGTC |
| | FMC63id | | CCTGGGCCAGAGGGCCACAATCTCTTGCAGAGCCAGCGAG |
| | VL-IgK C | | TCCGTGGACGATTACGGCATCTCTTTCATGAACTGGTTTCA |
| | | | GCAGAAGCCAGGCCAGCCCCCTAAGCTGCTGATCTATGCC |
| | | | GCCCCAAATCAGGGCAGCGGAGTGCCAGCACGGTTCTCTG |
| | | | GCAGCGGCTCCGGCACCGACTTTTCCCTGAACATCCACCCC |
| | | | ATGGAGGAGGACGATACAGCCATGTACTTCTGTCAGCAGA |
| | | | GCAAGGATGTGAGATGGAGACACCAGGCAGGGGACCAGA |
| | | | CAGGAAGAACCGTGGCGGCGCCCAGTGTCTTCATTTTTCCC |
| | | | CCTAGCGACGAACAGCTGAAGTCTGGGACAGCCAGTGTGG |
| | | | TCTGTCTGCTGAACAACTTCTACCCTAGAGAGGCTAAAGTG |
| | | | CAGTGGAAGGTCGATAACGCACTGCAGTCCGGAAATTCTC |
| | | | AGGAGAGTGTGACTGAACAGGACTCAAAAGATAGCACCTA |
| | | | TTCCCTGTCAAGCACACTGACTCTGAGCAAGGCCGACTAC |
| | | | GAGAAGCATAAAGTGTATGCTTGTGAAGTCACCCACCAGG |
| | | | GGCTGAGTTCACCAGTCACAAAATCATTCAACAGAGGGGA |
| | | | GTGC |
| 19 | Anti- | VL | DIVLTQSPASLAVSLGQRATISCRASESVDDYGISFMNWFQQK |
| | FMC63id | (D1-G109) | PGQPPKLLIYAAPNQGSGVPARFSGSGSGTDFSLNIHPMEEDD |
| | VL-IgK C | | TAMYFCQQSKDVRWRHQAGDQTG |
| 20 | Anti- | L1 | ESVDDYGISF |
| | FMC63id | (E27-F36) | |
| | VL-IgK C | | |
| 21 | Anti- | L3 | QQSKDVRWRHQA |
| | FMC63id | (Q93- | |
| | VL-IgK C | A104) | |
| 22 | Anti- | L2 | AAP |
| | FMC63id | (A54-P56) | |
| | VL-IgK C | | |
| 23 | Anti- | CL | RTVAAPSVFIFPPSDEQLKSGTASVVCLLNNFYPREAKVQWK |
| | FMC63id | (R110- | VDNALQSGNSQESVTEQDSKDSTYSLSSTLTLSKADYEKHKV |
| | VL-IgK C | C216) | YACEVTHQGLSSPVTKSFNRGEC |
| 24 | Anti- | Full | DVLMTQAPLTLPVSLGDQASISCRSSQAIVHANGNTYLEWYL |
| | FLAGVL- | | QKPGQSPALLIYKVANRFSGVPDRFSGSGSGTDFTLKISRVEA |
| | VH-anti- | | EDLGVYYCFQGAHAPYTFGGGTKLEIKGGGGSGGGGSGGGG |
| | CD19VH- | | SEVQLQQSGGELAKPGASVKMSCKSSGYTFTAYAIHWAKQA |
| | VL | | AGAGLEWIGYIAPAAGAAAYNAAFKGKATLAADKSSSTAYM |
| | | | AAAALTSEDSAVYYCARAAAAGADYWGQGTTLTVSSGGGG |
| | | | SEVKLQESGPGLVAPSQSLSVTCTVSGVSLPDYGVSWIRQPPR |
| | | | KGLEWLGVIWGSETTYYNSALKSRLTIIKDNSKSQVFLKMNS |
| | | | LQTDDTAIYYCAKHYYYGGSYAMDYWGQGTSVTVSSVEGG |
| | | | SGGSGGSGGSGGVDDIQMTQTTSSLSASLGDRVTISCRASQDI |
| | | | SKYLNWYQQKPDGTVKLLIYHTSRLHSGVPSRFSGSGSGTDY |
| | | | SLTISNLEQEDIATYFCQQGNTLPYTFGGGTKLEIT |
| 25 | Anti- | Full | GATGTGCTGATGACCCAGGCCCCACTGACACTGCCCGTGT |
| | FLAGVL- | | CCCTGGGCGACCAGGCCTCCATCTCTTGCCGGAGCTCCCAG |
| | VH-anti- | | GCAATCGTGCACGCAAACGGCAATACCTATCTGGAGTGGT |
| | CD19VH- | | ACCTGCAGAAGCCTGGCCAGTCCCCAGCCCTGCTGATCTAT |
| | VL | | AAGGTGGCCAACCGGTTCAGCGGAGTGCCTGACCGGTTCA |
| | | | GCGGCTCCGGCTCTGGAACCGATTTCACACTGAAGATCTCC |
| | | | AGAGTGGAGGCCGAGGATCTGGGCGTGTACTATTGCTTCC |
| | | | AGGGAGCCCACGCACCATACACCTTTGGCGGAGGAACAAA |
| | | | GCTGGAGATCAAGGGAGGAGGAGGCAGCGGCGGAGGAGG |
| | | | CTCCGGCGGCGGCGGCTCTGAGGTGCAGCTGCAGCAGAGC |
| | | | GGAGGAGAGCTGGCCAAGCCAGGGGCCAGCGTGAAGATG |
| | | | TCCTGTAAGTCTAGCGGCTATACCTTCACAGCCTACGCCAT |
| | | | CCACTGGGCAAAGCAGGCCGCCGGGGCAGGGCTGGAGTG |
| | | | GATCGGATATATCGCCCCCGCCGCCGGAGCCGCCGCCTAC |
| | | | AATGCCGCCTTTAAGGGCAAGGCCACCCTGGCCGCCGACA |
| | | | AGTCCTCTAGCACAGCATATATGGCCGCCGCCGCCCTGAC |
| | | | CAGCGAGGACTCTGCCGTGTACTATTGCGCAAGGGCCGCC |
| | | | GCCGCCGGAGCCGATTACTGGGGCCAGGGCACCACACTGA |
| | | | CCGTGTCCTCTGGAGGAGGAGGCAGCGAGGTGAAGCTGCA |
| | | | GGAGTCCGGACCAGGCCTGGTGGCCCCTAGCCAGTCCCTG |
| | | | TCTGTGACCTGTACAGTGAGCGGCGTGTCCCTGCCCGATTA |
| | | | CGGCGTGTCCTGGATCAGACAGCCCCCTAGAAAGGGCCTG |
| | | | GAGTGGCTGGGCGTGATCTGGGGCAGCGAGACAACATACT |
| | | | ATAACTCTGCCCTGAAGAGCAGACTGACCATCATCAAGGA |
| | | | CAACAGCAAGTCCCAGGTGTTTCTGAAGATGAATAGCCTG |
| | | | CAGACCGACGATACAGCCATCTACTATTGTGCCAAGCACT |
| | | | ACTATTACGGCGGCTCTTATGCCATGGACTATTGGGGCCAG |
| | | | GGCACCAGCGTGACAGTGAGCTCCGTGGAGGGAGGCTCTG |
| | | | GAGGCAGCGGAGGCTCCGGAGGCTCTGGAGGAGTGGACG |
| | | | ATATCCAGATGACACAGACCACATCTAGCCTGTCTGCCAG |
| | | | CCTGGGCGACAGGGTGACCATCTCCTGCAGGGCCTCTCAG |
| | | | GATATCAGCAAGTATCTGAATTGGTACCAGCAGAAGCCAG |
| | | | ACGGCACCGTGAAGCTGCTGATCTACCACACATCCAGGCT |
| | | | GCACTCTGGAGTGCCAAGCCGCTTCTCCGGCTCTGGCAGC |
| | | | GGCACCGACTATTCCCTGACAATCTCTAACCTGGAGCAGG |
| | | | AGGATATCGCCACCTACTTTTGTCAGCAGGGCAATACACT |
| | | | GCCATACACCTTCGGGGGAGGAACAAAACTGGAAATCACC |
| 26 | Anti- | VL | DVLMTQAPLTLPVSLGDQASISCRSSQAIVHANGNTYLEWYL |
| | FLAGVL- | (D1-K112) | QKPGQSPALLIYKVANRFSGVPDRFSGSGSGTDFTLKISRVEA |
| | VH-anti- | | EDLGVYYCFQGAHAPYTFGGGTKLEIK |
| | CD19VH- | | |
| | VL | | |
| 27 | Anti- | L1 | QAIVHANGNTY |
| | FLAGVL- | (Q27-Y37) | |
| | VH-anti- | | |
| | CD19VH- | | |
| | VL | | |
| 28 | Anti- | L3 | FQGAHAPYT |
| | FLAGVL- | (F94- | |
| | VH-anti- | T102) | |
| | CD19VH- | | |
| | VL | | |
| 29 | Anti- | L2 | KVA |
| | FLAGVL- | (K55-A57) | |
| | VH-anti- | | |
| | CD19VH- | | |
| | VL | | |
| 30 | Anti- | VH | EVQLQQSGGELAKPGASVKMSCKSSGYTFTAYAIHWAKQAA |
| | FLAGVL- | (E128- | GAGLEWIGYIAPAAGAAAYNAAFKGKATLAADKSSSTAYMA |
| | VH-anti- | S244) | AAALTSEDSAVYYCARAAAAGADYWGQGTTLTVSS |
| | CD19VH- | | |
| | VL | | |
| 31 | Anti- | H1 | GYTFTAYA |
| | FLAGVL- | (G153- | |
| | VH-anti- | A160) | |
| | CD19VH- | | |
| | VL | | |
| 32 | Anti- | H3 | ARAAAAGADY |
| | FLAGVL- | (A224- | |
| | VH-anti- | Y233) | |
| | CD19VH- | | |
| | VL | | |
| 33 | Anti- | H2 | IAPAAGAA |
| | FLAGVL- | (I178- | |
| | VH-anti- | A185) | |
| | CD19VH- | | |
| | VL | | |
| 34 | Anti- | VH | EVKLQESGPGLVAPSQSLSVTCTVSGVSLPDYGVSWIRQPPRK |
| | FLAGVL- | (E250- | GLEWLGVIWGSETTYYNSALKSRLTIIKDNSKSQVFLKMNSL |
| | VH-anti- | S369) | QTDDTAIYYCAKHYYYGGSYAMDYWGQGTSVTVSS |
| | CD19VH- | | |
| | VL | | |
| 35 | Anti- | H1 | GVSLPDYG |
| | FLAGVL- | (G275- | |
| | VH-anti- | G282) | |
| | CD19VH- | | |
| | VL | | |
| 36 | Anti- | H3 | AKHYYYGGSYAMDY |
| | FLAGVL- | (A345- | |
| | VH-anti- | Y358) | |
| | CD19VH- | | |
| | VL | | |
| 37 | Anti- | H2 | IWGSETT |
| | FLAGVL- | (I300- | |
| | VH-anti- | T306) | |
| | CD19VH- | | |
| | VL | | |
| 38 | Anti- | VL | DIQMTQTTSSLSASLGDRVTISCRASQDISKYLNWYQQKPDG |
| | FLAGVL- | (D388- | TVKLLIYHTSRLHSGVPSRFSGSGSGTDYSLTISNLEQEDIATY |
| | VH-anti- | T494) | FCQQGNTLPYTFGGGTKLEIT |
| | CD19VH- | | |
| | VL | | |
| 39 | Anti- | L1 | QDISKY |
| | FLAGVL- | (Q414- | |
| | VH-anti- | Y419) | |
| | CD19VH- | | |
| | VL | | |
| 40 | Anti- | L3 | QQGNTLPYT |
| | FLAGVL- | (Q476- | |
| | VH-anti- | T484) | |
| | CD19VH- | | |
| | VL | | |
| 41 | Anti- | L2 | HTS |
| | FLAGVL- | (H437- | |
| | VH-anti- | S439) | |
| | CD19VH- | | |
| | VL | | |
| 42 | Anti- | Full | DVLMTQAPLTLPVSLGDQASISCRSSQAIVHANGNTYLEWYL |
| | FLAGVL- | | QKPGQSPALLIYKVANRFSGVPDRFSGSGSGTDFTLKISRVEA |
| | VH-anti- | | EDLGVYYCFQGAHAPYTFGGGTKLEIKGGGGSGGGGSGGGG |
| | CD79bVH- | | SEVQLQQSGGELAKPGASVKMSCKSSGYTFTAYAIHWAKQA |
| | VL | | AGAGLEWIGYIAPAAGAAAYNAAFKGKATLAADKSSSTAYM |
| | | | AAAALTSEDSAVYYCARAAAAGADYWGQGTTLTVSSGGGG |
| | | | SEVQLVESGGGLVQPGGSLRLSCAASGYTFSSYWIEWVRQAP |
| | | | GKGLEWIGEILPGGGDTNYNEIFKGRATFSADTSKNTAYLQM |
| | | | NSLRAEDTAVYYCTRRVPIRLDYWGQGTLVTVSSVEGGSGG |
| | | | SGGSGGSGGVDDIQLTQSPSSLSASVGDRVTITCKASQSVDYE |
| | | | GDSFLNWYQQKPGKAPKLLIYAASNLESGVPSRFSGSGSGTD |
| | | | FTLTISSLQPEDFATYYCQQSNEDPLTFGQGTKVEIK |
| 43 | Anti- | Full | GATGTGCTGATGACCCAGGCCCCCCTGACACTGCCTGTGA |
| | FLAGVL- | | GCCTGGGCGATCAGGCCTCTATCAGCTGCAGGAGCTCCCA |
| | VH-anti- | | GGCCATCGTGCACGCCAACGGCAATACCTACCTGGAGTGG |
| | CD79bVH- | | TATCTGCAGAAGCCAGGCCAGTCTCCCGCCCTGCTGATCTA |
| | VL | | CAAGGTGGCCAACAGGTTCTCCGGCGTGCCTGACCGCTTTT |
| | | | CCGGCTCTGGCAGCGGCACCGATTTCACACTGAAGATCAG |
| | | | CCGCGTGGAGGCAGAGGACCTGGGCGTGTACTATTGCTTC |
| | | | CAGGGAGCCCACGCCCCATATACCTTTGGCGGCGGCACAA |
| | | | AGCTGGAGATCAAGGGAGGAGGAGGCAGCGGCGGAGGAG |
| | | | GCTCCGGAGGCGGCGGCTCTGAGGTGCAGCTGCAGCAGTC |
| | | | CGGAGGAGAGCTGGCCAAGCCAGGGGCCAGCGTGAAGAT |
| | | | GAGCTGTAAGTCTAGCGGCTACACCTTCACAGCCTATGCC |
| | | | ATCCACTGGGCAAAGCAGGCCGCCGGGGCAGGGCTGGAGT |
| | | | GGATCGGATACATCGCCCCCGCCGCCGGAGCCGCCGCCTA |
| | | | TAATGCCGCCTTTAAGGGCAAGGCCACCCTGGCCGCCGAT |
| | | | AAGTCCTCTAGCACAGCATACATGGCCGCCGCCGCCCTGA |
| | | | CCAGCGAGGATAGCGCCGTGTACTATTGCGCAAGGGCCGC |
| | | | CGCCGCCGGAGCCGACTATTGGGGCCAGGGCACCACACTG |
| | | | ACAGTGTCCTCTGGCGGCGGCGGCAGCGAGGTGCAGCTGG |
| | | | TGGAGTCCGGAGGAGGCCTGGTGCAGCCTGGAGGCTCCCT |
| | | | GAGGCTGTCTTGTGCAGCCAGCGGCTACACCTTTAGCTCCT |
| | | | ATTGGATCGAGTGGGTGCGCCAGGCCCCCGGCAAGGGCCT |
| | | | GGAGTGGATCGGAGAGATCCTGCCTGGAGGAGGCGATACA |
| | | | AACTACAATGAGATCTTCAAGGGCAGAGCCACCTTTTCCG |
| | | | CCGACACCTCTAAGAACACAGCCTATCTGCAGATGAATAG |
| | | | CCTGCGGGCCGAGGATACCGCCGTGTACTATTGCACACGG |
| | | | AGAGTGCCAATCAGACTGGACTACTGGGGCCAGGGCACCC |
| | | | TGGTGACAGTGTCTAGCGTGGAGGGAGGCTCCGGAGGCTC |
| | | | TGGAGGCAGCGGAGGCTCCGGAGGCGTGGACGATATCCAG |
| | | | CTGACCCAGAGCCCATCCTCTCTGTCCGCCTCTGTGGGCGA |
| | | | CCGGGTGACCATCACCTGTAAGGCCAGCCAGTCCGTGGAC |
| | | | TACGAGGGCGATTCCTTCCTGAACTGGTATCAGCAGAAGC |
| | | | CTGGCAAGGCCCCAAAGCTGCTGATCTACGCAGCCAGCAA |
| | | | TCTGGAGTCCGGAGTGCCATCTAGATTCTCTGGCAGCGGCT |
| | | | CCGGCACAGACTTTACCCTGACAATCAGCTCCCTGCAGCCC |
| | | | GAGGATTTTGCCACCTACTATTGTCAGCAGAGCAACGAGG |
| | | | ACCCTCTGACATTCGGACAGGGGACTAAGGTGGAAATCAA |
| | | | G |
| 44 | Anti- | VL | DVLMTQAPLTLPVSLGDQASISCRSSQAIVHANGNTYLEWYL |
| | FLAGVL- | (D1-K112) | QKPGQSPALLIYKVANRFSGVPDRFSGSGSGTDFTLKISRVEA |
| | VH-anti- | | EDLGVYYCFQGAHAPYTFGGGTKLEIK |
| | CD79bVH- | | |
| | VL | | |
| 45 | Anti- | L1 | QAIVHANGNTY |
| | FLAGVL- | (Q27-Y37) | |
| | VH-anti- | | |
| | CD79bVH- | | |
| | VL | | |
| 46 | Anti- | L3 | FQGAHAPYT |
| | FLAGVL- | (F94- | |
| | VH-anti- | T102) | |
| | CD79bVH- | | |
| | VL | | |
| 47 | Anti- | L2 | KVA |
| | FLAGVL- | (K55-A57) | |
| | VH-anti- | | |
| | CD79bVH- | | |
| | VL | | |
| 48 | Anti- | VH | EVQLQQSGGELAKPGASVKMSCKSSGYTFTAYAIHWAKQAA |
| | FLAGVL- | (E128- | GAGLEWIGYIAPAAGAAAYNAAFKGKATLAADKSSSTAYMA |
| | VH-anti- | S244) | AAALTSEDSAVYYCARAAAAGADYWGQGTTLTVSS |
| | CD79bVH- | | |
| | VL | | |
| 49 | Anti- | H1 | GYTFTAYA |
| | FLAGVL- | (G153- | |
| | VH-anti- | A160) | |
| | CD79bVH- | | |
| | VL | | |
| 50 | Anti- | H3 | ARAAAAGADY |
| | FLAGVL- | (A224- | |
| | VH-anti- | Y233) | |
| | CD79bVH- | | |
| | VL | | |
| 51 | Anti- | H2 | IAPAAGAA |
| | FLAGVL- | (I178- | |
| | VH-anti- | A185) | |
| | CD79bVH- | | |
| | VL | | |
| 52 | Anti- | VH | EVQLVESGGGLVQPGGSLRLSCAASGYTFSSYWIEWVRQAPG |
| | FLAGVL- | (E250- | KGLEWIGEILPGGGDTNYNEIFKGRATFSADTSKNTAYLQMN |
| | VH-anti- | S366) | SLRAEDTAVYYCTRRVPIRLDYWGQGTLVTVSS |
| | CD79bVH- | | |
| | VL | | |
| 53 | Anti- | H1 | GYTFSSYW |
| | FLAGVL- | (G275- | |
| | VH-anti- | W282) | |
| | CD79bVH- | | |
| | VL | | |
| 54 | Anti- | H3 | TRRVPIRLDY |
| | FLAGVL- | (T346- | |
| | VH-anti- | Y355) | |
| | CD79bVH- | | |
| | VL | | |
| 55 | Anti- | H2 | ILPGGGDT |
| | FLAGVL- | (I300- | |
| | VH-anti- | T307) | |
| | CD79bVH- | | |
| | VL | | |
| 56 | Anti- | VL | DIQLTQSPSSLSASVGDRVTITCKASQSVDYEGDSFLNWYQQ |
| | FLAGVL- | (D385- | KPGKAPKLLIYAASNLESGVPSRFSGSGSGTDFTLTISSLQPED |
| | VH-anti- | K495) | FATYYCQQSNEDPLTFGQGTKVEIK |
| | CD79bVH- | | |
| | VL | | |
| 57 | Anti- | L1 | QSVDYEGDSF |
| | FLAGVL- | (Q411- | |
| | VH-anti- | F420) | |
| | CD79bVH- | | |
| | VL | | |
| 58 | Anti- | L3 | QQSNEDPLT |
| | FLAGVL- | (Q477- | |
| | VH-anti- | T485) | |
| | CD79bVH- | | |
| | VL | | |
| 59 | Anti- | L2 | AAS |
| | FLAGVL- | (A438- | |
| | VH-anti- | S440) | |
| | CD79bVH- | | |
| | VL | | |
| 60 | Anti- | Full | DVLMTQAPLTLPVSLGDQASISCRSSQAIVHANGNTYLEWYL |
| | FLAGVL- | | QKPGQSPALLIYKVANRFSGVPDRFSGSGSGTDFTLKISRVEA |
| | VH-anti- | | EDLGVYYCFQGAHAPYTFGGGTKLEIKGGGGSGGGGSGGGG |
| | BCMAVH- | | SEVQLQQSGGELAKPGASVKMSCKSSGYTFTAYAIHWAKQA |
| | VL | | AGAGLEWIGYIAPAAGAAAYNAAFKGKATLAADKSSSTAYM |
| | | | AAAALTSEDSAVYYCARAAAAGADYWGQGTTLTVSSGGGG |
| | | | SEVQLVESGGGLVKPGGSLRLSCAASGFTFGDYALSWFRQAP |
| | | | GKGLEWVGVSRSKAYGGTTDYAASVKGRFTISRDDSKSTAY |
| | | | LQMNSLKTEDTAVYYCASSGYSSGWTPFDYWGQGTLVTVSS |
| | | | VEGGSGGSGGSGGSGGVDQSVLTQPPSASGTPGQRVTISCSGS |
| | | | SSNIGSNTVNWYQQLPGTAPKLLIFNYHQRPSGVPDRFSGSKS |
| | | | GSSASLAISGLQSEDEADYYCAAWDDSLNGWVFGGGTKLTV |
| | | | L |
| 61 | Anti- | Full | GATGTGCTGATGACCCAGGCCCCACTGACACTGCCCGTGT |
| | FLAGVL- | | CCCTGGGCGACCAGGCCTCTATCAGCTGCAGGAGCTCCCA |
| | VH-anti- | | GGCCATCGTGCACGCCAACGGCAATACCTACCTGGAGTGG |
| | BCMAVH- | | TATCTGCAGAAGCCTGGCCAGAGCCCAGCCCTGCTGATCT |
| | VL | | ACAAGGTGGCCAACAGGTTCTCCGGAGTGCCAGACCGCTT |
| | | | TTCCGGCTCTGGCAGCGGCACCGATTTCACACTGAAGATCT |
| | | | CCCGCGTGGAGGCAGAGGATCTGGGCGTGTACTATTGCTT |
| | | | CCAGGGAGCCCACGCCCCTTATACCTTTGGCGGCGGCACA |
| | | | AAGCTGGAGATCAAGGGCGGCGGCGGCTCTGGAGGAGGA |
| | | | GGCAGCGGCGGAGGAGGCTCCGAGGTGCAGCTGCAGCAG |
| | | | AGCGGCGGCGAGCTGGCCAAGCCAGGGGCCAGCGTGAAG |
| | | | ATGTCCTGTAAGTCTAGCGGCTACACCTTCACAGCCTATGC |
| | | | CATCCACTGGGCAAAGCAGGCCGCCGGGGCAGGGCTGGA |
| | | | GTGGATCGGATACATCGCCCCCGCCGCCGGAGCCGCCGCC |
| | | | TATAATGCCGCCTTTAAGGGCAAGGCCACCCTGGCCGCCG |
| | | | ACAAGTCCTCTAGCACAGCATACATGGCCGCCGCCGCCCT |
| | | | GACCAGCGAGGACTCCGCCGTGTACTATTGCGCAAGGGCC |
| | | | GCCGCCGCCGGAGCCGATTATTGGGGCCAGGGCACCACAC |
| | | | TGACAGTGTCCTCTGGAGGAGGAGGCTCTGAGGTGCAGCT |
| | | | GGTGGAGAGCGGAGGAGGCCTGGTGAAGCCTGGAGGCTCT |
| | | | CTGAGACTGAGCTGTGCCGCCTCCGGCTTCACCTTTGGCGA |
| | | | CTACGCCCTGTCCTGGTTCAGGCAGGCCCCAGGCAAGGGC |
| | | | CTGGAGTGGGTGGGCGTGTCCCGCTCTAAGGCATACGGAG |
| | | | GCACCACAGATTATGCCGCCTCCGTGAAGGGCCGGTTTAC |
| | | | AATCTCTAGAGACGATAGCAAGTCCACCGCCTACCTGCAG |
| | | | ATGAACAGCCTGAAGACCGAGGACACAGCCGTGTACTATT |
| | | | GCGCCAGCTCCGGCTACTCTAGCGGCTGGACACCTTTTGAT |
| | | | TACTGGGGACAGGGCACCCTGGTGACAGTGTCCTCTGTGG |
| | | | AGGGAGGCTCTGGAGGCAGCGGAGGCTCCGGCGGCTCTGG |
| | | | AGGAGTGGACCAGTCCGTGCTGACCCAGCCACCTTCTGCC |
| | | | AGCGGAACCCCAGGCCAGCGGGTGACAATCTCCTGTTCTG |
| | | | GCAGCTCCTCTAACATCGGCTCTAACACAGTGAATTGGTAC |
| | | | CAGCAGCTGCCAGGAACCGCCCCTAAGCTGCTGATCTTCA |
| | | | ATTATCACCAGCGGCCAAGCGGAGTGCCAGATCGGTTCAG |
| | | | CGGCTCCAAGTCTGGCAGCTCCGCCTCTCTGGCCATCAGCG |
| | | | GCCTGCAGTCCGAGGACGAGGCAGATTACTATTGTGCCGC |
| | | | CTGGGACGATAGCCTGAATGGGTGGGTCTTCGGGGGAGGG |
| | | | ACAAAACTGACTGTGCTG |
| 62 | Anti- | VL | DVLMTQAPLTLPVSLGDQASISCRSSQAIVHANGNTYLEWYL |
| | FLAGVL- | (D1-K112) | QKPGQSPALLIYKVANRFSGVPDRFSGSGSGTDFTLKISRVEA |
| | VH-anti- | | EDLGVYYCFQGAHAPYTFGGGTKLEIK |
| | BCMAVH- | | |
| | VL | | |
| 63 | Anti- | L1 | QAIVHANGNTY |
| | FLAGVL- | (Q27-Y37) | |
| | VH-anti- | | |
| | BCMAVH- | | |
| | VL | | |
| 64 | Anti- | L3 | FQGAHAPYT |
| | FLAGVL- | (F94- | |
| | VH-anti- | T102) | |
| | BCMAVH- | | |
| | VL | | |
| 65 | Anti- | L2 | KVA |
| | FLAGVL- | (K55-A57) | |
| | VH-anti- | | |
| | BCMAVH- | | |
| | VL | | |
| 66 | Anti- | VH | EVQLQQSGGELAKPGASVKMSCKSSGYTFTAYAIHWAKQAA |
| | FLAGVL- | (E128- | GAGLEWIGYIAPAAGAAAYNAAFKGKATLAADKSSSTAYMA |
| | VH-anti- | S244) | AAALTSEDSAVYYCARAAAAGADYWGQGTTLTVSS |
| | BCMAVH- | | |
| | VL | | |
| 67 | Anti- | H1 | GYTFTAYA |
| | FLAGVL- | (G153- | |
| | VH-anti- | A160) | |
| | BCMAVH- | | |
| | VL | | |
| 68 | Anti- | H3 | ARAAAAGADY |
| | FLAGVL- | (A224- | |
| | VH-anti- | Y233) | |
| | BCMAVH- | | |
| | VL | | |
| 69 | Anti- | H2 | IAPAAGAA |
| | FLAGVL- | (I178- | |
| | VH-anti- | A185) | |
| | BCMAVH- | | |
| | VL | | |
| 70 | Anti- | VH | EVQLVESGGGLVKPGGSLRLSCAASGFTFGDYALSWFRQAPG |
| | FLAGVL- | (E250- | KGLEWVGVSRSKAYGGTTDYAASVKGRFTISRDDSKSTAYL |
| | VH-anti- | S372) | QMNSLKTEDTAVYYCASSGYSSGWTPFDYWGQGTLVTVSS |
| | BCMAVH- | | |
| | VL | | |
| 71 | Anti- | H1 | GFTFGDYA |
| | FLAGVL- | (G275- | |
| | VH-anti- | A282) | |
| | BCMAVH- | | |
| | VL | | |
| 72 | Anti- | H3 | ASSGYSSGWTPFDY |
| | FLAGVL- | (A348- | |
| | VH-anti- | Y361) | |
| | BCMAVH- | | |
| | VL | | |
| 73 | Anti- | H2 | SRSKAYGGTT |
| | FLAGVL- | (S300- | |
| | VH-anti- | T309) | |
| | BCMAVH- | | |
| | VL | | |
| 74 | Anti- | VL | QSVLTQPPSASGTPGQRVTISCSGSSSNIGSNTVNWYQQLPGT |
| | FLAGVL- | (Q391- | APKLLIFNYHQRPSGVPDRFSGSKSGSSASLAISGLQSEDEAD |
| | VH-anti- | L500) | YYCAAWDDSLNGWVFGGGTKLTVL |
| | BCMAVH- | | |
| | VL | | |
| 75 | Anti- | L1 | SSNIGSNT |
| | FLAGVL- | (S416- | |
| | VH-anti- | T423) | |
| | BCMAVH- | | |
| | VL | | |
| 76 | Anti- | L3 | AAWDDSLNGWV |
| | FLAGVL- | (A480- | |
| | VH-anti- | V490) | |
| | BCMAVH- | | |
| | VL | | |
| 77 | Anti- | L2 | NYH |
| | FLAGVL- | (N441- | |
| | VH-anti- | H443) | |
| | BCMAVH- | | |
| | VL | | |
| 78 | Anti- | Full | DVLMTQAPLTLPVSLGDQASISCRSSQAIVHANGNTYLEWYL |
| | FLAGVL- | | QKPGQSPALLIYKVANRFSGVPDRFSGSGSGTDFTLKISRVEA |
| | VH-anti- | | EDLGVYYCFQGAHAPYTFGGGTKLEIKGGGGSGGGGSGGGG |
| | mesothelin | | SEVQLQQSGGELAKPGASVKMSCKSSGYTFTAYAIHWAKQA |
| | VH-VL | | AGAGLEWIGYIAPAAGAAAYNAAFKGKATLAADKSSSTAYM |
| | | | AAAALTSEDSAVYYCARAAAAGADYWGQGTTLTVSSGGGG |
| | | | SQVELVQSGAEVKKPGESLKISCKGSGYSFTSYWIGWVRQAP |
| | | | GKGLEWMGIIDPGDSRTRYSPSFQGQVTISADKSISTAYLQWS |
| | | | SLKASDTAMYYCARGQLYGGTYMDGWGQGTLVTVSSVEGG |
| | | | SGGSGGSGGSGGVDDIALTQPASVSGSPGQSITISCTGTSSDIG |
| | | | GYNSVSWYQQHPGKAPKLMIYGVNNRPSGVSNRFSGSKSGN |
| | | | TASLTISGLQAEDEADYYCSSYDIESATPVFGGGTKLTVL |
| 79 | Anti- | Full | GATGTCCTGATGACCCAGGCCCCCCTGACACTGCCTGTGA |
| | FLAGVL- | | GCCTGGGCGACCAGGCCTCTATCAGCTGCAGGAGCTCCCA |
| | VH-anti- | | GGCCATCGTGCACGCCAACGGCAATACCTACCTGGAGTGG |
| | mesothelin | | TATCTGCAGAAGCCAGGACAGTCCCCCGCCCTGCTGATCT |
| | VH-VL | | ACAAGGTGGCCAACAGGTTCTCTGGAGTGCCAGACCGCTT |
| | | | TTCCGGCTCTGGCAGCGGCACCGATTTCACACTGAAGATC |
| | | | AGCCGCGTGGAGGCAGAGGATCTGGGCGTGTACTATTGCT |
| | | | TCCAGGGAGCCCACGCACCTTACACCTTTGGCGGAGGAAC |
| | | | AAAGCTGGAGATCAAGGGCGGCGGCGGCTCTGGAGGAGG |
| | | | AGGCAGCGGCGGAGGAGGCTCCGAGGTGCAGCTGCAGCA |
| | | | GTCCGGCGGCGAGCTGGCCAAGCCAGGGGCCAGCGTGAA |
| | | | GATGTCCTGTAAGTCTAGCGGCTACACCTTCACAGCCTATG |
| | | | CCATCCACTGGGCAAAGCAGGCCGCCGGGGCAGGGCTGGA |
| | | | GTGGATCGGATACATCGCCCCCGCCGCCGGAGCCGCCGCC |
| | | | TATAATGCCGCCTTTAAGGGCAAGGCCACCCTGGCCGCCG |
| | | | ACAAGTCCTCTAGCACAGCATACATGGCCGCCGCCGCCCT |
| | | | GACCAGCGAGGACTCTGCCGTGTACTATTGCGCAAGAGCC |
| | | | GCCGCCGCCGGAGCCGATTATTGGGGACAGGGCACCACAC |
| | | | TGACCGTGTCCTCTGGAGGAGGAGGCTCTCAGGTGGAGCT |
| | | | GGTGCAGAGCGGAGCCGAGGTGAAGAAGCCTGGCGAGTC |
| | | | TCTGAAGATCAGCTGTAAGGGCAGCGGCTACTCCTTCACA |
| | | | TCTTATTGGATCGGATGGGTGCGGCAGGCCCCAGGCAAGG |
| | | | GCCTGGAGTGGATGGGCATCATCGACCCAGGCGATAGCCG |
| | | | GACCAGATACTCCCCCTCTTTTCAGGGCCAGGTGACAATCT |
| | | | CCGCCGACAAGAGCATCTCCACCGCCTATCTGCAGTGGAG |
| | | | CTCCCTGAAGGCCAGCGATACAGCCATGTACTATTGCGCC |
| | | | AGAGGCCAGCTGTACGGAGGAACCTATATGGACGGATGGG |
| | | | GACAGGGCACCCTGGTGACAGTGTCTAGCGTGGAGGGAGG |
| | | | CAGCGGAGGCTCCGGAGGCTCTGGAGGCAGCGGAGGAGT |
| | | | GGACGATATCGCCCTGACACAGCCCGCCTCTGTGAGCGGC |
| | | | TCCCCTGGACAGTCCATCACCATCTCTTGTACCGGCACATC |
| | | | CTCTGATATCGGCGGCTACAACTCTGTGAGCTGGTATCAGC |
| | | | AGCACCCTGGCAAGGCCCCAAAGCTGATGATCTACGGCGT |
| | | | GAACAATCGGCCTTCCGGCGTGTCTAACAGATTTTCCGGCT |
| | | | CTAAGAGCGGCAATACCGCCAGCCTGACAATCTCCGGCCT |
| | | | GCAGGCAGAGGACGAGGCAGATTACTATTGTAGCTCCTAT |
| | | | GATATCGAGTCCGCCACTCCTGTCTTTGGCGGGGGCACTAA |
| | | | ACTGACTGTCCTG |
| 80 | Anti- | VL | DVLMTQAPLTLPVSLGDQASISCRSSQAIVHANGNTYLEWYL |
| | FLAGVL- | (D1-K112) | QKPGQSPALLIYKVANRFSGVPDRFSGSGSGTDFTLKISRVEA |
| | VH-anti- | | EDLGVYYCFQGAHAPYTFGGGTKLEIK |
| | mesothelin | | |
| | VH-VL | | |
| 81 | Anti- | L1 | QAIVHANGNTY |
| | FLAGVL- | (Q27-Y37) | |
| | VH-anti- | | |
| | mesothelin | | |
| | VH-VL | | |
| 82 | Anti- | L3 | FQGAHAPYT |
| | FLAGVL- | (F94-T102) | |
| | VH-anti- | | |
| | mesothelin | | |
| | VH-VL | | |
| 83 | Anti- | L2 | KVA |
| | FLAGVL- | (K55-A57) | |
| | VH-anti- | | |
| | mesothelin | | |
| | VH-VL | | |
| 84 | Anti- | VH | EVQLQQSGGELAKPGASVKMSCKSSGYTFTAYAIHWAKQAA |
| | FLAGVL- | (E128- | GAGLEWIGYIAPAAGAAAYNAAFKGKATLAADKSSSTAYMA |
| | VH-anti- | S244) | AAALTSEDSAVYYCARAAAAGADYWGQGTTLTVSS |
| | mesothelin | | |
| | VH-VL | | |
| 85 | Anti- | H1 | GYTFTAYA |
| | FLAGVL- | (G153- | |
| | VH-anti- | A160) | |
| | mesothelin | | |
| | VH-VL | | |
| 86 | Anti- | H3 | ARAAAAGADY |
| | FLAGVL- | (A224- | |
| | VH-anti- | Y233) | |
| | mesothelin | | |
| | VH-VL | | |
| 87 | Anti- | H2 | IAPAAGAA |
| | FLAGVL- | (I178- | |
| | VH-anti- | A185) | |
| | mesothelin | | |
| | VH-VL | | |
| 88 | Anti- | VH | QVELVQSGAEVKKPGESLKISCKGSGYSFTSYWIGWVRQAPG |
| | FLAGVL- | (Q250- | KGLEWMGIIDPGDSRTRYSPSFQGQVTISADKSISTAYLQWSS |
| | VH-anti- | S369) | LKASDTAMYYCARGQLYGGTYMDGWGQGTLVTVSS |
| | mesothelin | | |
| | VH-VL | | |
| 89 | Anti- | H1 | GYSFTSYW |
| | FLAGVL- | (G275- | |
| | VH-anti- | W282) | |
| | mesothelin | | |
| | VH-VL | | |
| 90 | Anti- | H3 | ARGQLYGGTYMDG |
| | FLAGVL- | (A346- | |
| | VH-anti- | G358) | |
| | mesothelin | | |
| | VH-VL | | |
| 91 | 10632 | H2 | IDPGDSRT |
| | (I300- | | |
| | T307) | | |
| 92 | Anti- | VL | DIALTQPASVSGSPGQSITISCTGTSSDIGGYNSVSWYQQHPGK |
| | FLAGVL- | (D388- | APKLMIYGVNNRPSGVSNRFSGSKSGNTASLTISGLQAEDEA |
| | VH-anti- | L498) | DYYCSSYDIESATPVFGGGTKLTVL |
| | mesothelin | | |
| | VH-VL | | |
| 93 | Anti- | L1 | SSDIGGYNS |
| | FLAGVL- | (S413- | |
| | VH-anti- | S421) | |
| | mesothelin | | |
| | VH-VL | | |
| 94 | Anti- | L3 | SSYDIESATPV |
| | FLAGVL- | (S478- | |
| | VH-anti- | V488) | |
| | mesothelin | | |
| | VH-VL | | |
| 95 | Anti- | L2 | GVN |
| | FLAGVL- | (G439- | |
| | VH-anti- | N441) | |
| | mesothelin | | |
| | VH-VL | | |
| 96 | Anti- | Full | DIVLTQSPASLAVSLGQRATISCRASESVDDYGISFMNWFQQK |
| | FMC63id | | PGQPPKLLIYAAPNQGSGVPARFSGSGSGTDFSLNIHPMEEDD |
| | VL-VH- | | TAMYFCQQSKDVRWRHQAGDQTGGGGGSGGGGSGGGGSE |
| | anti- | | VKLVESGGGLVQPGGSLKLSCAASGFDFSRYWMSWVRQAPG |
| | CD79bVH- | | KGLEWIGEINLDSSTINYTPSLKDKFIISRDNAKNTLYLQMSK |
| | VL | | VRSEDTALYYCARRYDAMDYWGQGTSVTVSSGGGGSEVQL |
| | | | VESGGGLVQPGGSLRLSCAASGYTFSSYWIEWVRQAPGKGLE |
| | | | WIGEILPGGGDTNYNEIFKGRATFSADTSKNTAYLQMNSLRA |
| | | | EDTAVYYCTRRVPIRLDYWGQGTLVTVSSVEGGSGGSGGSG |
| | | | GSGGVDDIQLTQSPSSLSASVGDRVTITCKASQSVDYEGDSFL |
| | | | NWYQQKPGKAPKLLIYAASNLESGVPSRFSGSGSGTDFTLTIS |
| | | | SLQPEDFATYYCQQSNEDPLTFGQGTKVEIK |
| 97 | Anti- | Full | GATATTGTGCTGACCCAGAGCCCCGCCTCCCTGGCCGTGTC |
| | FMC63id | | TCTGGGCCAGAGGGCAACAATCAGCTGCAGGGCCAGCGAG |
| | VL-VH- | | TCCGTGGACGATTACGGCATCAGCTTCATGAACTGGTTTCA |
| | anti- | | GCAGAAGCCTGGCCAGCCCCCTAAGCTGCTGATCTATGCC |
| | CD79bVH- | | GCCCCTAATCAGGGCAGCGGAGTGCCAGCCAGGTTCTCTG |
| | VL | | GCAGCGGCTCCGGAACCGATTTTTCCCTGAACATCCACCCT |
| | | | ATGGAGGAGGACGATACAGCCATGTACTTCTGCCAGCAGA |
| | | | GCAAGGACGTGCGGTGGAGACACCAGGCCGGGGACCAGA |
| | | | CCGGAGGAGGAGGAGGCTCCGGAGGAGGAGGCTCTGGCG |
| | | | GCGGCGGCAGCGAGGTGAAGCTGGTGGAGTCCGGAGGAG |
| | | | GCCTGGTGCAGCCAGGAGGCAGCCTGAAGCTGTCCTGTGC |
| | | | AGCCTCTGGCTTCGATTTTTCCCGGTATTGGATGTCTTGGG |
| | | | TGAGACAGGCCCCAGGCAAGGGCCTGGAGTGGATCGGCG |
| | | | AGATCAACCTGGACAGCTCCACCATCAATTACACACCCTC |
| | | | CCTGAAGGACAAGTTCATCATCTCTAGGGATAACGCCAAG |
| | | | AATACCCTGTATCTGCAGATGAGCAAGGTGCGCTCCGAGG |
| | | | ACACAGCCCTGTACTATTGCGCCCGGAGATACGACGCCAT |
| | | | GGATTATTGGGGCCAGGGCACCAGCGTGACAGTGTCTTCC |
| | | | GGAGGAGGCGGCAGCGAGGTGCAGCTGGTCGAAAGCGGC |
| | | | GGCGGCCTGGTCCAGCCAGGAGGCTCTCTGAGGCTGAGCT |
| | | | GTGCCGCCTCCGGCTACACCTTTTCCTCTTATTGGATCGAG |
| | | | TGGGTGCGCCAGGCCCCCGGCAAGGGCCTGGAATGGATCG |
| | | | GAGAGATCCTGCCTGGAGGAGGCGATACCAACTACAATGA |
| | | | GATCTTCAAGGGCAGAGCCACATTTTCTGCCGACACCAGC |
| | | | AAGAACACAGCCTATCTGCAGATGAACAGCCTGCGGGCCG |
| | | | AGGATACCGCCGTGTACTATTGCACAAGGCGCGTGCCAAT |
| | | | CAGACTGGACTACTGGGGCCAGGGCACCCTGGTGACAGTG |
| | | | AGCTCCGTGGAGGGAGGCTCTGGAGGCAGCGGAGGCTCCG |
| | | | GAGGCTCTGGAGGAGTGGACGATATCCAGCTGACCCAGTC |
| | | | TCCCTCTAGCCTGTCTGCCAGCGTGGGCGATCGGGTGACCA |
| | | | TCACCTGTAAGGCCTCCCAGTCTGTGGACTACGAGGGCGA |
| | | | TTCCTTCCTGAACTGGTATCAGCAGAAGCCAGGCAAGGCC |
| | | | CCCAAGCTGCTGATCTACGCCGCCTCCAATCTGGAGTCTGG |
| | | | CGTGCCTAGCAGATTCAGCGGCTCCGGCTCTGGCACCGAC |
| | | | TTTACCCTGACAATCTCCTCTCTGCAGCCAGAGGATTTTGC |
| | | | CACATACTATTGTCAGCAGAGCAATGAGGACCCTCTGACA |
| | | | TTCGGACAGGGAACTAAGGTGGAAATCAAA |
| 98 | Anti- | VL | DIVLTQSPASLAVSLGQRATISCRASESVDDYGISFMNWFQQK |
| | FMC63id | (D1-G109) | PGQPPKLLIYAAPNQGSGVPARFSGSGSGTDFSLNIHPMEEDD |
| | VL-VH- | | TAMYFCQQSKDVRWRHQAGDQTG |
| | anti- | | |
| | CD79bVH- | | |
| | VL | | |
| 99 | Anti- | L1 | ESVDDYGISF |
| | FMC63id | (E27-F36) | |
| | VL-VH- | | |
| | anti- | | |
| | CD79bVH- | | |
| | VL | | |
| 100 | Anti- | L3 | QQSKDVRWRHQA |
| | FMC63id | (Q93- | |
| | VL-VH- | A104) | |
| | anti- | | |
| | CD79bVH- | | |
| | VL | | |
| 101 | Anti- | L2 | AAP |
| | FMC63id | (A54-P56) | |
| | VL-VH- | | |
| | anti- | | |
| | CD79bVH- | | |
| | VL | | |
| 102 | Anti- | VH | EVKLVESGGGLVQPGGSLKLSCAASGFDFSRYWMSWVRQAP |
| | FMC63id | (E125- | GKGLEWIGEINLDSSTINYTPSLKDKFIISRDNAKNTLYLQMS |
| | VL-VH- | S240) | KVRSEDTALYYCARRYDAMDYWGQGTSVTVSS |
| | anti- | | |
| | CD79bVH- | | |
| | VL | | |
| 103 | Anti- | H1 | GFDFSRYW |
| | FMC63id | (G150- | |
| | VL-VH- | W157) | |
| | anti- | | |
| | CD79bVH- | | |
| | VL | | |
| 104 | Anti- | H3 | ARRYDAMDY |
| | FMC63id | (A221- | |
| | VL-VH- | Y229) | |
| | anti- | | |
| | CD79bVH- | | |
| | VL | | |
| 105 | Anti- | H2 | INLDSSTI |
| | FMC63id | (I175- | |
| | VL-VH- | I182) | |
| | anti- | | |
| | CD79bVH- | | |
| | VL | | |
| 106 | Anti- | VH | EVQLVESGGGLVQPGGSLRLSCAASGYTFSSYWIEWVRQAPG |
| | FMC63id | (E246- | KGLEWIGEILPGGGDTNYNEIFKGRATFSADTSKNTAYLQMN |
| | VL-VH- | S362) | SLRAEDTAVYYCTRRVPIRLDYWGQGTLVTVSS |
| | anti- | | |
| | CD79bVH- | | |
| | VL | | |
| 107 | Anti- | H1 | GYTFSSYW |
| | FMC63id | (G271- | |
| | VL-VH- | W278) | |
| | anti- | | |
| | CD79bVH- | | |
| | VL | | |
| 108 | Anti- | H3 | TRRVPIRLDY |
| | FMC63id | (T342- | |
| | VL-VH- | Y351) | |
| | anti- | | |
| | CD79bVH- | | |
| | VL | | |
| 109 | Anti- | H2 | ILPGGGDT |
| | FMC63id | (I296- | |
| | VL-VH- | T303) | |
| | anti- | | |
| | CD79bVH- | | |
| | VL | | |
| 110 | Anti- | VL | DIQLTQSPSSLSASVGDRVTITCKASQSVDYEGDSFLNWYQQ |
| | FMC63id | (D381- | KPGKAPKLLIYAASNLESGVPSRFSGSGSGTDFTLTISSLQPED |
| | VL-VH- | K491) | FATYYCQQSNEDPLTFGQGTKVEIK |
| | anti- | | |
| | CD79bVH- | | |
| | VL | | |
| 111 | Anti- | L1 | QSVDYEGDSF |
| | FMC63id | (Q407- | |
| | VL-VH- | F416) | |
| | anti- | | |
| | CD79bVH- | | |
| | VL | | |
| 112 | Anti- | L3 | QQSNEDPLT |
| | FMC63id | (Q473- | |
| | VL-VH- | T481) | |
| | anti- | | |
| | CD79bVH- | | |
| | VL | | |
| 113 | Anti- | L2 | AAS |
| | FMC63id | (A434- | |
| | VL-VH- | S436) | |
| | anti- | | |
| | CD79bVH- | | |
| | VL | | |
| 114 | Anti- | Full | DIVLTQSPASLAVSLGQRATISCRASESVDDYGISFMNWFQQK |
| | FMC63id | | PGQPPKLLIYAAPNQGSGVPARFSGSGSGTDFSLNIHPMEEDD |
| | VL-VH- | | TAMYFCQQSKDVRWRHQAGDQTGGGGGSGGGGSGGGGSE |
| | anti- | | VKLVESGGGLVQPGGSLKLSCAASGFDFSRYWMSWVRQAPG |
| | BCMAVH- | | KGLEWIGEINLDSSTINYTPSLKDKFIISRDNAKNTLYLQMSK |
| | VL | | VRSEDTALYYCARRYDAMDYWGQGTSVTVSSGGGGSEVQL |
| | | | VESGGGLVKPGGSLRLSCAASGFTFGDYALSWFRQAPGKGLE |
| | | | WVGVSRSKAYGGTTDYAASVKGRFTISRDDSKSTAYLQMNS |
| | | | LKTEDTAVYYCASSGYSSGWTPFDYWGQGTLVTVSSVEGGS |
| | | | GGSGGSGGSGGVDQSVLTQPPSASGTPGQRVTISCSGSSSNIG |
| | | | SNTVNWYQQLPGTAPKLLIFNYHQRPSGVPDRFSGSKSGSSA |
| | | | SLAISGLQSEDEADYYCAAWDDSLNGWVFGGGTKLTVL |
| 115 | Anti- | Full | GATATTGTGCTGACCCAGTCCCCAGCCTCTCTGGCCGTGTC |
| | FMC63id | | CCTGGGCCAGAGGGCCACAATCTCTTGCCGCGCCAGCGAG |
| | VL-VH- | | TCCGTGGACGATTACGGCATCAGCTTCATGAACTGGTTTCA |
| | anti- | | GCAGAAGCCCGGCCAGCCCCCTAAGCTGCTGATCTATGCC |
| | BCMAVH- | | GCCCCAAATCAGGGCTCCGGAGTGCCCGCCCGGTTCTCTG |
| | VL | | GCAGCGGCTCCGGCACCGACTTTTCTCTGAACATCCACCCC |
| | | | ATGGAGGAGGACGATACAGCCATGTACTTCTGCCAGCAGT |
| | | | CCAAGGACGTGAGGTGGCGGCACCAGGCCGGGGACCAGA |
| | | | CCGGAGGAGGAGGAGGCAGCGGAGGAGGAGGCTCCGGCG |
| | | | GCGGCGGCTCTGAGGTGAAGCTGGTGGAGAGCGGAGGAG |
| | | | GCCTGGTGCAGCCTGGAGGCTCCCTGAAGCTGTCTTGTGCC |
| | | | GCCAGCGGCTTCGACTTTAGCCGGTACTGGATGTCCTGGGT |
| | | | GAGACAGGCCCCTGGCAAGGGCCTGGAGTGGATCGGCGA |
| | | | GATCAACCTGGATAGCTCCACCATCAATTACACACCAAGC |
| | | | CTGAAGGACAAGTTTATCATCTCCAGGGATAACGCCAAGA |
| | | | ATACCCTGTATCTGCAGATGTCCAAGGTGCGCTCTGAGGAT |
| | | | ACAGCCCTGTACTATTGCGCCCGGAGATACGACGCCATGG |
| | | | ATTATTGGGGCCAGGGCACCTCCGTGACAGTGTCTAGCGG |
| | | | AGGAGGAGGCTCTGAGGTGCAGCTGGTCGAATCCGGCGGA |
| | | | GGCCTGGTGAAGCCAGGAGGCAGCCTGCGGCTGTCCTGTG |
| | | | CCGCCTCTGGCTTCACCTTTGGCGACTACGCCCTGAGCTGG |
| | | | TTCAGGCAGGCCCCTGGCAAGGGCCTGGAATGGGTGGGCG |
| | | | TGTCTAGAAGCAAGGCCTACGGCGGCACCACAGATTATGC |
| | | | CGCCTCTGTGAAGGGCCGGTTTACCATCAGCAGAGACGAT |
| | | | TCCAAGTCTACAGCCTATCTGCAGATGAACTCCCTGAAGA |
| | | | CCGAGGACACAGCCGTGTACTATTGCGCCTCCTCTGGCTAC |
| | | | AGCTCCGGCTGGACCCCTTTCGATTACTGGGGACAGGGCA |
| | | | CCCTGGTGACAGTGTCTAGCGTGGAGGGAGGCAGCGGAGG |
| | | | CTCCGGAGGCTCTGGCGGCAGCGGAGGAGTGGACCAGAGC |
| | | | GTGCTGACACAGCCACCAAGCGCCTCCGGAACCCCAGGAC |
| | | | AGAGGGTGACAATCTCTTGTAGCGGCTCCTCTAGCAACAT |
| | | | CGGCTCCAACACCGTGAATTGGTACCAGCAGCTGCCTGGC |
| | | | ACAGCCCCAAAGCTGCTGATCTTCAATTATCACCAGAGGC |
| | | | CCAGCGGAGTGCCTGATCGCTTTTCCGGCTCTAAGAGCGG |
| | | | CTCCTCTGCCAGCCTGGCCATCTCCGGCCTGCAGTCTGAGG |
| | | | ACGAGGCCGATTACTATTGTGCCGCCTGGGACGATAGCCT |
| | | | GAATGGCTGGGTCTTTGGGGGGGGGACTAAACTGACTGTG |
| | | | CTG |
| 116 | Anti- | VL | DIVLTQSPASLAVSLGQRATISCRASESVDDYGISFMNWFQQK |
| | FMC63id | (D1-G109) | PGQPPKLLIYAAPNQGSGVPARFSGSGSGTDFSLNIHPMEEDD |
| | VL-VH- | | TAMYFCQQSKDVRWRHQAGDQTG |
| | anti- | | |
| | BCMAVH- | | |
| | VL | | |
| 117 | Anti- | L1 | ESVDDYGISF |
| | FMC63id | (E27-F36) | |
| | VL-VH- | | |
| | anti- | | |
| | BCMAVH- | | |
| | VL | | |
| 118 | Anti- | L3 | QQSKDVRWRHQA |
| | FMC63id | (Q93- | |
| | VL-VH- | A104) | |
| | anti- | | |
| | BCMAVH- | | |
| | VL | | |
| 119 | Anti- | L2 | AAP |
| | FMC63id | (A54-P56) | |
| | VL-VH- | | |
| | anti- | | |
| | BCMAVH- | | |
| | VL | | |
| 120 | Anti- | VH | EVKLVESGGGLVQPGGSLKLSCAASGFDFSRYWMSWVRQAP |
| | FMC63id | (E125- | GKGLEWIGEINLDSSTINYTPSLKDKFIISRDNAKNTLYLQMS |
| | VL-VH- | S240) | KVRSEDTALYYCARRYDAMDYWGQGTSVTVSS |
| | anti- | | |
| | BCMAVH- | | |
| | VL | | |
| 121 | Anti- | H1 | GFDFSRYW |
| | FMC63id | (G150- | |
| | VL-VH- | W157) | |
| | anti- | | |
| | BCMAVH- | | |
| | VL | | |
| 122 | Anti- | H3 | ARRYDAMDY |
| | FMC63id | (A221- | |
| | VL-VH- | Y229) | |
| | anti- | | |
| | BCMAVH- | | |
| | VL | | |
| 123 | Anti- | H2 | INLDSSTI |
| | FMC63id | (I175- | |
| | VL-VH- | I182) | |
| | anti- | | |
| | BCMAVH- | | |
| | VL | | |
| 124 | Anti- | VH | EVQLVESGGGLVKPGGSLRLSCAASGFTFGDYALSWFRQAPG |
| | FMC63id | (E246- | KGLEWVGVSRSKAYGGTTDYAASVKGRFTISRDDSKSTAYL |
| | VL-VH- | S368) | QMNSLKTEDTAVYYCASSGYSSGWTPFDYWGQGTLVTVSS |
| | anti- | | |
| | BCMAVH- | | |
| | VL | | |
| 125 | Anti- | H1 | GFTFGDYA |
| | FMC63id | (G271- | |
| | VL-VH- | A278) | |
| | anti- | | |
| | BCMAVH- | | |
| | VL | | |
| 126 | Anti- | H3 | ASSGYSSGWTPFDY |
| | FMC63id | (A344- | |
| | VL-VH- | Y357) | |
| | anti- | | |
| | BCMAVH- | | |
| | VL | | |
| 127 | Anti- | H2 | SRSKAYGGTT |
| | FMC63id | (S296- | |
| | VL-VH- | T305) | |
| | anti- | | |
| | BCMAVH- | | |
| | VL | | |
| 128 | Anti- | VL | QSVLTQPPSASGTPGQRVTISCSGSSSNIGSNTVNWYQQLPGT |
| | FMC63id | (Q387- | APKLLIFNYHQRPSGVPDRFSGSKSGSSASLAISGLQSEDEAD |
| | VL-VH- | L496) | YYCAAWDDSLNGWVFGGGTKLTVL |
| | anti- | | |
| | BCMAVH- | | |
| | VL | | |
| 129 | Anti- | L1 | SSNIGSNT |
| | FMC63id | (S412- | |
| | VL-VH- | T419) | |
| | anti- | | |
| | BCMAVH- | | |
| | VL | | |
| 130 | Anti- | L3 | AAWDDSLNGWV |
| | FMC63id | (A476- | |
| | VL-VH- | V486) | |
| | anti- | | |
| | BCMAVH- | | |
| | VL | | |
| 134 | Anti- | L2 | NYH |
| | FMC63id | (N437- | |
| | VL-VH- | H439) | |
| | anti- | | |
| | BCMAVH- | | |
| | VL | | |
| 135 | Anti- | Full | DIVLTQSPASLAVSLGQRATISCRASESVDDYGISFMNWFQQK |
| | FMC63id | | PGQPPKLLIYAAPNQGSGVPARFSGSGSGTDFSLNIHPMEEDD |
| | VL-VH- | | TAMYFCQQSKDVRWRHQAGDQTGGGGGSGGGGSGGGGSE |
| | anti- | | VKLVESGGGLVQPGGSLKLSCAASGFDFSRYWMSWVRQAPG |
| | mesothelin | | KGLEWIGEINLDSSTINYTPSLKDKFIISRDNAKNTLYLQMSK |
| | VH-VL | | VRSEDTALYYCARRYDAMDYWGQGTSVTVSSGGGGSQVEL |
| | | | VQSGAEVKKPGESLKISCKGSGYSFTSYWIGWVRQAPGKGLE |
| | | | WMGIIDPGDSRTRYSPSFQGQVTISADKSISTAYLQWSSLKAS |
| | | | DTAMYYCARGQLYGGTYMDGWGQGTLVTVSSVEGGSGGS |
| | | | GGSGGSGGVDDIALTQPASVSGSPGQSITISCTGTSSDIGGYNS |
| | | | VSWYQQHPGKAPKLMIYGVNNRPSGVSNRFSGSKSGNTASL |
| | | | TISGLQAEDEADYYCSSYDIESATPVFGGGTKLTVL |
| 136 | Anti- | Full | GACATTGTGCTGACCCAGTCTCCAGCCAGCCTGGCCGTGTC |
| | FMC63id | | CCTGGGCCAGAGGGCCACAATCTCTTGCCGCGCCAGCGAG |
| | VL-VH- | | TCCGTGGACGATTACGGCATCAGCTTCATGAACTGGTTTCA |
| | anti- | | GCAGAAGCCCGGCCAGCCCCCTAAGCTGCTGATCTATGCC |
| | mesothelin | | GCCCCTAATCAGGGCAGCGGAGTGCCAGCCCGGTTCTCTG |
| | VH-VL | | GCAGCGGCTCCGGCACCGACTTTTCCCTGAACATCCACCCT |
| | | | ATGGAGGAGGACGATACAGCCATGTACTTCTGCCAGCAGA |
| | | | GCAAGGACGTGAGGTGGCGGCACCAGGCCGGGGACCAGA |
| | | | CCGGAGGAGGAGGAGGCAGCGGAGGAGGAGGCTCCGGCG |
| | | | GCGGCGGCTCTGAGGTGAAGCTGGTGGAGTCCGGAGGAGG |
| | | | CCTGGTGCAGCCAGGAGGCTCCCTGAAGCTGTCTTGTGCC |
| | | | GCCAGCGGCTTCGACTTTAGCCGGTACTGGATGTCCTGGGT |
| | | | GAGACAGGCCCCTGGCAAGGGCCTGGAGTGGATCGGCGA |
| | | | GATCAACCTGGATAGCTCCACCATCAATTACACACCAAGC |
| | | | CTGAAGGACAAGTTTATCATCTCCCGGGATAACGCCAAGA |
| | | | ATACCCTGTATCTGCAGATGTCCAAGGTGAGATCTGAGGA |
| | | | TACAGCCCTGTACTATTGCGCCCGGAGATACGACGCCATG |
| | | | GATTATTGGGGCCAGGGCACCAGCGTGACAGTGTCTAGCG |
| | | | GAGGAGGAGGCTCTCAGGTGGAGCTGGTGCAGAGCGGAG |
| | | | CCGAGGTGAAGAAGCCCGGCGAGAGCCTGAAGATCTCCTG |
| | | | TAAGGGCTCCGGCTACTCTTTCACCAGCTATTGGATCGGAT |
| | | | GGGTGAGGCAGGCCCCTGGCAAGGGCCTGGAATGGATGG |
| | | | GCATCATCGACCCAGGCGATTCTCGGACCAGATACTCTCCC |
| | | | AGCTTTCAGGGCCAGGTGACCATCTCCGCCGACAAGTCCA |
| | | | TCTCTACAGCCTATCTGCAGTGGTCCTCTCTGAAGGCCTCC |
| | | | GATACCGCCATGTACTATTGCGCCAGAGGCCAGCTGTACG |
| | | | GCGGCACATATATGGACGGATGGGGACAGGGCACCCTGGT |
| | | | GACAGTGAGCTCCGTGGAGGGAGGCTCCGGAGGCTCTGGA |
| | | | GGCAGCGGCGGCTCCGGAGGAGTGGACGATATCGCCCTGA |
| | | | CCCAGCCCGCCAGCGTGTCCGGCTCTCCTGGCCAGTCTATC |
| | | | ACAATCAGCTGTACCGGCACATCTAGCGATATCGGCGGCT |
| | | | ACAATAGCGTGTCCTGGTATCAGCAGCACCCAGGCAAGGC |
| | | | CCCCAAGCTGATGATCTACGGCGTGAACAATAGGCCCTCT |
| | | | GGCGTGAGCAACCGCTTCTCTGGCAGCAAGTCCGGCAATA |
| | | | CCGCCTCCCTGACAATCTCTGGCCTGCAGGCAGAGGACGA |
| | | | GGCAGATTACTATTGTTCCTCTTATGACATCGAGAGCGCCA |
| | | | CACCCGTCTTCGGAGGAGGAACCAAACTGACCGTGCTG |
| 137 | Anti- | VL | DIVLTQSPASLAVSLGQRATISCRASESVDDYGISFMNWFQQK |
| | FMC63id | (D1-G109) | PGQPPKLLIYAAPNQGSGVPARFSGSGSGTDFSLNIHPMEEDD |
| | VL-VH- | | TAMYFCQQSKDVRWRHQAGDQTG |
| | anti- | | |
| | mesothelin | | |
| | VH-VL | | |
| 138 | Anti- | L1 | ESVDDYGISF |
| | FMC63id | (E27-F36) | |
| | VL-VH- | | |
| | anti- | | |
| | mesothelin | | |
| | VH-VL | | |
| 139 | Anti- | L3 | QQSKDVRWRHQA |
| | FMC63id | (Q93- | |
| | VL-VH- | A104) | |
| | anti- | | |
| | mesothelin | | |
| | VH-VL | | |
| 140 | Anti- | L2 | AAP |
| | FMC63id | (A54-P56) | |
| | VL-VH- | | |
| | anti- | | |
| | mesothelin | | |
| | VH-VL | | |
| 141 | Anti- | VH | EVKLVESGGGLVQPGGSLKLSCAASGFDFSRYWMSWVRQAP |
| | FMC63id | (E125- | GKGLEWIGEINLDSSTINYTPSLKDKFIISRDNAKNTLYLQMS |
| | VL-VH- | S240) | KVRSEDTALYYCARRYDAMDYWGQGTSVTVSS |
| | anti- | | |
| | mesothelin | | |
| | VH-VL | | |
| 142 | Anti- | H1 | GFDFSRYW |
| | FMC63id | (G150- | |
| | VL-VH- | W157) | |
| | anti- | | |
| | mesothelin | | |
| | VH-VL | | |
| 143 | Anti- | H3 | ARRYDAMDY |
| | FMC63id | (A221- | |
| | VL-VH- | Y229) | |
| | anti- | | |
| | mesothelin | | |
| | VH-VL | | |
| 144 | Anti- | H2 | INLDSSTI |
| | FMC63id | (I175- | |
| | VL-VH- | I182) | |
| | anti- | | |
| | mesothelin | | |
| | VH-VL | | |
| 145 | Anti- | VH | QVELVQSGAEVKKPGESLKISCKGSGYSFTSYWIGWVRQAPG |
| | FMC63id | (Q246- | KGLEWMGIIDPGDSRTRYSPSFQGQVTISADKSISTAYLQWSS |
| | VL-VH- | S365) | LKASDTAMYYCARGQLYGGTYMDGWGQGTLVTVSS |
| | anti- | | |
| | mesothelin | | |
| | VH-VL | | |
| 146 | Anti- | H1 | GYSFTSYW |
| | FMC63id | (G271- | |
| | VL-VH- | W278) | |
| | anti- | | |
| | mesothelin | | |
| | VH-VL | | |
| 147 | Anti- | H3 | ARGQLYGGTYMDG |
| | FMC63id | (A342- | |
| | VL-VH- | G354) | |
| | anti- | | |
| | mesothelin | | |
| | VH-VL | | |
| 148 | Anti- | H2 | IDPGDSRT |
| | FMC63id | (I296- | |
| | VL-VH- | T303) | |
| | anti- | | |
| | mesothelin | | |
| | VH-VL | | |
| 149 | Anti- | VL | DIALTQPASVSGSPGQSITISCTGTSSDIGGYNSVSWYQQHPGK |
| | FMC63id | (D384- | APKLMIYGVNNRPSGVSNRFSGSKSGNTASLTISGLQAEDEA |
| | VL-VH- | L494) | DYYCSSYDIESATPVFGGGTKLTVL |
| | anti- | | |
| | mesothelin | | |
| | VH-VL | | |
| 150 | Anti- | L1 | SSDIGGYNS |
| | FMC63id | (S409- | |
| | VL-VH- | S417) | |
| | anti- | | |
| | mesothelin | | |
| | VH-VL | | |
| 151 | Anti- | L3 | SSYDIESATPV |
| | FMC63id | (S474- | |
| | VL-VH- | V484) | |
| | anti- | | |
| | mesothelin | | |
| | VH-VL | | |
| 152 | Anti- | L2 | GVN |
| | FMC63id | (G435- | |
| | VL-VH- | N437) | |
| | anti- | | |
| | mesothelin | | |
| | VH-VL | | |
| 153 | Anti- | Full | DIQMTQTTSSLSASLGDRVTISCRASQDISKYLNWYQQKPDG |
| | CD19VL- | | TVKLLIYHTSRLHSGVPSRFSGSGSGTDYSLTISNLEQEDIATY |
| | VH-anti- | | FCQQGNTLPYTFGGGTKLEITGGGGSGGGGSGGGGSEVKLQE |
| | FLAGVH- | | SGPGLVAPSQSLSVTCTVSGVSLPDYGVSWIRQPPRKGLEWL |
| | VL | | GVIWGSETTYYNSALKSRLTIIKDNSKSQVFLKMNSLQTDDT |
| | | | AIYYCAKHYYYGGSYAMDYWGQGTSVTVSSGGGGSEVQLQ |
| | | | QSGGELAKPGASVKMSCKSSGYTFTAYAIHWAKQAAGAGLE |
| | | | WIGYIAPAAGAAAYNAAFKGKATLAADKSSSTAYMAAAALT |
| | | | SEDSAVYYCARAAAAGADYWGQGTTLTVSSVEGGSGGSGG |
| | | | SGGSGGVDDVLMTQAPLTLPVSLGDQASISCRSSQAIVHANG |
| | | | NTYLEWYLQKPGQSPALLIYKVANRFSGVPDRFSGSGSGTDF |
| | | | TLKISRVEAEDLGVYYCFQGAHAPYTFGGGTKLEIK |
| 154 | Anti- | Full | GATATTCAGATGACACAGACCACAAGCTCCCTGTCCGCCT |
| | CD19VL- | | CTCTGGGCGACAGGGTGACCATCAGCTGCAGGGCCTCCCA |
| | VH-anti- | | GGATATCTCTAAGTATCTGAACTGGTACCAGCAGAAGCCA |
| | FLAGVH- | | GACGGCACCGTGAAGCTGCTGATCTATCACACAAGCAGGC |
| | VL | | TGCACTCCGGAGTGCCATCTCGCTTCAGCGGCTCCGGCTCT |
| | | | GGAACCGACTACAGCCTGACAATCTCCAACCTGGAGCAGG |
| | | | AGGATATCGCCACCTATTTCTGCCAGCAGGGCAATACCCT |
| | | | GCCCTACACATTTGGCGGCGGCACCAAGCTGGAGATCACA |
| | | | GGAGGAGGAGGCAGCGGCGGAGGAGGCTCCGGCGGCGGC |
| | | | GGCTCTGAGGTGAAGCTGCAGGAGTCCGGACCAGGCCTGG |
| | | | TGGCCCCTAGCCAGTCCCTGTCTGTGACCTGTACAGTGTCC |
| | | | GGCGTGTCTCTGCCTGATTACGGCGTGTCCTGGATCAGACA |
| | | | GCCCCCTAGAAAGGGCCTGGAGTGGCTGGGCGTGATCTGG |
| | | | GGCAGCGAGACAACATACTATAACTCTGCCCTGAAGAGCA |
| | | | GGCTGACCATCATCAAGGACAACAGCAAGTCCCAGGTGTT |
| | | | TCTGAAGATGAATAGCCTGCAGACCGACGATACAGCCATC |
| | | | TACTATTGCGCCAAGCACTACTATTACGGCGGCTCTTATGC |
| | | | CATGGATTACTGGGGCCAGGGCACCAGCGTGACAGTGTCT |
| | | | AGCGGAGGAGGAGGCAGCGAGGTGCAGCTGCAGCAGTCC |
| | | | GGCGGCGAGCTGGCCAAGCCTGGGGCCAGCGTGAAGATGT |
| | | | CTTGTAAGTCCTCTGGCTATACCTTCACAGCCTACGCCATC |
| | | | CACTGGGCAAAGCAGGCCGCCGGGGCAGGGCTGGAGTGG |
| | | | ATCGGATATATCGCCCCCGCCGCCGGAGCCGCCGCCTACA |
| | | | ATGCCGCCTTTAAGGGCAAGGCCACCCTGGCCGCCGACAA |
| | | | GAGCTCCTCTACAGCATATATGGCCGCCGCCGCCCTGACC |
| | | | AGCGAGGACTCCGCCGTGTATTACTGCGCAAGGGCCGCCG |
| | | | CCGCCGGAGCCGACTATTGGGGCCAGGGCACCACACTGAC |
| | | | AGTGAGCTCCGTGGAGGGAGGCTCTGGAGGCAGCGGAGG |
| | | | CTCCGGCGGCTCTGGCGGCGTGGACGATGTGCTGATGACC |
| | | | CAGGCCCCACTGACACTGCCCGTGTCCCTGGGCGACCAGG |
| | | | CCTCTATCAGCTGTCGGTCTAGCCAGGCCATCGTGCACGCC |
| | | | AACGGCAATACCTATCTGGAGTGGTACCTGCAGAAGCCTG |
| | | | GCCAGTCCCCAGCCCTGCTGATCTACAAGGTGGCCAATCG |
| | | | GTTCAGCGGCGTGCCCGACAGATTTTCCGGCTCTGGCAGC |
| | | | GGCACCGATTTCACACTGAAGATCAGCAGAGTGGAGGCCG |
| | | | AGGATCTGGGCGTGTATTACTGTTTTCAGGGAGCCCACGCC |
| | | | CCCTACACCTTCGGGGGAGGAACTAAACTGGAAATCAAG |
| 155 | Anti- | VL | DIQMTQTTSSLSASLGDRVTISCRASQDISKYLNWYQQKPDG |
| | CD19VL- | (D1-T107) | TVKLLIYHTSRLHSGVPSRFSGSGSGTDYSLTISNLEQEDIATY |
| | VH-anti- | | FCQQGNTLPYTFGGGTKLEIT |
| | FLAGVH- | | |
| | VL | | |
| 156 | Anti- | L1 | QDISKY |
| | CD19VL- | (Q27-Y32) | |
| | VH-anti- | | |
| | FLAGVH- | | |
| | VL | | |
| 157 | Anti- | L3 | QQGNTLPYT |
| | CD19VL- | (Q89-T97) | |
| | VH-anti- | | |
| | FLAGVH- | | |
| | VL | | |
| 158 | Anti- | L2 | HTS |
| | CD19VL- | (H50-S52) | |
| | VH-anti- | | |
| | FLAGVH- | | |
| | VL | | |
| 159 | Anti- | VH | EVKLQESGPGLVAPSQSLSVTCTVSGVSLPDYGVSWIRQPPRK |
| | CD19VL- | (E123- | GLEWLGVIWGSETTYYNSALKSRLTIIKDNSKSQVFLKMNSL |
| | VH-anti- | S242) | QTDDTAIYYCAKHYYYGGSYAMDYWGQGTSVTVSS |
| | FLAGVH- | | |
| | VL | | |
| 160 | Anti- | H1 | GVSLPDYG |
| | CD19VL- | (G148- | |
| | VH-anti- | G155) | |
| | FLAGVH- | | |
| | VL | | |
| 161 | Anti- | H3 | AKHYYYGGSYAMDY |
| | CD19VL- | (A218- | |
| | VH-anti- | Y231) | |
| | FLAGVH- | | |
| | VL | | |
| 162 | Anti- | H2 | IWGSETT |
| | CD19VL- | (I173- | |
| | VH-anti- | T179) | |
| | FLAGVH- | | |
| | VL | | |
| 163 | Anti- | VH | EVQLQQSGGELAKPGASVKMSCKSSGYTFTAYAIHWAKQAA |
| | CD19VL- | (E248- | GAGLEWIGYIAPAAGAAAYNAAFKGKATLAADKSSSTAYMA |
| | FLAGVH- | | |
| | VL | | |
| 164 | Anti- | H1 | GYTFTAYA |
| | CD19VL- | (G273- | |
| | VH-anti- | A280) | |
| | FLAGVH- | | |
| | VL | | |
| 165 | Anti- | H3 | ARAAAAGADY |
| | CD19VL- | (A344- | |
| | VH-anti- | Y353) | |
| | FLAGVH- | | |
| | VL | | |
| 166 | Anti- | H2 | IAPAAGAA |
| | CD19VL- | (I298- | |
| | VH-anti- | A305) | |
| | FLAGVH- | | |
| | VL | | |
| 167 | Anti- | VL | DVLMTQAPLTLPVSLGDQASISCRSSQAIVHANGNTYLEWYL |
| | CD19VL- | (D383- | QKPGQSPALLIYKVANRFSGVPDRFSGSGSGTDFTLKISRVEA |
| | VH-anti- | K494) | EDLGVYYCFQGAHAPYTFGGGTKLEIK |
| | FLAGVH- | | |
| | VL | | |
| 168 | Anti- | L1 | QAIVHANGNTY |
| | CD19VL- | (Q409- | |
| | VH-anti- | Y419) | |
| | FLAGVH- | | |
| | VL | | |
| 169 | Anti- | L3 | FQGAHAPYT |
| | CD19VL- | (F476- | |
| | VH-anti- | T484) | |
| | FLAGVH- | | |
| | VL | | |
| 170 | Anti- | L2 | KVA |
| | CD19VL- | (K437- | |
| | VH-anti- | A439) | |
| | FLAGVH- | | |
| | VL | | |
| 171 | Anti- | Full | DIQLTQSPSSLSASVGDRVTITCKASQSVDYEGDSFLNWYQQ |
| | CD79bVL- | | KPGKAPKLLIYAASNLESGVPSRFSGSGSGTDFTLTISSLQPED |
| | VH-anti- | | FATYYCQQSNEDPLTFGQGTKVEIKGGGGSGGGGSGGGGSE |
| | FLAGVH- | | VQLVESGGGLVQPGGSLRLSCAASGYTFSSYWIEWVRQAPG |
| | VL | | KGLEWIGEILPGGGDTNYNEIFKGRATFSADTSKNTAYLQMN |
| | | | SLRAEDTAVYYCTRRVPIRLDYWGQGTLVTVSSGGGGSEVQ |
| | | | LQQSGGELAKPGASVKMSCKSSGYTFTAYAIHWAKQAAGAG |
| | | | LEWIGYIAPAAGAAAYNAAFKGKATLAADKSSSTAYMAAAA |
| | | | LTSEDSAVYYCARAAAAGADYWGQGTTLTVSSVEGGSGGSG |
| | | | GSGGSGGVDDVLMTQAPLTLPVSLGDQASISCRSSQAIVHAN |
| | | | GNTYLEWYLQKPGQSPALLIYKVANRFSGVPDRFSGSGSGTD |
| | | | FTLKISRVEAEDLGVYYCFQGAHAPYTFGGGTKLEIK |
| 172 | Anti- | Full | GATATTCAGCTGACCCAGAGCCCAAGCTCCCTGTCTGCCA |
| | CD79bVL- | | GCGTGGGCGATCGGGTGACCATCACATGCAAGGCCTCCCA |
| | VH-anti- | | GTCTGTGGACTACGAGGGCGATTCCTTCCTGAACTGGTATC |
| | FLAGVH- | | AGCAGAAGCCCGGCAAGGCCCCTAAGCTGCTGATCTACGC |
| | VL | | CGCCTCTAATCTGGAGAGCGGCGTGCCTTCCAGATTCAGC |
| | | | GGCTCCGGCTCTGGCACAGACTTTACCCTGACAATCTCTAG |
| | | | CCTGCAGCCAGAGGATTTCGCCACCTACTATTGCCAGCAG |
| | | | AGCAACGAGGACCCCCTGACCTTTGGCCAGGGCACAAAGG |
| | | | TGGAGATCAAGGGAGGAGGAGGCAGCGGCGGAGGAGGCT |
| | | | CCGGCGGCGGCGGCTCTGAGGTGCAGCTGGTGGAGTCCGG |
| | | | AGGAGGCCTGGTGCAGCCTGGAGGCTCTCTGAGGCTGAGC |
| | | | TGTGCAGCCTCCGGCTACACCTTTTCCTCTTATTGGATCGA |
| | | | GTGGGTGCGCCAGGCCCCCGGCAAGGGCCTGGAGTGGATC |
| | | | GGAGAGATCCTGCCTGGAGGAGGCGATACAAACTACAATG |
| | | | AGATCTTCAAGGGCCGGGCCACCTTTTCTGCCGACACCAG |
| | | | CAAGAACACAGCCTATCTGCAGATGAATAGCCTGCGGGCC |
| | | | GAGGATACCGCCGTGTACTATTGCACACGGAGAGTGCCTA |
| | | | TCAGACTGGACTACTGGGGCCAGGGCACCCTGGTGACAGT |
| | | | GAGCTCCGGAGGAGGAGGCAGCGAGGTGCAGCTGCAGCA |
| | | | GTCCGGCGGCGAGCTGGCCAAGCCAGGGGCCAGCGTGAA |
| | | | GATGTCTTGTAAGTCTAGCGGCTACACCTTCACAGCCTATG |
| | | | CCATCCACTGGGCAAAGCAGGCCGCCGGGGCAGGGCTGGA |
| | | | GTGGATCGGATACATCGCCCCCGCCGCCGGAGCCGCCGCC |
| | | | TATAACGCCGCCTTTAAGGGCAAGGCCACCCTGGCCGCCG |
| | | | ACAAGTCCTCTAGCACAGCATACATGGCCGCCGCCGCCCT |
| | | | GACCAGCGAGGATAGCGCCGTGTACTATTGCGCAAGGGCC |
| | | | GCCGCCGCCGGAGCCGACTATTGGGGCCAGGGCACCACAC |
| | | | TGACAGTGTCCTCTGTGGAGGGAGGCTCCGGAGGCTCTGG |
| | | | AGGCAGCGGAGGCTCCGGAGGCGTGGACGATGTGCTGATG |
| | | | ACCCAGGCCCCACTGACACTGCCCGTGAGCCTGGGCGATC |
| | | | AGGCCAGCATCTCCTGTAGGAGCTCCCAGGCCATCGTGCA |
| | | | CGCCAACGGCAATACCTACCTGGAGTGGTATCTGCAGAAG |
| | | | CCTGGCCAGTCTCCAGCCCTGCTGATCTACAAGGTGGCCA |
| | | | ATAGGTTCTCCGGAGTGCCAGACCGCTTTTCTGGCAGCGGC |
| | | | TCCGGCACCGATTTCACACTGAAGATCAGCCGCGTGGAGG |
| | | | CAGAGGACCTGGGCGTGTACTATTGTTTTCAGGGAGCCCA |
| | | | CGCCCCCTACACCTTTGGGGGAGGAACTAAACTGGAAATC |
| | | | AAG |
| 173 | Anti- | VL | DIQLTQSPSSLSASVGDRVTITCKASQSVDYEGDSFLNWYQQ |
| | CD79bVL- | (D1-K111) | KPGKAPKLLIYAASNLESGVPSRFSGSGSGTDFTLTISSLQPED |
| | VH-anti- | | FATYYCQQSNEDPLTFGQGTKVEIK |
| | FLAGVH- | | |
| | VL | | |
| 174 | Anti- | L1 | QSVDYEGDSF |
| | CD79bVL- | (Q27-F36) | |
| | VH-anti- | | |
| | FLAGVH- | | |
| | VL | | |
| 175 | Anti- | L3 | QQSNEDPLT |
| | CD79bVL- | (Q93- | |
| | VH-anti- | T101) | |
| | FLAGVH- | | |
| | VL | | |
| 176 | Anti- | L2 | AAS |
| | CD79bVL- | (A54-S56) | |
| | VH-anti- | | |
| | FLAGVH- | | |
| | VL | | |
| 177 | Anti- | VH | EVQLVESGGGLVQPGGSLRLSCAASGYTFSSYWIEWVRQAPG |
| | CD79bVL- | (E127- | KGLEWIGEILPGGGDTNYNEIFKGRATFSADTSKNTAYLQMN |
| | VH-anti- | S243) | SLRAEDTAVYYCTRRVPIRLDYWGQGTLVTVSS |
| | FLAGVH- | | |
| | VL | | |
| 178 | Anti- | H1 | GYTFSSYW |
| | CD79bVL- | (G152- | |
| | VH-anti- | W159) | |
| | FLAGVH- | | |
| | VL | | |
| 179 | Anti- | H3 | TRRVPIRLDY |
| | CD79bVL- | (T223- | |
| | VH-anti- | Y232) | |
| | FLAGVH- | | |
| | VL | | |
| 180 | Anti- | H2 | ILPGGGDT |
| | CD79bVL- | (I177- | |
| | VH-anti- | T184) | |
| | FLAGVH- | | |
| | VL | | |
| 181 | Anti- | VH | EVQLQQSGGELAKPGASVKMSCKSSGYTFTAYAIHWAKQAA |
| | CD79bVL- | (E249- | GAGLEWIGYIAPAAGAAAYNAAFKGKATLAADKSSSTAYMA |
| | VH-anti- | S365) | AAALTSEDSAVYYCARAAAAGADYWGQGTTLTVSS |
| | FLAGVH- | | |
| | VL | | |
| 182 | Anti- | H1 | GYTFTAYA |
| | CD79bVL- | (G274- | |
| | VH-anti- | A281) | |
| | FLAGVH- | | |
| | VL | | |
| 183 | Anti- | H3 | ARAAAAGADY |
| | CD79bVL- | (A345- | |
| | VH-anti- | Y354) | |
| | FLAGVH- | | |
| | VL | | |
| 184 | Anti- | H2 | IAPAAGAA |
| | CD79bVL- | (I299- | |
| | VH-anti- | A306) | |
| | FLAGVH- | | |
| | VL | | |
| 185 | Anti- | VL | DVLMTQAPLTLPVSLGDQASISCRSSQAIVHANGNTYLEWYL |
| | CD79bVL- | (D384- | QKPGQSPALLIYKVANRFSGVPDRFSGSGSGTDFTLKISRVEA |
| | VH-anti- | K495) | EDLGVYYCFQGAHAPYTFGGGTKLEIK |
| | FLAGVH- | | |
| | VL | | |
| 186 | Anti- | L1 | QAIVHANGNTY |
| | CD79bVL- | (Q410- | |
| | VH-anti- | Y420) | |
| | FLAGVH- | | |
| | VL | | |
| 187 | Anti- | L3 | FQGAHAPYT |
| | CD79bVL- | (F477- | |
| | VH-anti- | T485) | |
| | FLAGVH- | | |
| | VL | | |
| 188 | Anti- | L2 | KVA |
| | CD79bVL- | (K438- | |
| | VH-anti- | A440) | |
| | FLAGVH- | | |
| | VL | | |
| 189 | Anti- | Full | QSVLTQPPSASGTPGQRVTISCSGSSSNIGSNTVNWYQQLPGT |
| | BCMAVL- | | APKLLIFNYHQRPSGVPDRFSGSKSGSSASLAISGLQSEDEAD |
| | VH-anti- | | YYCAAWDDSLNGWVFGGGTKLTVLGGGGSGGGGSGGGGSE |
| | FLAGVH- | | VQLVESGGGLVKPGGSLRLSCAASGFTFGDYALSWFRQAPG |
| | VL | | KGLEWVGVSRSKAYGGTTDYAASVKGRFTISRDDSKSTAYL |
| | | | QMNSLKTEDTAVYYCASSGYSSGWTPFDYWGQGTLVTVSSG |
| | | | GGGSEVQLQQSGGELAKPGASVKMSCKSSGYTFTAYAIHWA |
| | | | KQAAGAGLEWIGYIAPAAGAAAYNAAFKGKATLAADKSSST |
| | | | AYMAAAALTSEDSAVYYCARAAAAGADYWGQGTTLTVSSV |
| | | | EGGSGGSGGSGGSGGVDDVLMTQAPLTLPVSLGDQASISCRS |
| | | | SQAIVHANGNTYLEWYLQKPGQSPALLIYKVANRFSGVPDRF |
| | | | SGSGSGTDFTLKISRVEAEDLGVYYCFQGAHAPYTFGGGTKL |
| | | | EIK |
| 190 | Anti- | Full | CAGAGTGTGCTGACCCAGCCACCTTCTGCCAGCGGAACCC |
| | BCMAVL- | | CTGGACAGAGGGTGACAATCTCCTGCTCTGGCAGCTCCTCT |
| | VH-anti- | | AACATCGGCTCTAACACAGTGAATTGGTACCAGCAGCTGC |
| | FLAGVH- | | CAGGAACCGCCCCCAAGCTGCTGATCTTCAATTATCACCA |
| | VL | | GAGGCCTAGCGGAGTGCCAGACCGCTTTAGCGGCTCCAAG |
| | | | TCTGGCAGCTCCGCCAGCCTGGCCATCTCCGGCCTGCAGTC |
| | | | TGAGGACGAGGCCGATTACTATTGCGCCGCCTGGGACGAT |
| | | | TCCCTGAACGGATGGGTGTTCGGAGGAGGAACCAAGCTGA |
| | | | CAGTGCTGGGCGGCGGCGGCTCTGGAGGAGGAGGCAGCG |
| | | | GCGGAGGAGGCTCCGAGGTGCAGCTGGTGGAGTCCGGCGG |
| | | | CGGCCTGGTGAAGCCTGGAGGCAGCCTGCGCCTGTCCTGT |
| | | | GCAGCCTCTGGCTTCACATTTGGCGACTACGCCCTGAGCTG |
| | | | GTTCAGGCAGGCCCCAGGCAAGGGCCTGGAGTGGGTGGGC |
| | | | GTGAGCCGCTCCAAGGCATACGGAGGAACCACAGATTATG |
| | | | CCGCCTCCGTGAAGGGCCGGTTTACCATCTCTAGAGACGA |
| | | | TTCTAAGAGCACAGCCTACCTGCAGATGAACAGCCTGAAG |
| | | | ACCGAGGACACAGCCGTGTACTATTGCGCCTCTAGCGGCT |
| | | | ACTCCTCTGGCTGGACCCCCTTTGATTATTGGGGCCAGGGC |
| | | | ACCCTGGTGACAGTGAGCTCCGGAGGAGGAGGCTCTGAGG |
| | | | TGCAGCTGCAGCAGAGCGGAGGAGAGCTGGCCAAGCCTG |
| | | | GGGCCAGCGTGAAGATGTCCTGTAAGTCTAGCGGCTACAC |
| | | | CTTCACAGCCTATGCCATCCACTGGGCAAAGCAGGCCGCC |
| | | | GGGGCAGGGCTGGAGTGGATCGGATACATCGCCCCCGCCG |
| | | | CCGGAGCCGCCGCCTATAATGCCGCCTTTAAGGGCAAGGC |
| | | | CACCCTGGCCGCCGATAAGTCCTCTAGCACAGCATACATG |
| | | | GCCGCCGCCGCCCTGACCAGCGAGGACTCCGCCGTGTACT |
| | | | ATTGCGCAAGGGCCGCCGCCGCCGGAGCCGACTACTGGGG |
| | | | CCAGGGCACCACACTGACAGTGTCCTCTGTGGAGGGAGGC |
| | | | TCTGGAGGCAGCGGAGGCTCCGGCGGCTCTGGCGGCGTGG |
| | | | ACGATGTGCTGATGACCCAGGCCCCCCTGACACTGCCCGT |
| | | | GAGCCTGGGCGACCAGGCCTCCATCTCTTGTCGGAGCTCCC |
| | | | AGGCCATCGTGCACGCCAACGGCAATACCTACCTGGAGTG |
| | | | GTATCTGCAGAAGCCAGGACAGAGCCCCGCCCTGCTGATC |
| | | | TACAAGGTGGCCAATCGGTTCTCCGGAGTGCCAGACCGGT |
| | | | TCAGCGGCTCCGGCTCTGGCACCGATTTCACACTGAAGATC |
| | | | AGCAGAGTGGAGGCCGAGGATCTGGGCGTGTACTATTGTT |
| | | | TTCAGGGAGCCCACGCCCCATACACCTTCGGGGGCGGGAC |
| | | | CAAACTGGAAATCAAG |
| 191 | Anti- | VL | QSVLTQPPSASGTPGQRVTISCSGSSSNIGSNTVNWYQQLPGT |
| | BCMAVL- | (Q1-L110) | APKLLIFNYHQRPSGVPDRFSGSKSGSSASLAISGLQSEDEAD |
| | VH-anti- | | YYCAAWDDSLNGWVFGGGTKLTVL |
| | FLAGVH- | | |
| | VL | | |
| 192 | Anti- | L1 | SSNIGSNT |
| | BCMAVL- | (S26-T33) | |
| | VH-anti- | | |
| | FLAGVH- | | |
| | VL | | |
| 193 | Anti- | L3 | AAWDDSLNGWV |
| | BCMAVL- | (A90- | |
| | VH-anti- | V100) | |
| | FLAGVH- | | |
| | VL | | |
| 194 | Anti- | L2 | NYH |
| | BCMAVL- | (N51-H53) | |
| | VH-anti- | | |
| | FLAGVH- | | |
| | VL | | |
| 195 | Anti- | VH | EVQLVESGGGLVKPGGSLRLSCAASGFTFGDYALSWFRQAPG |
| | BCMAVL- | (E126- | KGLEWVGVSRSKAYGGTTDYAASVKGRFTISRDDSKSTAYL |
| | VH-anti- | S248) | QMNSLKTEDTAVYYCASSGYSSGWTPFDYWGQGTLVTVSS |
| | FLAGVH- | | |
| | VL | | |
| 196 | Anti- | H1 | GFTFGDYA |
| | BCMAVL- | (G151- | |
| | VH-anti- | A158) | |
| | FLAGVH- | | |
| | VL | | |
| 197 | Anti- | H3 | ASSGYSSGWTPFDY |
| | BCMAVL- | (A224- | |
| | VH-anti- | Y237) | |
| | FLAGVH- | | |
| | VL | | |
| 198 | Anti- | H2 | SRSKAYGGTT |
| | BCMAVL- | (S176- | |
| | VH-anti- | T185) | |
| | FLAGVH- | | |
| | VL | | |
| 199 | Anti- | VH | EVQLQQSGGELAKPGASVKMSCKSSGYTFTAYAIHWAKQAA |
| | BCMAVL- | (E254- | GAGLEWIGYIAPAAGAAAYNAAFKGKATLAADKSSSTAYMA |
| | VH-anti- | S370) | AAALTSEDSAVYYCARAAAAGADYWGQGTTLTVSS |
| | FLAGVH- | | |
| | VL | | |
| 200 | Anti- | H1 | GYTFTAYA |
| | BCMAVL- | (0279- | |
| | VH-anti- | A286) | |
| | FLAGVH- | | |
| | VL | | |
| 201 | Anti- | H3 | ARAAAAGADY |
| | BCMAVL- | (A350- | |
| | VH-anti- | Y359) | |
| | FLAGVH- | | |
| | VL | | |
| 202 | Anti- | H2 | IAPAAGAA |
| | BCMAVL- | (1304- | |
| | VH-anti- | A311) | |
| | FLAGVH- | | |
| | VL | | |
| 203 | Anti- | VL | DVLMTQAPLTLPVSLGDQASISCRSSQAIVHANGNTYLEWYL |
| | BCMAVL- | (D389- | QKPGQSPALLIYKVANRFSGVPDRFSGSGSGTDFTLKISRVEA |
| | VH-anti- | K500) | EDLGVYYCFQGAHAPYTFGGGTKLEIK |
| | FLAGVH- | | |
| | VL | | |
| 204 | Anti- | L1 | QAIVHANGNTY |
| | BCMAVL- | (Q415- | |
| | VH-anti- | Y425) | |
| | FLAGVH- | | |
| | VL | | |
| 205 | Anti- | L3 | FQGAHAPYT |
| | BCMAVL- | (F482- | |
| | VH-anti- | T490) | |
| | FLAGVH- | | |
| | VL | | |
| 206 | Anti- | L2 | KVA |
| | BCMAVL- | (K443- | |
| | VH-anti- | A445) | |
| | FLAGVH- | | |
| | VL | | |
| 207 | Anti- | Full | DIALTQPASVSGSPGQSITISCTGTSSDIGGYNSVSWYQQHPGK |
| | mesothelin | | APKLMIYGVNNRPSGVSNRFSGSKSGNTASLTISGLQAEDEA |
| | VL-VH- | | DYYCSSYDIESATPVFGGGTKLTVLGGGGSGGGGSGGGGSQ |
| | anti- | | VELVQSGAEVKKPGESLKISCKGSGYSFTSYWIGWVRQAPGK |
| | FLAGVH- | | GLEWMGIIDPGDSRTRYSPSFQGQVTISADKSISTAYLQWSSL |
| | VL | | KASDTAMYYCARGQLYGGTYMDGWGQGTLVTVSSGGGGS |
| | | | EVQLQQSGGELAKPGASVKMSCKSSGYTFTAYAIHWAKQAA |
| | | | GAGLEWIGYIAPAAGAAAYNAAFKGKATLAADKSSSTAYMA |
| | | | AAALTSEDSAVYYCARAAAAGADYWGQGTTLTVSSVEGGS |
| | | | GGSGGSGGSGGVDDVLMTQAPLTLPVSLGDQASISCRSSQAI |
| | | | VHANGNTYLEWYLQKPGQSPALLIYKVANRFSGVPDRFSGS |
| | | | GSGTDFTLKISRVEAEDLGVYYCFQGAHAPYTFGGGTKLEIK |
| 208 | Anti- | Full | GATATTGCACTGACACAGCCCGCCTCTGTGAGCGGCTCCCC |
| | mesothelin | | TGGACAGAGCATCACCATCTCCTGCACCGGCACAAGCTCC |
| | VL-VH- | | GACATCGGCGGCTACAACTCTGTGAGCTGGTATCAGCAGC |
| | anti- | | ACCCCGGCAAGGCCCCTAAGCTGATGATCTACGGCGTGAA |
| | FLAGVH- | | CAATAGGCCATCCGGCGTGTCTAACCGCTTCTCCGGCTCTA |
| | VL | | AGAGCGGCAATACCGCCTCTCTGACAATCAGCGGCCTGCA |
| | | | GGCAGAGGACGAGGCAGATTACTATTGCTCTAGCTACGAT |
| | | | ATCGAGAGCGCCACCCCCGTGTTTGGAGGAGGAACCAAGC |
| | | | TGACAGTGCTGGGCGGCGGCGGCTCTGGAGGAGGAGGCA |
| | | | GCGGCGGAGGAGGCTCCCAGGTGGAGCTGGTGCAGTCCGG |
| | | | AGCCGAGGTGAAGAAGCCTGGCGAGTCCCTGAAGATCTCT |
| | | | TGTAAGGGCAGCGGCTACTCCTTCACATCTTATTGGATCGG |
| | | | ATGGGTGCGGCAGGCCCCAGGCAAGGGCCTGGAGTGGATG |
| | | | GGCATCATCGACCCAGGCGATAGCCGGACCAGATACTCCC |
| | | | CCTCTTTTCAGGGCCAGGTGACCATCTCCGCCGACAAGAG |
| | | | CATCTCCACAGCCTATCTGCAGTGGTCCTCTCTGAAGGCCA |
| | | | GCGATACAGCCATGTACTATTGCGCCAGAGGCCAGCTGTA |
| | | | CGGAGGAACCTATATGGACGGATGGGGACAGGGCACCCTG |
| | | | GTGACAGTGAGCTCCGGAGGAGGAGGCTCTGAGGTGCAGC |
| | | | TGCAGCAGAGCGGAGGAGAGCTGGCCAAGCCAGGGGCCA |
| | | | GCGTGAAGATGTCCTGTAAGTCTAGCGGCTACACCTTCAC |
| | | | AGCCTATGCCATCCACTGGGCAAAGCAGGCCGCCGGGGCA |
| | | | GGGCTGGAGTGGATCGGATACATCGCCCCCGCCGCCGGAG |
| | | | CCGCCGCCTATAACGCCGCCTTTAAGGGCAAGGCCACCCT |
| | | | GGCCGCCGATAAGTCCTCTAGCACAGCATACATGGCCGCC |
| | | | GCCGCCCTGACCAGCGAGGACTCCGCCGTGTACTATTGCG |
| | | | CAAGAGCCGCCGCCGCCGGAGCCGATTATTGGGGACAGGG |
| | | | CACCACACTGACAGTGTCCTCTGTGGAGGGAGGCTCTGGA |
| | | | GGCAGCGGAGGCTCCGGCGGCTCTGGCGGCGTGGACGATG |
| | | | TGCTGATGACCCAGGCCCCACTGACACTGCCCGTGAGCCT |
| | | | GGGCGACCAGGCCTCTATCAGCTGTAGGAGCTCCCAGGCC |
| | | | ATCGTGCACGCCAACGGCAATACCTACCTGGAGTGGTATC |
| | | | TGCAGAAGCCTGGCCAGTCCCCAGCCCTGCTGATCTACAA |
| | | | GGTGGCCAATCGGTTCTCTGGCGTGCCTGACAGATTTTCCG |
| | | | GCTCTGGCAGCGGCACCGATTTCACACTGAAGATCTCCCG |
| | | | CGTGGAGGCAGAGGATCTGGGCGTGTACTATTGTTTTCAG |
| | | | GGAGCCCACGCCCCCTACACCTTCGGGGGGGGCACAAAAC |
| | | | TGGAAATCAAG |
| 209 | Anti- | VL | DIALTQPASVSGSPGQSITISCTGTSSDIGGYNSVSWYQQHPGK |
| | mesothelin | (D1-L111) | APKLMIYGVNNRPSGVSNRFSGSKSGNTASLTISGLQAEDEA |
| | VL-VH- | | DYYCSSYDIESATPVFGGGTKLTVL |
| | anti- | | |
| | FLAGVH- | | |
| | VL | | |
| 210 | Anti- | L1 | SSDIGGYNS |
| | mesothelin | (S26-S34) | |
| | VL-VH- | | |
| | anti- | | |
| | FLAGVH- | | |
| | VL | | |
| 211 | Anti- | L3 | SSYDIESATPV |
| | mesothelin | (S91- | |
| | VL-VH- | V101) | |
| | anti- | | |
| | FLAGVH- | | |
| | VL | | |
| 212 | Anti- | L2 | GVN |
| | mesothelin | (G52-N54) | |
| | VL-VH- | | |
| | anti- | | |
| | FLAGVH- | | |
| | VL | | |
| 213 | Anti- | VH | QVELVQSGAEVKKPGESLKISCKGSGYSFTSYWIGWVRQAPG |
| | mesothelin | (Q127- | KGLEWMGIIDPGDSRTRYSPSFQGQVTISADKSISTAYLQWSS |
| | VL-VH- | S246) | LKASDTAMYYCARGQLYGGTYMDGWGQGTLVTVSS |
| | anti- | | |
| | FLAGVH- | | |
| | VL | | |
| 214 | Anti- | H1 | GYSFTSYW |
| | mesothelin | (W52- | |
| | VL-VH- | W159) | |
| | anti- | | |
| | FLAGVH- | | |
| | VL | | |
| 215 | Anti- | H3 | ARGQLYGGTYMDG |
| | mesothelin | (A223 - | |
| | VL-VH- | G235) | |
| | anti- | | |
| | FLAGVH- | | |
| | VL | | |
| 216 | Anti- | H2 | IDPGDSRT |
| | mesothelin | (I177- | |
| | VL-VH- | T184) | |
| | anti- | | |
| | FLAGVH- | | |
| | VL | | |
| 217 | Anti- | VH | EVQLQQSGGELAKPGASVKMSCKSSGYTFTAYAIHWAKQAA |
| | mesothelin | (E252- | GAGLEWIGYIAPAAGAAAYNAAFKGKATLAADKSSSTAYMA |
| | VL-VH- | S368) | AAALTSEDSAVYYCARAAAAGADYWGQGTTLTVSS |
| | anti- | | |
| | FLAGVH- | | |
| | VL | | |
| 218 | Anti- | H1 | GYTFTAYA |
| | mesothelin | (G277- | |
| | VL-VH- | A284) | |
| | anti- | | |
| | FLAGVH- | | |
| | VL | | |
| 219 | Anti- | H3 | ARAAAAGADY |
| | mesothelin | (A348- | |
| | VL-VH- | Y357) | |
| | anti- | | |
| | FLAGVH- | | |
| | VL | | |
| 220 | Anti- | H2 | IAPAAGAA |
| | mesothelin | (I302- | |
| | VL-VH- | A309) | |
| | anti- | | |
| | FLAGVH- | | |
| | VL | | |
| 221 | Anti- | VL | DVLMTQAPLTLPVSLGDQASISCRSSQAIVHANGNTYLEWYL |
| | mesothelin | (D387- | QKPGQSPALLIYKVANRFSGVPDRFSGSGSGTDFTLKISRVEA |
| | VL-VH- | K498) | EDLGVYYCFQGAHAPYTFGGGTKLEIK |
| | anti- | | |
| | FLAGVH- | | |
| | VL | | |
| 222 | Anti- | L1 | QAIVHANGNTY |
| | mesothelin | (Q413 - | |
| | VL-VH- | Y423) | |
| | anti- | | |
| | FLAGVH- | | |
| | VL | | |
| 223 | Anti- | L3 | FQGAHAPYT |
| | mesothelin | (F480- | |
| | VL-VH- | T488) | |
| | anti- | | |
| | FLAGVH- | | |
| | VL | | |
| 224 | Anti- | L2 | KVA |
| | mesothelin | (K441- | |
| | VL-VH- | A443) | |
| | anti- | | |
| | FLAGVH- | | |
| | VL | | |
| 225 | Anti- | Full | DIQLTQSPSSLSASVGDRVTITCKASQSVDYEGDSFLNWYQQ |
| | CD79bVL- | | KPGKAPKLLIYAASNLESGVPSRFSGSGSGTDFTLTISSLQPED |
| | VH-anti- | | FATYYCQQSNEDPLTFGQGTKVEIKGGGGSGGGGSGGGGSE |
| | FMC63id | | VQLVESGGGLVQPGGSLRLSCAASGYTFSSYWIEWVRQAPG |
| | VH-VL | | KGLEWIGEILPGGGDTNYNEIFKGRATFSADTSKNTAYLQMN |
| | | | SLRAEDTAVYYCTRRVPIRLDYWGQGTLVTVSSGGGGSEVK |
| | | | LVESGGGLVQPGGSLKLSCAASGFDFSRYWMSWVRQAPGKG |
| | | | LEWIGEINLDSSTINYTPSLKDKFIISRDNAKNTLYLQMSKVRS |
| | | | EDTALYYCARRYDAMDYWGQGTSVTVSSVEGGSGGSGGSG |
| | | | GSGGVDDIVLTQSPASLAVSLGQRATISCRASESVDDYGISFM |
| | | | NWFQQKPGQPPKLLIYAAPNQGSGVPARFSGSGSGTDFSLNIH |
| | | | PMEEDDTAMYFCQQSKDVRWRHQAGDQTG |
| 226 | Anti- | Full | GATATTCAGCTGACCCAGTCTCCTAGCTCCCTGAGCGCCTC |
| | CD79bVL- | | CGTGGGCGATAGGGTGACCATCACATGCAAGGCCTCTCAG |
| | VH-anti- | | AGCGTGGACTACGAGGGCGATTCCTTCCTGAACTGGTATC |
| | FMC63id | | AGCAGAAGCCAGGCAAGGCCCCCAAGCTGCTGATCTACGC |
| | VH-VL | | AGCCAGCAATCTGGAGTCCGGAGTGCCATCTCGCTTCTCCG |
| | | | GCTCTGGCAGCGGAACCGACTTTACCCTGACAATCTCTAGC |
| | | | CTGCAGCCAGAGGATTTCGCCACATACTATTGCCAGCAGA |
| | | | GCAACGAGGACCCCCTGACCTTTGGCCAGGGCACAAAGGT |
| | | | GGAGATCAAGGGAGGAGGAGGCTCCGGCGGAGGAGGCTC |
| | | | TGGCGGCGGCGGCAGCGAGGTGCAGCTGGTGGAGTCCGGC |
| | | | GGCGGCCTGGTGCAGCCCGGCGGCAGCCTGCGGCTGTCCT |
| | | | GTGCCGCCTCTGGCTACACCTTTTCCTCTTATTGGATCGAG |
| | | | TGGGTGAGACAGGCCCCCGGCAAGGGCCTGGAGTGGATCG |
| | | | GAGAGATCCTGCCTGGAGGAGGCGATACCAACTACAATGA |
| | | | GATCTTCAAGGGAAGGGCCACCTTCAGCGCCGACACCTCC |
| | | | AAGAACACAGCCTATCTGCAGATGAATAGCCTGAGGGCCG |
| | | | AGGATACCGCCGTGTACTATTGCACACGGAGAGTGCCAAT |
| | | | CAGGCTGGACTACTGGGGACAGGGCACCCTGGTGACAGTG |
| | | | AGCTCCGGAGGAGGAGGCAGCGAGGTGAAGCTGGTGGAG |
| | | | TCCGGAGGAGGCCTGGTGCAGCCTGGAGGCTCTCTGAAGC |
| | | | TGAGCTGTGCCGCCTCCGGCTTCGATTTTTCCAGGTATTGG |
| | | | ATGTCTTGGGTGCGCCAGGCCCCTGGCAAGGGCCTGGAAT |
| | | | GGATCGGCGAGATCAACCTGGACTCTAGCACCATCAATTA |
| | | | CACACCATCTCTGAAGGACAAGTTCATCATCAGCCGGGAT |
| | | | AACGCCAAGAATACCCTGTATCTGCAGATGTCTAAGGTGA |
| | | | GAAGCGAGGATACAGCCCTGTACTATTGCGCCAGGCGCTA |
| | | | CGACGCCATGGATTATTGGGGCCAGGGCACCAGCGTGACA |
| | | | GTGTCCTCTGTGGAGGGAGGCAGCGGAGGCTCCGGAGGCT |
| | | | CTGGAGGCAGCGGAGGAGTGGACGATATCGTGCTGACCCA |
| | | | GTCCCCAGCCTCTCTGGCCGTGTCCCTGGGCCAGCGGGCCA |
| | | | CAATCTCTTGTAGAGCCTCCGAGTCTGTGGACGATTACGGC |
| | | | ATCTCCTTCATGAACTGGTTTCAGCAGAAGCCCGGCCAGCC |
| | | | CCCTAAGCTGCTGATCTATGCCGCCCCTAATCAGGGCAGC |
| | | | GGAGTGCCAGCCAGGTTCAGCGGCTCCGGCTCTGGAACCG |
| | | | ACTTTTCCCTGAATATCCACCCTATGGAGGAGGACGATAC |
| | | | AGCCATGTACTTTTGTCAGCAGAGCAAGGACGTGAGGTGG |
| | | | AGACATCAGGCAGGCGACCAGACAGGA |
| 227 | Anti- | VL | DIQLTQSPSSLSASVGDRVTITCKASQSVDYEGDSFLNWYQQ |
| | CD79bVL- | (D1-K111) | KPGKAPKLLIYAASNLESGVPSRFSGSGSGTDFTLTISSLQPED |
| | VH-anti- | | FATYYCQQSNEDPLTFGQGTKVEIK |
| | FMC63id | | |
| | VH-VL | | |
| 228 | Anti- | L1 | QSVDYEGDSF |
| | CD79bVL- | (Q27-F36) | |
| | VH-anti- | | |
| | FMC63id | | |
| | VH-VL | | |
| 229 | Anti- | L3 | QQSNEDPLT |
| | CD79bVL- | (Q93- | |
| | VH-anti- | T101) | |
| | FMC63id | | |
| | VH-VL | | |
| 230 | Anti- | L2 | AAS |
| | CD79bVL- | (A54-S56) | |
| | VH-anti- | | |
| | FMC63id | | |
| | VH-VL | | |
| 231 | Anti- | VH | EVQLVESGGGLVQPGGSLRLSCAASGYTFSSYWIEWVRQAPG |
| | CD79bVL- | (E127- | KGLEWIGEILPGGGDTNYNEIFKGRATFSADTSKNTAYLQMN |
| | VH-anti- | S243) | SLRAEDTAVYYCTRRVPIRLDYWGQGTLVTVSS |
| | FMC63id | | |
| | VH-VL | | |
| 232 | Anti- | H1 | GYTFSSYW |
| | CD79bVL- | (G152- | |
| | VH-anti- | W159) | |
| | FMC63id | | |
| | VH-VL | | |
| 233 | Anti- | H3 | TRRVPIRLDY |
| | CD79bVL- | (T223- | |
| | VH-anti- | Y232) | |
| | FMC63id | | |
| | VH-VL | | |
| 234 | Anti- | H2 | ILPGGGDT |
| | CD79bVL- | (I177- | |
| | VH-anti- | T184) | |
| | FMC63id | | |
| | VH-VL | | |
| 235 | Anti- | VH | EVKLVESGGGLVQPGGSLKLSCAASGFDFSRYWMSWVRQAP |
| | CD79bVL- | (E249- | GKGLEWIGEINLDSSTINYTPSLKDKFIISRDNAKNTLYLQMS |
| | VH-anti- | S364) | KVRSEDTALYYCARRYDAMDYWGQGTSVTVSS |
| | FMC63id | | |
| | VH-VL | | |
| 236 | Anti- | H1 | GFDFSRYW |
| | CD79bVL- | (G274- | |
| | VH-anti- | W281) | |
| | FMC63id | | |
| | VH-VL | | |
| 237 | Anti- | H3 | ARRYDAMDY |
| | CD79bVL- | (A345- | |
| | VH-anti- | Y353) | |
| | FMC63id | | |
| | VH-VL | | |
| 238 | Anti- | H2 | INLDSSTI |
| | CD79bVL- | (I299- | |
| | VH-anti- | I306) | |
| | FMC63id | | |
| | VH-VL | | |
| 239 | Anti- | VL | DIVLTQSPASLAVSLGQRATISCRASESVDDYGISFMNWFQQK |
| | CD79bVL- | (D383- | PGQPPKLLIYAAPNQGSGVPARFSGSGSGTDFSLNIHPMEEDD |
| | VH-anti- | G491) | TAMYFCQQSKDVRWRHQAGDQTG |
| | FMC63id | | |
| | VH-VL | | |
| 240 | Anti- | L1 | ESVDDYGISF |
| | CD79bVL- | (E409- | |
| | VH-anti- | F418) | |
| | FMC63id | | |
| | VH-VL | | |
| 241 | Anti- | L3 | QQSKDVRWRHQA |
| | CD79bVL- | (Q475- | |
| | VH-anti- | A486) | |
| | FMC63id | | |
| | VH-VL | | |
| 242 | Anti- | L2 | AAP |
| | CD79bVL- | (A436- | |
| | VH-anti- | P438) | |
| | FMC63id | | |
| | VH-VL | | |
| 243 | Anti- | Full | QSVLTQPPSASGTPGQRVTISCSGSSSNIGSNTVNWYQQLPGT |
| | BCMAVL- | | APKLLIFNYHQRPSGVPDRFSGSKSGSSASLAISGLQSEDEAD |
| | VH-anti- | | YYCAAWDDSLNGWVFGGGTKLTVLGGGGSGGGGSGGGGSE |
| | FMC63id | | VQLVESGGGLVKPGGSLRLSCAASGFTFGDYALSWFRQAPG |
| | VH-VL | | KGLEWVGVSRSKAYGGTTDYAASVKGRFTISRDDSKSTAYL |
| | | | QMNSLKTEDTAVYYCASSGYSSGWTPFDYWGQGTLVTVSSG |
| | | | GGGSEVKLVESGGGLVQPGGSLKLSCAASGFDFSRYWMSWV |
| | | | RQAPGKGLEWIGEINLDSSTINYTPSLKDKFIISRDNAKNTLYL |
| | | | QMSKVRSEDTALYYCARRYDAMDYWGQGTSVTVSSVEGGS |
| | | | GGSGGSGGSGGVDDIVLTQSPASLAVSLGQRATISCRASESVD |
| | | | DYGISFMNWFQQKPGQPPKLLIYAAPNQGSGVPARFSGSGSG |
| | | | TDFSLNIHPMEEDDTAMYFCQQSKDVRWRHQAGDQTG |
| 244 | Anti- | Full | CAGAGCGTGCTGACCCAGCCACCTAGCGCCTCCGGAACCC |
| | BCMAVL- | | CAGGCCAGAGGGTGACAATCTCTTGCAGCGGCAGCTCCTC |
| | VH-anti- | | TAACATCGGCTCCAACACCGTGAATTGGTACCAGCAGCTG |
| | FMC63id | | CCTGGCACAGCCCCAAAGCTGCTGATCTTCAATTATCACCA |
| | VH-VL | | GAGGCCCAGCGGAGTGCCTGACCGCTTTTCCGGCTCTAAG |
| | | | AGCGGCAGCTCCGCCTCCCTGGCCATCTCTGGCCTGCAGA |
| | | | GCGAGGACGAGGCCGATTACTATTGCGCCGCCTGGGACGA |
| | | | TTCCCTGAACGGATGGGTGTTCGGAGGAGGAACCAAGCTG |
| | | | ACAGTGCTGGGCGGAGGAGGCAGCGGAGGAGGAGGCTCC |
| | | | GGCGGCGGCGGCTCTGAGGTGCAGCTGGTGGAATCCGGAG |
| | | | GAGGCCTGGTGAAGCCAGGAGGCTCCCTGCGCCTGTCTTG |
| | | | TGCCGCCAGCGGCTTCACCTTTGGCGACTACGCCCTGAGCT |
| | | | GGTTCAGGCAGGCCCCTGGCAAGGGCCTGGAGTGGGTGGG |
| | | | CGTGTCCCGCTCTAAGGCATACGGAGGCACCACAGATTAT |
| | | | GCCGCCTCCGTGAAGGGCAGGTTTACCATCAGCCGGGACG |
| | | | ATAGCAAGTCCACAGCCTATCTGCAGATGAATAGCCTGAA |
| | | | GACCGAGGACACAGCCGTGTACTATTGCGCCTCTAGCGGC |
| | | | TACTCCTCTGGCTGGACCCCATTCGATTATTGGGGCCAGGG |
| | | | CACCCTGGTGACAGTGAGCTCCGGAGGAGGAGGCTCTGAG |
| | | | GTGAAGCTGGTGGAGAGCGGAGGAGGCCTGGTGCAGCCA |
| | | | GGAGGCTCCCTGAAGCTGTCCTGCGCCGCCAGCGGCTTCG |
| | | | ACTTTAGCCGGTACTGGATGTCCTGGGTGAGACAGGCCCC |
| | | | TGGCAAGGGCCTGGAATGGATCGGCGAGATCAACCTGGAT |
| | | | TCTAGCACCATCAATTACACACCAAGCCTGAAGGACAAGT |
| | | | TTATCATCTCCCGGGATAACGCCAAGAATACCCTGTATCTG |
| | | | CAGATGTCCAAGGTGAGATCTGAGGACACAGCCCTGTACT |
| | | | ATTGCGCCCGGAGATACGACGCCATGGACTACTGGGGCCA |
| | | | GGGCACCTCCGTGACAGTGTCCTCTGTGGAGGGAGGCTCC |
| | | | GGAGGCTCTGGAGGCAGCGGCGGCTCCGGCGGCGTGGACG |
| | | | ATATCGTGCTGACCCAGTCTCCTGCCAGCCTGGCCGTGTCT |
| | | | CTGGGCCAGAGGGCCACAATCAGCTGTAGAGCCTCTGAGA |
| | | | GCGTGGACGATTACGGCATCAGCTTCATGAACTGGTTTCA |
| | | | GCAGAAGCCAGGCCAGCCACCCAAGCTGCTGATCTATGCC |
| | | | GCCCCAAATCAGGGCTCCGGAGTGCCCGCCCGGTTCTCCG |
| | | | GCTCTGGCAGCGGCACCGATTTTTCTCTGAACATCCACCCT |
| | | | ATGGAGGAGGACGATACAGCCATGTACTTTTGTCAGCAGA |
| | | | GCAAGGACGTGCGCTGGAGACATCAGGCAGGAGACCAGA |
| | | | CAGGA |
| 245 | Anti- | VL | QSVLTQPPSASGTPGQRVTISCSGSSSNIGSNTVNWYQQLPGT |
| | BCMAVL- | (Q1-L110) | APKLLIFNYHQRPSGVPDRFSGSKSGSSASLAISGLQSEDEAD |
| | VH-anti- | | YYCAAWDDSLNGWVFGGGTKLTVL |
| | FMC63id | | |
| | VH-VL | | |
| 246 | Anti- | L1 | SSNIGSNT |
| | BCMAVL- | (S26-T33) | |
| | VH-anti- | | |
| | FMC63id | | |
| | VH-VL | | |
| 247 | Anti- | L3 | AAWDDSLNGWV |
| | BCMAVL- | (A90- | |
| | VH-anti- | V100) | |
| | FMC63id | | |
| | VH-VL | | |
| 248 | Anti- | L2 | NYH |
| | BCMAVL- | (N51-H53) | |
| | VH-anti- | | |
| | FMC63id | | |
| | VH-VL | | |
| 249 | Anti- | VH | EVQLVESGGGLVKPGGSLRLSCAASGFTFGDYALSWFRQAPG |
| | BCMAVL- | (E126- | KGLEWVGVSRSKAYGGTTDYAASVKGRFTISRDDSKSTAYL |
| | VH-anti- | S248) | QMNSLKTEDTAVYYCASSGYSSGWTPFDYWGQGTLVTVSS |
| | FMC63id | | |
| | VH-VL | | |
| 250 | Anti- | H1 | GFTFGDYA |
| | BCMAVL- | (G151- | |
| | VH-anti- | A158) | |
| | FMC63id | | |
| | VH-VL | | |
| 251 | Anti- | H3 | ASSGYSSGWTPFDY |
| | BCMAVL- | (A224- | |
| | VH-anti- | Y237) | |
| | FMC63id | | |
| | VH-VL | | |
| 252 | Anti- | H2 | SRSKAYGGTT |
| | BCMAVL- | (S176- | |
| | VH-anti- | T185) | |
| | FMC63id | | |
| | VH-VL | | |
| 253 | Anti- | VH | EVKLVESGGGLVQPGGSLKLSCAASGFDFSRYWMSWVRQAP |
| | BCMAVL- | (E254- | GKGLEWIGEINLDSSTINYTPSLKDKFIISRDNAKNTLYLQMS |
| | VH-anti- | S369) | KVRSEDTALYYCARRYDAMDYWGQGTSVTVSS |
| | FMC63id | | |
| | VH-VL | | |
| 254 | Anti- | H1 | GFDFSRYW |
| | BCMAVL- | (G279- | |
| | VH-anti- | W286) | |
| | FMC63id | | |
| | VH-VL | | |
| 255 | Anti- | H3 | ARRYDAMDY |
| | BCMAVL- | (A350- | |
| | VH-anti- | Y358) | |
| | FMC63id | | |
| | VH-VL | | |
| 256 | Anti- | H2 | INLDSSTI |
| | BCMAVL- | (I304- | |
| | VH-anti- | I311) | |
| | FMC63id | | |
| | VH-VL | | |
| 257 | Anti- | VL | DIVLTQSPASLAVSLGQRATISCRASESVDDYGISFMNWFQQK |
| | BCMAVL- | (D388- | PGQPPKLLIYAAPNQGSGVPARFSGSGSGTDFSLNIHPMEEDD |
| | VH-anti- | G496) | TAMYFCQQSKDVRWRHQAGDQTG |
| | FMC63id | | |
| | VH-VL | | |
| 258 | Anti- | L1 | ESVDDYGISF |
| | BCMAVL- | (E414- | |
| | VH-anti- | F423) | |
| | FMC63id | | |
| | VH-VL | | |
| 259 | Anti- | L3 | QQSKDVRWRHQA |
| | BCMAVL- | (Q480- | |
| | VH-anti- | A491) | |
| | FMC63id | | |
| | VH-VL | | |
| 260 | Anti- | L2 | AAP |
| | BCMAVL- | (A441 - | |
| | VH-anti- | P443) | |
| | FMC63id | | |
| | VH-VL | | |
| 261 | Anti- | Full | DIALTQPASVSGSPGQSITISCTGTSSDIGGYNSVSWYQQHPGK |
| | mesothelin | | APKLMIYGVNNRPSGVSNRFSGSKSGNTASLTISGLQAEDEA |
| | VL-VH- | | DYYCSSYDIESATPVFGGGTKLTVLGGGGSGGGGSGGGGSQ |
| | anti- | | VELVQSGAEVKKPGESLKISCKGSGYSFTSYWIGWVRQAPGK |
| | FMC63id | | GLEWMGIIDPGDSRTRYSPSFQGQVTISADKSISTAYLQWSSL |
| | VH-VL | | KASDTAMYYCARGQLYGGTYMDGWGQGTLVTVSSGGGGS |
| | | | EVKLVESGGGLVQPGGSLKLSCAASGFDFSRYWMSWVRQAP |
| | | | GKGLEWIGEINLDSSTINYTPSLKDKFIISRDNAKNTLYLQMS |
| | | | KVRSEDTALYYCARRYDAMDYWGQGTSVTVSSVEGGSGGS |
| | | | GGSGGSGGVDDIVLTQSPASLAVSLGQRATISCRASESVDDY |
| | | | GISFMNWFQQKPGQPPKLLIYAAPNQGSGVPARFSGSGSGTD |
| | | | FSLNIHPMEEDDTAMYFCQQSKDVRWRHQAGDQTG |
| 262 | Anti- | Full | GACATCGCACTGACCCAGCCTGCCAGCGTGTCCGGCTCTCC |
| | mesothelin | | AGGACAGTCCATCACAATCTCTTGCACCGGCACAAGCTCC |
| | VL-VH- | | GACATCGGCGGCTACAACAGCGTGTCCTGGTATCAGCAGC |
| | anti- | | ACCCAGGCAAGGCCCCCAAGCTGATGATCTACGGCGTGAA |
| | FMC63id | | CAATAGGCCTTCTGGCGTGAGCAACCGCTTCTCTGGCAGC |
| | VH-VL | | AAGTCCGGCAATACCGCCAGCCTGACAATCTCCGGCCTGC |
| | | | AGGCAGAGGACGAGGCAGATTACTATTGCTCTAGCTATGA |
| | | | TATCGAGAGCGCCACCCCAGTGTTTGGAGGAGGAACCAAG |
| | | | CTGACAGTGCTGGGCGGAGGAGGCAGCGGAGGAGGAGGC |
| | | | TCCGGCGGCGGCGGCTCTCAGGTGGAGCTGGTGCAGTCCG |
| | | | GAGCCGAGGTGAAGAAGCCCGGCGAGTCTCTGAAGATCAG |
| | | | CTGTAAGGGCTCCGGCTACTCTTTCACCAGCTATTGGATCG |
| | | | GATGGGTGCGGCAGGCCCCTGGCAAGGGCCTGGAGTGGAT |
| | | | GGGCATCATCGACCCAGGCGATTCTAGGACCCGCTACTCT |
| | | | CCCAGCTTTCAGGGCCAGGTGACCATCTCCGCCGACAAGT |
| | | | CCATCTCTACAGCCTATCTGCAGTGGTCCTCTCTGAAGGCC |
| | | | AGCGATACCGCCATGTACTATTGCGCCAGAGGCCAGCTGT |
| | | | ACGGCGGCACATATATGGACGGATGGGGACAGGGCACCCT |
| | | | GGTGACAGTGAGCTCCGGAGGAGGAGGCTCTGAGGTGAA |
| | | | GCTGGTGGAGAGCGGAGGAGGCCTGGTGCAGCCAGGAGG |
| | | | CTCCCTGAAGCTGTCTTGTGCCGCCAGCGGCTTCGACTTTA |
| | | | GCCGGTACTGGATGTCCTGGGTGAGACAGGCCCCTGGCAA |
| | | | GGGCCTGGAATGGATCGGCGAGATCAACCTGGATTCTAGC |
| | | | ACCATCAATTACACACCATCCCTGAAGGACAAGTTCATCA |
| | | | TCTCTAGGGATAACGCCAAGAATACCCTGTATCTGCAGAT |
| | | | GTCCAAGGTGCGCTCTGAGGATACAGCCCTGTACTATTGC |
| | | | GCCCGGAGATACGACGCCATGGATTATTGGGGCCAGGGCA |
| | | | CCAGCGTGACAGTGTCCTCTGTGGAGGGAGGCTCCGGAGG |
| | | | CTCTGGAGGCAGCGGCGGCTCCGGCGGCGTGGACGATATC |
| | | | GTGCTGACCCAGTCTCCAGCCAGCCTGGCCGTGAGCCTGG |
| | | | GCCAGAGGGCCACAATCTCCTGTAGAGCCAGCGAGTCCGT |
| | | | GGACGATTACGGCATCTCCTTCATGAACTGGTTTCAGCAGA |
| | | | AGCCCGGCCAGCCCCCTAAGCTGCTGATCTATGCCGCCCCT |
| | | | AATCAGGGCAGCGGAGTGCCTGCCCGGTTCTCTGGCAGCG |
| | | | GCTCCGGCACCGACTTTTCCCTGAATATCCACCCTATGGAG |
| | | | GAGGACGATACAGCCATGTACTTTTGTCAGCAGAGCAAGG |
| | | | ACGTGCGGTGGAGGCATCAGGCAGGGGACCAGACAGGA |
| 263 | Anti- | VL | DIALTQPASVSGSPGQSITISCTGTSSDIGGYNSVSWYQQHPGK |
| | mesothelin | (D1-L111) | APKLMIYGVNNRPSGVSNRFSGSKSGNTASLTISGLQAEDEA |
| | VL-VH- | | DYYCSSYDIESATPVFGGGTKLTVL |
| | anti- | | |
| | FMC63id | | |
| | VH-VL | | |
| 264 | Anti- | L1 | SSDIGGYNS |
| | mesothelin | (S26-S34) | |
| | VL-VH- | | |
| | anti- | | |
| | FMC63id | | |
| | VH-VL | | |
| 265 | Anti- | L3 | SSYDIESATPV |
| | mesothelin | (S91- | |
| | VL-VH- | V101) | |
| | anti- | | |
| | FMC63id | | |
| | VH-VL | | |
| 266 | Anti- | L2 | GVN |
| | mesothelin | (G52-N54) | |
| | VL-VH- | | |
| | anti- | | |
| | FMC63id | | |
| | VH-VL | | |
| 267 | Anti- | VH | QVELVQSGAEVKKPGESLKISCKGSGYSFTSYWIGWVRQAPG |
| | mesothelin | (Q127- | KGLEWMGIIDPGDSRTRYSPSFQGQVTISADKSISTAYLQWSS |
| | VL-VH- | S246) | LKASDTAMYYCARGQLYGGTYMDGWGQGTLVTVSS |
| | anti- | | |
| | FMC63id | | |
| | VH-VL | | |
| 268 | Anti- | H1 | GYSFTSYW |
| | mesothelin | (W52- | |
| | VL-VH- | W159) | |
| | anti- | | |
| | FMC63id | | |
| | VH-VL | | |
| 269 | Anti- | H3 | ARGQLYGGTYMDG |
| | mesothelin | (A223- | |
| | VL-VH- | G235) | |
| | anti- | | |
| | FMC63id | | |
| | VH-VL | | |
| 270 | Anti- | H2 | IDPGDSRT |
| | mesothelin | (I177- | |
| | VL-VH- | T184) | |
| | anti- | | |
| | FMC63id | | |
| | VH-VL | | |
| 271 | Anti- | VH | EVKLVESGGGLVQPGGSLKLSCAASGFDFSRYWMSWVRQAP |
| | mesothelin | (E252- | GKGLEWIGEINLDSSTINYTPSLKDKFIISRDNAKNTLYLQMS |
| | VL-VH- | S367) | KVRSEDTALYYCARRYDAMDYWGQGTSVTVSS |
| | anti- | | |
| | FMC63id | | |
| | VH-VL | | |
| 272 | Anti- | H1 | GFDFSRYW |
| | mesothelin | (G277- | |
| | VL-VH- | W284) | |
| | anti- | | |
| | FMC63id | | |
| | VH-VL | | |
| 273 | Anti- | H3 | ARRYDAMDY |
| | mesothelin | (A348- | |
| | VL-VH- | Y356) | |
| | anti- | | |
| | FMC63id | | |
| | VH-VL | | |
| 274 | Anti- | H2 | INLDSSTI |
| | mesothelin | (I302- | |
| | VL-VH- | I309) | |
| | anti- | | |
| | FMC63id | | |
| | VH-VL | | |
| 275 | Anti- | VL | DIVLTQSPASLAVSLGQRATISCRASESVDDYGISFMNWFQQK |
| | mesothelin | (D386- | PGQPPKLLIYAAPNQGSGVPARFSGSGSGTDFSLNIHPMEEDD |
| | VL-VH- | G494) | TAMYFCQQSKDVRWRHQAGDQTG |
| | anti- | | |
| | FMC63id | | |
| | VH-VL | | |
| 276 | Anti- | L1 | ESVDDYGISF |
| | mesothelin | (E412- | |
| | VL-VH- | F421) | |
| | anti- | | |
| | FMC63id | | |
| | VH-VL | | |
| 277 | Anti- | L3 | QQSKDVRWRHQA |
| | mesothelin | (Q478- | |
| | VL-VH- | A489) | |
| | anti- | | |
| | FMC63id | | |
| | VH-VL | | |
| 278 | Anti- | L2 | AAP |
| | mesothelin | (A439- | |
| | VL-VH- | P441) | |
| | anti- | | |
| | FMC63id | | |
| | VH-VL | | |
| 279 | Anti- | Full | EVQLVESGGGLVQPGGSLRLSCAASGYTFSSYWIEWVRQAPG |
| | CD79bscFv- | | KGLEWIGEILPGGGDTNYNEIFKGRATFSADTSKNTAYLQMN |
| | HetFcB | | SLRAEDTAVYYCTRRVPIRLDYWGQGTLVTVSSVEGGSGGS |
| | | | GGSGGSGGVDDIQLTQSPSSLSASVGDRVTITCKASQSVDYEG |
| | | | DSFLNWYQQKPGKAPKLLIYAASNLESGVPSRFSGSGSGTDF |
| | | | TLTISSLQPEDFATYYCQQSNEDPLTFGQGTKVEIKAAEPKSS |
| | | | DKTHTCPPCPAPEAAGGPSVFLFPPKPKDTLMISRTPEVTCVV |
| | | | VSVSHEDPEVKFNWYVDGVEVHNAKTKPREEQYNSTYRVVS |
| | | | VLTVLHQDWLNGKEYKCKVSNKALPAPIEKTISKAKGQPREP |
| | | | QVYVLPPSRDELTKNQVSLLCLVKGFYPSDIAVEWESNGQPE |
| | | | NNYLTWPPVLDSDGSFFLYSKLTVDKSRWQQGNVFSCSVMH |
| | | | EALHNHYTQKSLSLSPG |
| 280 | Anti- | Full | GAGGTCCAGCTGGTGGAGTCTGGAGGAGGCCTGGTGCAGC |
| | CD79bscFv- | | CAGGAGGCTCCCTGCGGCTGTCTTGCGCAGCCAGCGGATA |
| | HetFcB | | CACCTTCAGCTCCTATTGGATCGAGTGGGTGAGACAGGCC |
| | | | CCAGGCAAGGGCCTGGAGTGGATCGGAGAGATCCTGCCAG |
| | | | GAGGAGGCGATACCAACTACAATGAGATCTTCAAGGGCCG |
| | | | GGCCACATTTTCCGCCGACACCTCTAAGAACACAGCCTATC |
| | | | TGCAGATGAATAGCCTGAGGGCCGAGGATACCGCCGTGTA |
| | | | CTATTGCACACGGAGAGTGCCAATCAGGCTGGACTACTGG |
| | | | GGACAGGGCACCCTGGTGACAGTGTCTAGCGTGGAGGGAG |
| | | | GCAGCGGAGGCTCCGGAGGCTCTGGAGGCAGCGGAGGAG |
| | | | TGGACGATATCCAGCTGACCCAGAGCCCTTCCTCTCTGTCT |
| | | | GCCAGCGTGGGCGATAGGGTGACCATCACCTGTAAGGCCT |
| | | | CCCAGTCTGTGGACTACGAGGGCGATTCCTTTCTGAACTGG |
| | | | TATCAGCAGAAGCCCGGCAAGGCCCCTAAGCTGCTGATCT |
| | | | ATGCAGCCAGCAATCTGGAGTCCGGAGTGCCATCTCGCTT |
| | | | CAGCGGCTCCGGCTCTGGAACCGACTTTACCCTGACAATC |
| | | | AGCTCCCTGCAGCCTGAGGATTTCGCCACATACTATTGTCA |
| | | | GCAGTCCAACGAGGACCCACTGACCTTTGGCCAGGGCACA |
| | | | AAGGTGGAAATCAAAGCAGCAGAGCCAAAGTCATCCGAT |
| | | | AAGACCCATACCTGTCCCCCTTGCCCGGCGCCAGAGGCAG |
| | | | CAGGAGGACCAAGCGTGTTCCTGTTTCCACCCAAGCCCAA |
| | | | AGACACCCTGATGATTAGCCGAACCCCTGAAGTCACATGC |
| | | | GTGGTCGTGTCCGTGTCTCACGAGGACCCAGAAGTCAAGT |
| | | | TCAACTGGTACGTGGATGGCGTCGAGGTGCATAATGCCAA |
| | | | GACAAAACCCCGGGAGGAACAGTACAACAGCACCTATAG |
| | | | AGTCGTGTCCGTCCTGACAGTGCTGCACCAGGATTGGCTG |
| | | | AACGGCAAGGAATATAAGTGCAAAGTGTCCAATAAGGCCC |
| | | | TGCCCGCTCCTATCGAGAAAACCATTTCTAAGGCAAAAGG |
| | | | CCAGCCTCGCGAACCACAGGTCTACGTGCTGCCTCCATCCC |
| | | | GGGACGAGCTGACAAAGAACCAGGTCTCTCTGCTGTGCCT |
| | | | GGTGAAAGGCTTCTATCCATCAGATATTGCTGTGGAGTGG |
| | | | GAAAGCAATGGGCAGCCCGAGAACAATTACCTGACTTGGC |
| | | | CCCCTGTGCTGGACTCTGATGGGAGTTTCTTTCTGTATTCT |
| | | | AAGCTGACCGTGGATAAAAGTAGGTGGCAGCAGGGAAAT |
| | | | GTCTTTAGTTGTTCAGTGATGCATGAAGCCCTGCATAACCA |
| | | | CTACACCCAGAAAAGCCTGTCCCTGTCCCCCGGA |
| 281 | Anti- | VH | EVQLVESGGGLVQPGGSLRLSCAASGYTFSSYWIEWVRQAPG |
| | CD79bscFv- | (E1-S117) | KGLEWIGEILPGGGDTNYNEIFKGRATFSADTSKNTAYLQMN |
| | HetFcB | | SLRAEDTAVYYCTRRVPIRLDYWGQGTLVTVSS |
| 282 | Anti- | H1 | GYTFSSYW |
| | CD79bscFv- | (G26- | |
| | HetFcB | W33) | |
| 283 | Anti- | H3 | TRRVPIRLDY |
| | CD79bscFv- | (T97- | |
| | HetFcB | Y106) | |
| 284 | Anti- | H2 | ILPGGGDT |
| | CD79bscFv- | (I51-T58) | |
| | HetFcB | | |
| 285 | Anti- | VL | DIQLTQSPSSLSASVGDRVTITCKASQSVDYEGDSFLNWYQQ |
| | CD79bscFv- | (D136- | KPGKAPKLLIYAASNLESGVPSRFSGSGSGTDFTLTISSLQPED |
| | HetFcB | K246) | FATYYCQQSNEDPLTFGQGTKVEIK |
| 286 | Anti- | L1 | QSVDYEGDSF |
| | CD79bscFv- | (Q162- | |
| | HetFcB | F171) | |
| 287 | Anti- | L3 | QQSNEDPLT |
| | CD79bscFv- | (Q228- | |
| | HetFcB | T236) | |
| 288 | Anti- | L2 | AAS |
| | CD79bscFv- | (A189- | |
| | HetFcB | S191) | |
| 289 | Anti- | CH2 | APEAAGGPSVFLFPPKPKDTLMISRTPEVTCVVVSVSHEDPEV |
| | CD79bscFv- | (A264- | KFNWYVDGVEVHNAKTKPREEQYNSTYRVVSVLTVLHQDW |
| | HetFcB | K373) | LNGKEYKCKVSNKALPAPIEKTISKAK |
| 290 | Anti- | CH3 | GQPREPQVYVLPPSRDELTKNQVSLLCLVKGFYPSDIAVEWE |
| | CD79bscFv- | (G374- | SNGQPENNYLTWPPVLDSDGSFFLYSKLTVDKSRWQQGNVF |
| | HetFcB | G479) | SCSVMHEALHNHYTQKSLSLSPG |
| 291 | Anti- | Full | EVQLVESGGGLVKPGGSLRLSCAASGFTFGDYALSWFRQAPG |
| | BCMAscFv- | | KGLEWVGVSRSKAYGGTTDYAASVKGRFTISRDDSKSTAYL |
| | HetFcB | | QMNSLKTEDTAVYYCASSGYSSGWTPFDYWGQGTLVTVSSV |
| | | | EGGSGGSGGSGGSGGVDQSVLTQPPSASGTPGQRVTISCSGSS |
| | | | SNIGSNTVNWYQQLPGTAPKLLIFNYHQRPSGVPDRFSGSKSG |
| | | | SSASLAISGLQSEDEADYYCAAWDDSLNGWVFGGGTKLTVL |
| | | | AAEPKSSDKTHTCPPCPAPEAAGGPSVFLFPPKPKDTLMISRTP |
| | | | EVTCVVVSVSHEDPEVKFNWYVDGVEVHNAKTKPREEQYNS |
| | | | TYRVVSVLTVLHQDWLNGKEYKCKVSNKALPAPIEKTISKAK |
| | | | GQPREPQVYVLPPSRDELTKNQVSLLCLVKGFYPSDIAVEWE |
| | | | SNGQPENNYLTWPPVLDSDGSFFLYSKLTVDKSRWQQGNVF |
| | | | SCSVMHEALHNHYTQKSLSLSPG |
| 292 | Anti- | Full | GAGGTCCAGCTGGTGGAGAGCGGAGGAGGCCTGGTGAAG |
| | BCMAscFv- | | CCAGGAGGCTCTCTGAGGCTGAGCTGCGCAGCCTCCGGCT |
| | HetFcB | | TCACCTTTGGCGACTACGCCCTGTCCTGGTTCAGGCAGGCC |
| | | | CCTGGCAAGGGCCTGGAGTGGGTGGGCGTGTCTAGAAGCA |
| | | | AGGCCTACGGCGGCACCACAGATTATGCCGCCTCTGTGAA |
| | | | GGGCCGGTTTACCATCAGCAGAGACGATTCCAAGTCTACA |
| | | | GCCTATCTGCAGATGAACAGCCTGAAGACCGAGGACACAG |
| | | | CCGTGTACTATTGCGCCAGCTCCGGCTACTCTAGCGGCTGG |
| | | | ACCCCATTCGATTATTGGGGCCAGGGCACCCTGGTGACAG |
| | | | TGTCCTCTGTGGAGGGAGGCTCCGGAGGCTCTGGAGGCAG |
| | | | CGGCGGCTCCGGAGGAGTGGACCAGTCCGTGCTGACACAG |
| | | | CCACCTAGCGCCTCCGGAACCCCAGGACAGAGAGTGACAA |
| | | | TCTCTTGTAGCGGCAGCTCCTCTAACATCGGCTCCAACACC |
| | | | GTGAATTGGTACCAGCAGCTGCCAGGCACAGCCCCCAAGC |
| | | | TGCTGATCTTCAATTATCACCAGAGGCCTTCTGGCGTGCCA |
| | | | GATCGCTTTTCCGGCTCTAAGAGCGGCAGCTCCGCCTCTCT |
| | | | GGCCATCAGCGGCCTGCAGTCCGAGGACGAGGCAGATTAC |
| | | | TATTGTGCCGCCTGGGACGATAGCCTGAATGGCTGGGTGTT |
| | | | TGGCGGCGGCACCAAGCTGACTGTCCTGGCTGCTGAACCA |
| | | | AAATCATCCGATAAGACCCACACTTGCCCACCCTGCCCGG |
| | | | CGCCAGAGGCAGCAGGAGGACCAAGCGTGTTCCTGTTTCC |
| | | | ACCCAAGCCCAAAGACACCCTGATGATTAGCCGAACCCCT |
| | | | GAAGTCACATGCGTGGTCGTGTCCGTGTCTCACGAGGACC |
| | | | CAGAAGTCAAGTTCAACTGGTACGTGGATGGCGTCGAGGT |
| | | | GCATAATGCCAAGACAAAACCCCGGGAGGAACAGTACAA |
| | | | CAGCACCTATAGAGTCGTGTCCGTCCTGACAGTGCTGCACC |
| | | | AGGATTGGCTGAACGGCAAGGAATATAAGTGCAAAGTGTC |
| | | | CAATAAGGCCCTGCCCGCTCCTATCGAGAAAACCATTTCTA |
| | | | AGGCAAAAGGCCAGCCTCGCGAACCACAGGTCTACGTGCT |
| | | | GCCTCCATCCCGGGACGAGCTGACAAAGAACCAGGTCTCT |
| | | | CTGCTGTGCCTGGTGAAAGGCTTCTATCCATCAGATATTGC |
| | | | TGTGGAGTGGGAAAGCAATGGGCAGCCCGAGAACAATTAC |
| | | | CTGACTTGGCCCCCTGTGCTGGACTCTGATGGGAGTTTCTT |
| | | | TCTGTATTCTAAGCTGACCGTGGATAAAAGTAGGTGGCAG |
| | | | CAGGGAAATGTCTTTAGTTGTTCAGTGATGCATGAAGCCCT |
| | | | GCATAACCACTACACCCAGAAAAGCCTGTCCCTGTCCCCC |
| | | | GGA |
| 293 | Anti- | VH | EVQLVESGGGLVKPGGSLRLSCAASGFTFGDYALSWFRQAPG |
| | BCMAscFv- | (E1-S123) | KGLEWVGVSRSKAYGGTTDYAASVKGRFTISRDDSKSTAYL |
| | HetFcB | | QMNSLKTEDTAVYYCASSGYSSGWTPFDYWGQGTLVTVSS |
| 294 | Anti- | H1 | GFTFGDYA |
| | BCMAscFv- | (G26-A33) | |
| | HetFcB | | |
| 295 | Anti- | H3 | ASSGYSSGWTPFDY |
| | BCMAscFv- | (A99- | |
| | HetFcB | Y112) | |
| 296 | Anti- | H2 | SRSKAYGGTT |
| | BCMAscFv- | (S51-T60) | |
| | HetFcB | | |
| 297 | Anti- | VL | QSVLTQPPSASGTPGQRVTISCSGSSSNIGSNTVNWYQQLPGT |
| | BCMAscFv- | (Q142- | APKLLIFNYHQRPSGVPDRFSGSKSGSSASLAISGLQSEDEAD |
| | HetFcB | L251) | YYCAAWDDSLNGWVFGGGTKLTVL |
| 298 | Anti- | L1 | SSNIGSNT |
| | BCMAscFv- | (S167- | |
| | HetFcB | T174) | |
| 299 | Anti- | L3 | AAWDDSLNGWV |
| | BCMAscFv- | (A231- | |
| | HetFcB | V241) | |
| 300 | Anti- | L2 | NYH |
| | BCMAscFv- | (N192- | |
| | HetFcB | H194) | |
| 301 | Anti- | CH2 | APEAAGGPSVFLFPPKPKDTLMISRTPEVTCVVVSVSHEDPEV |
| | BCMAscFv- | (A269- | KFNWYVDGVEVHNAKTKPREEQYNSTYRVVSVLTVLHQDW |
| | HetFcB | K378) | LNGKEYKCKVSNKALPAPIEKTISKAK |
| 302 | Anti- | CH3 | GQPREPQVYVLPPSRDELTKNQVSLLCLVKGFYPSDIAVEWE |
| | BCMAscFv- | (G379- | SNGQPENNYLTWPPVLDSDGSFFLYSKLTVDKSRWQQGNVF |
| | HetFcB | G484) | SCSVMHEALHNHYTQKSLSLSPG |
| 303 | Anti- | Full | QVELVQSGAEVKKPGESLKISCKGSGYSFTSYWIGWVRQAPG |
| | mesothelin | | KGLEWMGIIDPGDSRTRYSPSFQGQVTISADKSISTAYLQWSS |
| | scFv- | | LKASDTAMYYCARGQLYGGTYMDGWGQGTLVTVSSVEGGS |
| | HetFcB | | GGSGGSGGSGGVDDIALTQPASVSGSPGQSITISCTGTSSDIGG |
| | | | YNSVSWYQQHPGKAPKLMIYGVNNRPSGVSNRFSGSKSGNT |
| | | | ASLTISGLQAEDEADYYCSSYDIESATPVFGGGTKLTVLAAEP |
| | | | KSSDKTHTCPPCPAPEAAGGPSVFLFPPKPKDTLMISRTPEVTC |
| | | | VVVSVSHEDPEVKFNWYVDGVEVHNAKTKPREEQYNSTYR |
| | | | VVSVLTVLHQDWLNGKEYKCKVSNKALPAPIEKTISKAKGQP |
| | | | REPQVYVLPPSRDELTKNQVSLLCLVKGFYPSDIAVEWESNG |
| | | | QPENNYLTWPPVLDSDGSFFLYSKLTVDKSRWQQGNVFSCS |
| | | | VMHEALHNHYTQKSLSLSPG |
| 304 | Anti- | Full | CAGGTCGAGCTGGTGCAGTCCGGAGCCGAGGTGAAGAAGC |
| | mesothelin | | CCGGCGAGTCTCTGAAGATCAGCTGCAAGGGCTCTGGCTA |
| | scFv- | | CAGCTTCACCTCCTATTGGATCGGATGGGTGCGGCAGGCC |
| | HetFcB | | CCTGGCAAGGGCCTGGAGTGGATGGGCATCATCGACCCTG |
| | | | GCGATTCTCGGACCAGATACTCTCCAAGCTTTCAGGGCCA |
| | | | GGTGACCATCAGCGCCGACAAGTCCATCTCTACAGCCTAT |
| | | | CTGCAGTGGAGCTCCCTGAAGGCCAGCGATACCGCCATGT |
| | | | ACTATTGCGCCAGGGGCCAGCTGTACGGAGGAACATATAT |
| | | | GGACGGATGGGGACAGGGCACCCTGGTGACAGTGTCTAGC |
| | | | GTGGAGGGAGGCTCTGGAGGCAGCGGAGGCTCCGGAGGC |
| | | | TCTGGAGGAGTGGACGATATCGCCCTGACCCAGCCAGCCA |
| | | | GCGTGTCCGGCTCTCCCGGCCAGTCCATCACAATCTCTTGT |
| | | | ACCGGCACATCCTCTGATATCGGCGGCTACAACAGCGTGT |
| | | | CCTGGTATCAGCAGCACCCCGGCAAGGCCCCTAAGCTGAT |
| | | | GATCTACGGCGTGAACAATAGGCCAAGCGGCGTGTCCAAC |
| | | | CGCTTCTCTGGCAGCAAGTCCGGCAATACCGCCAGCCTGA |
| | | | CAATCTCCGGCCTGCAGGCAGAGGACGAGGCAGATTACTA |
| | | | TTGTAGCTCCTATGACATCGAGTCCGCCACCCCCGTGTTTG |
| | | | GAGGAGGCACAAAGCTGACAGTCCTGGCTGCTGAACCAAA |
| | | | ATCATCCGATAAGACCCATACCTGCCCCCCCTGCCCGGCGC |
| | | | CAGAGGCAGCAGGAGGACCAAGCGTGTTCCTGTTTCCACC |
| | | | CAAGCCCAAAGACACCCTGATGATTAGCCGAACCCCTGAA |
| | | | GTCACATGCGTGGTCGTGTCCGTGTCTCACGAGGACCCAG |
| | | | AAGTCAAGTTCAACTGGTACGTGGATGGCGTCGAGGTGCA |
| | | | TAATGCCAAGACAAAACCCCGGGAGGAACAGTACAACAG |
| | | | CACCTATAGAGTCGTGTCCGTCCTGACAGTGCTGCACCAG |
| | | | GATTGGCTGAACGGCAAGGAATATAAGTGCAAAGTGTCCA |
| | | | ATAAGGCCCTGCCCGCTCCTATCGAGAAAACCATTTCTAA |
| | | | GGCAAAAGGCCAGCCTCGCGAACCACAGGTCTACGTGCTG |
| | | | CCTCCATCCCGGGACGAGCTGACAAAGAACCAGGTCTCTC |
| | | | TGCTGTGCCTGGTGAAAGGCTTCTATCCATCAGATATTGCT |
| | | | GTGGAGTGGGAAAGCAATGGGCAGCCCGAGAACAATTAC |
| | | | CTGACTTGGCCCCCTGTGCTGGACTCTGATGGGAGTTTCTT |
| | | | TCTGTATTCTAAGCTGACCGTGGATAAAAGTAGGTGGCAG |
| | | | CAGGGAAATGTCTTTAGTTGTTCAGTGATGCATGAAGCCCT |
| | | | GCATAACCACTACACCCAGAAAAGCCTGTCCCTGTCCCCC |
| | | | GGA |
| 305 | Anti- | VH | QVELVQSGAEVKKPGESLKISCKGSGYSFTSYWIGWVRQAPG |
| | mesothelin | (Q1-S120) | KGLEWMGIIDPGDSRTRYSPSFQGQVTISADKSISTAYLQWSS |
| | scFv- | | LKASDTAMYYCARGQLYGGTYMDGWGQGTLVTVSS |
| | HetFcB | | |
| 306 | Anti- | H1 | GYSFTSYW |
| | mesothelin | (G26-W33) | |
| | scFv- | | |
| | HetFcB | | |
| 307 | Anti- | H3 | ARGQLYGGTYMDG |
| | mesothelin | (A97- | |
| | scFv- | G109) | |
| | HetFcB | | |
| 308 | Anti- | H2 | IDPGDSRT |
| | mesothelin | (I51-T58) | |
| | scFv- | | |
| | HetFcB | | |
| 309 | Anti- | VL | DIALTQPASVSGSPGQSITISCTGTSSDIGGYNSVSWYQQHPGK |
| | mesothelin | (D139- | APKLMIYGVNNRPSGVSNRFSGSKSGNTASLTISGLQAEDEA |
| | scFv- | L249) | DYYCSSYDIESATPVFGGGTKLTVL |
| | HetFcB | | |
| 310 | Anti- | L1 | SSDIGGYNS |
| | mesothelin | (S164- | |
| | scFv- | S172) | |
| | HetFcB | | |
| 311 | Anti- | L3 | SSYDIESATPV |
| | mesothelin | (S229- | |
| | scFv- | V239) | |
| | HetFcB | | |
| 312 | Anti- | L2 | GVN |
| | mesothelin | (G190- | |
| | scFv- | N192) | |
| | HetFcB | | |
| 313 | Anti- | CH2 | APEAAGGPSVFLFPPKPKDTLMISRTPEVTCVVVSVSHEDPEV |
| | mesothelin | (A267- | KFNWYVDGVEVHNAKTKPREEQYNSTYRVVSVLTVLHQDW |
| | scFv- | K376) | LNGKEYKCKVSNKALPAPIEKTISKAK |
| | HetFcB | | |
| 314 | Anti- | CH3 | GQPREPQVYVLPPSRDELTKNQVSLLCLVKGFYPSDIAVEWE |
| | mesothelin | (G377- | SNGQPENNYLTWPPVLDSDGSFFLYSKLTVDKSRWQQGNVF |
| | scFv- | G482) | SCSVMHEALHNHYTQKSLSLSPG |
| | HetFcB | | |
| 315 | Anti- | Full | EVQLQQSGGELAKPGASVKMSCKSSGYTFTAYAIHWAKQAA |
| | FLAGVH- | | GAGLEWIGYIAPAAGAAAYNAAFKGKATLAADKSSSTAYMA |
| | CH-HetFcA | | AAALTSEDSAVYYCARAAAAGADYWGQGTTLTVSSASTKGP |
| | | | SVFPLAPSSKSTSGGTAALGCLVKDYFPEPVTVSWNSGALTS |
| | | | GVHTFPAVLQSSGLYSLSSVVTVPSSSLGTQTYICNVNHKPSN |
| | | | TKVDKKVEPKSCDKTHTCPPCPAPEAAGGPSVFLFPPKPKDTL |
| | | | MISRTPEVTCVVVSVSHEDPEVKFNWYVDGVEVHNAKTKPR |
| | | | EEQYNSTYRVVSVLTVLHQDWLNGKEYKCKVSNKALPAPIE |
| | | | KTISKAKGQPREPQVYVYPPSRDELTKNQVSLTCLVKGFYPS |
| | | | DIAVEWESNGQPENNYKTTPPVLDSDGSFALVSKLTVDKSRW |
| | | | QQGNVFSCSVMHEALHNHYTQKSLSLSPG |
| 316 | Anti- | Full | GAGGTCCAGCTGCAGCAGTCCGGAGGAGAGCTGGCCAAGC |
| | FLAGVH- | | CAGGGGCCAGCGTGAAGATGTCTTGCAAGAGCTCCGGCTA |
| | CH-HetFcA | | CACCTTCACAGCCTATGCCATCCACTGGGCAAAGCAGGCC |
| | | | GCCGGAGCTGGCCTGGAGTGGATCGGATACATCGCACCCG |
| | | | CCGCCGGAGCCGCCGCCTATAACGCCGCCTTTAAGGGCAA |
| | | | GGCCACCCTGGCCGCCGACAAGTCTAGCTCCACAGCATAC |
| | | | ATGGCCGCCGCCGCCCTGACCAGCGAGGATAGCGCCGTGT |
| | | | ACTATTGTGCCAGGGCAGCAGCAGCAGGAGCCGACTACTG |
| | | | GGGGCAGGGGACTACTCTGACTGTGAGCTCCGCTAGCACC |
| | | | AAGGGACCTTCCGTGTTCCCACTGGCACCAAGCTCCAAGT |
| | | | CTACAAGCGGAGGAACCGCCGCCCTGGGATGTCTGGTGAA |
| | | | GGATTACTTCCCAGAGCCCGTGACCGTGTCTTGGAACAGC |
| | | | GGGGCCCTGACCAGCGGAGTGCACACCTTTCCTGCCGTGC |
| | | | TGCAGTCTAGCGGCCTGTATTCCCTGTCCTCTGTGGTCACA |
| | | | GTGCCAAGCTCCTCTCTGGGCACACAGACCTACATCTGCA |
| | | | ACGTGAATCACAAGCCATCCAATACCAAGGTCGACAAGAA |
| | | | GGTGGAGCCCAAGTCTTGTGATAAGACACACACCTGCCCA |
| | | | CCTTGTCCGGCGCCAGAGGCAGCAGGAGGACCAAGCGTGT |
| | | | TCCTGTTTCCACCCAAGCCTAAGGACACACTGATGATCTCC |
| | | | AGGACACCAGAGGTGACCTGCGTGGTGGTGTCCGTGTCTC |
| | | | ACGAGGACCCCGAGGTGAAGTTCAACTGGTACGTGGATGG |
| | | | CGTGGAGGTGCACAATGCCAAGACCAAGCCCAGGGAGGA |
| | | | GCAGTATAACTCTACATACCGCGTGGTGAGCGTGCTGACC |
| | | | GTGCTGCACCAGGATTGGCTGAACGGCAAGGAGTACAAGT |
| | | | GCAAGGTGAGCAATAAGGCCCTGCCCGCCCCTATCGAGAA |
| | | | GACCATCTCCAAGGCCAAGGGCCAGCCTCGCGAACCACAG |
| | | | GTGTACGTGTACCCTCCATCTAGAGACGAGCTGACAAAGA |
| | | | ACCAGGTGAGCCTGACCTGTCTGGTGAAGGGCTTTTATCCC |
| | | | AGCGATATCGCCGTGGAGTGGGAGTCCAATGGCCAGCCTG |
| | | | AGAACAATTACAAGACAACCCCCCCTGTGCTGGACTCCGA |
| | | | TGGCTCTTTCGCCCTGGTGTCCAAGCTGACCGTGGACAAGT |
| | | | CTCGGTGGCAGCAGGGCAACGTGTTCAGCTGTTCCGTGAT |
| | | | GCACGAGGCACTGCACAATCACTACACCCAGAAGTCACTG |
| | | | TCACTGTCCCCAGGC |
| 317 | Anti- | VH | EVQLQQSGGELAKPGASVKMSCKSSGYTFTAYAIHWAKQAA |
| | FLAGVH- | (E1-S117) | GAGLEWIGYIAPAAGAAAYNAAFKGKATLAADKSSSTAYMA |
| | CH-HetFcA | | AAALTSEDSAVYYCARAAAAGADYWGQGTTLTVSS |
| 318 | Anti- | H1 | GYTFTAYA |
| | FLAGVH- | (G26-A33) | |
| | CH-HetFcA | | |
| 319 | Anti- | H3 | ARAAAAGADY |
| | FLAGVH- | (A97- | |
| | CH-HetFcA | Y106) | |
| 320 | Anti- | H2 | IAPAAGAA |
| | FLAGVH- | (I51-A58) | |
| | CH-HetFcA | | |
| 321 | Anti- | CH1 | ASTKGPSVFPLAPSSKSTSGGTAALGCLVKDYFPEPVTVSWNS |
| | FLAGVH- | (A118- | GALTSGVHTFPAVLQSSGLYSLSSVVTVPSSSLGTQTYICNVN |
| | CH-HetFcA | V215) | HKPSNTKVDKKV |
| 322 | Anti- | CH2 | APEAAGGPSVFLFPPKPKDTLMISRTPEVTCVVVSVSHEDPEV |
| | FLAGVH- | (A231- | KFNWYVDGVEVHNAKTKPREEQYNSTYRVVSVLTVLHQDW |
| | CH-HetFcA | K340) | LNGKEYKCKVSNKALPAPIEKTISKAK |
| 323 | Anti- | CH3 | GQPREPQVYVYPPSRDELTKNQVSLTCLVKGFYPSDIAVEWE |
| | FLAGVH- | (G341- | SNGQPENNYKTTPPVLDSDGSFALVSKLTVDKSRWQQGNVF |
| | CH-HetFcA | G446) | SCSVMHEALHNHYTQKSLSLSPG |
| 324 | Anti- | Full | EVKLVESGGGLVQPGGSLKLSCAASGFDFSRYWMSWVRQAP |
| | FMC63id | | GKGLEWIGEINLDSSTINYTPSLKDKFIISRDNAKNTLYLQMS |
| | VH-CH- | | KVRSEDTALYYCARRYDAMDYWGQGTSVTVSSASTKGPSVF |
| | HetFcA | | PLAPSSKSTSGGTAALGCLVKDYFPEPVTVSWNSGALTSGVH |
| | | | TFPAVLQSSGLYSLSSVVTVPSSSLGTQTYICNVNHKPSNTKV |
| | | | DKKVEPKSCDKTHTCPPCPAPEAAGGPSVFLFPPKPKDTLMIS |
| | | | RTPEVTCVVVSVSHEDPEVKFNWYVDGVEVHNAKTKPREEQ |
| | | | YNSTYRVVSVLTVLHQDWLNGKEYKCKVSNKALPAPIEKTIS |
| | | | KAKGQPREPQVYVYPPSRDELTKNQVSLTCLVKGFYPSDIAV |
| | | | EWESNGQPENNYKTTPPVLDSDGSFALVSKLTVDKSRWQQG |
| | | | NVFSCSVMHEALHNHYTQKSLSLSPG |
| 325 | Anti- | Full | GAGGTCAAGCTGGTGGAGTCTGGAGGAGGCCTGGTGCAGC |
| | FMC63id | | CAGGAGGCTCTCTGAAGCTGAGCTGCGCCGCCTCCGGCTT |
| | VH-CH- | | CGACTTTTCCCGGTACTGGATGTCTTGGGTGAGACAGGCCC |
| | HetFcA | | CCGGCAAGGGCCTGGAGTGGATCGGCGAGATCAACCTGGA |
| | | | TAGCTCCACCATCAATTACACACCTAGCCTGAAGGACAAG |
| | | | TTCATCATCTCCAGGGATAACGCCAAGAATACCCTGTATCT |
| | | | GCAGATGTCTAAGGTGCGGAGCGAGGACACAGCCCTGTAC |
| | | | TATTGTGCACGCAGATACGATGCTATGGATTATTGGGGGC |
| | | | AGGGAACCTCAGTCACCGTCTCTTCTGCTAGCACCAAGGG |
| | | | ACCTTCCGTGTTCCCACTGGCACCAAGCTCCAAGTCTACAA |
| | | | GCGGAGGAACCGCCGCCCTGGGATGTCTGGTGAAGGATTA |
| | | | CTTCCCAGAGCCCGTGACCGTGTCTTGGAACAGCGGGGCC |
| | | | CTGACCAGCGGAGTGCACACCTTTCCTGCCGTGCTGCAGTC |
| | | | TAGCGGCCTGTATTCCCTGTCCTCTGTGGTCACAGTGCCAA |
| | | | GCTCCTCTCTGGGCACACAGACCTACATCTGCAACGTGAAT |
| | | | CACAAGCCATCCAATACCAAGGTCGACAAGAAGGTGGAGC |
| | | | CCAAGTCTTGTGATAAGACACACACCTGCCCACCTTGTCCG |
| | | | GCGCCAGAGGCAGCAGGAGGACCAAGCGTGTTCCTGTTTC |
| | | | CACCCAAGCCTAAGGACACACTGATGATCTCCAGGACACC |
| | | | AGAGGTGACCTGCGTGGTGGTGTCCGTGTCTCACGAGGAC |
| | | | CCCGAGGTGAAGTTCAACTGGTACGTGGATGGCGTGGAGG |
| | | | TGCACAATGCCAAGACCAAGCCCAGGGAGGAGCAGTATA |
| | | | ACTCTACATACCGCGTGGTGAGCGTGCTGACCGTGCTGCA |
| | | | CCAGGATTGGCTGAACGGCAAGGAGTACAAGTGCAAGGTG |
| | | | AGCAATAAGGCCCTGCCCGCCCCTATCGAGAAGACCATCT |
| | | | CCAAGGCCAAGGGCCAGCCTCGCGAACCACAGGTGTACGT |
| | | | GTACCCTCCATCTAGAGACGAGCTGACAAAGAACCAGGTG |
| | | | AGCCTGACCTGTCTGGTGAAGGGCTTTTATCCCAGCGATAT |
| | | | CGCCGTGGAGTGGGAGTCCAATGGCCAGCCTGAGAACAAT |
| | | | TACAAGACAACCCCCCCTGTGCTGGACTCCGATGGCTCTTT |
| | | | CGCCCTGGTGTCCAAGCTGACCGTGGACAAGTCTCGGTGG |
| | | | CAGCAGGGCAACGTGTTCAGCTGTTCCGTGATGCACGAGG |
| | | | CACTGCACAATCACTACACCCAGAAGTCACTGTCACTGTCC |
| | | | CCAGGC |
| 326 | Anti- | VH | EVKLVESGGGLVQPGGSLKLSCAASGFDFSRYWMSWVRQAP |
| | FMC63id | (E1-S116) | GKGLEWIGEINLDSSTINYTPSLKDKFIISRDNAKNTLYLQMS |
| | VH-CH- | | KVRSEDTALYYCARRYDAMDYWGQGTSVTVSS |
| | HetFcA | | |
| 327 | Anti- | H1 | GFDFSRYW |
| | FMC63id | (G26-W33) | |
| | VH-CH- | | |
| | HetFcA | | |
| 328 | Anti- | H3 | ARRYDAMDY |
| | FMC63id | (A97- | |
| | VH-CH- | Y105) | |
| | HetFcA | | |
| 329 | Anti- | H2 | INLDSSTI |
| | FMC63id | (I51-I58) | |
| | VH-CH- | | |
| | HetFcA | | |
| 330 | Anti- | CH1 | ASTKGPSVFPLAPSSKSTSGGTAALGCLVKDYFPEPVTVSWNS |
| | FMC63id | (A117- | GALTSGVHTFPAVLQSSGLYSLSSVVTVPSSSLGTQTYICNVN |
| | VH-CH- | V214) | HKPSNTKVDKKV |
| | HetFcA | | |
| 331 | Anti- | CH2 | APEAAGGPSVFLFPPKPKDTLMISRTPEVTCVVVSVSHEDPEV |
| | FMC63id | (A230- | KFNWYVDGVEVHNAKTKPREEQYNSTYRVVSVLTVLHQDW |
| | VH-CH- | K339) | LNGKEYKCKVSNKALPAPIEKTISKAK |
| | HetFcA | | |
| 332 | Anti- | CH3 | GQPREPQVYVYPPSRDELTKNQVSLTCLVKGFYPSDIAVEWE |
| | FMC63id | (G340- | SNGQPENNYKTTPPVLDSDGSFALVSKLTVDKSRWQQGNVF |
| | VH-CH- | G445) | SCSVMHEALHNHYTQKSLSLSPG |
| | HetFcA | | |
| 333 | Anti- | Full | EVKLQESGPGLVAPSQSLSVTCTVSGVSLPDYGVSWIRQPPRK |
| | CD19scFv- | | GLEWLGVIWGSETTYYNSALKSRLTIIKDNSKSQVFLKMNSL |
| | HetFcB | | QTDDTAIYYCAKHYYYGGSYAMDYWGQGTSVTVSSVEGGS |
| | | | GGSGGSGGSGGVDDIQMTQTTSSLSASLGDRVTISCRASQDIS |
| | | | KYLNWYQQKPDGTVKLLIYHTSRLHSGVPSRFSGSGSGTDYS |
| | | | LTISNLEQEDIATYFCQQGNTLPYTFGGGTKLEITAAEPKSSDK |
| | | | THTCPPCPAPEAAGGPSVFLFPPKPKDTLMISRTPEVTCVVVS |
| | | | VSHEDPEVKFNWYVDGVEVHNAKTKPREEQYNSTYRVVSVL |
| | | | TVLHQDWLNGKEYKCKVSNKALPAPIEKTISKAKGQPREPQV |
| | | | YVLPPSRDELTKNQVSLLCLVKGFYPSDIAVEWESNGQPENN |
| | | | YLTWPPVLDSDGSFFLYSKLTVDKSRWQQGNVFSCSVMHEA |
| | | | LHNHYTQKSLSLSPG |
| 334 | Anti- | Full | GAGGTCAAGCTGCAGGAGAGCGGACCAGGCCTGGTGGCCC |
| | CD19scFv- | | CCTCCCAGTCTCTGAGCGTGACCTGCACAGTGTCTGGCGTG |
| | HetFcB | | AGCCTGCCCGACTACGGCGTGTCTTGGATCAGACAGCCCC |
| | | | CTAGAAAGGGCCTGGAGTGGCTGGGCGTGATCTGGGGCTC |
| | | | CGAGACAACATACTATAACTCTGCCCTGAAGAGCAGACTG |
| | | | ACCATCATCAAGGACAACTCCAAGTCTCAGGTGTTCCTGA |
| | | | AGATGAACAGCCTGCAGACCGACGATACAGCCATCTACTA |
| | | | TTGTGCCAAGCACTACTATTACGGCGGCAGCTATGCCATG |
| | | | GATTACTGGGGCCAGGGCACCTCCGTGACAGTGAGCTCCG |
| | | | TGGAGGGAGGCTCCGGAGGCTCTGGAGGCAGCGGCGGCTC |
| | | | CGGCGGCGTGGACGATATCCAGATGACCCAGACCACATCT |
| | | | AGCCTGAGCGCCTCCCTGGGCGACAGGGTGACAATCTCCT |
| | | | GCCGCGCCTCTCAGGATATCAGCAAGTATCTGAATTGGTA |
| | | | CCAGCAGAAGCCTGATGGCACCGTGAAGCTGCTGATCTAT |
| | | | CACACATCCCGGCTGCACTCTGGCGTGCCAAGCAGGTTTTC |
| | | | TGGCAGCGGCTCCGGAACCGACTACTCCCTGACAATCTCT |
| | | | AACCTGGAGCAGGAGGATATCGCCACCTATTTCTGTCAGC |
| | | | AGGGCAATACCCTGCCTTACACATTTGGCGGCGGCACAAA |
| | | | GCTGGAAATCACCGCAGCAGAACCAAAATCCTCCGATAAA |
| | | | ACTCACACTTGCCCCCCTTGCCCGGCGCCAGAGGCAGCAG |
| | | | GAGGACCAAGCGTGTTCCTGTTTCCACCCAAGCCCAAAGA |
| | | | CACCCTGATGATTAGCCGAACCCCTGAAGTCACATGCGTG |
| | | | GTCGTGTCCGTGTCTCACGAGGACCCAGAAGTCAAGTTCA |
| | | | ACTGGTACGTGGATGGCGTCGAGGTGCATAATGCCAAGAC |
| | | | AAAACCCCGGGAGGAACAGTACAACAGCACCTATAGAGTC |
| | | | GTGTCCGTCCTGACAGTGCTGCACCAGGATTGGCTGAACG |
| | | | GCAAGGAATATAAGTGCAAAGTGTCCAATAAGGCCCTGCC |
| | | | CGCTCCTATCGAGAAAACCATTTCTAAGGCAAAAGGCCAG |
| | | | CCTCGCGAACCACAGGTCTACGTGCTGCCTCCATCCCGGG |
| | | | ACGAGCTGACAAAGAACCAGGTCTCTCTGCTGTGCCTGGT |
| | | | GAAAGGCTTCTATCCATCAGATATTGCTGTGGAGTGGGAA |
| | | | AGCAATGGGCAGCCCGAGAACAATTACCTGACTTGGCCCC |
| | | | CTGTGCTGGACTCTGATGGGAGTTTCTTTCTGTATTCTAAG |
| | | | CTGACCGTGGATAAAAGTAGGTGGCAGCAGGGAAATGTCT |
| | | | TTAGTTGTTCAGTGATGCATGAAGCCCTGCATAACCACTAC |
| | | | ACCCAGAAAAGCCTGTCCCTGTCCCCCGGA |
| 335 | Anti- | VH | EVKLQESGPGLVAPSQSLSVTCTVSGVSLPDYGVSWIRQPPRK |
| | CD19scFv- | (E1-S120) | GLEWLGVIWGSETTYYNSALKSRLTIIKDNSKSQVFLKMNSL |
| | HetFcB | | QTDDTAIYYCAKHYYYGGSYAMDYWGQGTSVTVSS |
| 336 | Anti- | H1 | GVSLPDYG |
| | CD19scFv- | (G26-G33) | |
| | HetFcB | | |
| 337 | Anti- | H3 | AKHYYYGGSYAMDY |
| | CD19scFv- | (A96- | |
| | HetFcB | Y109) | |
| 338 | Anti- | H2 | IWGSETT |
| | CD19scFv- | (I51-T57) | |
| | HetFcB | | |
| 339 | Anti- | VL | DIQMTQTTSSLSASLGDRVTISCRASQDISKYLNWYQQKPDG |
| | CD19scFv- | (D139- | TVKLLIYHTSRLHSGVPSRFSGSGSGTDYSLTISNLEQEDIATY |
| | HetFcB | T245) | FCQQGNTLPYTFGGGTKLEIT |
| 340 | Anti- | L1 | QDISKY |
| | CD19scFv- | (Q165- | |
| | HetFcB | Y170) | |
| 341 | Anti- | L3 | QQGNTLPYT |
| | CD19scFv- | (Q227- | |
| | HetFcB | T235) | |
| 342 | Anti- | L2 | HTS |
| | CD19scFv- | (H188- | |
| | HetFcB | S190) | |
| 343 | Anti- | CH2 | APEAAGGPSVFLFPPKPKDTLMISRTPEVTCVVVSVSHEDPEV |
| | CD19scFv- | (A263- | KFNWYVDGVEVHNAKTKPREEQYNSTYRVVSVLTVLHQDW |
| | HetFcB | K372) | LNGKEYKCKVSNKALPAPIEKTISKAK |
| 344 | Anti- | CH3 | GQPREPQVYVLPPSRDELTKNQVSLLCLVKGFYPSDIAVEWE |
| | CD19scFv- | (G373- | SNGQPENNYLTWPPVLDSDGSFFLYSKLTVDKSRWQQGNVF |
| | HetFcB | G478) | SCSVMHEALHNHYTQKSLSLSPG |

1. A method of re-directing tumour cell binding by an immunotherapeutic from a second tumour-associated antigen epitope to a first tumour-associated antigen epitope, the method comprising contacting the immunotherapeutic with a multi-specific antigen-binding construct comprising a first antigen-binding polypeptide construct that binds to the immunotherapeutic and a second antigen-binding polypeptide construct that binds to the first tumour-associated antigen epitope,
wherein the immunotherapeutic is a T-cell or NK cell that expresses an engineered receptor comprising an antigen-binding domain that binds to the second tumour-associated antigen epitope,
wherein the first antigen-binding polypeptide construct binds to an epitope on an extracellular portion of the engineered receptor,
wherein the first and second tumour-associated antigen epitopes are different, and
wherein binding of the multi-specific antigen-binding construct to the immunotherapeutic and to the first tumour-associated antigen epitope activates the T-cell or NK cell.

2. The method according to claim 1, wherein the first and second antigen-binding polypeptide constructs are each independently an antibody or an antigen-binding fragment thereof.

3. The method according to claim 1 or 2, wherein the first and second antigen-binding polypeptide constructs are each independently a Fab, an scFv or a single domain antibody (sdAb).

4. The method according to any one of claims 1 to 3, wherein the engineered receptor is a chimeric antigen receptor (CAR).

5. A method of extending the therapeutic effect of an immunotherapeutic in a patient who is undergoing or has undergone treatment with the immunotherapeutic, the method comprising administering to the patient an effective amount of a multi-specific antigen-binding construct comprising a first antigen-binding polypeptide construct that binds to the immunotherapeutic and a second antigen-binding polypeptide construct that binds to a first tumour-associated antigen epitope,
wherein the immunotherapeutic is a T-cell or NK cell that expresses an engineered receptor comprising an antigen-binding domain that binds to a second tumour-associated antigen epitope,
wherein the first antigen-binding polypeptide construct binds to an epitope on an extracellular portion of the engineered receptor,
wherein the first and second tumour-associated antigen epitopes are different, and
wherein binding of the multi-specific antigen-binding construct to the immunotherapeutic and to the first tumour-associated antigen epitope activates the T-cell or NK cell.

6. A method of treating cancer in a patient who is undergoing or has undergone treatment with an immunotherapeutic, the method comprising administering an effective amount of a multi-specific antigen-binding construct to the patient, the multi-specific antigen-binding construct comprising a first antigen-binding polypeptide construct that binds to the immunotherapeutic and a second antigen-binding polypeptide construct that binds to a first tumour-associated antigen epitope,
wherein the immunotherapeutic is a T-cell or NK cell that expresses an engineered receptor comprising an antigen-binding domain that binds to a second tumour-associated antigen epitope,
wherein the first antigen-binding polypeptide construct binds to an epitope on an extracellular portion of the engineered receptor,
wherein the first and second tumour-associated antigen epitopes are different, and
wherein binding of the multi-specific antigen-binding construct to the immunotherapeutic and to the first tumour-associated antigen epitope activates the T-cell or NK cell.

7. The method according to claim 5 or 6, wherein the first and second antigen-binding polypeptide constructs are each independently an antibody or an antigen-binding fragment thereof.

8. The method according to any one of claims 5 to 7, wherein the first and second antigen-binding polypeptide constructs are each independently a Fab, an scFv or a single domain antibody (sdAb).

9. The method according to any one of claims 5 to 8, wherein the engineered receptor is a chimeric antigen receptor (CAR).

10. The method according to any one of claims 5 to 9, wherein the patient has undergone prior treatment with the immunotherapeutic.

11. The method according to claim 10, wherein the patient has relapsed from or failed to respond to the prior treatment.

12. The method according to claim 11, wherein the patient has relapsed from or failed to respond to the prior treatment due to:
(a) a decrease in, or loss of expression of, the second tumour-associated antigen epitope, or
(b) heterogeneity of expression of the second tumour-associated antigen epitope.

13. The method according to any one of claims 5 to 9, wherein the patient is undergoing treatment with the immunotherapeutic and the multi-specific antigen-binding construct is administered as an adjunctive treatment to the immunotherapeutic.

14. The method according to claim 13, wherein the T-cell or NK cell is further engineered to co-express the multi-specific antigen-binding construct.

15. The method according to any one of claims 1 to 14, wherein the first antigen-binding polypeptide construct binds to an epitope on the antigen-binding domain of the engineered receptor.

16. The method according to any one of claims 1 to 14, wherein the first antigen-binding polypeptide construct binds to an epitope on a region of the engineered receptor that is not involved in antigen-binding.

17. A method of activating a T-cell or NK cell engineered to express a chimeric antigen receptor (CAR) or a T-cell receptor (TCR), the method comprising:
(i) contacting the T-cell or NK cell with a multi-specific antigen-binding construct comprising a first antigen-binding polypeptide construct that binds to an epitope on an extracellular portion of the CAR or TCR and a second antigen-binding polypeptide construct that binds to a first tumour-associated antigen epitope, wherein the CAR or TCR comprises an antigen-binding domain that binds to a second tumour-associated antigen epitope, wherein the first and second tumour-associated antigen epitopes are different, and
(ii) contacting the T-cell or NK cell and the multi-specific antigen-binding construct with a cell expressing the first tumour-associated antigen epitope,
wherein binding of the multi-specific antigen-binding construct to the T-cell or NK cell and to the first tumour-associated antigen epitope activates the T-cell or NK cell.

18. The method according to claim 17, wherein the first and second antigen-binding polypeptide constructs are each independently an antibody or an antigen-binding fragment thereof.

19. The method according to claim 17 or 18, wherein the first and second antigen-binding polypeptide constructs are each independently a Fab, an scFv or a single domain antibody (sdAb).

20. The method according to any one of claims 17 to 19, wherein the T-cell or NK cell is engineered to express a CAR.

21. The method according to any one of claims 17 to 20, comprising activating a T-cell.

22. The method according to any one of claims 1 to 21, wherein the first and second tumour-associated antigen epitopes are epitopes of the same antigen.

23. The method according to any one of claims 1 to 21, wherein the first and second tumour-associated antigen epitopes are epitopes of different antigens.

24. The method according to any one of claims 1 to 23, wherein the first and second tumour-associated antigen epitopes are associated with a hematological cancer.

25. The method according to any one of claims 1 to 23, wherein the first and second tumour-associated antigen epitopes are expressed by malignant B-cells.

26. The method according to any one of claims 1 to 23, wherein the first and second tumour-associated antigen epitopes are associated with a solid tumour.

27. The method according to any one of claims 1 to 23, wherein the second tumour-associated antigen epitope is an epitope of CD19, CD22 or BCMA.

28. The method according to any one of claims 1 to 27, wherein the multi-specific antigen binding construct further comprises a scaffold and the first and second antigen-binding polypeptide constructs are linked to the scaffold.

29. The method according to claim 28, wherein the scaffold comprises an Fc.

30. The method according to claim 29, wherein the Fc comprises a first Fc polypeptide and second Fc polypeptide, each comprising a CH3 sequence.

31. The method according to claim 30, wherein the first antigen-binding polypeptide construct is linked to the first Fc polypeptide and the second antigen-binding polypeptide construct is linked to the second Fc polypeptide.

32. The method according to claim 30 or 31, wherein the Fc is a heterodimeric Fc comprising amino acid modifications in at least one CH3 sequence.

33. The method according to any one of claims 1 to 27, wherein the first and second antigen-binding polypeptide constructs are joined by a linker.

34. The method according to any one of claims 1 to 33, wherein the multi-specific antigen-binding construct further comprises one or more additional antigen-binding polypeptide constructs.

35. A multi-specific antigen-binding construct comprising:
a first antigen-binding polypeptide construct that binds to an immunotherapeutic, and
a second antigen binding polypeptide construct that binds to a first tumour-associated antigen epitope,
wherein the immunotherapeutic is a T-cell or NK cell that expresses an engineered receptor comprising an antigen-binding domain that binds to a second tumour-associated antigen epitope,
wherein the first antigen-binding polypeptide construct binds to an epitope on an extracellular portion of the engineered receptor,
wherein the first and second tumour-associated antigen epitopes are different, and
wherein binding of the multi-specific antigen-binding construct to the immunotherapeutic and to the first tumour-associated antigen epitope activates the T-cell or NK cell.

36. The multi-specific antigen-binding construct according to claim 35, wherein the first and second antigen-binding polypeptide constructs are each independently an antibody or an antigen-binding fragment thereof.

37. The multi-specific antigen-binding construct according to claim 35 or 36, wherein the engineered receptor is a chimeric antigen receptor (CAR).

38. A multi-specific antigen-binding construct comprising:
a first antigen-binding polypeptide construct that binds to an immunotherapeutic, and
a second antigen binding polypeptide construct that binds to a first tumour-associated antigen epitope,
wherein the immunotherapeutic is a T-cell engineered to express a chimeric antigen receptor (CAR) comprising an antigen-binding domain that binds to a second tumour-associated antigen epitope,
wherein the first antigen-binding polypeptide construct binds to an epitope on an extracellular portion of the CAR,
wherein the first and second antigen-binding polypeptide constructs are each independently an antibody or an antigen-binding fragment thereof,
wherein the first and second tumour-associated antigen epitopes are different, and
wherein binding of the multi-specific antigen-binding construct to the immunotherapeutic and to the first tumour-associated antigen epitope activates the T-cell.

39. The multi-specific antigen-binding construct according to any one of claims 35 to 38, wherein the first and second antigen-binding polypeptide constructs are each independently a Fab, an scFv or a single domain antibody (sdAb).

40. The multi-specific antigen-binding construct according to any one of claims 35 to 39, wherein the first and second tumour-associated antigen epitopes are epitopes of the same antigen.

41. The multi-specific antigen-binding construct according to any one of claims 35 to 39, wherein the first and second tumour-associated antigen epitopes are epitopes of different antigens.

42. The multi-specific antigen-binding construct according to any one of claims 35 to 41, wherein the first antigen-binding polypeptide construct binds to an epitope on the antigen-binding domain of the receptor.

43. The multi-specific antigen-binding construct according to any one of claims 35 to 41, wherein the first antigen-binding polypeptide construct binds to an epitope on a region of the receptor that is not involved in antigen-binding.

44. The multi-specific antigen-binding construct according to any one of claims 35 to 43, wherein the multi-specific antigen-binding construct further comprises a scaffold and the first and second antigen-binding polypeptide constructs are linked to the scaffold.

45. The multi-specific antigen-binding construct according to claim 44, wherein the scaffold is an Fc.

46. The multi-specific antigen-binding construct according to claim 45, wherein the Fc comprises a first Fc polypeptide and second Fc polypeptide, each comprising a CH3 sequence.

47. The multi-specific antigen-binding construct according to claim 46, wherein the first antigen-binding polypeptide construct is linked to the first Fc polypeptide and the second antigen-binding polypeptide construct is linked to the second Fc polypeptide.

48. The multi-specific antigen-binding construct according to claim 46 or 47, wherein the Fc is a heterodimeric Fc comprising amino acid modifications in at least one CH3 sequence.

49. The multi-specific antigen-binding construct according to any one of claims 35 to 43, wherein the first and second antigen-binding polypeptide constructs are joined by a linker.

50. The multi-specific antigen-binding construct according to any one of claims 35 to 49, wherein the multi-specific antigen-binding construct further comprises one or more additional antigen-binding polypeptide constructs.

51. The multi-specific antigen-binding construct according to any one of claims 35 to 50, wherein the first and second tumour-associated antigen epitopes are associated with a hematological cancer.

52. The multi-specific antigen-binding construct according to any one of claims 35 to 50, wherein the first and second tumour-associated antigen epitopes are expressed by malignant B cells.

53. The multi-specific antigen-binding construct according to any one of claims 35 to 50, wherein the first and second tumour-associated antigen epitopes are associated with a solid tumour.

54. The multi-specific antigen-binding construct according to any one of claims 35 to 50, wherein the second tumour-associated antigen epitope is an epitope of CD19, CD22 or BCMA.

55. A pharmaceutical composition comprising the multi-specific antigen-binding construct according to any one of claims 35 to 54, and a pharmaceutically acceptable carrier.

56. Nucleic acid encoding the multi-specific antigen-binding construct according to any one of claims 35 to 54.

57. A host cell comprising nucleic acid encoding the multi-specific antigen-binding construct according to any one of claims 35 to 54.

58. Use of the multi-specific antigen-binding construct according to any one of claims 35 to 54 in the manufacture of a medicament.

59. The use according to claim 58, wherein the medicament is for re-directing tumour cell binding by the immunotherapeutic from the second tumour-associated antigen epitope to the first tumour-associated antigen epitope.

60. The use according to claim 58, wherein the medicament is for extending the therapeutic effect of the immunotherapeutic in a patient who is undergoing or has undergone treatment with the immunotherapeutic.

61. The use according to claim 58, wherein the medicament is for treating cancer in a patient who is undergoing or has undergone treatment with the immunotherapeutic.
